(19) United States
(12) Patent Application Publication
(10) Pub. No.: US 2025/0353828 A1
(43) Pub. Date: Nov. 20, 2025

(54) SUBSTITUTED BIARYL ENDOCHIN-LIKE QUINOLONES WITH ENHANCED ANTIPARASITIC ACTIVITY

(71) Applicants: Oregon Health & Science University, Portland, OR (US); The United States Government as Represented by the Dept of Veterans Affairs, Washington, DC (US)

(72) Inventors: Michael K. RISCOE, Portland, OR (US); Aaron NILSEN, Portland, OR (US); J. Stone DOGGETT, Portland, OR (US); Holland ALDAY, Portland, OR (US); Katherine LIEBMAN, Portland, OR (US); Sovitj POU, Portland, OR (US); Rozalia DODEAN, Portland, OR (US); Rolf W. WINTER, Portland, OR (US)

(73) Assignees: Oregon Health & Science University, Portland, OR (US); The United States Government as Represented by the Dept of Veterans Affairs, Washington, DC (US)

(21) Appl. No.: 18/871,987

(22) PCT Filed: Jun. 7, 2023
(86) PCT No.: PCT/US23/24746

§ 371 Date: Dec. 5, 2024

Related U.S. Application Data
(60) Provisional application No. 63/349,930, filed on Jun. 7, 2022.
(60) Provisional application No. 63/471,701, filed on Jun. 7, 2023.

(51) Int. Cl. C07D 401/10; A61K 31/4709; A61P 33/06; C07D 215/233

(52) U.S. Cl. CPC C07D 401/10; A61K 31/4709; A61P 33/06; C07D 215/233

(57) ABSTRACT

Provided herein are Endochin-Like Quinolone (ELQ) compounds of Formula (I): or a pharmaceutically acceptable salt thereof, along with pharmaceutical compositions comprising them and methods for their use in treating or preventing parasitic diseases, including malaria, toxoplasmosis, and babesiosis.

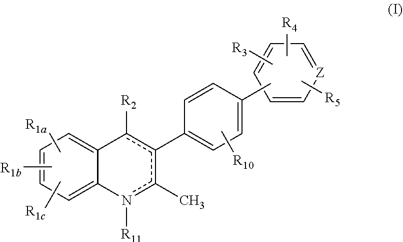

### CROSS-REFERENCE TO RELATED APPLICATIONS

This is the 371 National Phase of International Application No. PCT/US23/24746, filed Jun. 7, 2023, which claims priority to and the benefit of the earlier filing of both U.S. Provisional Application No. 63/471,701, filed Jun. 7, 2023, and U.S. Provisional Application No. 63/349,930, filed Jun. 7, 2022, each of which is incorporated by reference herein in its entirety.

### STATEMENT OF GOVERNMENT SUPPORT

This invention was made with government support under R01 AI100569 and R01 AI141412 awarded by the National Institutes of Health and W81XWH-19-2-0031 awarded by the U.S. Department of Defense. The government has certain rights in the invention.

### FIELD OF THE INVENTION

The present invention concerns novel biaryl Endochin-Like Quinolone (ELQ) compounds useful in treating or preventing parasitic diseases, including malaria, toxoplasmosis, and babesiosis.

### BACKGROUND OF THE INVENTION

In 2020 an estimated 241 million cases of malaria occurred worldwide with roughly 93% of cases occurring on the African continent. In the same year, there were an estimated 627,000 deaths from malaria around the globe, with children accounting for roughly 77% of all malaria deaths worldwide. These figures represent an uptick in case numbers and deaths over previous years because of disruptions in health care delivery due to the ongoing COVID pandemic¹. Before the pandemic and over the past two decades the World Health Organization noted steady reductions in cases and deaths worldwide primarily due to an increase in vector control measures and use of mosquito bed nets, as well as the introduction of artemisinin-combined-therapies (ACTs). Now the situation is complicated not only by the surging COVID-19 pandemic but also by resistance emerging to ACTs in Asia² and Africa³ where resistance to frontline antimalarials such as chloroquine, mefloquine, amodiaquine, antifolates and quinine is already firmly entrenched. Thus, even though the trend for malaria deaths has generally been on the decline, there is an urgent need for new drugs to address multidrug resistance and to service global efforts toward disease eradication.

In order to tackle the challenge of today's dynamic antimalarial drug resistance landscape and to make advances on the goal of worldwide eradication of the disease, the Medicines for Malaria Venture (MMV) have created a list of desirable Target Product Profiles (TPP) and associated Target Candidate Profiles (TCP) that provide valuable guidance (or “road-maps”) for what is needed to achieve the ultimate goal of eradication^{4, 5}. The list is comprehensive and includes new oral medications that can be used for treatment of acute but uncomplicated malaria, as well as for severe and complicated disease where a fast-acting parenteral formulation would be appropriate. There is also a TPP for drugs that can be used for chemoprevention where the drug would be given to subjects moving into regions of high malaria endemicity or during epidemics or to especially vulnerable populations, e.g., pregnant women and children. And within these TPPs there are described drug molecules with TCPs to fill particular niches within the treatment and/or prophylaxis pharmacopoeia of new and available drugs. Such TCPs include drugs that clear asexual blood-stage parasites (TCP-1) or molecules that target the latent liver stage hypnozoites of vivax and ovale (TCP-3) or replicating liver schizonts of all malaria species (TCP-4), as well as drugs that interfere with transmission in blood or within the insect vector (TCP-5). More recently, MMV described a new TPP for a long-acting injectable (LAI-C) to be used in treatment and chemoprevention for 2 to 4 months of protection against seasonal malaria or in the case of malaria epidemics⁵.

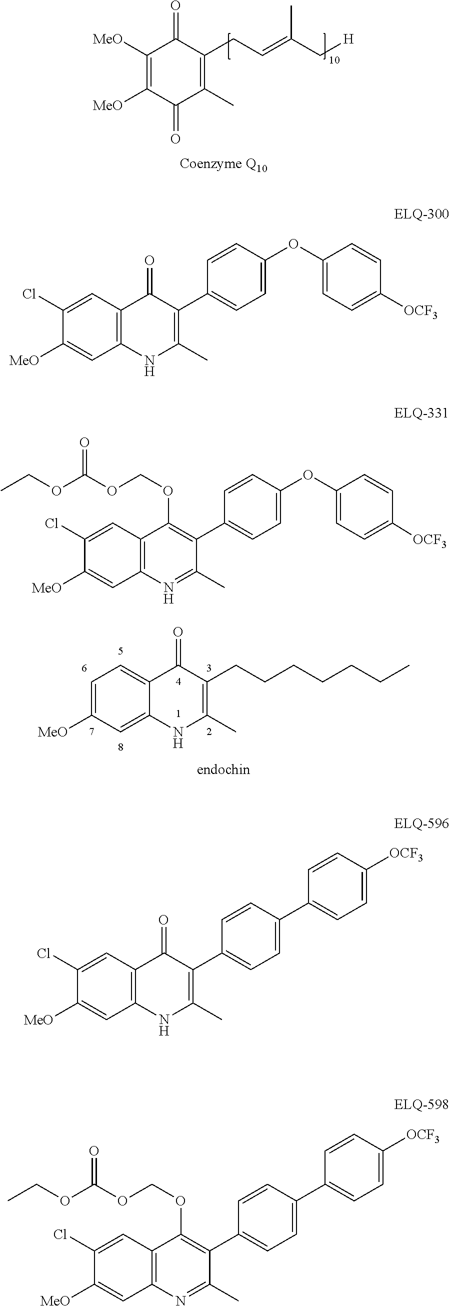

FIG. 1. Structures of Coenzyme Q10, endochin, ELQ-300, ELQ-331, ELQ-596 and ELQ-598.

Over 10 years ago ELQ-300 (FIG. 1) was discovered as part of a research consortium with the MMV to optimize the historical lead endochin for human use⁶. Like all known cytochrome bc₁ inhibitors, ELQ-300 is an analog of Coenzyme Q₁₀, a native ligand of electron transport chain enzymes. Since its discovery, nearly everything that we have learned about ELQ-300 shows that it would be a highly valuable tool to add to the antimalarial toolbox for prevention and treatment of malaria and for transmission blocking⁶. Distinguishing characteristics of the drug include: low nM IC₅₀'s vs. multidrug resistant strains of P. falciparum including field isolates, pan-antimalarial activity against the various Plasmodium species that infect humans⁷, potent activity against replicating parasites in the liver⁶, blood and vector stages of infection⁶, novel and selective targeting of the Qᵢ site of P. falciparum cytochrome bc₁ complex⁸, excellent metabolic stability and extended pharmacokinetics in preclinical species (mouse, rat, and dog), and a clean safety profile⁶. While this was sufficient for ELQ-300 to be selected as a preclinical candidate by the MMV in 2012, further development was derailed in 2014 when it was dropped from the pipeline due to high crystallinity which limited absorption and prevented determination of an in vivo therapeutic index necessary for regulatory approval. Fortunately, we were able to address this issue and revive interest in ELQ-300 by introduction of a prodrug (ELQ-331, FIG. 1) with significantly reduced crystallinity that gave improved oral bioavailability and enhanced overall antimalarial performance⁹. ELQ-331 was accepted as a preclinical candidate by the MMV in October of 2020. Since this time, an oral formulation of ELQ-331 has been developed by the MMV, and we recently described a low cost and scalable synthetic route to the core molecule ELO-300 adding to the feasibility of developing this drug for human use¹⁰. Thus, prodrug ELQ-331 continues to move forward through the MMV clinical development pipeline.

There remains a need for discovery and development of new ELQ compounds with improvements in intrinsic potency, selectivity, pharmacokinetic properties and/or efficacy.

### SUMMARY OF THE INVENTION

One embodiment provides a compound of Formula (I):

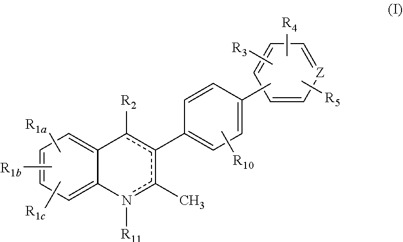

wherein:
each of R₁ₐ, R_{1b}, and R_{1c} is independently selected from the group of H, halogen, CN, C₁-C₆ alkyl, and C₁-C₆ alkoxy;
R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆;
g) —O—CH₂—O—C(═O)—R₆;
h) —O—(C═O)—R₇;
i) —O—(C═O)—O—R₇;
j) —O—C(O)—NR₈R₉;
k) —O—CH₂—O—C(O)—O—(CH₂)ₙ₁—NR₈R₉;
l) —O—CH₂—O—C(O)—O—(CH₂)ₙ₁—NR₈—C(═O)—O—R₉; and
m) —O—(CH₂)—O—PO₃;
the dashed lines ( [symbol] ) in each instance represent an optional single or double bond;
Z is selected from the group of N and C; and
R₃, R₄, and R₅ are each independently selected from the group of H, halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), —C(O)N(C₁-C₄ alkyl)₂, 2-pyrrolidinone, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₆ is selected from the group of C₁-C₁₀ alkyl, C₂-C₁₀ alkenyl, C₂-C₁₀ alkynyl, C₃-C₆ cycloalkyl, —(CH₂)ₙ₁—C₃-C₆ cycloalkyl, 3-6-membered heterocyclyl, —(CH₂)ₙ₁-3-6-membered heterocyclyl, phenyl, —(CH₂)ₙ₁-phenyl, and —(CH₂)ₙ₁—NR₈R₉;
R₇ is selected from the group of C₁-C₁₀ alkyl, C₂-C₁₀ alkenyl, C₂-C₁₀ alkynyl, C₃-C₆ cycloalkyl, —(CH₂)ₙ₃—(C₃-C₆ cycloalkyl), —(CH₂)ₙ₃-(3-6 membered heterocyclyl), phenyl, —(CH₂)ₙ₁-phenyl, —(CH₂)ₙ₁—O—(CH₂)ₙ₂—C₁-C₂ alkyl, —(CH₂—CH₂—O)ₙ₁—C₁-C₂ alkyl, and —(CH₂)ₙ₁—NR₈R₉;
R₈ and R₉ are each independently selected from the group of H and C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
R₁₁ is selected from the group of H, OH, and O⁻;
n1 and n2 are each integers independently selected from the group of 1, 2, 3, 4, 5, and 6; and
n3 is an integer selected from the group of 0, 1, 2, 3, 4, 5, and 6;
with the proviso that the compound is not a compound selected from the group of 3-[1,1′-Biphenyl]-4-yl-2-methyl-4(1H)-quinolinone (CAS Reg. No. 1354745-30-4), 3-[1,1-Biphenyl]-4-yl-7-methoxy-2-methyl-4(1H)-quinolinone (CAS Reg. No. 1354745-39-3), 3-[1,1′-Biphenyl]-4-yl-6-chloro-2-methyl-4(1H)-quinolinone (CAS Reg. No. 1354745-40-6), 3-[1,1′-Biphenyl]-4-yl-6-fluoro-2-methyl-4(1H)-quinolinone (CAS Reg. No. 1354745-28-0), 3-[1,1′-Biphenyl]-4-yl-5,7-difluoro-2-methyl-4(1H)-quinolinone (CAS Reg. No. 2251119-93-2), 3-[1,1′-Biphenyl]-4-yl-6-fluoro-7-methoxy-2-methyl-4(1H)-quinolinone (CAS Reg. No. 1354745-27-9), 3-[1,1′-Biphenyl]-4-yl-6-chloro-7-methoxy-2-methyl-4(1H-quinolinone (CAS Reg. No. 1636139-73-5), and 6-fluoro-7-methoxy-2-methyl-3-(3″-(trifluoromethyl)-[1,1′:4′,1″-terphenyl]-4-yl)quinolin-4(1H)-one (CAS Reg. No. 1374758-04-9);
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

### DETAILED DESCRIPTION OF THE INVENTION

One embodiment provides a compound of Formula (I), wherein R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆; and
g) —O—CH₂—O—C(═O)—R₆; and
R₁ₐ, R_{1b}, R_{1c}, R₃, R₄, R₅, R₆, R₈, R₉, R₁₀, and R₁₁ are as defined above for Formula (I); or a pharmaceutically acceptable salt thereof.

It is understood that the floating bond crossed with a wavy line ( [symbol] ) in the X variable of the formula

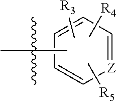

represents a phenyl ring, a pyridine ring, or a pyridazine ring bound to the adjacent phenyl ring through one of its five available carbon atoms, as seen in the following examples:

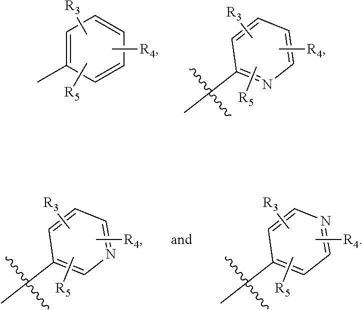

It is also understood that the dashed lines between the ring nitrogen and 2-carbon atom, between the 2-carbon and 3-carbon atoms, and between the 3-carbon and 4-carbon atoms of the quinoline ring represent, in each instance, an optional single bond or an optional double, depending upon the valence of the R₂ substituent, as represented by oxo and hydroxy groups in the non-limiting exemplary structures below.

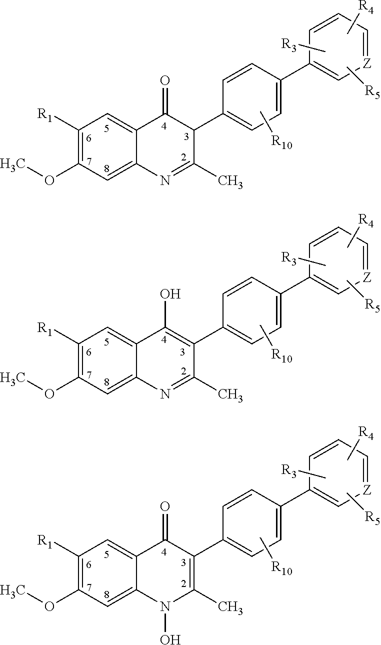

Another embodiment provides a compound of Formula (I), wherein R₂ is selected from the group of:
a) oxo (═O);
b) —OH;
c) —O—CH₂—O—C(═O)—O—R₆;
d) —O—CH₂—CH₂—O—C(═O)—O—R₆; and
e) —O—CH₂(CH₃)—O—C(═O)—O—R₆; and
R₁, R₃, R₄, R₅, R₈, R₉, R₁₀, and R₁ are as defined above for Formula (I); or a pharmaceutically acceptable salt thereof.

A further embodiment provides a compound of Formula (I), wherein:
R₁ is selected from the group of H, F, and Cl;
R₂ is selected from the group of oxo (═O) and a moiety of the formula —O—CH₂—O—(═O)—O—(C₁-C₆ alkyl);
Z is selected from the group of N and C; and
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆, R₇, R₈, R₉, R₁₀, and R₁₁ are as defined for Formula (I), above; and
with the proviso that, when R₂ is selected from the group of oxo (═O), then at least one of R₃, R₄, and R₅ is not H;
or a pharmaceutically acceptable salt thereof.

Another embodiment provides a compound of Formula (I), wherein:
R₁ is selected from the group of H, F, and Cl;
R₂ is selected from the group of oxo (═O) and a moiety of the formula —O—CH₂—O—C(═O)—O—(C₁-C₄ alkyl);
Z is selected from the group of N and C;
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆, R₇, R₈, R₉, R₁₀, and R₁₁ are as defined for Formula (I), above; and
with the proviso that, when R₂ is selected from the group of oxo (═O), then at least one of R₃, R₄, and R₅ is not H;
or a pharmaceutically acceptable salt thereof.

Another embodiment provides a compound of Formula (I), wherein:
R₁ is selected from the group of H, F, and Cl;
R₂ is selected from the group of oxo (═O) and a moiety of the formula —O—CH₂—O—C(═O)—O—(C₁-C₃ alkyl);
Z is selected from the group of N and C; and
R₃, R₄, and R₅ are each independently selected from the group of H, halogen, methyl, methoxy (—O—CH₃), CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆, R₇, R₈, R₉, R₁₀, and R₁₁ are as defined for Formula (I), above; and
with the proviso that, when R₂ is selected from the group of oxo (═O), then at least one of R₃, R₄, and R₅ is not H;
or a pharmaceutically acceptable salt thereof.

An additional embodiment provides a compound of Formula (II):

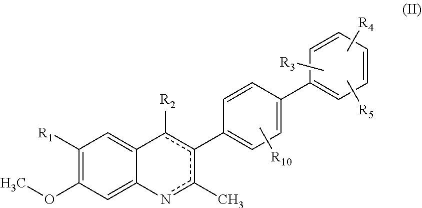

wherein:
R₁ is selected from the group of H, F, and Cl;
R₂ is selected from the group of oxo (═O) and a moiety of the formula —O—CH₂—O—C(═O)—O—(C₁-C₁₀ alkyl);
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl)), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₂ is selected from the group of oxo (═O), then at least one of R₃, R₄, and R₅ is not H;
or a pharmaceutically acceptable salt thereof.

A further embodiment provides a compound of Formula (II), wherein:
R₁ is selected from the group of H, F, and Cl;
R₂ is selected from the group of oxo (═O) and a moiety of the formula —O—CH₂—O—C(═O)—O—(C₁-C₆ alkyl);
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
the dashed lines ( [symbol] ) in each instance represent an optional single or double bond;
R₁₀ is selected from the group of H, C₁-C₆ alkyl, —O—C₁-C₆ alkyl, C₁-C₄ haloalkyl, —O—C₁-C₄ haloalkyl, and —S—C₁-C₄ haloalkyl;
with the proviso that, when R₂ is selected from the group of oxo (═O), then at least one of R₃, R₄, and R₅ is not H;
or a pharmaceutically acceptable salt thereof.

Another embodiment provides a compound of Formula (II), wherein:
R₁ is selected from the group of H, F, and Cl;
R₂ is selected from the group of oxo (═O) and a moiety of the formula —O—CH₂—O—C(═O)—O—(C₁-C₄ alkyl); and
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₂ is selected from the group of oxo (═O), then at least one of R₃, R₄, and R₅ is not H;
or a pharmaceutically acceptable salt thereof.

Another embodiment provides a compound of Formula (II), wherein:
R₁ is selected from the group of H, F, and Cl;
R₂ is selected from the group of oxo (═O) and a moiety of the formula —O—CH₂—O—C(═O)—O—(C₁-C₃ alkyl); and
R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₂ is selected from the group of oxo (═O), then at least one of R₃, R₄, and R₅ is not H;
or a pharmaceutically acceptable salt thereof.

A different embodiment provides a compound of Formula (III):

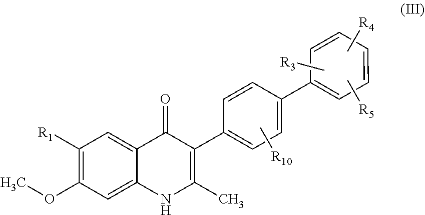

wherein:
R₁ is selected from the group of H, F, and Cl;
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt thereof.

A further embodiment provides a compound of Formula (III), wherein:
R₁ is selected from the group of H, F, and Cl;
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt thereof.

Another embodiment provides a compound of Formula (III), wherein:
R₁ is selected from the group of H, F, and Cl; and
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt thereof.

Another embodiment provides a compound of Formula (III), wherein:
R₁ is selected from the group of H, F, and Cl; and
R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt thereof.

Two additional separate embodiments provide, respectively, a compound of Formula (III-A) and a compound of Formula (III-B):

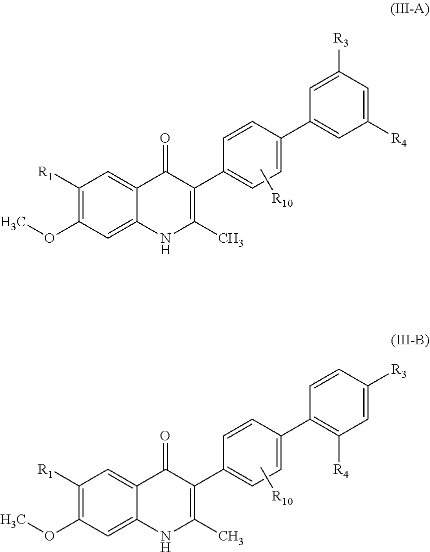

wherein, in each separate embodiment:
R₁ is selected from the group of H, F, and Cl;
R₃ and R₄ are each independently selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
with the proviso that, when R₁₀ is H, at least one of R₃ and R₄ is not H;
or a pharmaceutically acceptable salt thereof.

Two further embodiments provide, respectively, a compound of Formula (III-A) and a compound of Formula (III-B), wherein in each separate embodiment:
R₁ is selected from the group of H, F, and Cl;
R₃ and R₄ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₁₀ is selected from the group of H, halogen, C₁-C₃ alkyl, —O—C₁-C₆ alkyl, C₁-C₄ haloalkyl, —O—C₁-C₄ haloalkyl, and —S—C₁-C₄ haloalkyl;
with the proviso that, when R₁₀ is H, at least one of R₃ and R₄ is not H;
or a pharmaceutically acceptable salt thereof.

Two further embodiments provide, respectively, a compound of Formula (III-A) and a compound of Formula (III-B), wherein in each separate embodiment:
R₁ is selected from the group of H, F, and Cl; and
R₃ and R₄ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₂ alkyl, —O—C₁-C₆ alkyl, C₁-C₄ haloalkyl, —O—C₁-C₄ haloalkyl, and —S—C₁-C₄ haloalkyl;
with the proviso that, when R₁₀ is H, at least one of R₃ and R₄ is not H;
or a pharmaceutically acceptable salt thereof.

Two further embodiments provide, respectively, a compound of Formula (III-A) and a compound of Formula (III-B), wherein in each separate embodiment:
R₁ is selected from the group of H, F, and Cl; and
R₃ and R₄ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl;
R₁₀ is selected from the group of H, F, methyl, methoxy, CH₂F, CHF₂, CF₃, —O—CH₂F, —O—CHF₂, —O—CF₃, and —S—CF₃;
with the proviso that, when R₁₀ is H, at least one of R₃ and R₄ is not H;
or a pharmaceutically acceptable salt thereof.

Two further embodiments provide, respectively, a compound of Formula (III-A) and a compound of Formula (III-B), wherein in each separate embodiment:
R₁ is selected from the group of H, F, and Cl; and
R₃ and R₄ are each independently selected from the group of H, halogen, CH₂F, CHF₂, CF₃, —O—CH₂F, —O—CHF₂, —O—CF₃, and —S—CF₃;
R₁₀ is selected from the group of H, F, methyl, methoxy, CH₂F, CHF₂, CF₃, —O—CH₂F, —O—CHF₂, —O—CF₃, and —S—CF₃;
with the proviso that, when R₁₀ is H, at least one of R₃ and R₄ is not H;
or a pharmaceutically acceptable salt thereof.

Two additional embodiments provide, respectively, a compound of Formula (III-A) and a compound of Formula (III-B), wherein in each separate embodiment:
R₁ is selected from the group of H, F, and Cl; and
R₃ and R₄ are each independently selected from the group of F, CH₂F, CHF₂, CF₃, —O—CH₂F, —O—CHF₂, —O—CF₃, and —S—CF₃;
R₁₀ is selected from the group of H, F, methyl, methoxy, CH₂F, CHF₂, CF₃, —O—CH₂F, —O—CHF₂, —O—CF₃, and —S—CF₃;
with the proviso that, when R₁₀ is H, at least one of R₃ and R₄ is not H;
or a pharmaceutically acceptable salt thereof.

Two more separate embodiments provide, respectively, a compound of Formula (III-A2) and a compound of Formula (III-B2):

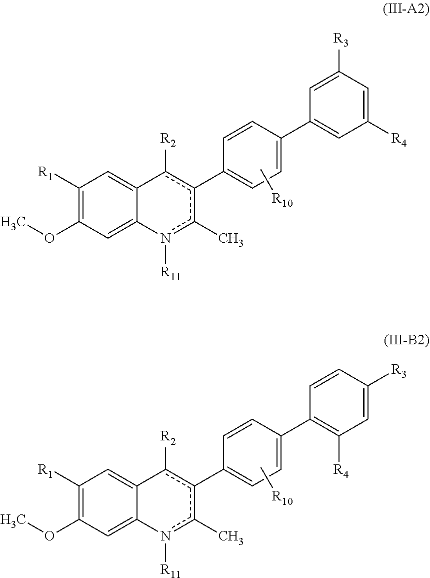

wherein, in each separate embodiment:
R₁ is selected from the group of H, F, and Cl;
R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆;
g) —O—CH₂—O—C(═O)—R₆;
h) —O—(C═O)—R₇;
i) —O—(C═O)—O—R₇;
j) —O—C(O)—NR₈R₉;
k) —O—CH₂—O—C(O)—O—(CH₂)ₙ₁—NR₈R₉;
l) —O—CH₂—O—C(O)—O—(CH₂)ₙ₁—NR_{B}—C(═O)—O—R₉; and
m) —O—(CH₂)—O—PO₃;
the dashed lines ( [symbol] ) in each instance represent an optional single or double bond;
R₃ and R₄ are each independently selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₆ is selected from the group of C₁-C₁₀ alkyl, C₁-C₁₀ alkenyl, C₁-C₁₀ alkynyl, C₃-C₆ cycloalkyl, —(CH₂)ₙ₁—C₃-C₆ cycloalkyl, 3-6-membered heterocyclyl, —(CH₂)ₙ₁-3-6-membered heterocyclyl, phenyl, —(CH₂)ₙ₁-phenyl, and —(CH₂)ₙ₁—NR₈R₉;
R₇ is selected from the group of C₁-C₁₀ alkyl, C₁-C₁₀ alkenyl, C₁-C₁₀ alkynyl, C₃-C₆ cycloalkyl, —(CH₂)ₙ₃—(C₃-C₆ cycloalkyl), —(CH₂)ₙ₃-(3-6 membered heterocyclyl), phenyl, —(CH₂)ₙ₁-phenyl, —(CH₂)ₙ₁—O—(CH₂)ₙ₂—C₁-C₂ alkyl, —(CH₂—CH₂—O)ₙ₁—C₁-C₂ alkyl, and —(CH₂)ₙ₁—NR₈R₉;
R₈ and R₉ are each independently selected from the group of H and C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
R₁₁ is selected from the group of H, OH, and O⁻;
n1 and n2 are each integers independently selected from the group of 1, 2, 3, 4, 5, and 6; and
n3 is an integer selected from the group of 0, 1, 2, 3, 4, 5, and 6;
or a pharmaceutically acceptable salt thereof.

Two further embodiments provide, respectively, a compound of Formula (III-A2) and a compound of Formula (III-B2), wherein in each separate embodiment:
each of R₁, R₂, R₆, R₇, R₈, R₉, R₁₀, R₁₁, n1, n2, n3, and the dashed lines are as defined above for Formulas (III-A2) and (III-B2);
R₃ and R₄ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₁₀ is selected from the group of H, halogen, C₁-C₃ alkyl, —O—C₁-C₆ alkyl, C₁-C₄ haloalkyl, —O—C₁-C₄ haloalkyl, and —S—C₁-C₄ haloalkyl;
with the proviso that, when R₁₀ is H, at least one of R₃ and R₄ is not H;
or a pharmaceutically acceptable salt thereof.

Two further embodiments provide, respectively, a compound of Formula (III-A2) and a compound of Formula (III-B2), wherein in each separate embodiment:
each of R₁, R₂, R₆, R₇, R₈, R₉, R₁₀, R₁₁, n1, n2, n3, and the dashed lines are as defined above for Formulas (III-A2) and (III-B2);
R₃ and R₄ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₂ alkyl, —O—C₁-C₆ alkyl, C₁-C₄ haloalkyl, —O—C₁-C₄ haloalkyl, and —S—C₁-C₄ haloalkyl;
with the proviso that, when R₁₀ is H, at least one of R₃ and R₄ is not H;
or a pharmaceutically acceptable salt thereof.

Two further embodiments provide, respectively, a compound of Formula (III-A2) and a compound of Formula (III-B2), wherein in each separate embodiment:
each of R₁, R₂, R₆, R₇, R₈, R₉, R₁₀, R₁₁, n1, n2, n3, and the dashed lines are as defined above for Formulas (III-A2) and (III-B2);
R₃ and R₄ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, F, methyl, methoxy, CH₂F, CHF₂, CF₃, —O—CH₂F, —O—CHF₂, —O—CF₃, and —S—CF₃;
with the proviso that, when R₁₀ is H, at least one of R₃ and R₄ is not H;
or a pharmaceutically acceptable salt thereof.

Two further embodiments provide, respectively, a compound of Formula (III-A2) and a compound of Formula (III-B2), wherein in each separate embodiment:
each of R₁, R₂, R₆, R₇, R₈, R₉, R₁₀, R₁₁, n1, n2, n3, and the dashed lines are as defined above for Formulas (III-A2) and (III-B2);
R₃ and R₄ are each independently selected from the group of H, halogen, CH₂F, CHF₂, CF₃, —O—CH₂F, —O—CHF₂, —O—CF₃, and —S—CF₃;
R₁₀ is selected from the group of H, F, methyl, methoxy, CH₂F, CHF₂, CF₃, —O—CH₂F, —O—CHF₂, —O—CF₃, and —S—CF₃;
with the proviso that, when R₁₀ is H, at least one of R₃ and R₄ is not H;
or a pharmaceutically acceptable salt thereof.

Two additional embodiments provide, respectively, a compound of Formula (III-A2) and a compound of Formula (III-B2), wherein in each separate embodiment:
each of R₁, R₂, R₆, R₇, R₈, R₉, R₁₀, R₁₁, n1, n2, n3, and the dashed lines are as defined above for Formulas (III-A2) and (III-B2);
R₃ and R₄ are each independently selected from the group of F, CH₂F, CHF₂, CF₃, —O—CH₂F, —O—CHF₂, —O—CF₃, and —S—CF₃;
R₁₀ is selected from the group of H, F, methyl, methoxy, CH₂F, CHF₂, CF₃, —O—CH₂F, —O—CHF₂, —O—CF₃, and —S—CF₃;
with the proviso that, when R₁₀ is H, at least one of R₃ and R₄ is not H;
or a pharmaceutically acceptable salt thereof.

A further embodiment provides a compound of Formula (IV):

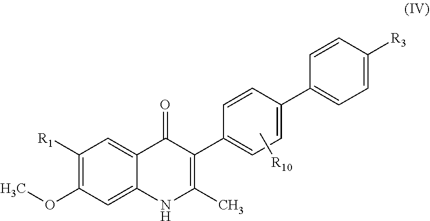

wherein:
R₁ is selected from the group of H, F, and Cl; and
R₃ is selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (IV), wherein:
R₁ is selected from the group of H, F, and Cl; and
R₃ is selected from the group of halogen, C₁-C₃ alkyl, —O—C₁-C₃ alkyl, C₁-C₃ haloalkyl, —O—C₁-C₃ haloalkyl, —S—CF₃, —SF₅, CN, -2-pyrrolidinone, C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
or a pharmaceutically acceptable salt thereof.

A further embodiment provides a compound of Formula (IV), wherein:
R₁ is selected from the group of H, F, and Cl; and
R₃ is selected from the group of halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another further embodiment provides a compound of Formula (IV), wherein:
R₁ is Cl; and
R₃ is selected from the group of halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Still another embodiment provides a compound of Formula (IV), wherein:
R₁ is Cl; and
R₃ is selected from the group of halogen, C₁-C₂ alkyl, —O—C₁-C₂ alkyl, C₁-C₂ haloalkyl, —O—C₁-C₂ haloalkyl, —SF₅, and CN;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

An additional embodiment provides a compound of Formula (IV), wherein:
R₁ is Cl; and
R₃ is selected from the group of F, Cl, C₁-C₂ alkyl, —O—C₁-C₂ alkyl, C₁-C₂ haloalkyl, —O—C₁-C₂ haloalkyl, —SF₅, and CN;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (V):

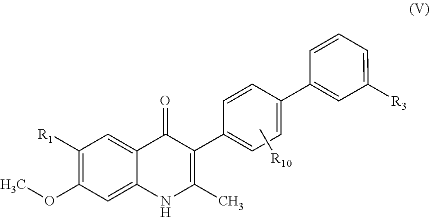

wherein:
R₁ is selected from the group of H, F, and Cl; and
R₃ is selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (V), wherein:
R₁ is selected from the group of H, F, and Cl; and
R₃ is selected from the group of halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (V), wherein:
R₁ is selected from the group of H, F, and Cl; and
R₃ is selected from the group of halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another further embodiment provides a compound of Formula (V), wherein:
R₁ is Cl; and
R₃ is selected from the group of halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Still another embodiment provides a compound of Formula (V), wherein:
R₁ is Cl; and
R₃ is selected from the group of halogen, C₁-C₂ alkyl, —O—C₁-C₂ alkyl, C₁-C₂ haloalkyl, —O—C₁-C₂ haloalkyl, —SF₅, and CN;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

An additional embodiment provides a compound of Formula (V), wherein:
R₁ is Cl; and
R₃ is selected from the group of F, Cl, C₁-C₂ alkyl, —O—C₁-C₂ alkyl, C₁-C₂ haloalkyl, —O—C₁-C₂ haloalkyl, —SF₅, and CN;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A different embodiment provides a compound of Formula (VI):

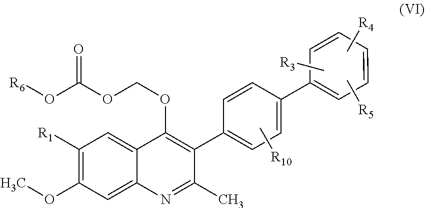

wherein:
R₁ is selected from the group of H, F, and Cl;
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₁₀ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (VI), wherein:
R₁ is selected from the group of H, F, and Cl; and
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VI), wherein:
R₁ is selected from the group of H, F, and Cl; and
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₄ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VI), wherein:
R₁ is selected from the group of H, F, and Cl;
R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₃ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A different embodiment provides a compound of Formula (VII):

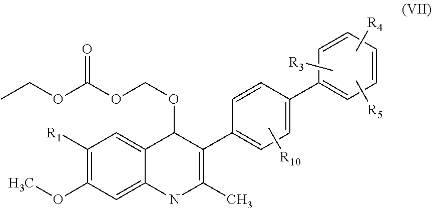

wherein:
R₁ is selected from the group of H, F, and Cl; and
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VII), wherein:
R₁ is Cl; and
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (VII), wherein:
R₁ is selected from the group of H, F, and Cl; and
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (VII), wherein:
R₁ is Cl; and
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VII), wherein:
R₁ is selected from the group of H, F, and Cl; and
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VII), wherein:
R₁ is Cl; and
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VII), wherein:
R₁ is selected from the group of H, F, and Cl; and
R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VII), wherein:
R₁ is Cl; and
R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

An additional embodiment provides a compound of Formula (VII), wherein:
R₁ is Cl; and
R₃ is selected from the group of F, Cl, C₁-C₂ alkyl, —O—C₁-C₂ alkyl, C₁-C₂ haloalkyl, —O—C₁-C₂ haloalkyl, —SF₅, and CN;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A different embodiment provides a compound of Formula (VIII):

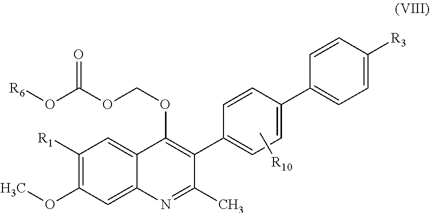

wherein:
R₁ is selected from the group of H, F, and Cl;
R₃ is selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₁₀ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (VIII), wherein:
R₁ is selected from the group of H, F, and Cl;
R₃ is selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (VIII), wherein:
R₁ is selected from the group of H, F, and Cl;
R₃ is selected from the group of halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VIII), wherein:
R₁ is selected from the group of H, F, and Cl; and
R₃ is selected from the group of halogen, C₁-C₃ alkyl, —O—C₁-C₃ alkyl, C₁-C₃ haloalkyl, —O—C₁-C₃ haloalkyl, —SF₅, and CN;
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VIII), wherein:
R₁ is selected from the group of H, F, and Cl; and
R₃ is selected from the group of halogen, C₁-C₂ alkyl, —O—C₁-C₂ alkyl, C₁-C₂ haloalkyl, —O—C₁-C₂ haloalkyl, —SF₅, and CN;
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VIII), wherein:
R₁ is Cl;
R₃ is selected from the group of halogen, C₁-C₂ alkyl, —O—C₁-C₂ alkyl, C₁-C₂ haloalkyl, —O—C₁-C₂ haloalkyl, —SF₅, and CN; and
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VIII), wherein:
R₁ is Cl;
R₃ is selected from the group of halogen, C₁-C₂ alkyl, —O—C₁-C₂ alkyl, C₁-C₂ haloalkyl, —O—C₁-C₂ haloalkyl, —SF₅, and CN; and
R₆ is C₁-C₄ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (VIII), wherein:
R₁ is Cl;
R₃ is selected from the group of halogen, C₁-C₂ alkyl, —O—C₁-C₂ alkyl, C₁-C₂ haloalkyl, —O—C₁-C₂ haloalkyl, —SF₅, and CN; and
R₆ is ethyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A different embodiment provides a compound of Formula (IX):

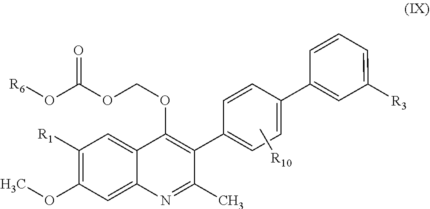

wherein:
R₁ is selected from the group of H, F, and Cl;
R₃ is selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₁₀ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (IX), wherein:
R₁ is selected from the group of H, F, and Cl;
R₃ is selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

A further embodiment provides a compound of Formula (IX), wherein:
R₁ is selected from the group of H, F, and Cl;
R₃ is selected from the group of halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is C₁-C₆ alkyl;
or a pharmaceutically acceptable salt thereof.

Another embodiment provides a compound of Formula (IX), wherein:
R₁ is selected from the group of H, F, and Cl; and
R₃ is selected from the group of halogen, C₁-C₃ alkyl, —O—C₁-C₃ alkyl, C₁-C₃ haloalkyl, —O—C₁-C₃ haloalkyl, —SF₅, and CN;
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (IX), wherein:
R₁ is selected from the group of H, F, and Cl; and
R₃ is selected from the group of halogen, C₁-C₂ alkyl, —O—C₁-C₂ alkyl, C₁-C₂ haloalkyl, —O—C₁-C₂ haloalkyl, —SF₅, and CN;
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (IX), wherein:
R₁ is Cl;
R₃ is selected from the group of halogen, C₁-C₂ alkyl, —O—C₁-C₂ alkyl, C₁-C₂ haloalkyl, —O—C₁-C₂ haloalkyl, —SF₅, and CN; and
R₆ is C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (IX), wherein:
R₁ is Cl;
R₃ is selected from the group of halogen, C₁-C₂ alkyl, —O—C₁-C₂ alkyl, C₁-C₂ haloalkyl, —O—C₁-C₂ haloalkyl, —SF₅, and CN; and
R₆ is C₁-C₄ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (IX), wherein:
R₁ is Cl;
R₃ is selected from the group of halogen, C₁-C₂ alkyl, —O—C₁-C₂ alkyl, C₁-C₂ haloalkyl, —O—C₁-C₂ haloalkyl, —SF₅, and CN; and
R₆ is ethyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
or a pharmaceutically acceptable salt thereof.

Another embodiment provides a compound of Formula (X):

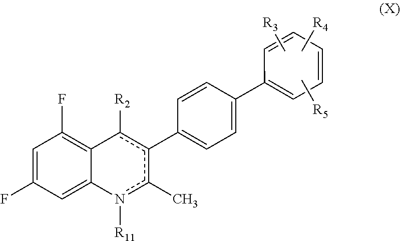

wherein:
R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆;
g) —O—CH₂—O—C(═O)—R₆;
h) —O—(C═O)—R₇;
i) —O—(C═O)—O—R₇;
j) —O—C(O)—NR₈R₉;
k) —O—CH₂—O—C(O)—O—(CH₂)ₙ₁—NR₈R₉;
l) —O—CH₂—O—C(O)—O—(CH₂)ₙ₁—NR_{B}—C(═O)—O—R₉; and
m) —O—(CH₂)—O—PO₃;
the dashed lines ( [symbol] ) in each instance represent an optional single or double bond;
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₆ is selected from the group of C₁-C₁₀ alkyl, C₁-C₁₀ alkenyl, C₁-C₁₀ alkynyl, C₃-C₆ cycloalkyl, —(CH₂)ₙ₁—C₃-C₆ cycloalkyl, 3-6-membered heterocyclyl, —(CH₂)ₙ₁-3-6-membered heterocyclyl, phenyl, —(CH₂)ₙ₁-phenyl, and —(CH₂)ₙ₁—NR₈R₉;
R₇ is selected from the group of C₁-C₁₀ alkyl, C₁-C₁₀ alkenyl, C₁-C₁₀ alkynyl, C₃-C₆ cycloalkyl, —(CH₂)ₙ₃—(C₃-C₆ cycloalkyl), —(CH₂)ₙ₃-(3-6 membered heterocyclyl), phenyl, —(CH₂)ₙ₁-phenyl, —(CH₂)ₙ₁—O—(CH₂)ₙ₂—C₁-C₂ alkyl, —(CH₂—CH₂—O)ₙ₁—C₁-C₂ alkyl, and —(CH₂)ₙ₁—NR₈R₉;
R₈ and R₉ are each independently selected from the group of H and C₁-C₆ alkyl;
R₁₁ is selected from the group of H, OH, and O⁻;
n1 and n2 are each integers independently selected from the group of 1, 2, 3, 4, 5, and 6; and
n3 is an integer selected from the group of 0, 1, 2, 3, 4, 5, and 6;
or a pharmaceutically acceptable salt thereof.

An additional embodiment comprises a compound of Formula (X), wherein:
R₂ is selected from the group of:
a) oxo (═O);
b) —OH; and
c) —O—CH₂—O—C(═O)—O—R₆;
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, and —SF₅;
R₆ is selected from the group of C₁-C₁₀ alkyl, C₁-C₁₀ alkenyl, C₁-C₁₀ alkynyl, C₃-C₆ cycloalkyl, and —(CH₂)ₙ₁—C₃-C₆ cycloalkyl; and
R₁₁ is selected from the group of H, OH, and O⁻;
or a pharmaceutically acceptable salt thereof.

A further embodiment comprises a compound of Formula (X), wherein:
R₂ is selected from the group of:
d) oxo (═O);
e) —OH; and
f) —O—CH₂—O—C(═O)—O—R₆;
R₃, R₄, and R₅ are each independently selected from the group of H, halogen, C₁-C₃ alkyl, —O—C₁-C₃ alkyl, C₁-C₃ haloalkyl, and —O—C₁-C₃ haloalkyl;
R₆ is selected from the group of C₁-C₁₀ alkyl, C₁-C₁₀ alkenyl, C₁-C₁₀ alkynyl, C₃-C₆ cycloalkyl, and —(CH₂)ₙ₁—C₃-C₆ cycloalkyl; and
R₁₁ is selected from the group of H, OH, and O⁻;
or a pharmaceutically acceptable salt thereof.

A still further embodiment comprises a compound of Formula (X), wherein:
R₂ is selected from the group of:
g) oxo (═O);
h) —OH; and
i) —O—CH₂—O—C(═O)—O—R₆;
R₃ and R₄ are each independently selected from the group of H, halogen, C₁-C₃ alkyl, —O—C₁-C₃ alkyl, CF₃, and —O—CF₃;
R₅ is H;
R₆ is C₁-C₆ alkyl; and
R₁₁ is selected from the group of H, OH, and O⁻;
or a pharmaceutically acceptable salt thereof.

Still another embodiment provides a compound of Formula (XI):

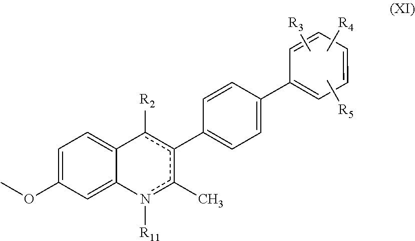

wherein:
R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆;
g) —O—CH₂—O—C(═O)—R₆;
h) —O—(C═O)—R₇;
i) —O—(C═O)—O—R₇;
j) —O—C(O)—NR₈R₉;
k) —O—CH₂—O—C(O)—O—(CH₂)ₙ₁—NR₈R₉;
l) —O—CH₂—O—C(O)—O—(CH₂)ₙ₁—NR₈—C(═O)—O—R₉; and
m) —O—(CH₂)—O—PO₃;
the dashed lines ( [symbol] ) in each instance represent an optional single or double bond; R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is selected from the group of C₁-C₁₀ alkyl, C₁-C₁₀ alkenyl, C₁-C₁₀ alkynyl, C₃-C₆ cycloalkyl, —(CH₂)ₙ₁—C₃-C₆ cycloalkyl, 3-6-membered heterocyclyl, —(CH₂)ₙ₁-3-6-membered heterocyclyl, phenyl, —(CH₂)ₙ₁-phenyl, and —(CH₂)ₙ₁—NR₈R₉;
R₇ is selected from the group of C₁-C₁₀ alkyl, C₁-C₁₀ alkenyl, C₁-C₁₀ alkynyl, C₃-C₆ cycloalkyl, —(CH₂)ₙ₃—(C₃-C₆ cycloalkyl), —(CH₂)ₙ₃-(3-6 membered heterocyclyl), phenyl, —(CH₂)ₙ₁-phenyl, —(CH₂)ₙ₁—O—(CH₂)ₙ₂—C₁-C₂ alkyl, —(CH₂—CH₂—O)ₙ₁—C₁-C₂ alkyl, and —(CH₂)ₙ₁—NR₈R₉;
R₈ and R₉ are each independently selected from the group of H and C₁-C₆ alkyl;
R₁₁ is selected from the group of H, OH, and O⁻;
n1 and n2 are each integers independently selected from the group of 1, 2, 3, 4, 5, and 6; and
n3 is an integer selected from the group of 0, 1, 2, 3, 4, 5, and 6;
or a pharmaceutically acceptable salt thereof.

An additional embodiment comprises a compound of Formula (X), wherein:
R₂ is selected from the group of:
a) oxo (═O);
b) —OH; and
c) —O—CH₂—O—C(═O)—O—R₆;
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, and —SF₅;
R₆ is selected from the group of C₁-C₁₀ alkyl, C₁-C₁₀ alkenyl, C₁-C₁₀ alkynyl, C₃-C₆ cycloalkyl, and —(CH₂)ₙ₁—C₃-C₆ cycloalkyl; and
R₁₁ is selected from the group of H, OH, and O⁻;
or a pharmaceutically acceptable salt thereof.

A further embodiment comprises a compound of Formula (XI), wherein:
R₂ is selected from the group of:
a) oxo (═O);
b) —OH; and
c) —O—CH₂—O—C(═O)—O—R₆;
R₃, R₄, and R₅ are each independently selected from the group of H, halogen, C₁-C₃ alkyl, —O—C₁-C₃ alkyl, C₁-C₃ haloalkyl, and —O—C₁-C₃ haloalkyl;
R₆ is selected from the group of C₁-C₁₀ alkyl, C₁-C₁₀ alkenyl, C₁-C₁₀ alkynyl, C₃-C₆ cycloalkyl, and —(CH₂)ₙ₁—C₃-C₆ cycloalkyl; and
R₁₁ is selected from the group of H, OH, and O⁻;
or a pharmaceutically acceptable salt thereof.

A still further embodiment comprises a compound of Formula (X), wherein:
R₂ is selected from the group of:
a) oxo (═O);
b) —OH; and
c) —O—CH₂—O—C(═O)—O—R₆;
R₃ and R₄ are each independently selected from the group of H, halogen, C₁-C₃ alkyl, —O—C₁-C₃ alkyl, CF₃, and —O—CF₃;
R₅ is H;
R₆ is C₁-C₆ alkyl; and
R₁₁ is selected from the group of H, OH, and O⁻;
or a pharmaceutically acceptable salt thereof.

Another embodiment provides a compound of Formula (XII):

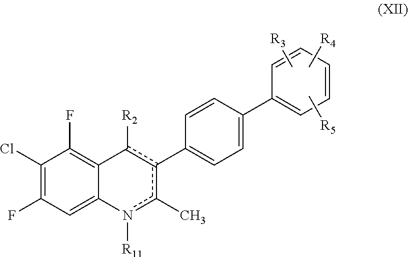

wherein:
R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆;
g) —O—CH₂—O—C(═O)—R₆;
h) —O—(C═O)—R₇;
i) —O—(C═O)—O—R₇;
j) —O—C(O)—NR₈R₉;
k) —O—CH₂—O—C(O)—O—(CH₂)ₙ₁—NR₈R₉;
l) —O—CH₂—O—C(O)—O—(CH₂)ₙ₁—NR₈—C(═O)—O—R₉; and
m) —O—(CH₂)—O—PO₃;
the dashed lines ( [symbol] ) in each instance represent an optional single or double bond;
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is selected from the group of C₁-C₁₀ alkyl, C₁-C₁₀ alkenyl, C₁-C₁₀ alkynyl, C₃-C₆ cycloalkyl, —(CH₂)ₙ₁—C₃-C₆ cycloalkyl, 3-6-membered heterocyclyl, —(CH₂)ₙ₁-3-6-membered heterocyclyl, phenyl, —(CH₂)ₙ₁-phenyl, and —(CH₂)ₙ₁—NR₈R₉;
R₇ is selected from the group of C₁-C₁₀ alkyl, C₁-C₁₀ alkenyl, C₁-C₁₀ alkynyl, C₃-C₆ cycloalkyl, —(CH₂)ₙ₃—(C₃-C₆ cycloalkyl), —(CH₂)ₙ₃-(3-6 membered heterocyclyl), phenyl, —(CH₂)ₙ₁-phenyl, —(CH₂)ₙ₁—O—(CH₂)ₙ₂—C₁-C₂ alkyl, —(CH₂—CH₂—O)ₙ₁—C₁-C₂ alkyl, and —(CH₂)ₙ₁—NR₈R₉;
R₈ and R₉ are each independently selected from the group of H and C₁-C₆ alkyl;
R₁₁ is selected from the group of H, OH, and O⁻;
n1 and n2 are each integers independently selected from the group of 1, 2, 3, 4, 5, and 6; and
n3 is an integer selected from the group of 0, 1, 2, 3, 4, 5, and 6;
or a pharmaceutically acceptable salt thereof.

An additional embodiment comprises a compound of Formula (XII), wherein:
R₂ is selected from the group of:
a) oxo (═O);
b) —OH; and
c) —O—CH₂—O—C(═O)—O—R₆;
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, and —SF₅;
R₆ is selected from the group of C₁-C₁₀ alkyl, C₁-C₁₀ alkenyl, C₁-C₁₀ alkynyl, C₃-C₆ cycloalkyl, and —(CH₂)ₙ₁—C₃-C₆ cycloalkyl; and
R₁₁ is selected from the group of H, OH, and O⁻;
or a pharmaceutically acceptable salt thereof.

A further embodiment comprises a compound of Formula (XII), wherein:
R₂ is selected from the group of:
a) oxo (═O);
b) —OH; and
c) —O—CH₂—O—C(═O)—O—R₆;
R₃, R₄, and R₅ are each independently selected from the group of H, halogen, C₁-C₃ alkyl, —O—C₁-C₃ alkyl, C₁-C₃ haloalkyl, and —O—C₁-C₃ haloalkyl;
R₆ is selected from the group of C₁-C₁₀ alkyl, C₁-C₁₀ alkenyl, C₁-C₁₀ alkynyl, C₃-C₆ cycloalkyl, and —(CH₂)ₙ₁—C₃-C₆ cycloalkyl; and
R₁₁ is selected from the group of H, OH, and O⁻;
or a pharmaceutically acceptable salt thereof.

A still further embodiment comprises a compound of Formula (XII), wherein:
R₂ is selected from the group of:
a) oxo (═O);
b) —OH; and
c) —O—CH₂—O—C(═O)—O—R₆;
R₃ and R₄ are each independently selected from the group of H, halogen, C₁-C₃ alkyl, —O—C₁-C₃ alkyl, CF₃, and —O—CF₃;
R₅ is H;
R₆ is C₁-C₆ alkyl; and
R₁₁ is selected from the group of H, OH, and O⁻;
or a pharmaceutically acceptable salt thereof.

Three additional embodiments provide, respectively, a compound of Formula (XIII), Formula (XIV), and Formula (XV):

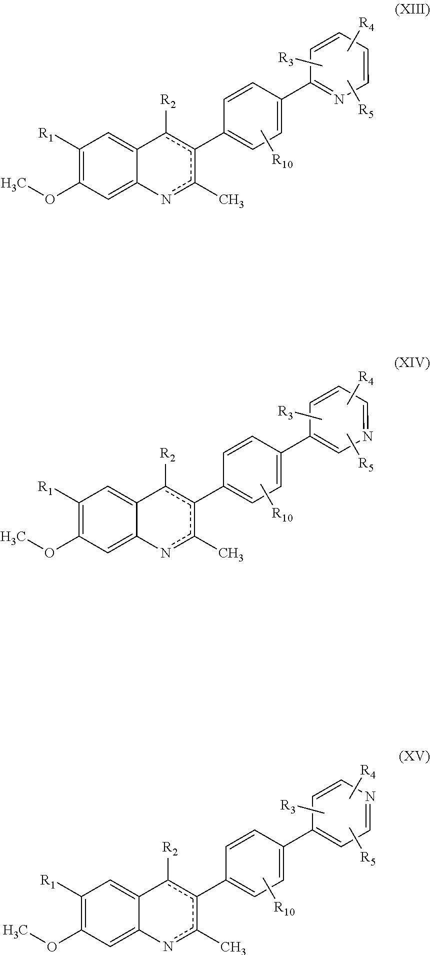

wherein in each embodiment:
R₁ is selected from the group of H, F, and Cl;
R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆;
g) —O—CH₂—O—C(═O)—R₆;
h) —O—(C═O)—R₇;
i) —O—(C═O)—O—R₇;
j) —O—C(O)—NR₈R₉;
k) —O—CH₂—O—C(O)—O—(CH₂)ₙ₁—NR₈R₉;
l) —O—CH₂—O—C(O)—O—(CH₂)ₙ₁—NR₆—C(═O)—O—R₉; and
m) —O—(CH₂)—O—PO₃;
the dashed lines ( [symbol] ) in each instance represent an optional single or double bond;
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆ is selected from the group of C₁-C₁₀ alkyl, C₂-C₁₀ alkenyl, C₂-C₁₀ alkynyl, C₃-C₆ cycloalkyl, —(CH₂)ₙ₁—C₃-C₆ cycloalkyl, 3-6-membered heterocyclyl, —(CH₂)ₙ₁-3-6-membered heterocyclyl, phenyl, —(CH₂)ₙ₁-phenyl, and —(CH₂)ₙ₁—NR₈R₉;
R₇ is selected from the group of C₁-C₁₀ alkyl, C₁-C₁₀ alkenyl, C₂-C₁₀ alkynyl, C₃-C₆ cycloalkyl, —(CH₂)ₙ₃—(C₃-C₆ cycloalkyl), —(CH₂)ₙ₃-(3-6 membered heterocyclyl), phenyl, —(CH₂)ₙ₁-phenyl, —(CH₂)ₙ₁—O—(CH₂)ₙ₁—C₁-C₂ alkyl, —(CH₂—CH₂—O)ₙ₁—C₁-C₂ alkyl, and —(CH₂)ₙ₁—NR₈R₉;
R₈ and R₉ are each independently selected from the group of H and C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
R₁₁ is selected from the group of H, OH, and O⁻;
n1 and n2 are each integers independently selected from the group of 1, 2, 3, 4, 5, and 6; and
n3 is an integer selected from the group of 0, 1, 2, 3, 4, 5, and 6;
or a pharmaceutically acceptable salt thereof.

Another embodiment provides a pharmaceutical composition comprising a pharmaceutically effective amount of a compound of Formula (I), or a pharmaceutically acceptable salt thereof, and a pharmaceutically acceptable carrier or excipient. Additional embodiments comprise different pharmaceutical compositions comprising, respectively, a pharmaceutically effective amount of a compound of Formula (II), Formula (III), Formula (IV), Formula (V), Formula (VI), Formula (VII), Formula (VIII), and Formula (IX), Formula (X), Formula (XI), Formula (XII), Formula (XIII), Formula (XIV), and Formula (XV), as well as each of the subgeneric groups describing subsets of those formulas, and the individual ELQ compounds described herein.

Also provided herein is a method for treating malaria in a human subject, the method comprising administering to the human in need thereof a pharmaceutically effective amount of a compound of Formula (I), or a pharmaceutically acceptable salt thereof.

Also provided herein is a method for inhibiting malaria in a human subject, the method comprising administering to the human in need thereof a pharmaceutically effective amount of a compound of Formula (I), or a pharmaceutically acceptable salt thereof.

Methods for treating or inhibiting malaria in a human subject include the treatment or inhibition of infections caused by Plasmodium falciparum, Plasmodium vivax, Plasmodium ovale, and Plasmodium malariae.

Also provided herein is a method for treating multidrug-resistant malaria in a human subject, the method comprising administering to the human in need thereof a pharmaceutically effective amount of a compound of Formula (I), or a pharmaceutically acceptable salt thereof.

Multidrug-resistant malaria infections that may be treated using the compounds and methods herein include malaria resistant to treatment with one or more agents selected from the group of chloroquine, sulfadoxine-pyrimethamine, quinine, piperaquine, mefloquine, artemisinin-based combination therapy (ACT, including artemether-lumefantrine (COARTEM™) and artesunate-mefloquine), pyrimethamine, dapsone, atovaquone, and a P. falciparum dihydroorotate dehydrogenase (DHOD inhibitor or PfDHOD inhibitor). DHOD inhibitors include, but are not limited to DSM265 (Coteron J M et al, J Med Chem 54, 5540-5561 (2011)).

Also provided herein is a method for treating chloroquine-resistant malaria in a human subject, the method comprising administering to the human in need thereof a pharmaceutically effective amount of a compound of Formula (I), or a pharmaceutically acceptable salt thereof.

Also provided herein is a method for treating a latent malaria infection in a human subject, the method comprising administering to the human in need thereof a pharmaceutically effective amount of a compound of Formula (I), or a pharmaceutically acceptable salt thereof.

The methods for treating malarial infections described herein may also further comprise co-administering with the compound of Formula (I), or a pharmaceutically acceptable salt thereof, to the human in need thereof a therapeutically effective amount of one or more compounds selected from the group of quinine, chloroquine, atovaquone, proguanil, primaquine, amodiaquine, mefloquine, piperaquine, artemisinin, artesunate, methylene blue, pyrimethamine, sulfadoxine, artemether-lumefantrine, dapsone-chlorproguanil, quinidine, clopidol, and dihydroartemisinin, or a pharmaceutically acceptable salt thereof.

Also provided herein is a method for treating toxoplasmosis (Toxoplasma gondii infection) in a human subject, the method comprising administering to the human in need thereof a pharmaceutically effective amount of a compound of Formula (I), or a pharmaceutically acceptable salt thereof.

Also provided herein is a method for treating babesiosis in a human subject, the method comprising administering to the human in need thereof a pharmaceutically effective amount of a compound of Formula (I), or a pharmaceutically acceptable salt thereof. Methods of treatment for babesiosis in a human subject includes that for infections by Babesia microti.

Also provided herein is a method for treating babesiosis in a non-human subject, the method comprising administering to the non-human subject in need thereof a pharmaceutically effective amount of a compound of Formula (I), or a pharmaceutically acceptable salt thereof.

Such methods include bovine babesiosis, including infections caused by Babesia bovis and B. bigemina, and equine babesiosis, including infections caused by B. caballi and Theileria equi.

### Definitions

The term “alkyl” refers to a straight or branched hydrocarbon. For example, an alkyl group can include those having 1 to 4 carbon atoms (i.e., C₁-C₄ alkyl or C₁₋₄ alkyl). Examples of suitable alkyl groups include, but are not limited to, methyl, ethyl, n-propyl, isopropyl (—CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), and 2-methyl-2-propyl (t-Bu, t-butyl, —C(CH₃)₃). Similarly, “alkenyl” refers to a straight or branched hydrocarbon comprising at least one carbon-to-carbon double bond, such as a prop-1-enyl or penta-1,3-dienyl group. The term “alkynyl” refers to a straight or branched hydrocarbon comprising at least one carbon-to-carbon triple bond, such as a pent-3-ynyl group.

The term “alkoxy” refers to a group having the formula —O-alkyl, in which an alkyl group, as defined above, is attached to a parent molecule via an oxygen atom, such as seen in variables R₃, R₄, and R₅. Examples of the alkyl portion of an alkoxy group can have 1 to 4 carbon atoms (i.e., —O—C₁-C₄ alkyl or C₁-C₄ alkoxy), 1 to 3 carbon atoms (i.e., —O—C₁-C₃ alkyl or C₁-C₃ alkoxy), or 1 to 2 carbon atoms (i.e., —O—C₁-C₂ alkyl or C₁-C₂ alkoxy). Examples of suitable alkoxy groups include, but are not limited to, methoxy (—O—CH₃ or —OMe), ethoxy (—OCH₂CH₃ or —OEt), n-propoxy (—CH₂—CH₂—CH₃), isopropoxy (—CH(CH₃)₂), n-butyl (—CH₂—CH₂—CH₂—CH₃), isobutoxy (—CH₂—CH(CH₃)₂), sec-butoxy (—CH(CH₃)CH₂—CH₃), t-butoxy (—O—C(CH₃)₃ or —OtBu), and the like.

The term “haloalkyl” refers to an alkyl group, as defined above, in which one or more hydrogen atoms of the alkyl group is replaced with a halogen atom. The alkyl portion of a haloalkyl group can have, for instance, 1 to 4 carbon atoms (i.e., C₁-C₄ haloalkyl or C₄ haloalkyl). Non-limiting examples of suitable haloalkyl groups, which may also be referred to as fluoroalkyl groups include, but are not limited to, trifluoromethyl (—CF₃), difluoromethyl (—CHF₂), fluoromethyl (—CFH₂), 2-fluoroethyl (—CH₂CH₂F), 2-fluoropropyl (—CH₂CHF₂), 2,2,2-trifluoroethyl (—CH₂CF₃), 1,1-difluoroethyl (—CF₂CH₃), 2-fluoropropyl (—CH₂CHFCH₃), 1,1-difluoropropyl (—CF₂CH₂CH₃), 2,2-difluoropropyl (—CH₂CF₂CH₃), 3,3-difluoropropyl (—CH₂CH₂CHF₂), 3,3,3-trifluoropropyl (—CH₂CH₂CHF₃), 1,1-difluorobutyl (—CF₂CH₂CH₂CH₃), perfluoroethyl (—CF₂CF₃), perfluoropropyl (—CF₂CF₂CF₃), 1,1,2,2,3,3-hexafluorobutyl (—CF₂—CF₂CF₂CH₃), perfluorobutyl (—CF₂CF₂CF₂CF₃), 1,1,1,3,3,3-hexafluoropropan-2-yl (—CH₂(CF₃)₂) groups, and the like. Additional groups wherein the halogen substitution is with bromine, iodine, or chlorine atoms are also understood for use herein.

The term “cycloalkyl” refers to a saturated or partially unsaturated ring having 3 to 6 carbon atoms (C₃-C₆ cycloalkyl or C₃₋₆ cycloalkyl), as a monocycle, including cyclopropyl, cyclobutyl, cyclopentyl, and cyclohexyl rings.

The term “halogen” or “halo” refers and element or substituent selected from the group of F, Cl, Br, and I.

A “heterocycle” or “heterocyclic group” herein refers to a chemical ring containing carbon atoms and at least one ring heteroatom selected from O, S, and N. Examples of 5-membered and 6-membered heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, 4-piperidinyl, pyrrolidinyl, 2-pyrrolidonyl, pyrrolinyl, tetrahydrofuranyl, triazinyl, 6H-1,2,5-thiadiazinyl, 2H,6H-1,5,2-dithiazinyl, thienyl, thianthrenyl, pyranyl, 2H-pyrrolyl, isothiazolyl, isoxazolyl, pyrazinyl, pyridazinyl, imidazolidinyl, imidazolinyl, pyrazolidinyl, pyrazolinyl, piperazinyl, morpholinyl, and oxazolidinyl.

“Multidrug-resistant” or “drug-resistant” refers to malaria, or the parasites causing malaria, that have developed resistance to treatment by at least one therapeutic agent historically administered to treat malaria. For example, there are multidrug-resistant strains of Plasmodium falciparum that harbor high-level resistance to chloroquine, quinine, mefloquine, pyrimethamine, sulfadoxine and atovaquone.

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “Claims 2-10” or “C₂-C₁₀ alkyl” includes units 2, 3, 4, 5, 6, 7, 8, 9, and 10, as claims and atoms are numbered in sequential numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

“Subject” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from bovine and equine subjects. “Subject in need thereof” or “human in need thereof” refers to a subject, such as a human, who may have or is suspected to have diseases or conditions that would benefit from certain treatment; for example treatment with a compound of Formula (I), or a pharmaceutically acceptable salt or co-crystal thereof, as described herein. This includes a subject who may be determined to be at risk of or susceptible to such diseases or conditions, such that treatment would prevent the disease or condition from developing.

The terms “pharmaceutically acceptable salt(s)” or “pharmacologically acceptable salt(s)” refer to salts prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. A “pharmaceutically acceptable salt” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quatemary ammonium cations and the like. Such salts are known to those of skill in the art. For additional examples of “pharmacologically acceptable salts,” see Berge et al., J. Pharm. Sci. 66:1 (1977).

The compounds described herein include all pharmaceutically acceptable salts, co-crystals, esters, solvates, hydrates, isomers (including optical isomers, racemates, or other mixtures thereof), tautomers, isotopes, polymorphs, or pharmaceutically acceptable prodrugs thereof. In many instances, for the sake of brevity, a compound or formula of compounds may be described as including “or a pharmaceutically acceptable salt thereof.” In each instance, it is understood that each form of said compound(s) referred to in the prior sentence are contemplated and included in each description or definition in question.

The terms “pharmacologically active amount,” “pharmaceutically effective amount,” or “effective amount” relates to an amount of a compound that provides a detectable reduction in parasitic activity in vitro or in vivo, or diminishes the likelihood of emergence of drug resistance.

The terms “treatment” or “treating” refer to an approach for obtaining beneficial or desired results including clinical results. Beneficial or desired clinical results may include one or more of the following: (i) inhibiting the disease or condition, such as a malaria infection (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

The terms “inhibiting” or “inhibition” indicate a decrease, such as a significant decrease, in the baseline activity of a biological activity or process. For instance, “inhibition” of a malaria, babesiosis, or toxoplasmosis infection refers to a decrease in symptoms or progress of such an infection as a direct or indirect response to the presence of a compound of Formula (I), or a pharmaceutically acceptable salt or co-crystal thereof, relative to symptoms or progress of such an infection in the absence of such compound or a pharmaceutically acceptable salt or co-crystal thereof.

A “therapeutically effective amount” or “diagnostically effective amount” refers to a quantity of a specified agent sufficient to achieve a desired effect in a subject being treated with that agent. For example, this may be the amount of a compound disclosed herein useful in treating an infection in a subject, such as a malaria, babesiosis, or toxoplasmosis infection. Ideally, a therapeutically effective amount or diagnostically effective amount of an agent is an amount sufficient to inhibit or treat the disease without causing a substantial cytotoxic effect in the subject. The therapeutically effective amount or diagnostically effective amount of an agent will be dependent on the subject being treated, the severity of the affliction, and the manner of administration of the therapeutic composition.

The compounds and pharmaceutical compositions herein may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, a human daily dose may be from about 0.1 mg to about 1,000 mg. In other embodiments, a human daily dose may be from about 0.1 mg to about 500 mg. In other embodiments, the human daily dose may be from about 1 mg to about 250 mg. In still other embodiments, the human daily dose may be from about 1 mg to about 200 mg. In further embodiments, the human daily dose may be from the group of ranges of from a) about 1 mg to about 150 mg; b) about 1 mg to about 100 mg; c) about 1 mg to about 75 mg; d) about 1 mg to about 50 mg; e) about 5 mg to about 50 mg; f) about 5 mg to about 40 mg; g) about 1 mg to about 25 mg; and about 1 mg to about 20 mg.

In other embodiments, the doses listed above for daily use may be administered as once-weekly or twice-weekly (semi-weekly) doses. In some embodiments, the compound may be delivered bi-weekly (every other week) as a prophylaxis for the disease states described herein.

Non-limiting examples of once weekly, twice weekly, or bi-weekly doses include 5 mg, 10 mg, 20 mg, 30 mg, 40 mg, 50 mg, 75 mg, 100 mg, 125 mg, 150 mg, 175 mg, and 200 mg doses.

In each dosage range, the pharmaceutically effective amount of the compound, or a pharmaceutically acceptable salt thereof, may comprise a single daily administration or divided over two, three, or four administrations per day.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively.

Particular examples of the presently disclosed compounds include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

It is understood that substituents and substitution patterns of the compounds described herein can be selected by one of ordinary skill in the art to provide compounds that are chemically stable and that can be readily synthesized by techniques known in the art and further by the methods set forth in this disclosure.

The compounds and pharmaceutical compositions disclosed herein can be used for inhibiting or preventing parasitic diseases. For example, human or animal (non-human) parasitic diseases include malaria, toxoplasmosis, theileriosis, amebiasis, giardiasis, leishmaniasis, trypanosomiasis, neosporosis (Neospora caninum infection), and coccidiosis, caused by organisms such as Toxoplasma sp. (such as Toxoplasma gondii), Eimeria sp. (Eimeriosis), Babesia bovis (babesiosis), Theileria sp. (Theileria annulata—tropical theileriosis and Theileria parva—East Coast fever), and also includes infections by helminths, such as ascaris, schistosomes and filarial worms.

Provided is a method for treating coccidiosis in a non-human subject, the method comprising administering to the non-human subject in need thereof a pharmaceutically effective amount of Formula (I), or a pharmaceutically acceptable salt thereof. In some embodiments, the non-human subject is a poultry subject, including chickens, ducks, and turkeys. In some embodiments, the chickens in need of such treatment are infected with a pathogen selected from the group of Eimeria tenella, E. maxima, E. mitis, E. acervulina, E. brunetti, E. praecox, and E. necatrix.

In some embodiments, the non-human subject is a ruminant subject, including cattle, bison, sheep, and goats. In some embodiments, the coccidiosis in the ruminant concerns an infection of a pathogen selected from the group of Eimeria bovis, E. zuemii, E. auburnensis, E. alabamensis,

In goats, the coccidiosis may be associated with infection of a pathogen selected from the group of E. christenseni, E. arloingi, E. caprina, and E. ninakohlyakimovae.

The compounds and compositions described herein are also effective in the inhibition of fungal pathogens including Pneumocystis carinii, Aspergillus fumigatus, and others.

In particular embodiments, the parasitic diseases may be caused by parasites that cause malaria. Particular species of parasites that are included within this group include all species that are capable of causing human or animal infection. Illustrative species include Plasmodium falciparum, Plasmodium vivax, Plasmodium ovale, Plasmodium knowlesi, and Plasmodium malariae. The compounds and compositions disclosed herein are particularly useful for inhibiting drug-resistant malaria such as chloroquine-resistant malaria or multidrug-resistant malaria that is caused by organisms harboring resistance to chloroquine, quinine, mefloquine, pyrimethamine, dapsone, and/or atovaquone.

Toxoplasmosis is caused by a sporozoan parasite of the Apicomplexa called Toxoplasma gondii. It a common tissue parasite of humans and animals. Most of the infections appear to be asymptomatic (90%), however toxoplasmosis poses a serious health risk for immuno-compromised individuals, such as organ transplant recipients, cancer and AIDS patients, and the unborn children of infected mothers. The compounds disclosed herein may be used alone to treat toxoplasmosis or they may be co-administered with “antifolates” such as sulfonamides, pyrimethamine, trimethoprim, biguanides and/or atovaquone.

In further embodiments, the compounds disclosed herein may be co-administered with another pharmaceutically active compound. For example, the compounds may be co-administered with quinine, chloroquine, atovaquone, proguanil, primaquine, amodiaquine, mefloquine, piperaquine, artemisinin, methylene blue, pyrimethamine, sulfadoxine, artemether-lumefantrine (COARTEM®), dapsone-chlorproguanil (LAPDAP®), artesunate, quinidine, clopidol, pyridine/pyridinol analogs, 4(1H)-quinolone analogs, dihydroartemisinin, a mixture of atovaquone and proguanil, an endoperoxide, an acridone as disclosed in WO 2008/064011 (which is incorporated herein by reference in its entirety), a pharmachin as disclosed in U.S. Provisional Patent Application titled “Compounds for Treating Parasitic Disease” filed Nov. 18, 2008 (which is incorporated herein by reference in its entirety), or any combination of these.

The compounds disclosed herein may be included in pharmaceutical compositions (including therapeutic and prophylactic formulations), typically combined together with one or more pharmaceutically acceptable vehicles or carriers and, optionally, other therapeutic ingredients (for example, antibiotics, anti-inflammatories, or drugs that are used to reduce pruritus such as an antihistamine). The compositions disclosed herein may be advantageously combined and/or used in combination with other antimalarial agents as described above.

Such pharmaceutical compositions can be administered to subjects by a variety of mucosal administration modes, including by oral, rectal, intranasal, intrapulmonary, or transdermal delivery, or by topical delivery to other surfaces. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other alternative embodiments, the compound can be administered ex vivo by direct exposure to cells, tissues or organs originating from a subject.

In some embodiments, the antimalarial agent or combination of antimalarial agents, including the compounds described herein, or a pharmaceutically acceptable salt thereof, may be administered to animals, such as chickens, as an additive to their prepared feed or grain.

In some embodiments, a pharmaceutically effective amount of a compound herein (including those of Formula (I) and all other formulas and individual compounds described herein), or a pharmaceutically acceptable salt thereof, may be administered to a human in need thereof by injection. In some embodiments, the injection may be subcutaneous. In other embodiments, the injection is intramuscular.

The forms in which the compound of Formula I, or a pharmaceutically acceptable salt or co-crystal thereof, may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline may also conventionally be used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

In some embodiments, the compound, or a pharmaceutically acceptable salt thereof, may be administered using a formulation comprising sesame oil, preferably pharmaceutical grade sesame oil. Components of an injectable formulation may include additional polar compounds, such as those selected from the group of monoglycerides, diglycerides, free fatty acids, plant sterols, sesamin, and sesamolin. Some injectable formulations further comprise ethanol. In some embodiments, the injectable formulation comprises from about 5 weight % to about 10 weight % ethanol. In other embodiments, the injectable formulation comprises from about 7 weight % to about 8 weight % ethanol. In other embodiments, the injectable formulation comprises from about 7.25 weight % to about 7.75 weight % ethanol. In other embodiments, the injectable formulation comprises about 7.5 weight % ethanol.

Additional components that may be used for intramuscular injections include vegetable oils, such as peanut oil, almond oil, olive oil, castor oil, and soybean oil. Also suitable are synthetic oils, such as polyethylene glycol, triglycerides of higher saturated fatty acids, monoesters of higher fatty acids, etc.

The injectable composition may also comprise one or more excipients, such as benzyl alcohol or benzoic acid compounds, including benzyl benzoate or sodium benzoate. Other useful excipients include methyl cholate, hydrophobic colloidal anhydrous silica, colloidal silicon dioxide, cholesteryl fatty acid ester like cholesteryl oleate, cholesteryl nonanoate, cholesteryl stearate, polyoxyethylen(5)sorbitan monooleate, polyoxyethylen(6) stearate, polyvalent metal salts of fatty acids e.g. aluminum stearate, fatty acid ester of carbohydrates like Rheopearl®, sorbitan fatty acid esters like sorbitan monolaurate, sorbitan sesquioleate, and sorbitan monostearate, and glycerol fatty acid ester like glycerol monostearate.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. In some embodiments, for parenteral administration, sterile injectable solutions are prepared containing a therapeutically effective amount, e.g., 0.1 to 1000 mg, of the compound of Formula I, or a pharmaceutically acceptable salt or co-crystal thereof. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, the severity of the subject's symptoms, and the like.

To formulate the pharmaceutical compositions, the compound can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The compound can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The compound can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain embodiments, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-caprolactone-CO-lactic acid), poly(epsilon-caprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In some embodiments, the method of delivering a pharmaceutically effective amount of a compound described herein, or a pharmaceutically acceptable salt thereof, may be administered to a subject in need thereof through a medical implant, particularly an implant designed to provide a continuous release, sustained release, or timed release of the active compound. In some embodiments, the implant comprises an amount of the desired compound and an ethylene vinyl acetate (EVA) copolymer, such as the copolymer designs described in U.S. Pat. No. 7,736,665 (Patel et al.), 8,852,623 (Patel et al.), 9,278,163 (Patel et al.), 10,111,830 (Patel et al.), and 10,123,971 (Patel et al.), each granted to Titan Pharmaceuticals, Inc.

In some embodiments, an implant may comprise dimensions of from 0.5 to about 7 mm in diameter. In some embodiments the devices are about 0.5 to 10 cm in length. In one embodiment, the device is from about 1 to about 3 cm in length. In one embodiment, the device is about 2 cm to about 3 cm in length. In another embodiment, the device is about 2.6 cm in length. In one embodiment, the device is about 1 to about 3 mm in diameter. In another embodiment, the device is about 2 to about 3 mm in diameter. In one embodiment, the device is about 2.4 mm in diameter. In some embodiments in which devices comprises dimensions of about 2.4 mm in total diameter and about 2.6 cm in total length, the devices each release 1 mg of pharmaceutical substance per day.

In some embodiments the implantable devices comprise from about 10% by weight to about 85% by weight a compound of Formula (I), or a pharmaceutically acceptable salt thereof, and the remainder of the implant comprises an ethylene vinyl acetate (EVA) copolymer. In some embodiments, the implant comprises about 75% active drug (a compound of Formula I, or a pharmaceutically acceptable salt thereof) and about 25% EVA. In other separate embodiments the implant comprises, respectively about 10% active drug/about 90% EVA, about 20% active drug/about 80% EVA, about 30% active drug/about 70% EVA, about 40% active drug/about 60% EVA, about 50% active drug/about 50% EVA, about 60% active drug/about 40% EVA, about 70% active drug/about 30% EVA, and about 80% active drug/about 20% EVA.

Additional embodiments comprise methods in which the active drug described herein (a compound of Formula I, or a pharmaceutically acceptable salt thereof) is administered to a subject in need thereof in a continuous release, sustained release, or timed release gel formulation, such as a hydrogel formulation. Gel carriers useful for delivering a pharmaceutically effective amount of a compound described herein, or a pharmaceutically acceptable salt thereof, include thermally responsive hydrogel carriers, including, but not limited to injectable block copolymer-based thermally responsive hydrogels; carbapol (poly-acrylic acid) gels; chitosan gels, such as chitosan thermogels; nanoparticle-containing/nanocomposite hydrogels; modified poly(ethylene glycol) gels; carrageenan gels; and water-in-sorbitan-monostearate gels. Examples of useful gel carriers include those described in Sarah Gordon's chapter Gels as Vaccine Delivery Systems at pages 203-220 in Subunit Vaccine Delivery, Springer New York 2015 (Print ISBN: 1-4939-1416-2), U.S. Pat. No. 10,272,140 (Yu et al.), U.S. Pat. No. 9,526,787 (Ko et al.), and Bobbala et al., AAPS J. 2016 January, 18(1), pp. 261-269.

Oral gel, gel-bead, or gel droplet formulations may also be used for delivering effective amounts of the compounds herein, or pharmaceutically acceptable salts thereof, to animals, such as poultry. Examples of gel formulations that may be used with the active drugs described herein include those described in U.S. Pat. No. 10,155,034 (Lee) and U.S. Pat. No. 8,858,959 (Jenkins et al.).

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

Typical subjects intended for treatment with the compositions and methods of the present disclosure include humans, as well as non-human primates and other animals. To identify subjects for prophylaxis or treatment according to the methods of the disclosure, accepted screening methods are employed to determine risk factors associated with a parasitic infection to determine the status of an existing disease or condition in a subject. These screening methods include, for example, preparation of a blood smear from an individual suspected of having malaria. The blood smear is then fixed in methanol and stained with Giemsa and examined microscopically for the presence of Plasmodium infected red blood cells. These and other routine methods allow the clinician to select patients in need of therapy using the methods and pharmaceutical compositions of the disclosure.

The administration of the compound of the disclosure can be for either prophylactic or therapeutic purpose. When provided prophylactically, the compound is provided in advance of any symptom. The prophylactic administration of the compound serves to prevent or ameliorate any subsequent disease process. When provided therapeutically, the compound is provided at (or shortly after) the onset of a symptom of disease or infection.

For prophylactic and therapeutic purposes, the compound can be administered to the subject by the oral route or in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, porcine, feline, non-human primate, and other accepted animal model subjects known in the art. Alternatively, effective dosages can be determined using in vitro models (for example, whole cell assays that monitor the effect of various drugs on parasite growth rate). Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease). In alternative embodiments, an effective amount or effective dose of the compound may simply inhibit or enhance one or more selected biological activities correlated with a disease or condition, as set forth herein, for either therapeutic or diagnostic purposes.

The actual dosage of the compound will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and formulations of the disclosure is about 0.01 mg/kg body weight to about 20 mg/kg body weight, such as about 0.05 mg/kg to about 5 mg/kg body weight, or about 0.2 mg/kg to about 2 mg/kg body weight.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth.

The instant disclosure also includes kits, packages and multi-container units containing the herein described pharmaceutical compositions, active ingredients, and/or means for administering the same for use in the prevention and treatment of diseases and other conditions in mammalian subjects. Kits for diagnostic use are also provided. In one embodiment, these kits include a container or formulation that contains one or more of the conjugates described herein. In one example, this component is formulated in a pharmaceutical preparation for delivery to a subject. The conjugate is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

Initially, ELQ-596 was prepared using a previously reported approach.¹² The 4-OEt-quinolone 1 was prepared according to the literature (Scheme 1, below) and reacted with pinacol ester 2 as previously described¹². Finally, the 4(1H)-quinolone ELQ-596 was obtained after hydrolysis of the 4-chloro-quinoline using potassium acetate (KOAc) in glacial acetic acid (AcOH).

### Scheme 1. Synthesis of ELQ-596.^{a}

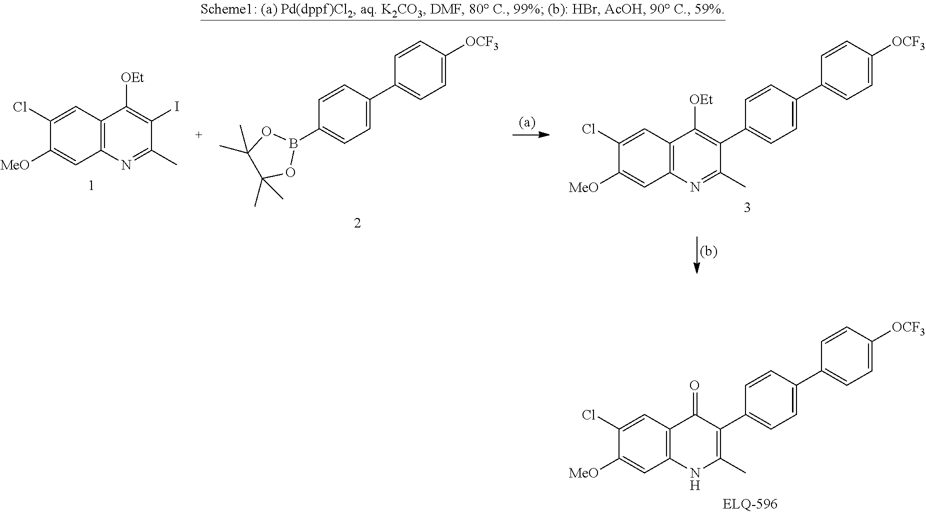 [SMILES: CCOc1c(I)c(C)nc2cc(OC)c(Cl)cc12 ; CC1(C)OB(c2ccc(-c3ccc(OC(F)(F)F)cc3)cc2)OC1(C)C ; CCOc1c(-c2ccc(-c3ccc(OC(F)(F)F)cc3)cc2)c(C)nc2cc(OC)c(Cl)cc12 ; COc1cc2[nH]c(C)c(-c3ccc(-c4ccc(OC(F)(F)F)cc4)cc3)c(=O)c2cc1Cl]

As exemplified in Scheme 1, provided herein is a method for the preparation of a compound of Formula (I):

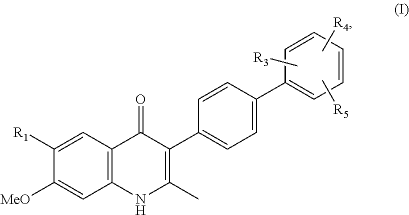

wherein R₁ is selected from the group of H, F, and Cl, and R₃, R₄, and R₅ are as defined above, the method comprising:
a) a first step of reacting a compound of the formula:

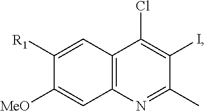

wherein R₁ is selected from the group of H, F, and Cl, with an optionally substituted 2-([1,1′-biphenyl]-4-yl)-4,4,5,5-tetramethyl-1,3,2-dioxaborolane compound of the formula

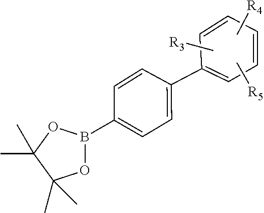

to form a first step product compound of the formula:

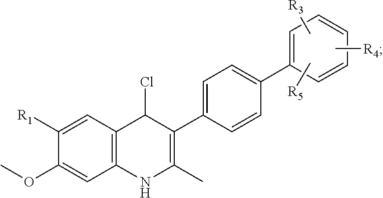

and
b) a second step of treating the first step product compound with an acidic medium to form the compound of Formula (I).

An additional embodiment comprises a method of producing the first step product compound as described above. Another embodiment provides a compound of Formula (XI):

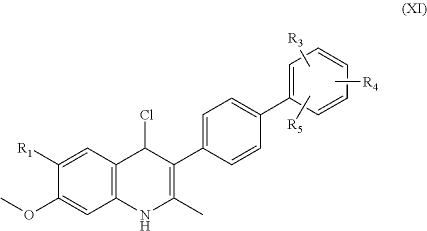

wherein R₁ is selected from the group of H, F, and Cl, and R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), and —C(O)NH(—CH₂—C₃-C₆ cycloalkyl).

Another embodiment provides a compound of Formula (XI), wherein R₁ is selected from the group of H, F, and Cl, and R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl).

Another embodiment provides a compound of Formula (XI), wherein R₁ is selected from the group of H, F, and Cl, and R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

Another embodiment provides a compound of Formula (XI), wherein R₁ is selected from the group of H, F, and Cl, and R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

Another embodiment provides a compound of Formula (XI), wherein R₁ is selected from the group of H, F, and Cl, and R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

Another embodiment provides a compound of Formula (XI), wherein R₁ is selected from the group of H, F, and Cl, and R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —SF₅, and CN.

Another embodiment provides a compound of Formula (XI), wherein R₁ is selected from the group of H, F, and Cl, and R₃, R₄, and R₅ are each independently selected from the group of H, F, C₁-C₂ alkyl, —O—C₁-C₂ alkyl, C₁-C₂ fluoroalkyl, —O—C₁-C₂ fluoroalkyl, —SF₅, and CN.

Another embodiment provides a compound of Formula (XI), wherein R₁ is selected from the group of H, F, and Cl, and R₃, R₄, and R₅ are each independently selected from the group of H, F, C₁-C₂ fluoroalkyl, —O—C₁-C₂ fluoroalkyl, and —SF₅.

Within each of the embodiments concerning a compound of Formula (XI), there is an additional embodiment wherein all variables are as defined for the specific embodiment, with the proviso that at least one of R₃, R₄, and R₅ is H. Within each of the embodiments concerning a compound of Formula (XI), there is also an additional embodiment wherein all variables are as defined for the specific embodiment, except R₁ is F. Within each of the embodiments concerning a compound of Formula (XI), there is also an additional embodiment wherein all variables are as defined for the specific embodiment, except R₁ is Cl.

Also provided are two separate embodiments comprising, respectively, a compound of Formula (X-1) and a compound of Formula (X-2):

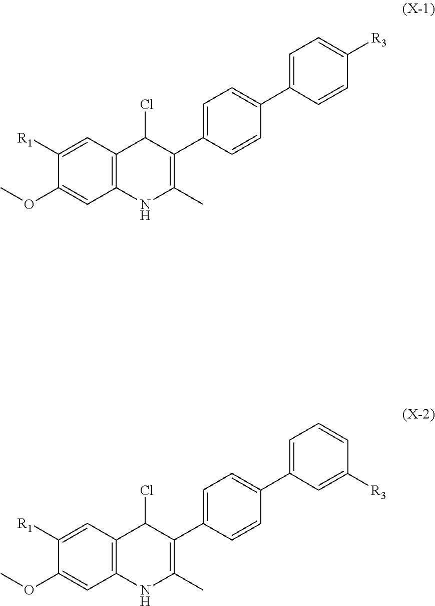

wherein, in each embodiment, R₁ is selected from the group of H, F, and Cl, and R₃ is selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl).

Two additional separate embodiments comprise, respectively, a compound of Formula (X-1) and a compound of Formula (X-2), wherein R₁ is selected from the group of H, F, and Cl, and R₃ is selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl).

Two additional separate embodiments comprise, respectively, a compound of Formula (X-1) and a compound of Formula (X-2), wherein R₁ is selected from the group of H, F, and Cl, and R₃ is selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

Two additional separate embodiments comprise, respectively, a compound of Formula (X-1) and a compound of Formula (X-2), wherein R₁ is selected from the group of H, F, and Cl, and R₃ is selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

Two additional separate embodiments comprise, respectively, a compound of Formula (X-1) and a compound of Formula (X-2), wherein R₁ is selected from the group of H, F, and Cl, and R₃ is selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

Two additional separate embodiments comprise, respectively, a compound of Formula (X-1) and a compound of Formula (X-2), wherein R₁ is selected from the group of H, F, and Cl, and R₃ is selected from the group of H, halogen, C₁-C₂ alkyl, —O—C₁-C₂ alkyl, C₁-C₂ haloalkyl, —O—C₁-C₂ haloalkyl, —SF₅, and CN.

Two additional separate embodiments comprise, respectively, a compound of Formula (X-1) and a compound of Formula (X-2), wherein R₁ is selected from the group of H, F, and Cl, and Ra is selected from the group of H, F, C₁-C₂ alkyl, —O—C₁-C₂ alkyl, C₁-C₂ fluoroalkyl, —O—C₁-C₂ fluoroalkyl, —SF₅, and CN.

Two additional separate embodiments comprise, respectively, a compound of Formula (X-1) and a compound of Formula (X-2), wherein R₁ is selected from the group of H, F, and Cl, and R₃ is selected from the group of H, F, C₁-C₂ fluoroalkyl, —O—C₁-C₂ fluoroalkyl, and —SF₅.

Within each of the embodiments concerning a compound of Formula (X-1) or Formula (X-2), there is also an additional embodiment wherein all variables are as defined for the specific embodiment, except R₁ is F. Within each of the embodiments concerning a compound of Formula (X-1) or Formula (X-2), there is also an additional embodiment wherein all variables are as defined for the specific embodiment, except R₁ is Cl.

In some embodiments, step a) of the preparation method above is completed in the presence of a palladium catalyst. In some embodiments, the palladium catalyst is selected from the group of dichloro-((bis-diphenylphosphino)ferrocenyl)-palladium (II) (Pd(dppf)Cl₂), dichloro-triphenylphosphino-palladium (II) (PdCl₂(PPh)₃), palladium (II) acetate (PD(OAc)₂), palladium (II) chloride (PDCl₂), tris(dibenzylideneacetone)dipalladium (PD₂(dba)₃), and tetrakis(triphenylphosphine)palladium (PD(PhP₃)₄). In some embodiments, the palladium catalyst is present at a concentration of from about 0.01 eq to about 0.1 eq. In other embodiments, the catalyst is present at a concentration of from about 0.03 eq to about 0.07 eq.

In some embodiments, the preparation is completed in an organic solvent in the presence of a base. In some embodiments, the organic solvent is selected from the group of DMF, THF, dioxane, acetone, and toluene. In some embodiments, the base is selected from the group of K₂CO₃, CsF, Cs₂CO₃, NaOH, Na₂CO₃, and K₂CO₃. In some embodiments, the base is present at a concentration of from about 0.5 eq to about 3 eq.

To vary the benzenoid substituents R₁ and X we decided to adopt approach I (Scheme 2, below), where the 4(1H)-quinolone is formed in the final reaction step. To vary the biphenyl substituents R₃, R₄, and R₅, approach II was used wherein the outer ring of the biphenyl side chain was introduced late in the 4(1H)-quinolone synthesis.

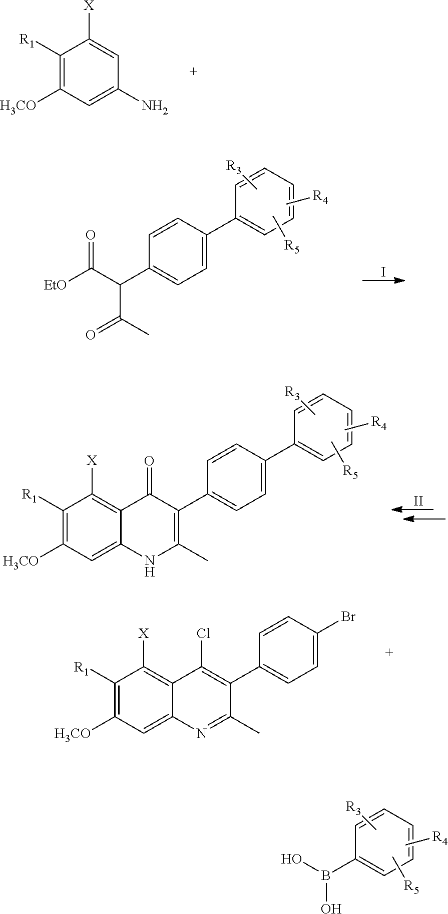

To prepare the β-keto ester intermediates 8a and 8b, we adapted a route that we recently developed for the large-scale synthesis of ELQ-300¹⁰. Suzuki reaction of commercially available ethyl 2-(4-bromophenyl) acetate 5 and (4-(trifluoromethoxy)phenyl) boronic acid with [1,1′-Bis(diphenylphosphino)-ferrocene]palladium(II) dichloride (Pd(dppf)Cl₂) and potassium carbonate (K₂CO₃) in dimethylformamide (DMF) provided ethyl 2-(4′-(trifluoromethoxy)-[1,1′-biphenyl]-4-yl)acetate 6 in 58% yield (Scheme 3). Acylation of 5 and 6 using freshly prepared lithium hexamethyldisilazide (LiHMDS) and excess acetic anhydride (Ac₂O) in tetrahydrofuran (THF) gave bis-acylated enol acetates 7a and 7b as mixtures of equally reactive E- and Z-isomers in quantitative yield and in sufficient purity (>95%) to be used in the next step without further purification. The two stereoisomers can be isolated by flash chromatography. However, we were not able to unambiguously assign the two stereoisomers using NOESY 2D NMR. GC-MS analysis indicated that the major stereoisomer of 7a was 80% of the mixture, whereas the major stereoisomer of 7b was 95% of the mixture. Using catalytic para-toluenesulfonic acid (p-TsOH) in AcOH, bis-acylated 7a and 7b were converted to β-keto ester intermediates 8a and 8b, which existed as mixtures of keto and enol tautomers as determined by ¹H-NMR. After concentration, the crude reaction mixture contained mainly β-keto esters 8a or 8b and catalytic p-TsOH, which was required in the next reaction and can be used without further purification.

Provided here are novel compounds useful in the synthesis of compounds of Formula (I), or a pharmaceutically acceptable salt thereof. One embodiment provides a compound of the Formula (A):

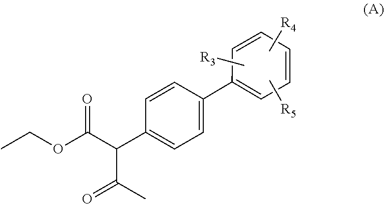

wherein R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), and —C(O)NH(—CH₂—C₃-C₆ cycloalkyl). In some embodiments, at least one of R₃, R₄, and R₅ is hydrogen. In some embodiments, R₃ is selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), and —C(O)NH(—CH₂—C₃-C₆ cycloalkyl); and R₄ is hydrogen; and R₅ is hydrogen.

An embodiment provides a compound of Formula (A), wherein R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl).

Another embodiment provides a compound of Formula (A), wherein R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

A further embodiment provides a compound of Formula (A), wherein R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl);

Another embodiment provides a compound of Formula (A), wherein R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl).

An additional embodiment provides a compound of Formula (A), wherein R₃, R₄, and R₅ are each independently selected from the group of halogen, C₁-C₂ alkyl, —O—C₁-C₂ alkyl, C₁-C₂ haloalkyl, —O—C₁-C₂ haloalkyl, —SF₅, and CN.

Two separate embodiments provide, respectively, a compound of Formula (A-1) and a compound of Formula (A-2):

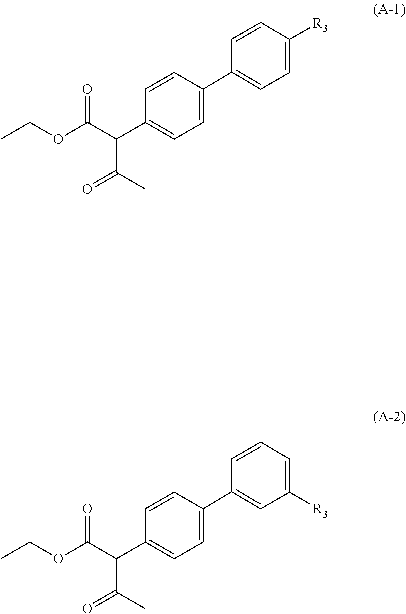

wherein, in each embodiment, R₃ is selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), and —C(O)NH(—CH₂—C₃-C₆ cycloalkyl). In some separate embodiments concerning, respectively, a compound of Formula (A-1) and a compound of Formula (A-2), R₃ is selected from the group of Cl, F, CH₂F, CHF₂, CF₃, —O—CF₃, and SF₅. In other separate embodiments, R₃ is selected from the group of F, CF₃, —O—CF₃, and SF₅.

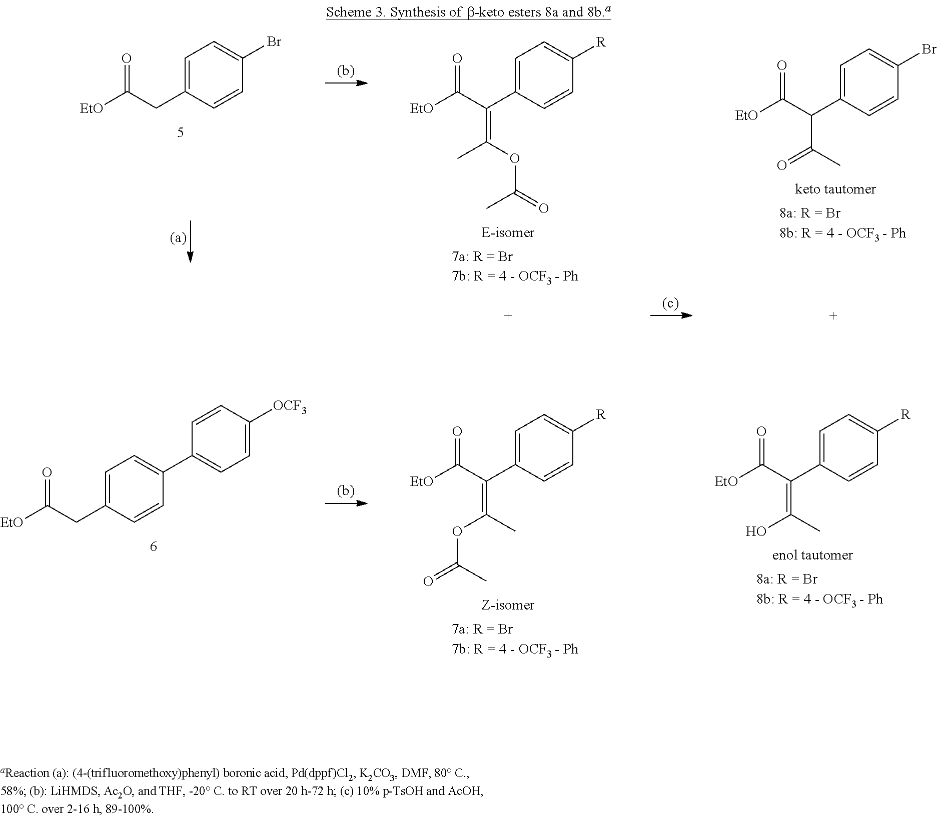

Relatively facile variation of benzenoid substituents X, Y and Z was accomplished by reaction of β-keto ester 8b with various anilines (10a-d). The crude β-keto ester mixture was reacted with anilines 10a-d under Dean-Stark conditions with refluxing benzene to provide Schiff bases 10a-d, which were used without further purification (Scheme 4). Formation of 4(1H)-quinolones ELQ-596, ELQ-601, ELQ-649 and ELQ-650 was accomplished via Conrad-Limpach cyclization^{15, 16} of Schiff bases 10a-d in Dowtherm A at 250° C.¹⁷. The crude products obtained from this reaction were >98% pure as determined by HPLC and ¹H-NMR. To allow detection and quantification of the ELQ-596 and ELQ-650 regioisomers the products were converted to their corresponding 4-chloro derivatives using POCl₃ and analyzed by GC-MS. The results indicated that negligible amounts (<0.5%) of ELQ-596 isomer and ELQ-650 isomer were formed under these conditions.

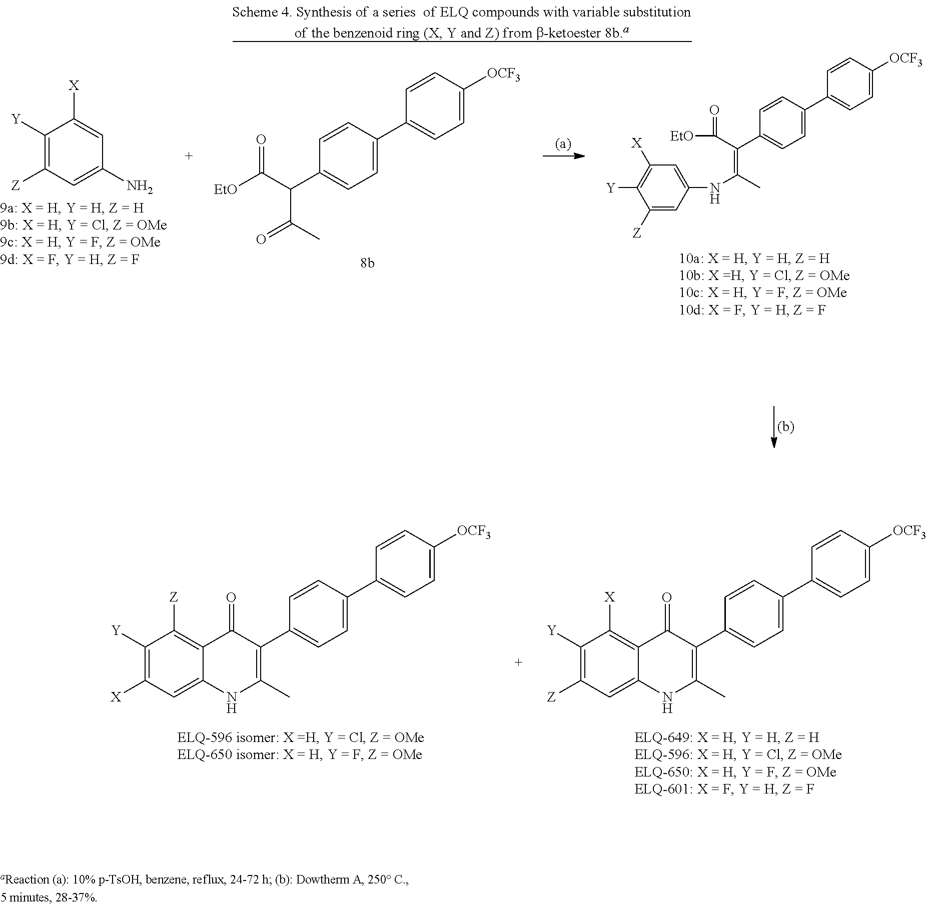

Synthesis of 4(1H)-quinolone 12 was accomplished using the same method described above. The crude β-keto ester 8a mixture was reacted with commercially available 4-chloro-3-methoxy aniline 9b under Dean-Stark conditions with refluxing benzene to provide Schiff base 11, which was used without further purification (Scheme 5). Formation of 4(1 M-quinolone 12 was accomplished via Conrad-Limpach cyclization^{15, 16} of Schiff base 11 in Dowtherm A at 250° C.¹⁷. 4-Chloroqinoline 13 was then prepared from 4(1H)-quinolone 12 using neat POCl₃¹³.

An embodiment provides a compound of Formula (XII):

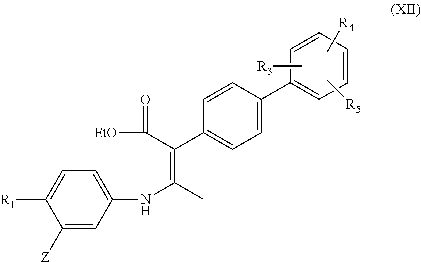

wherein R₁ is selected from the group of H, F, and Cl; and R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), and —C(O)NH(—CH₂—C₃-C₆ cycloalkyl). In some embodiments, at least one of R₃, R₄, and R₅ is hydrogen. In some embodiments, R₃ is selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), and —C(O)NH(—CH₂—C₃-C₆ cycloalkyl); R₄ is hydrogen; R₅ is hydrogen, and Z is selected from the group of H, F, and OMe.

An embodiment provides a compound of Formula (XII), wherein R₁ is selected from the group of H, F, and Cl; and R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), and Z is selected from the group of H, F, and OMe.

Another embodiment provides a compound of Formula (XII), wherein R₁ is selected from the group of H, F, and Cl; and R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), and Z is selected from the group of H, F, and OMe.

A further embodiment provides a compound of Formula (XII), wherein R₁ is selected from the group of H, F, and Cl; and R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), and Z is selected from the group of H, F, and OMe.

Another embodiment provides a compound of Formula (XII), wherein R₁ is selected from the group of H, F, and Cl; and R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), and Z is selected from the group of H, F, and OMe.

An additional embodiment provides a compound of Formula (XII), wherein R₁ is selected from the group of H, F, and Cl; and R₃, R₄, and R₅ are each independently selected from the group of halogen, C₁-C₂ alkyl, —O—C₁-C₂ alkyl, C₁-C₂ haloalkyl, —O—C₁-C₂ haloalkyl, —SF₅, 2-pyrrolidinone, and CN, and Z is selected from the group of H, F, and OMe.

Two separate embodiments provide, respectively, a compound of Formula (XI-1) and a compound of Formula (XI-2):

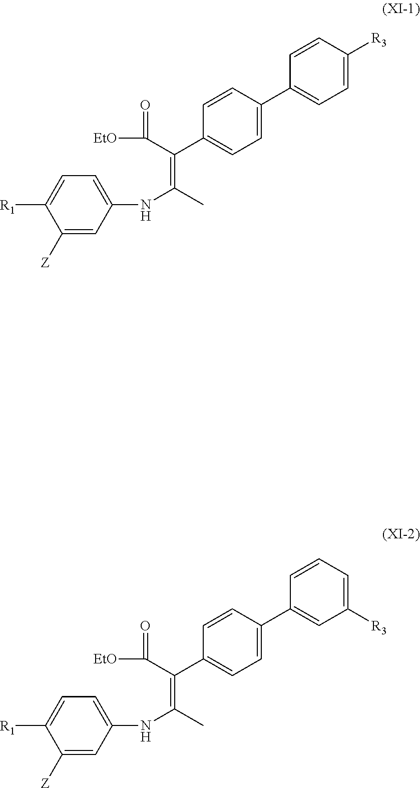

wherein, in each embodiment, R₁ is selected from the group of H, F, and Cl; and R₃ is selected from the group of halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), and —C(O)NH(—CH₂—C₃-C₆ cycloalkyl), and Z is selected from the group of H, F, and OMe.

In some separate embodiments concerning, respectively, a compound of Formula (A-1) and a compound of Formula (A-2), R₁ is selected from the group of H, F, and Cl; and R₃ is selected from the group of Cl, F, CH₂F, CHF₂, CF₃, —O—CF₃, and SF₅, and Z is selected from the group of H, F, and OMe.

In other separate embodiments, R₁ is selected from the group of H, F, and Cl; and R₃ is selected from the group of F, CF₃, —O—CF₃, and SF₅, and Z is selected from the group of H, F, and OMe.

Additional embodiments are provided corresponding to each embodiment for a compound of Formula (XI-1) and a compound of Formula (XI-2) as just described, wherein R₃ is as defined and R₁ is Cl. Additional embodiments are also provided corresponding to each embodiment for a compound of Formula (XI-1) and a compound of Formula (XI-2) as just described, wherein R₃ is as defined and R₁ is F.

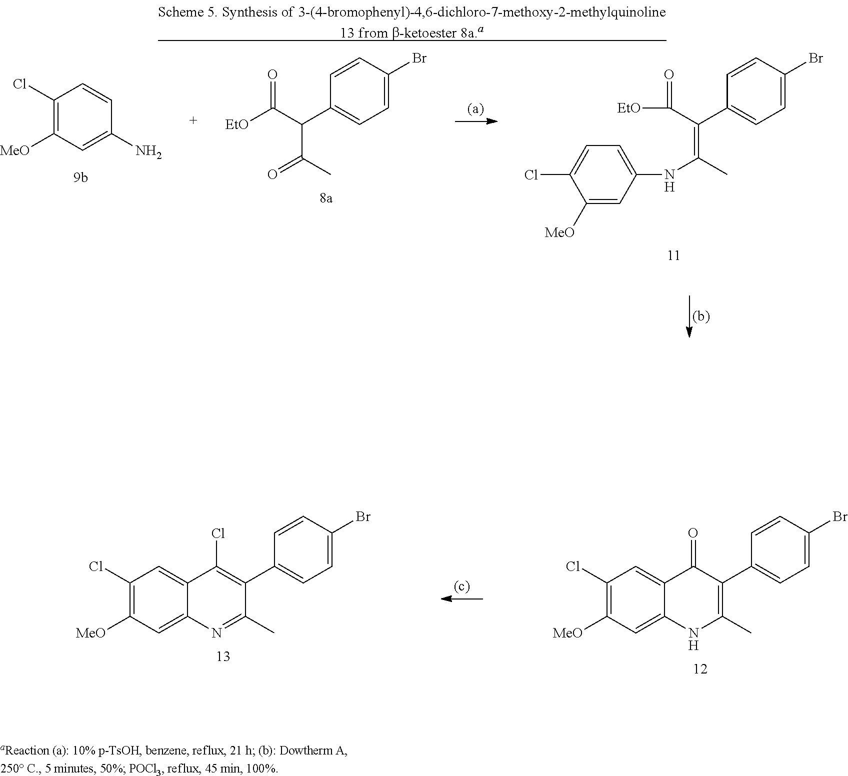 [SMILES: COc1cc(N)ccc1Cl ; CCOC(=O)C(C(C)=O)c1ccc(Br)cc1 ; CCOC(=O)/C(=C(/C)Nc1ccc(Cl)c(OC)c1)c1ccc(Br)cc1 ; COc1cc2nc(C)c(-c3ccc(Br)cc3)c(Cl)c2cc1Cl ; COc1cc2[nH]c(C)c(-c3ccc(Br)cc3)c(=O)c2cc1Cl]

Variation of biphenyl substituents R¹ and R² was then accomplished via selective Suzuki reaction of various boronic acids or pinacol esters 14a-k with 4-chloro-quinoline 13 using Pd(dppf)Cl₂ and K₂CO₃ in DMF (Scheme 6). The resulting 4-chloro-quinolines 15a-k were then converted to their corresponding 4(1H)-quinolones using KOAc in AcOH.

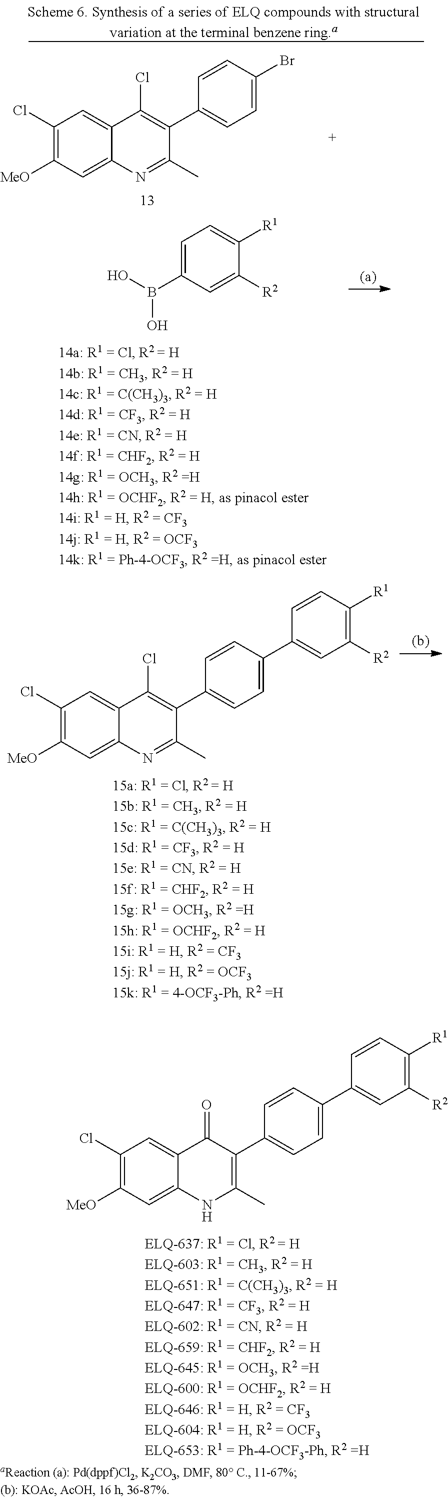

For our in vivo work, it was necessary to convert ELQ-596 to the corresponding alkoxycarbonate ester prodrug, ELQ-598. This was accomplished using tetra-n-butylammonium iodide (TBAI) and K₂CO₃ in DMF according to a previously published method (Scheme 7)⁹.

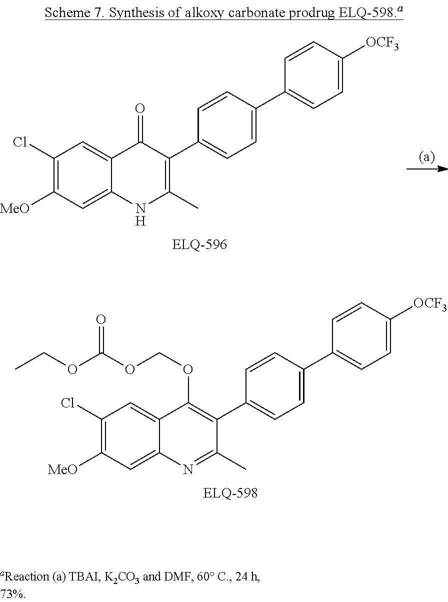 [SMILES: COc1cc2[nH]c(C)c(-c3ccc(-c4ccc(OC(F)(F)F)cc4)cc3)c(=O)c2cc1Cl ; CCOC(=O)OCOc1c(-c2ccc(-c3ccc(OC(F)(F)F)cc3)cc2)c(C)nc2cc(OC)c(Cl)cc12]

### Results and Discussion

Rationale for Synthesis of a 3-Biaryl Version of ELQ-300. We maintain a large chemical library of >600 ELQ derivatives and over the years we (and others) have evaluated these drugs for antiparasitic activity against a range of parasites including Plasmodium falciparum, Toxoplasma gondii, Babesia microti, B. duncani, and including related parasites that are of particular importance to veterinary medicine. All of this information is stored in a database with information relating to chemical structure, physical chemical properties, cross resistance patterns, mammalian cell cytotoxicity, enzyme inhibitor profiles, and synthesis details. Pharmacological data and in vivo efficacy data are also stored in the database for superior molecules of interest. Recently, we conducted a retrospective analysis of the compounds that we have made over the years and came to the realization that neither we, nor anyone else, had ever synthesized the biphenyl analog of ELQ-300, i.e., ELQ-596 (FIG. 1). We synthesized the compound in a batch size of about 800 mg as described above. ELQ-596 was then tested for anti-plasmodial activity vs. four lab strains of P. falciparum including the drug sensitive D6 strain, the multidrug resistant Dd2 strain, the atovaquone (ATV)-resistant clinical isolate Tm90-C2, and the ELQ-300 resistant D1 clone, previously isolated from Dd2. In vitro assays were conducted in quadruplicate in 96 well plates in a starting range of 250 nM to 2.5 nM and retested at a 10-fold lower range where drug potency extended below this initial range. We employed the SyBr Green assay and the plates were incubated for 72 hours before harvesting^{18, 19}. Fluorescence readings were captured in a fluorescence plate reader and processed using Graphpad Prism software yielding IC₅₀ values along with 95% confidence intervals for each determination. Stock solutions were freshly prepared in DMSO.

TABLE 1
Structure activity profile of 3-biaryl-ELQs vs. drug sensitive (D6) and drug resistant
(Dd2, C2B, and D1) strains of Plasmodium falciparum. IC₅₀ values represent the concentration
of drug that suppresses parasite growth by 50% relative to controls without addition of drug.

| | | | | P. falciparum IC₅₀ values, nM | | | |
|---|---|---|---|---|---|---|---|
| | | | | | | Tm90- | |
| Structure | Code | MW | cLogP | D6 | Dd2 | C2B | D1 |
| 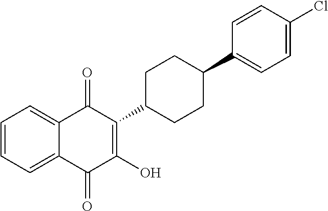 | Atovaquone | 367 | 6.1 | 0.08 (0.07 to 0.09) | 0.4 (0.3 to 0.4) | >1,000 | 0.4 (0.4 to 0.5) |
| 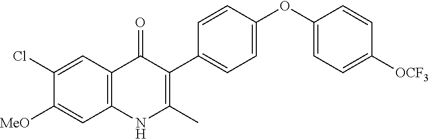 [SMILES: COc1cc2[nH]c(C)c(-c3ccc(Oc4ccc(OC(F)(F)F)cc4)cc3)c(=O)c2cc1Cl] | ELQ-300 | 476 | 5.2 | 3.3 (3.0 to 3.5) | 4.2 (3.6 to 4.9) | 2.5 (2.2 to 2.8) | 168 |
| 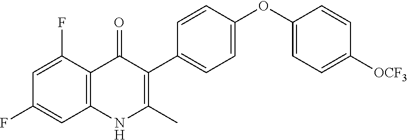 [SMILES: Cc1[nH]c2cc(F)cc(F)c2c(=O)c1-c1ccc(Oc2ccc(OC(F)(F)F)cc2)cc1] | ELQ-400 | 447 | 5.1 | 0.19 0.15 to 0.23 | 0.44 0.38 to 0.51 | 19.3 15.0 to 26.2 | 0.73 0.66 to 0.81 |
| 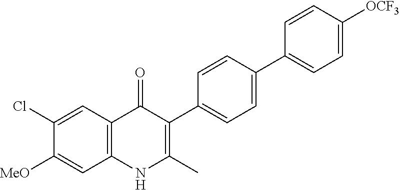 [SMILES: COc1cc2[nH]c(C)c(-c3ccc(-c4ccc(OC(F)(F)F)cc4)cc3)c(=O)c2cc1Cl] | ELQ-596 | 459 | 5.0 | 0.3 (0.3 to 0.4) | 0.4 (0.3 to 0.4) | 0.2 (0.2 to 0.2) | 449 (346 to 659) |
| 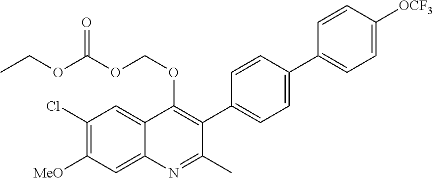 [SMILES: CCOC(=O)OCOc1c(-c2ccc(-c3ccc(OC(F)(F)F)cc3)cc2)c(C)nc2cc(OC)c(Cl)cc12] | ELQ-598 | 562 | 6.9 | 1.5 1.3 to 1.7 | 4.4 4.1 to 4.8 | 0.8 0.7 to 0.9 | 734 667 to 817 |
| 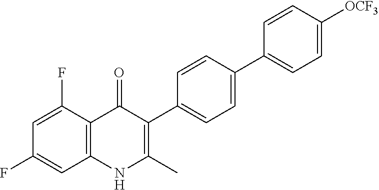 [SMILES: Cc1[nH]c2cc(F)cc(F)c2c(=O)c1-c1ccc(-c2ccc(OC(F)(F)F)cc2)cc1] | ELQ-601 | 431 | 4.9 | 5.9 (5.5 to 6.4 | 14.3 (13.5 to 15.0) | 24.5 (20.9 to 29.4) | 9.3 (8.8 to 10.0) |
| 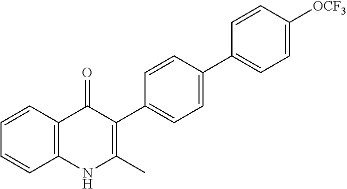 [SMILES: Cc1[nH]c2ccccc2c(=O)c1-c1ccc(-c2ccc(OC(F)(F)F)cc2)cc1] | ELQ-649 | 395 | 4.6 | 7.9 (6.7 to 9.3) | 8.3 (6.8 to 10.0) | 6.0 (4.9 to 7.4) | 15.8 (13.3 to 19.0) |
| 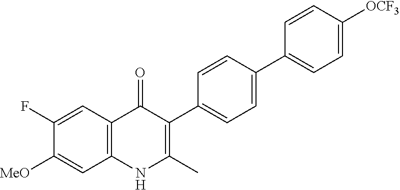 [SMILES: COc1cc2[nH]c(C)c(-c3ccc(-c4ccc(OC(F)(F)F)cc4)cc3)c(=O)c2cc1F] | ELQ-650 | 443 | 4.5 | 0.8 (0.7 to 0.8) | 1.1 (1.0 to 1.3 | 0.4 (0.4 to 0.5) | 14 (12.4 to 15.9) |
| 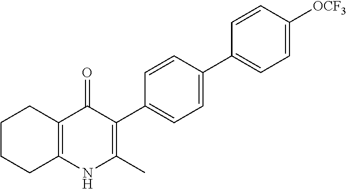 [SMILES: Cc1[nH]c2c(c(=O)c1-c1ccc(-c3ccc(OC(F)(F)F)cc3)cc1)CCCC2] | ELQ-660 | 399 | 4.6 | 15.6 (12.9 to 20.3) | 12.8 (10.8 to 17.7) | 7.9 (6.0 to 12.0) | 22.2 (16.20 to 45.45) |

Note: Values are the averages of assays performed in quadruplicate with (95% confidence intervals).

TABLE 2
Structure activity profile of 3-biaryl-ELQs vs. drug sensitive (D6) and drug resistant
(Dd2, C2B, and D1) strains of Plasmodium falciparum. IC₅₀ values represent the concentration
of drug that suppresses parasite growth by 50% relative to controls without addition of drug.

| | | | | P. falciparum IC₅₀ values, nM | | | |
|---|---|---|---|---|---|---|---|
| | | | | | | Tm90- | |
| Structure | Code | MW | cLogP | D6 | Dd2 | C2B | D1 |
| 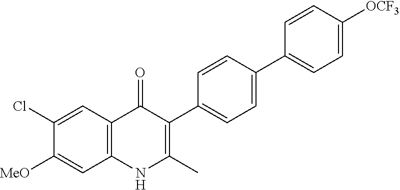 [SMILES: COc1cc2[nH]c(C)c(-c3ccc(-c4ccc(OC(F)(F)F)cc4)cc3)c(=O)c2cc1Cl] | ELQ- 596 | 459 | 5.0 | 0.3 (0.3 to 0.4) | 0.4 (0.3 to 0.4) | 0.2 (0.2 to 0.2) | 449 (346 to 659) |
| 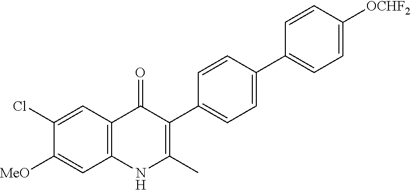 [SMILES: COc1cc2[nH]c(C)c(-c3ccc(-c4ccc(OC(F)F)cc4)cc3)c(=O)c2cc1Cl] | ELQ- 600 | 442 | 4.3 | 3.9 (3.4 to 4.5) | 13.4 (10.7 to 17.3) | 5.9 (4.5 to 7.9) | >250 |
| 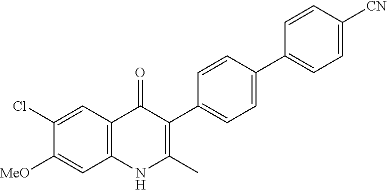 [SMILES: COc1cc2[nH]c(C)c(-c3ccc(-c4ccc(C#N)cc4)cc3)c(=O)c2cc1Cl] | ELQ- 602 | 401 | 3.4 | 4.8 (4.3 to 5.4) | 25.2 (22.9 to 27.9) | 10.9 (9.0 to 13.4) | >250 |
| 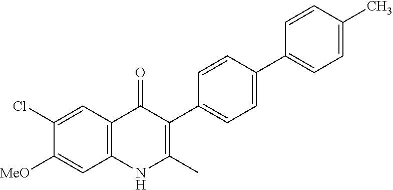 [SMILES: COc1cc2[nH]c(C)c(-c3ccc(-c4ccc(C)cc4)cc3)c(=O)c2cc1Cl] | ELQ- 603 | 390 | 4.5 | 1.0 (0.9 to 1.1) | 3.8 (3.2 to 4.6) | 1.5 (1.2 to 1.8) | >250 |
| 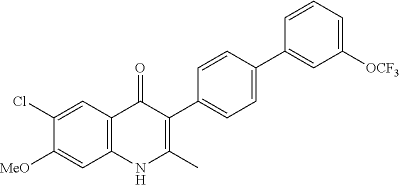 [SMILES: COc1cc2[nH]c(C)c(-c3ccc(-c4cccc(OC(F)(F)F)c4)cc3)c(=O)c2cc1Cl] | ELQ- 604 | 459 | 5.0 | 0.6 (0.5 to 0.7) | 0.5 (0.4 to 0.6) | 0.5 (0.5 to 0.6) | >250 |
| 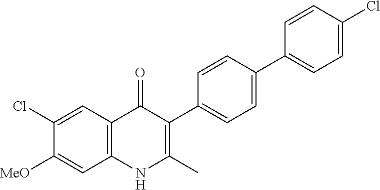 [SMILES: COc1cc2[nH]c(C)c(-c3ccc(-c4ccc(Cl)cc4)cc3)c(=O)c2cc1Cl] | ELQ- 637 | 410 | 4.7 | 3.4 (2.9 to 3.9) | 7.1 (5.7 to 9.2) | 3.0 (1.5 to 6.6) | >250 |
| 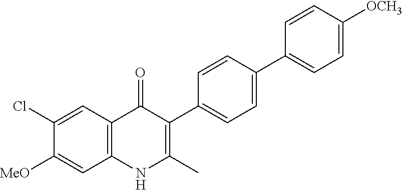 [SMILES: COc1ccc(-c2ccc(-c3c(C)[nH]c4cc(OC)c(Cl)cc4c3=O)cc2)cc1] | ELQ- 645 | 406 | 3.9 | 5.0 (4.7 to 5.5) | 10.2 (8.7 to 12.1) | 6.0 (5.4 to 6.8) | >250 |
| 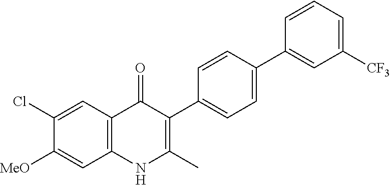 [SMILES: COc1cc2[nH]c(C)c(-c3ccc(-c4cccc(C(F)(F)F)c4)cc3)c(=O)c2cc1Cl] | ELQ- 646 | 443 | 4.9 | 1.1 (1.0 to 1.3) | 2.7 (2.2 to 3.3) | 1.0 (0.9 to 1.2) | >250 |
| 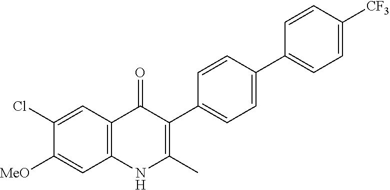 [SMILES: COc1cc2[nH]c(C)c(-c3ccc(-c4ccc(C(F)(F)F)cc4)cc3)c(=O)c2cc1Cl] | ELQ- 647 | 444 | 4.9 | 3.5 (3.3 to 3.8) | 7.8 (6.6 to 9.2) | 3.5 (3.1 to 3.9) | >250 |
| 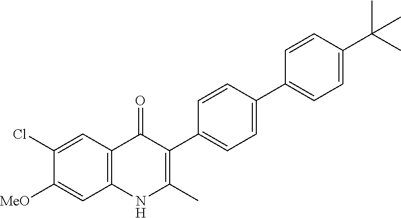 [SMILES: COc1cc2[nH]c(C)c(-c3ccc(-c4ccc(C(C)(C)C)cc4)cc3)c(=O)c2cc1Cl] | ELQ- 651 | 432 | X.X | 1.1 (0.9 to 1.4) | 1.4 (1.1 to 1.7) | 0.49 (0.34 to 0.68) | >250 |
| 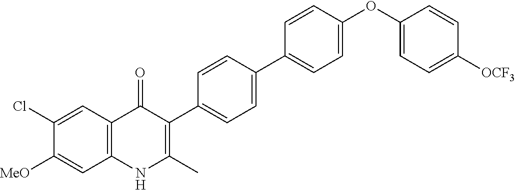 [SMILES: COc1cc2[nH]c(C)c(-c3ccc(-c4ccc(Oc5ccc(OC(F)(F)F)cc5)cc4)cc3)c(=O)c2cc1Cl] | ELQ- 653 | 552 | X.X | 1.5 (1.3 to 1.8) | 1.4 (1.1 to 1.8) | 0.88 (0.69 to 1.1) | >250 |
| 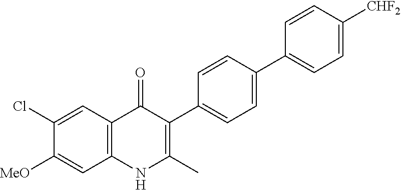 [SMILES: COc1cc2[nH]c(C)c(-c3ccc(-c4ccc(C(F)F)cc4)cc3)c(=O)c2cc1Cl] | ELQ- 659 | 426 | 4.2 | 1.8 (1.7 to 2.0) | 1.8 (1.6 to 2.1) | 0.57 (0.50 to 0.65) | >250 |

Note: Values are the averages of assays performed in quadruplicate with (95% confidence intervals).

In vitro Activities of Selected 3-Biaryl-ELQs vs. P. falciparum strains. IC₅₀ values are shown in Tables 1 and 2 together with 95% confidence intervals for a single experiment performed in quadruplicate. (Assays were repeated at least two times). We also prepared ELQ-598, the alkoxy-carbonate ester prodrug of ELQ-596, and included it in the assays along with historical controls atovaquone (ATV), ELQ-300 and ELQ-400. The latter is a drug that, like ATV, targets the Qo site of the Pf cyt bc₁ complex. Notice that the IC₅₀ values for ELQ-596 were improved over ELQ-300 by 8- to 10-fold for the D6 and Dd2 strains as well as the ATV, C2B (Table 1) while they were higher for the ELQ-300, D1 clone. We interpret these results to suggest higher inhibitory action by ELQ-596 vs. the wild type Pf cyt bc₁ as well as the mutated cyt bc₁ complex of the clinical isolate Tm90-C2B.

ELQ-596 Metabolic Stability. We then evaluated ELQ-596 for metabolic stability in the presence of pooled murine hepatic derived microsomes. Because of its close structural similarity to ELQ-300, we expected the new analog to be stable under the conditions of the assay. The drug was incubated in the presence of pooled murine liver microsomes (0.5 mg/ml) at 37° C. in the presence of NADPH to test for P450 drug dependent metabolism. Samples were taken over the interval of 45 minutes and analyzed by LC-MS/MS for the presence of test compound. Ketanserin served as an internal standard for the metabolic rate of a known drug with known intermediate stability. As shown in Table 3, tests demonstrated extreme stability of ELQ-596 to microsomal attack with negligible breakdown over the course of 45 minutes of incubation yielding an estimated T_{1/2} in this in vitro assay of >4,000 minutes.

TABLE 3
Metabolic stability of ELQ-596 in
the presence of murine microsomes.

| Test | | | Clᵢₙₜ |
|---|---|---|---|
| compound | Species | T_{1/2} (minutes) | (mL/min/kg) |
| Ketanserin | Mouse | 19.10 | 285.69 |
| ELQ-596 | Mouse | 4,260 | 1.15 |

In Vivo Efficacy of ELQ-596 and Alkoxycarbonate Ester Prodrug ELQ-598 against Murine Malaria. Next, we were interested in testing ELQ-596 in vivo. Because it is a highly crystalline compound like ELQ-300 we prepared an alkoxycarbonate ester prodrug, ELQ-598. And, like ELQ-331, ELQ-598 exhibits significantly reduced crystal lattice energy as evidenced by a 229° C. decrease in melting point (Table 3). We tested ELQ-598 in the 4-day test using a modified Peters protocol in which all test animals are first inoculated with 35,000 infected red cells from a donor mouse infected with P. yoelii via tail vein injection (Day 0). Animals were then dosed with ELQ-598 dissolved in PEG400 (100 μl) by oral gavage on Days 1, 2, 3 and 4. On Day 5 a drop of blood was taken from the tail and a blood smear was prepared, fixed with methanol, and stained with Giemsa. The operator then examined the stained smear microscopically to determine percent parasitemia. Dosages of 0.0025, 0.005, 0.01, 0.03, 0.1, 0.3, 1.0 and 10 mg/kg/day were used for the experiment. From two separate studies (4 mice per group) the average estimates for ED₅₀ and ED₉₀ were 0.006 and 0.01 mg/kg/day, respectively, with a non-recrudescence dose (NRD) of 0.1 mg/kg/day (Table 4). These values are roughly 3-fold lower than for ELQ-300 and ELQ-331. Gratifyingly, the superiority of ELQ-596 carried over to single dose cures (SDC) for prodrug ELQ-598. In this model animals were inoculated exactly as for the 4-day test on Day 0 however drug was administered only on Day 1 while the operator made smears on Day 5 and again weekly thereafter for animals that remained aparasitemic. Animals that remained aparasitemic out to Day were scored as cures. In this latter experiment the lowest fully protective single dose cure was at 0.5 mg/kg (0.6 mg/kg of prodrug)—the lowest dose tested to date. Thus, prodrug ELQ-598 is at least 6 times more effective as a single dose cure against blood stage malaria infections in mice compared directly to ELQ-331.

TABLE 4
Comparison of ELQ-300, prodrug ELQ-331,
and ELQ-596 and prodrug ELQ-598.

| | | In vivo efficacy, mg/kg/day | | | |
|---|---|---|---|---|---|
| | Melting Point, | 4-day Peters Test Results | | | Single |
| Code | ° C. | ED₅₀ | ED₉₀ | NRD | dose cure |
| ELQ-300 | 316 | 0.02 | 0.06 | 1.0 | >25 |
| ELQ-331 | 117 | 0.02 | 0.05 | 1.0 | 3 |
| ELQ-596 | 365 | 0.01 | 0.021 | 0.3 | NT |
| ELQ-598 | 136 | 0.006 | 0.01 | 0.1 | ≤0.5 |
| ATV | 217 | 0.1 | ND | 10 | 10 |
| CQ | NT | 1.6 | 2.7 | >64 | >64 |

MP = melting point; ED₅₀—dose required to suppress parasitemia by 50% relative to untreated controls (4-day Peters test), ED₉₀—dose required to suppress parasitemia by 90% relative to untreated controls (4-day Peters test, P. yoelii Kenya Strain), NRD—non-recrudescence dose (4-day Peters test), and SDC—single dose cure (lowest single dose that provides complete cures of all 4 mice in the group). NT = not tested. ND = Not determined.
Note:
Prodrugs were dosed based on molar equivalency to the parent drug.

Selective Inhibition of Parasite Cytochrome bc₁ complex by ELQ-596. The ability of ELQ-596 to inhibit cytochrome bc₁ activity from P. falciparum mitochondria was assessed. As shown in Table 5, ELQ-596 showed potent inhibitory action of the P. falciparum cytochrome bc₁ complex, with an ICs value of 0.1 nM. This value is much lower than IC₅₀ values previously cited for either atovaquone or ELQ-300. Notice that the prodrug ELQ-598 exhibits only feeble inhibitory activity against the parasite enzyme. We also evaluated ELQ-596 for inhibition of the human host cytochrome bc₁ complex isolated from human liver tissue and found no detectable inhibition at a concentration of 10,000 nM. Together, our data show that ELQ-596 is a highly selective inhibitor of plasmodial cytochrome bc₁ complexes with a selectivity index that is ≥18,000-fold based on enzyme inhibitory activity. Such a high level of selectivity suggests a low potential for side effects in humans due to inhibition of the host enzyme complex.

TABLE 5
Comparative inhibition of P. falciparum (parasite)
and human (host) cytochrome bc1 complex.

| | P. falciparum | Human liver | |
|---|---|---|---|
| | cytochrome | cytochrome | Selectivity Index |
| Compound | bc₁, nM | bc₁, nM | Human/Pf cyt. bc₁ |
| Atovaquone | 2.0^{a} | 460^{a} | 230 |
| ELQ-300 | 0.56^{a} | >10,000^{a} | ≥18,000 |
| ELQ-331 | 5,300^{b} | >10,000^{b} | NA |
| (prodrug) | | | |
| ELQ-596 | 0.1 | >10,000 | ≥100,000 |
| ELQ-598 | x | >10,000 | NA |
| (prodrug) | | | |

^{a}Data taken from Nilsen et al., 2013.
^{b}Data taken from Frueh et al., 2017.
Assay conditions are presented in the Methods section.

Safety and Mitochondrial Toxicity of ELQ-596 and Prodrug ELQ-598. Of course, enhanced potency is desirable only if unaccompanied by enhanced toxicity. Although our in vivo efficacy-testing model is not intended as a formal toxicity assessment, there were no appearance, behavioral or weight changes observed after dosing with ELQ-598 at any dose level. We also evaluated ELQ-596 for cytotoxicity using the TiterGlo luminescence assay kit, which determines cell viability by measuring cellular ATP. In the assay, ATP is consumed as a co-substrate of luciferase on reaction with its substrate luciferin with release of light. Using the human HepG2 cell line in culture medium in which glucose was replaced by galactose to promote reliance upon oxidative phosphorylation processes and to reverse the so-called “Crabtree effect”, we observed an EC₅₀ of >10 μM for ELQ-596 while the control drug, rotenone, proved quite cytotoxic under these conditions (EC₅₀=?) (Table 6). The incubation period for these experiments was 48 hours.

TABLE 6
Comparative inhibition of P. falciparum (parasite)
and human (host) cytochrome bc₁ complex.

| | Cytotoxicity, nM |
|---|---|
| Compound | HepG2 Cells |
| Atovaquone | NT |
| ELQ-300 | >10,000 |
| ELQ-331 (prodrug) | NT |
| ELQ-596 | >10,000 |
| ELQ-598 (prodrug) | NT |

Cytotoxicity experiments were performed in medium in which glucose was substituted by galactose to reverse the Crabtree effect. Cyt bc₁ assay conditions are presented in the Methods section. NT = Not tested.

Based on pharmacokinetics experiments that were performed previously in mice, rats, and dogs, pharmacology experts predict that a single 30 mg oral dose of formulated ELQ-331 will protect adults from malaria infection if taken weekly. We feel that our “backup plan” could deliver a more potent drug, perhaps prodrug ELQ-598 or variant thereof, that could provide the same degree of long-term protection but at a significantly lower dose, perhaps 5 to 10 mg on a weekly or biweekly schedule.

### Materials and Methods

### Chemical Synthesis Procedures.

Unless otherwise stated all chemicals and reagents were from Sigma-Aldrich Chemical Company in St. Louis, MO (USA), Combi-Blocks, San Diego (CA), or TCl America, Portland (OR) and were used as received. Quinolone 1 and 4,4,5,5-tetramethyl-2-(4-(4-(trifluoromethoxy)phenoxy)phenyl)-1,3,2-dioxaborolane (14k) were obtained as previously reported¹². Melting points were obtained in the Optimelt Automated Melting point system from Stanford Research Systems, Sunnyvale, CA (USA). Analytical TLC utilized Merck 60F-254 250 micron precoated silica gel plates and spots were visualized under 254 nm UV light. GC-MS was obtained using an Agilent Technologies 7890B gas chromatograph (30 m, DBS column set at either 100° C. or 200° C. for 2 min, then at 30° C./min to 300° C. with inlet temperature set at 250° C.) with an Agilent Technologies 5977A mass-selective detector operating at 70 eV. Flash chromatography over silica gel column was performed using an IsoleraOne flash chromatography system from Biotage, Uppsala, Sweden. ¹H-NMR spectra were obtained using a Bruker 400 MHz Avance NEO NanoBay NMR spectrometer operating at 400.14 MHz. The NMR raw data were analyzed using the iNMR Spectrum Analyst software. ¹H chemical shifts are reported in parts per million (ppm) relative to internal tetramethylsilane (TMS) standard or residual solvent peak. Coupling constant values (J) are reported in hertz (Hz). Decoupled ¹⁹F operating at 376 MHz was also obtained for compounds containing fluorine (data not shown). HPLC analyses were performed using an Agilent 1260 Infinity instrument with detection at 254 nm and a Phenomenex, Luna® 5 μm C8(2) 100 Å reverse phase LC column 150×4.6 mm at 40° C., and eluted with a gradient of A/B at 25%/75% to A/B at 25% to 90% (A: 0.05% formic acid in milliQ water, B: 0.05% formic acid in methanol). All compounds were >95% pure for in vitro testing and >98% pure for in vivo testing as determined by GC-MS, ¹H-NMR and HPLC.

4,6-dichloro-3-iodo-7-methoxy-2-methylquinoline (2). A stirred solution of 4(1H)-Quinolone 1 (10.0 g, 28.6 mmol, 1 eq) and POC (14 ml, 146 mmol, 5.1 eq) in DCM (100 ml) was refluxed for 72 h. After cooling to room temperature, the mixture was filtered and the precipitate washed with DCM (3×5 ml) and air dried to give pure 2 (9.8 g, 93% yield) as a white powder. GC-MS shows one peak M⁺=366.9 (100%). ¹H-NMR (400 MHz; DMSO-d₆): δ 8.23 (s, 1H), 7.70 (s, H), 7.08 (s, 1H), 4.07 (s, 3H), 2.42 (s, 3H).

### 4,4,5,5-tetramethyl-2-(4′-(trifluoromethoxy)-[1,1′-biphenyl]-4-yl)-1,3,2-dioxaborolane (4)

### 6-chloro-7-methoxy-2-methyl-3-(4′-(trifluoromethoxy)-[1,1′-biphenyl]-4-yl)quinolin-4(1H)-one (ELQ-596)

Ethyl 2-(4′-(trifluoromethoxy)-[1,1′-biphenyl]-4-yl)acetate (6). A stirred mixture of ethyl 2-(4-bromophenyl)-acetate 5 (24.3 g, 100.0 mmol, 1.0 eq), (4-(trifluoromethoxy)phenyl)boronic acid (24.72 g, 120.0 mmol, 1.2 eq), K₂CO₃ (27.6 g, 200.0 mmol, 1.2 eq) and (Pd(dppf)Cl₂) (3.65 g, 5.0 mmol, 0.05 eq) in DMF (250 ml) was deoxygenated by bubbling argon through the reaction mixture for 15 minutes. The stirred reaction mixture was then heated at 80° C. under argon for 18 hours, until no more starting material 5 remained as determined by GC-MS. The reaction was cooled to room temperature and filtered through celite, and DMF was removed in vacuo. The resulting black, oily solid was resuspended in DCM (500 ml) and stirred vigorously at room temperature for 30 minutes, filtered through celite, concentrated to dryness and purified by flash chromatography over silica gel using a gradient of ethyl acetate/hexane (1/9) as the eluting solvent mixture to give 6 (18.7 g, 58% yield) as a white solid. GC-MS shows one peak M⁺=324.1 (42%); 251.2 (100%). ¹H-NMR (400 MHz; CDCl₃): δ 7.63-7.59 (m, 2H), 7.56-7.53 (m, 2H), 7.41-7.38 (m, 2H), 7.32-7.29 (m, 2H), 4.21 (q, J=7.1 Hz, 2H), 3.69 (s, 2H), 1.32-1.29 (t, J=7.1 Hz, 3H).

Ethyl 3-acetoxy-2-(4-bromophenyl)but-2-enoate (7a). Temperatures given were recorded by an internal thermometer. A stirred solution of dry THF (50 ml) and HMDS (41.5 g, 257.0 mmol, 2.5 eq) under Ar was cooled to −20° C. in an 75% ethylene glycol, 25% ethanol and dry ice bath. While monitoring the temperature to ensure that it did not exceed −10° C., n-butyl-lithium (2.5 M) in hexane (n-BuLi) (98.8 mL, 247.0 mol, 2.4 eq) was added. The temperature of the mixture was then lowered to −30° C. and a solution of 5 (25.0 g, 103.0 mol, 1.0 eq) in THF (50 ml) was slowly added. The mixture was allowed to warm up to −10° C. and stirred for 35 min while maintaining this temperature. Next, acetic anhydride (31.5 g, 309.0 mmol, 3.0 eq) was added dropwise, then the mixture was allowed to slowly warm up to room temperature. The mixture turned cloudy as it warmed up, but did not jellify. The reaction progress was monitored by GC-MS, and after 1 h at 25° C. there was still 25% starting material present. An additional acetic anhydride (3.15 g, 30.0 mmol, 0.3 eq) was added. After stirring at room temperature for 72 hours 19% of starting material was still present as determined by GC-MS. The reaction was stopped and the mixture was poured into saturated ammonium chloride solution (100 ml), extracted with ethyl acetate (3×100 ml), then the organic layers were combined and concentrated to give 35.0 g of a brown oil. GC-MS analysis showed one major peak (100%) with M⁺=326 (2%), 238 (100%), one minor peak (27%) with M⁺=326 (2%), 238 (100%) and another minor peak (19%, corresponding to 5) with M⁺=242 (25%), 168.9 (100%). The peaks with M⁺=326 correspond to the stereoisomers E and Z of the desired product 7a. Two D NOESY NMR did not provide unambiguous assignment of the two stereoisomers. The percent of the major stereoisomer relative to the minor stereoisomer was estimated to be 80% by GC-MS. The product can be used without further purification in the next step.

For analysis and characterization purpose the two stereoisomers were purified by flash chromatography using hexane and ethyl acetate (5 to 15% gradient).

NMR of the major stereoisomer of 7a: ¹H-NMR (400 MHz; CDCl₃): δ 7.51-7.48 (m, 2H), 7.18-7.15 (m, 2H), 4.14 (q, J=7.1 Hz, 2H), 2.23 (s, 3H), 1.88 (s, 3H), 1.19 (t, J=7.1 Hz, 3H).

NMR of the minor stereoisomer of 7a: ¹H-NMR (400 MHz; CDCl₃): δ 7.47-7.44 (m, 2H), 7.08-7.05 (m, 2H), 4.20 (q, J=7.1 Hz, 2H), 2.40 (s, 3H), 1.88 (s, 3H), 1.23 (t, J=7.1 Hz, 3H).

Ethyl 3-acetoxy-2-(4′-(trifluoromethoxy)-[1,1′-biphenyl]-4-yl)but-2-enoate (7b). Temperatures given were recorded by an internal thermometer. A stirred solution of dry THF (50 ml) and HMDS (16.6 g, 102.9 mmol, 2.3 eq) under Ar was cooled to −20° C. in 75% ethylene glycol, 25% ethanol and dry ice bath. While monitoring the temperature to ensure that it did not exceed −10° C., n-butyl-lithium (2.5 M) in hexane (n-BuLi) (39.4 mL, 98.5 mmol, 2.2 eq), followed by a solution of 6 (14.5 g, 44.75 mmol, 1.0 eq) in THF (50 ml) were added dropwise. After stirring for 35 minutes at −15° C. to −10° C., acetic anhydride (10.05 g, 11.3 ml, 98.5 mmol, 2.2 eq) was added dropwise while monitoring the temperature not to exceed −10° C. The solution was then allowed to gradually warm to room temperature, when it turned into a light-yellow gel. After stirring 20 h at room temperature, the mixture was poured into saturated ammonium chloride solution (200 ml), extracted with ethyl acetate (3×100 ml), then the organic layers were combined and concentrated to give 17.3 g of a brown oil. GC-MS analysis showed one major peak (100%) with M⁺=408 (3%), 320 (100%), one minor peak (5%) with M⁺=408 (3%), 320 (100%), and another minor peak (5% corresponding to the starting material 6) with M⁺=324 (42%), 251 (100%). The peaks with M⁺=408 correspond to the mixture of the stereoisomers E and Z of the desired product 7b. Two D NOESY NMR did not provide unambiguous assignment of the two stereoisomers. The percent of the major stereoisomer relative to the minor stereoisomer was estimated to be 95% by GC-MS. The product can be used without further purification in the next step.

For analysis and characterization purpose the two stereoisomers were purified by flash chromatography using hexane and ethyl acetate (5 to 50% gradient).

NMR of the major stereoisomer of 7b: ¹H-NMR (400 MHz; CDCl₃): δ 7.66-7.62 (m, 2H), 7.60-7.57 (m, 2H), 7.41-7.38 (m, 2H), 7.32-7.30 (m, 2H), 4.20 (q, J=7.1 Hz, 2H), 2.27 (s, 3H), 1.98 (s, 3H), 1.25 (t, J=7.1 Hz, 3H).

NMR of the minor stereoisomer of 7b: ¹H-NMR (400 MHz; CDCl₃): δ 7.66-7.62 (m, 2H), 7.57-7.54 (m, 2H), 7.32-7.29 (m, 4H), 4.25 (q, J=7.1 Hz, 2H), 2.44 (s, 3H), 1.90 (s, 3H), 1.27 (t, J=7.1 Hz, 3H).

Ethyl 2-(4-bromophenyl)-3-oxobutanoate (8a). A stirred solution of the bis-acylated 7a (32.6 g, 0.10 mol, 1 eq) in glacial acetic acid (100 ml) and p-TsOH monohydrate 98% (1.9 g, 0.1 mol, 0.1 eq) was heated at 100° C. After 2 hours no more, starting material 7a was detected by TLC and GC-MS. The dark brown solution was cooled to room temperature and concentrated under vacuum. After most of the acetic acid was eliminated, cyclohexane (2×100 ml) was added to the brown oil and concentrated again to give 30.5 g of 8a as a dark brown oil. Because this material still contained 1.9 g of p-TsOH, the yield of 8a was 28.6 g (100% yield). The product can be used without purification in the following Conrad-Limpach reaction. Both the keto and enol forms can be detected by ¹H-NMR.

Ethyl 3-hydroxy-2-(4′-(trifluoromethoxy)-[1,1′-biphenyl]-4-yl)but-2-enoate (8b). A stirred solution of the bis-acylated 7b (11.8 g, 0.29 mol, 1 eq) in glacial acetic acid (25 ml) and p-TsOH monohydrate 98% (549 mg, 0.29 mol, 0.1 eq) was heated at 100° C. After 16 hours no more, starting material 7b was detected by TLC and GC-MS. Of note the β-keto ester 8b decomposed in injection port of the mass spectrometer to give a major peak with M⁺=294 (32%), 251 (100%). The dark brown solution was cooled to room temperature and concentrated under vacuum. After most of the acetic acid was eliminated, cyclohexane (2×50 ml) was added to the brown oil and concentrated again to give 10.0 g of 10b as a dark brown oil. Because this material still contained 549 mg of p-TsOH, the yield of 5 was 9.45 g (89% yield). The product can be used without purification in the following Conrad-Limpach reaction. Both the keto and enol forms can be detected by ¹H-NMR.

General procedure for the preparation of Schiff bases (10a-d and 11). A stock solution of β-keto ester 8a or 8b containing 0.1 eq of p-TsOH (0.25 mM) in benzene was prepared (0.92 g/10 ml=2.5 mM) and kept. A stirred solution of a substituted aniline (9a-d) in benzene and an aliquot of the β-keto ester 8a or 8b was heated at reflux for 24-72 h using a Dean-Stark trap to continuously remove water azeotropically and monitored for the disappearance of β-keto ester 8a or 8b by GC-MS. The solution was then concentrated in vacuo to give the product Schiff bases (10a-d and 11) as a yellow-brown, highly viscous oil.

General procedure for the Conrad-Limpach reaction (ELQ). The intermediate Schiff base (10a-d and 11) was diluted with 5 ml of warm Dowtherm A and added to 65 ml of boiling Dowtherm A (250° C.) in portions over approximately 5 minutes with vigorous stirring to maintain the boiling of Dowtherm A. The mixture was kept at boiling for another 5 minutes, then allowed to cool to room temperature and diluted with hexane (250 ml) resulting in the formation of a precipitate which was filtered and washed with ethyl acetate and acetone until a colorless filtrate was obtained.

### 2-methyl-3-(4′-(trifluoromethoxy)-[1,1′-biphenyl]-4-yl)quinolin-4(1H)-one (ELQ-649)

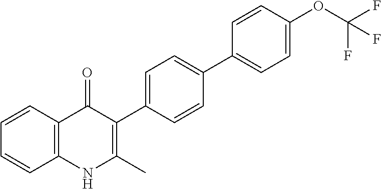 [SMILES: Cc1[nH]c2ccccc2c(=O)c1-c1ccc(-c2ccc(OC(F)(F)F)cc2)cc1]

Following the general procedure for the preparation of the Schiff base, a mixture of aniline 9a (0.47 g, 5.0 mol, 1 eq), benzene (125 ml), β-keto ester 8b (5.0 mmol, 1 eq) containing p-TsOH (0.5 mmol, 0.1 eq) was heated at reflux for 24 h. Then following the general procedure of the Conrad-Limpach reaction to give ELQ-649 (0.64 g, 32% yield) after crystallization using DMF as a white powder. ¹H-NMR (400 MHz; DMSO-d₆): δ 11.68 (s, 1H), 8.10 (dd, J=8.1, 1.5 Hz, 1H), 7.87-7.83 (m, 2H), 7.73-7.70 (m, 2H), 7.65 (ddd, J=8.4, 6.9, 1.5 Hz, 1H), 7.56-7.54 (m, 1H), 7.49-7.46 (m, 2H), 7.39-7.36 (m, 2H), 7.30 (ddd, J=8.1, 6.9, 1.1 Hz, 1H), 2.29 (s, 3H). The product is >98% pure by HPLC and ¹H-NMR.

### 6-chloro-7-methoxy-2-methyl-3-(4′-(trifluoromethoxy)-[1,1′-biphenyl]-4-yl)quinolin-4(1H)-one (ELQ-596)

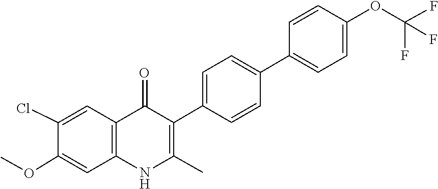 [SMILES: COc1cc2[nH]c(C)c(-c3ccc(-c4ccc(OC(F)(F)F)cc4)cc3)c(=O)c2cc1Cl]

Following the general procedure for the preparation of the Schiff base, a mixture of aniline 9b (0.78 g, 5.0 mmol, 1 eq), benzene (125 ml), β-keto ester 8b (5.0 mmol, 1 eq) containing p-TsOH (0.5 mmol, 0.1 eq) was heated at reflux for 72 h. Then following the general procedure of the Conrad-Limpach reaction to give ELQ-596 (0.77 g, 34% yield) as a white powder. ¹H-NMR (400 MHz; DMSO-d₆): δ 11.69 (s, 1H), 8.01 (s, 1H), 7.87-7.83 (m, 2H), 7.72-7.69 (m, 2H), 7.50-7.44 (m, 2H), 7.38-7.35 (m, 2H), 7.09 (s, 1H), 3.97 (s, 3H), 2.26 (s, 3H). The product is >98% pure by HPLC and ¹H-NMR.

### 6-fluoro-7-methoxy-2-methyl-3-(4′-(trifluoromethoxy)-[1,1′-biphenyl]-4-yl)quinolin-4(1H)-one (ELQ-650)

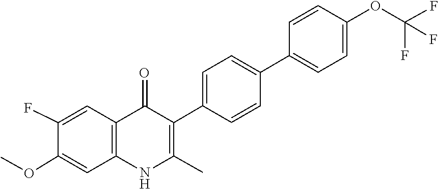 [SMILES: COc1cc2[nH]c(C)c(-c3ccc(-c4ccc(OC(F)(F)F)cc4)cc3)c(=O)c2cc1F]

Following the general procedure for the preparation of the Schiff base, a mixture of aniline 9c (0.71 g, 5.0 mmol, 1 eq), benzene (125 ml), β-keto ester 8b (5.0 mmol, 1 eq) containing p-TsOH (0.5 mmol, 0.1 eq) was heated at reflux for 46 h. Then following the general procedure of the Conrad-Limpach reaction to give ELQ-650 (0.61 g, 28% yield) as a white powder. ¹H-NMR (400 MHz; DMSO-d₆): δ 11.66 (s, 1H), 7.86-7.83 (m, 2H), 7.73-7.69 (m, 3H), 7.50-7.44 (m, 2H), 7.37-7.35 (m, 2H), 7.11 (d, J=7.4 Hz, 1H), 3.96 (s, 3H), 2.26 (s, 3H). The product is >98% pure by HPLC and ¹H-NMR.

### 5,7-difluoro-2-methyl-3-(4′-(trifluoromethoxy)-[1,1′-biphenyl]-4-yl)quinolin-4(1H)-one (ELQ-601)

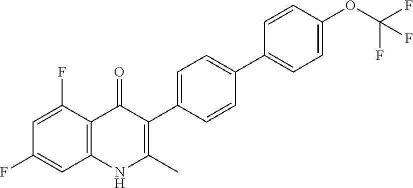 [SMILES: Cc1[nH]c2cc(F)cc(F)c2c(=O)c1-c1ccc(-c2ccc(OC(F)(F)F)cc2)cc1]

Following the general procedure for the preparation of the Schiff base, a mixture of aniline 9d (0.71 g, 5.0 mmol, 1 eq), benzene (125 ml), β-keto ester 8b (5.0 mmol, 1 eq) containing p-TsOH (0.5 mmol, 0.1 eq) was heated at reflux for 72 h. Then following the general procedure of the Conrad-Limpach reaction to give ELQ-601 (0.80 g, 37% yield) as a white powder. ¹H-NMR (400 MHz; DMSO-d₆): δ 11.76 (s, 1H), 7.87-7.83 (m, 2H), 7.73-7.69 (m, 2H), 7.48-7.46 (m, 2H), 7.36-7.32 (m, 2H), 7.11-7.08 (m, 1H), 7.04 (ddd, J=11.9, 9.6, 2.4 Hz, 1H), 2.22 (s, 3H). The product is >98% pure by HPLC and ¹H-NMR.

3-(4-bromophenyl)-6-chloro-7-methoxy-2-methylquinolin-4(1H)-one (12). Following the general procedure for the preparation of the Schiff base, a mixture of aniline 9b (13.0 g, 83.0 mmol, 1 eq), benzene (150 ml), β-keto ester 8a (23.7 g, 83.0 mmol, 1 eq) containing p-TsOH (8.3 mmol, 0.1 eq) was heated at reflux for 21 h. Then following the general procedure of the Conrad-Limpach reaction, using Dowtherm A (30 ml) to dilute the Schiff base and added to boiling Dowtherm A (200 ml). Upon cooling while stirring a precipitate was formed. Hexane (800 ml) was added resulting in the formation of a sticky solid which was filtered and stirred for 15 minutes with acetone (150 ml), filtered, washed with acetone (3×25 ml) and air dried to give pure 12 (14.7 g, 49.5% yield) as a white solid. ¹H-NMR (400 MHz; DMSO-d₆): δ 11.70 (s, 1H), 7.99 (s, 1H), 7.58-7.56 (m, 2H), 7.24-7.18 (m, 2H), 7.07 (s, 1H), 3.96 (s, 3H), 2.21 (s, 3H).

3-(4-bromophenyl)-4,6-dichloro-7-methoxy-2-methylquinoline (13). 4(1H)-Quinolone 12 (14.7 g, 39.0 mmol) was refluxed with POCl₃, (70 ml) for 45 minutes. After cooling to room temperature, the solution was poured slowly over 10 minutes in vigorously stirred water (800 ml) and stirred for an additional 5 minutes. The formed precipitate was washed with water (50 ml), acetone (2×25 ml) and air dried to give pure 13 (15.7 g, 100% yield). GC-MS shows one peak M⁺=395 (63%), 397 (100%), 399 (47%), 401 (10%). ¹H-NMR (400 MHz; DMSO-d₆): ¹H-NMR (400 MHz; DMSO-d₆): δ 8.18 (s, 1H), 7.76-7.73 (m, 2H), 7.64 (s, 1H), 7.37-7.34 (m, 2H), 4.05 (s, 3H), 2.38 (s, 3H).

General Procedure for the preparation of the biphenyl quinolines (15a-k). A stirred mixture of quinoline 13, substituted phenyl boronic acids 14a-g, 14i and 14j or pinacol esters 14 h and 14k, K₂CO₃ and Pd(dppf)Cl₂, in DMF was deoxygenated by bubbling argon through the solution for 15 minutes. The stirred reaction mixture was then heated at 80° C. under argon until almost no more starting material 13 remained as determined by GC-MS. The reaction was cooled to room temperature and filtered through celite, and DMF was removed in vacuo. The resulting black oily solid was resuspended in DCM and stirred vigorously at room temperature for 30 minutes, filtered through celite, and concentrated to dryness. The residue was taken up with 3-5 ml of DCM, if all the solid was dissolved then the product was purified by flash chromatography. In some instance the products were not soluble in methylene chloride they were filtered, washed with DCM and the filtrates were further purified by flash chromatography to give additional products.

General Procedure for the hydrolysis of the 4-chloro quinolines. A stirred mixture of the 4-chloro quinolines, potassium acetate (KOAc) and glacial acetic acid was heated at 120° C. in a loosely capped reaction vial for 16-26 h.

4,6-dichloro-3-(4′-chloro-[1,1′-biphenyl]-4-yl)-7-methoxy-2-methylquinoline (15a). Following the general procedure for the preparation of biphenyl quinolines, a mixture of 13 (740 mg, 1.86 mmol, 1 eq), 14a (435 mg, 2.79 mmol, 1.5 eq), K₂CO₃ (513 mg, 3.72 mmol, 2 eq) and Pd(dppf)Cl₂ (68 mg, 0.093 mmol, 0.05 eq) and DMF (75 ml) was heated for 16 h to give crude 25 (911 mg) as a black solid. DCM (5 ml) was added and the precipitate was filtered washed with methylene chloride (2×5 ml) to give pure 15a (158 mg) as a white solid. The filtrate was further purified by flash chromatography using a gradient of ethyl acetate/hexane (3/7) as the eluting solvent to yield additional 15a (99 mg) for a combined yield of 15a (257 mg, 32% yield). GC-MS shows one peak M⁺=427 (100%). ¹H-NMR (400 MHz; CDCl₃): δ 8.27 (s, 1H), 7.74-7.71 (m, 2H), 7.65-7.62 (m, 2H), 7.49-7.46 (m, 3H), 7.39-7.36 (m, 2H), 4.10 (s, 3H), 2.52 (s, 3H).

4,6-dichloro-7-methoxy-2-methyl-3-(4′-methyl-[1,1′-biphenyl]-4-yl)quinoline (15b). Following the general procedure for the preparation of biphenyl quinolines, a mixture 13 (794 mg, 2.0 mmol, 1 eq), 14b (326 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated for 16 h to give crude 15b (898 mg) as a black solid. The product was soluble in DCM and was purified by flash chromatography using a gradient of ethyl acetate/hexane (3/7) as the eluting solvent to give pure 15b (328 mg, 40% yield) as a white solid. GC-MS shows one peak M⁺=407 (100%). ¹H-NMR (400 MHz; CDCl₃): δ 8.27 (s, 1H), 7.76-7.73 (m, 2H), 7.62-7.59 (m, 2H), 7.49 (s, 1H), 7.37-7.30 (m, 4H), 4.09 (s, 3H), 2.53 (s, 3H), 2.45 (s, 3H).

3-(4′-(tert-butyl)-[1,1′-biphenyl]-4-yl)-4,6-dichloro-7-methoxy-2-methylquinoline (15c). Following the general procedure for the preparation of biphenyl quinolines, a mixture 13 (794 mg, 2.0 mmol, 1 eq), 14c (427 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated for 16 h to give crude 15c (1.032 g) as a black solid. The product was soluble in DCM and was purified by flash chromatography using a gradient of ethyl acetate/hexane (3/7) as the eluting solvent to give pure 15c (331 mg, 37% yield) as a white solid. GC-MS shows one peak M⁺=450 (63%), 434 (100%). ¹H-NMR (400 MHz; CDCl₃): δ 8.28 (s, 1H), 7.77-7.74 (m, 2H), 7.66-7.64 (m, 2H), 7.55-7.52 (m, 2H), 7.50 (s, 1H), 7.37-7.33 (m, 2H), 4.10 (s, 3H), 2.53 (s, 3H), 1.41 (s, 9H).

4,6-dichloro-7-methoxy-2-methyl-3-(4′-(trifluoromethyl)-[1,1′-biphenyl]-4-yl)quinoline (15d). Following the general procedure for the preparation of biphenyl quinolines, a mixture 13 (794 mg, 2.0 mmol, 1 eq), 14d (456 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated for 36 h to give crude 15d (1.12 g) as a reddish black solid. DCM (5 ml) was added and the precipitate was filtered washed with methylene chloride (2×5 ml) to give pure 15d (320 mg, 35% yield) as a white solid. GC-MS shows one peak M+=461 (100%). ¹H-NMR (400 MHz; CDCl₃): δ 8.28 (s, 1H), 7.83-7.80 (m, 2H), 7.78-7.76 (m, 4H), 7.50 (s, 1H), 7.42-7.40 (m, 2H), 4.10 (s, 3H), 2.53 (s, 3H).

4′-(4,6-dichloro-7-methoxy-2-methylquinolin-3-yl)-[1,1′-biphenyl]-4-carbonitrile (15e). Following the general procedure for the preparation of biphenyl quinolines, a mixture 13 (794 mg, 2.0 mmol, 1 eq), 14e (353 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated for 36 h to give crude 15e (769 mg) as a black solid. DCM (5 ml) was added and the precipitate was filtered washed with DCM (2×5 ml) to give 15e (384 mg) as a white solid. The product was further recrystallized from DMF to give pure 15e (290 mg) as a white solid. The filtrate was further purified by flash chromatography using a gradient of ethyl acetate/hexane (3/7) as the eluting solvent to yield an additional 29 (90 mg) for a combined yield of 15e (380 mg, 45% yield). GC-MS shows one peak M⁺=418 (100%). ¹H-NMR (400 MHz; CDCl₃): δ 8.26 (s, 1H), 7.82-7.79 (m, 4H), 7.77-7.75 (m, 2H), 7.49 (s, 1H), 7.44-7.41 (m, 2H), 4.09 (s, 3H), 2.51 (s, 3H).

4,6-dichloro-3-(4′-(difluoromethyl)-[1,1′-biphenyl]-4-yl)-7-methoxy-2-methylquinoline (15f). Following the general procedure for the preparation of biphenyl quinolines, a mixture 13 (397 mg, 1.0 mmol, 1 eq), 14f (206 mg, 1.2 mmol, 1.2 eq), K₂CO₃ (276 mg, 2.0 mmol, 2 eq) and Pd(dppf)Cl₂ (73 mg, 0.10 mmol, 0.1 eq) and DMF (3 ml) was heated for 36 h to give crude 15f as a brown solid. DCM (5 ml) was added and the precipitate was filtered washed with DCM (2×5 ml) to give 15f as a white solid. The product was further recrystallized from DMF to give pure 15f (220 mg, 50% yield) as a white solid. GC-MS shows one peak M⁺=443.1 (100%). ¹H-NMR (400 MHz; CDCl₃): δ 8.26 (s, 1H), 7.79-7.73 (m, 4H), 7.66-7.61 (m, 2H), 7.48 (s, 1H), 7.40-7.35 (m, 2H), 6.73 (t, 1H, J=57 Hz), 4.08 (s, 3H), 2.51 (s, 3H).

4,6-dichloro-7-methoxy-3-(4′-methoxy-[1,1′-biphenyl]-4-yl)-2-methylquinoline (15g). Following the general procedure for the preparation of biphenyl quinolines, a mixture 13 (794 mg, 2.0 mmol, 1 eq), 14g (365 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated for 16 h to give crude 15g (1.29 g) as a black solid. DCM (5 ml) was added and the precipitate was filtered washed with DCM (2×5 ml) to give pure 15g (255 mg) as a white solid. The filtrate was further purified by flash chromatography using a gradient of DCM/ethyl acetate (95/5) as the eluting solvent to yield an additional 15g (143 mg) for a combined yield of 15g (398 mg, 47% yield). GC-MS shows one peak M⁺=423 (100%). ¹H-NMR (400 MHz; CDCl₃): δ 8.27 (s, 1H), 7.73-7.71 (m, 2H), 7.66-7.64 (m, 2H), 7.49 (s, 1H), 7.35-7.33 (m, 2H), 7.05-7.03 (m, 2H), 4.09 (s, 3H), 3.90 (s, 3H), 2.53 (s, 3H).

4,6-dichloro-3-(4′-(difluoromethoxy)-[1,1′-biphenyl]-4-yl)-7-methoxy-2-methylquinoline (15h). Following the general procedure for the preparation the biphenyl quinolines, a mixture 13 (794 mg, 2.0 mmol, 1 eq), 14h (648 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated for 16 h to give crude 15h (1.073 g) as a black solid. DCM (5 ml) was added and the precipitate was filtered washed with DCM (2×5 ml) and then crystallize from DCM to give pure 15h (412 mg) as a white solid. The filtrate was further purified by flash chromatography using a gradient of ethyl acetate/hexane (3/7) as the eluting solvent to yield an additional 15h (140 mg) for a combined yield of 15h (552 mg, 60% yield). GC-MS shows one peak M⁺=459 (100%). ¹H-NMR (400 MHz; CDCl₃): δ 8.27 (s, 1H), 7.74-7.68 (m, 4H), 7.49 (s, 1H), 7.39-7.36 (m, 2H), 7.28-7.24 (m, 2H), 6.60 (t, J=73.8 Hz, 1H), 4.10 (s, 3H), 2.52 (d, J=2.9 Hz, 3H).

4,6-dichloro-7-methoxy-2-methyl-3-(3′-(trifluoromethyl)-[1,1′-biphenyl]-4-yl)quinoline (15i). Following the general procedure for the preparation the biphenyl quinolines, a mixture 13 (794 mg, 2.0 mmol, 1 eq), 14i (456 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated at 120° C. for 36 h to give crude 15i (932 mg) as a black solid. The product was soluble in DCM and was purified by flash chromatography using a gradient of ethyl acetate/hexane (6/4) as the eluting solvent to give about 95% pure 15i (100 mg, 11% yield) as a white solid. GC-MS shows one major peak M⁺=461 (100%). ¹H-NMR (400 MHz; CDCl₃): δ 8.28 (s, 1H), 7.96-7.88 (m, 2H), 7.79-7.76 (m, 2H), 7.69-7.63 (m, 2H), 7.50 (s, 1H), 7.43-7.40 (m, 2H), 4.10 (s, 3H), 2.53 (s, 3H).

4,6-dichloro-7-methoxy-2-methyl-3-(3′-(trifluoromethoxy)-[1,1′-biphenyl]-4-yl)quinoline (15j). Following the general procedure for the preparation the biphenyl quinolines, a mixture 13 (794 mg, 2.0 mmol, 1 eq), 14j (494 mg, 2.4 mmol, 1.2 eq), K₂CO₃ (552 mg, 4.0 mmol, 2 eq) and Pd(dppf)Cl₂ (73 mg, 0.10 mmol, 0.05 eq) and DMF (75 ml) was heated for 16 h to give crude 15j (1.249 g) as a black solid. The product was soluble in DCM and was purified by flash chromatography using a gradient of ethyl acetate/hexane (3/7) as the eluting solvent to give 15j (639 mg, 67% yield) as a white solid. GC-MS shows one peak M⁺=477 (100%) and one minor peak M⁺=397 (100%) corresponding to the starting material 13. GC-MS and NMR indicated that 15j is pure (˜95%) enough to use for the next step.

4,6-dichloro-7-methoxy-2-methyl-3-(4′-(4-(trifluoromethoxy)phenoxy)-[1,1′-biphenyl]-4-yl)quinoline (15k). Following the general procedure for the preparation the biphenyl quinolines, a mixture 13 (794 mg, 2.0 mmol, 1 eq), 14k (1.18 g, 3 mmol, 1.5 eq), K₂CO₃ (552 mg, 4.0 mmol, 2 eq) and Pd(dppf)Cl₂ (73 mg, 0.10 mmol, 0.05 eq) and DMF (75 ml) was heated for 48 h to give crude 15k (1.42 g) as a black solid. DCM (5 ml) was added and the precipitate was filtered washed with DCM (2×5 ml) to give pure 15k (110 mg) as a white solid. The filtrate was further purified by flash chromatography using a gradient of ethyl acetate/hexane (3/7) as the eluting solvent to yield an additional 15k (489 mg) for a combined yield of 15k (599 mg, 53% yield). GC-MS shows one peak M⁺=569 (100%). ¹H-NMR (400 MHz; CDCl₃): δ 8.28 (s, 1H), 7.75-7.72 (m, 2H), 7.71-7.69 (m, 2H), 7.50 (s, 1H), 7.39-7.35 (m, 2H), 7.26-7.23 (m, 2H), 7.17-7.13 (m, 2H), 7.12-7.08 (m, 2H), 4.10 (s, 3H), 2.53 (s, 3H).

### 6-chloro-3-(4′-chloro-[1,1′-biphenyl]-4-yl)-7-methoxy-2-methylquinolin-4(1H)-one (ELQ-637)

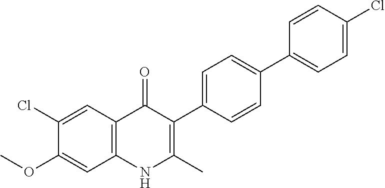 [SMILES: COc1cc2[nH]c(C)c(-c3ccc(-c4ccc(Cl)cc4)cc3)c(=O)c2cc1Cl]

Following the general procedure for the hydrolysis of the 4-chloro quinolines, a mixture of 15a (157 mg, 0.3 mmol, 1 eq), KOAc (360 mg, 3.7 mmol, 10 eq) and glacial acetic acid (10 ml) was heated for 26 h. After cooling to room temperature, the reaction mixture was further cooled to 4° C. The resulting solid was recovered by vacuum filtration, rinsing with excess water followed by acetone (3×5 ml) and airdried to give ELQ-637 as a pale taupe powder (0.104 g, yield 69%). ¹H-NMR (400 MHz; DMSO-d₆): δ 11.70 (s, 1H), 8.01 (s, 1H), 7.78-7.73 (m, 2H), 7.72-7.66 (m, 2H), 7.57-7.51 (m, 2H), 7.38-7.32 (m, 2H), 7.08 (s, 1H), 3.97 (s, 3H), 2.26 (s, 3H).

### 6-chloro-7-methoxy-2-methyl-3-(4′-methyl-[1,1′-biphenyl]-4-yl)quinolin-4(1H)-one (ELQ-603)

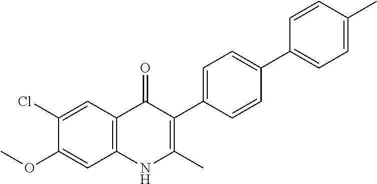

Following the general procedure for the hydrolysis of the 4-chloro quinolines, a mixture of 15b (204 mg, 0.5 mmol, 1 eq), KOAc (490 mg, 5.0 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give pure ELQ-603 (170 mg, 87% yield) as a white solid. ¹H-NMR (400 MHz; DMSO-d₆): δ 11.69-11.67 (s, 1H), 8.02 (s, 1H), 7.66-7.60 (m, 4H), 7.32-7.28 (m, 4H), 7.08 (s, 1H), 3.97 (s, 3H), 2.36-2.33 (s, 3H), 2.25 (s, 3H).

### 3-(4′-(tert-butyl)-[1,1′-biphenyl]-4-yl)-6-chloro-7-methoxy-2-methylquinolin-4(1H)-one (ELQ-651)

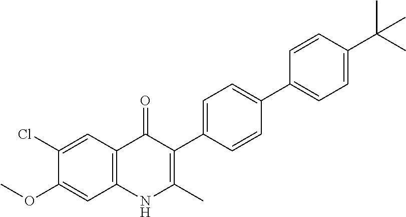 [SMILES: COc1cc2[nH]c(C)c(-c3ccc(-c4ccc(C(C)(C)C)cc4)cc3)c(=O)c2cc1Cl]

Following the general procedure for the hydrolysis of the 4-chloro quinolines, a mixture of 15c (225 mg, 0.5 mmol, 1 eq), KOAc (490 mg, 5.0 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give pure ELQ-651 (184 mg, 85% yield) as a white solid. ¹H-NMR (400 MHz; DMSO-d₆): δ 11.68 (s, 1H), 8.02 (s, 1H), 7.67-7.64 (m, 4H), 7.51 (m, 2H), 7.34-7.32 (m, 2H), 7.09 (s, 1H), 3.98 (s, 3H), 2.27 (s, 3H), 1.34 (s, 9H).

### 6-chloro-7-methoxy-2-methyl-3-(4′-(trifluoromethyl)-[1,1′-biphenyl]-4-yl)quinolin-4(1H)-one (ELQ-647)

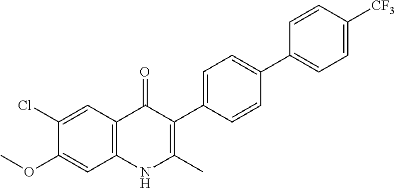 [SMILES: COc1cc2[nH]c(C)c(-c3ccc(-c4ccc(C(F)(F)F)cc4)cc3)c(=O)c2cc1Cl]

Following the general procedure for the hydrolysis of the 4-chloro quinolines, a mixture of 15d (231 mg, 0.5 mmol, 1 eq), KOAc (490 mg, 5.0 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give pure ELQ-647 (180 mg, 81% yield) as a white solid. ¹H-NMR (400 MHz; DMSO-d₆): δ 11.71 (s, J=0.2 Hz, 1H), 8.02 (s, J=1.7 Hz, 1H), 7.98-7.95 (m, 2H), 7.85-7.83 (m, 2H), 7.79-7.77 (m, 2H), 7.41-7.39 (m, 2H), 7.10 (s, 1H), 3.98 (s, 3H), 2.27 (s, 3H).

### 4′-(6-chloro-7-methoxy-2-methyl-4-oxo-1,4-dihydroquinolin-3-yl)-[1,1′-biphenyl]-4-carbonitrile (ELQ-602)

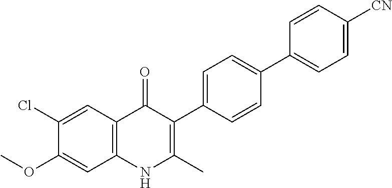 [SMILES: COc1cc2[nH]c(C)c(-c3ccc(-c4ccc(C#N)cc4)cc3)c(=O)c2cc1Cl]

Following the general procedure for the hydrolysis of the 4-chloro quinolines, a mixture of 15e (231 mg, 0.5 mmol, 1 eq), KOAc (490 mg, 5.0 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give pure ELQ-602 (172 mg, 86% yield) as a white solid. ¹H-NMR (400 MHz; DMSO-d₆): δ 11.70 (s, 1H), 7.96 (broad d, J=32.4 Hz, 4H), 7.77 (broad d, J=7.9 Hz, 2H), 7.39 (broad d, J=7.8 Hz, 2H), 7.07 (s, 1H), 3.97 (s, 3H), 2.26 (s, 3H).

### 6-chloro-3-(4′-(difluoromethyl)-[1,1′-biphenyl]-4-yl)-7-methoxy-2-methylquinolin-4(1H)-one (ELQ-659)

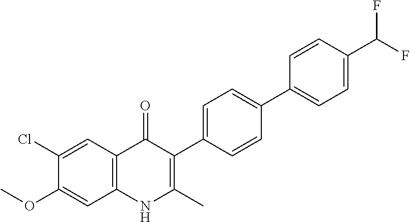 [SMILES: COc1cc2[nH]c(C)c(-c3ccc(-c4ccc(C(F)F)cc4)cc3)c(=O)c2cc1Cl]

Following the general procedure for the hydrolysis of the 4-chloro quinolines, a mixture of 15f (210 mg, 0.5 mmol, 1 eq), KOAc (490 mg, 5.0 mmol, 10 eq) and glacial acetic acid (5 ml) was heated for 16 h. After cooling to room temperature, the reaction mixture was poured into ice water (10 ml). The resulting precipitate was filtered washed with water (3×3 ml), acetone (2×3 ml), methylene chloride (2×3 ml), hexane (20 ml) and air-dried to give pure ELQ-659 (105 mg, 52% yield) as a white solid. ¹H-NMR (400 MHz; DMSO-d₆): δ 11.70 (s, 1H), 8.02 (s, 1H), 7.87 (d, J=7.9 Hz, 2H), 7.74 (d, J=7.9 Hz, 2H), 7.68 (d, J=7.9 Hz, 2H), 7.37 (d, J=7.9 Hz, 2H), 7.10 (t, J=56.3 Hz, 1H), 7.09 (s, 1H), 3.98 (s, 3H), 2.27 (s, 3H).

### 6-chloro-7-methoxy-3-(4′-methoxy-[1,1′-biphenyl]-4-yl)-2-methylquinolin-4(1H)-one (ELQ-645)

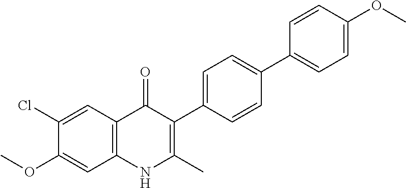

Following the general procedure for the hydrolysis of the 4-chloro quinolines, a mixture of 15g (212 mg, 0.5 mmol, 1 eq), KOAc (490 mg, 5.0 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give pure ELQ-645 (160 mg, 79% yield) as a white solid. ¹H-NMR (400 MHz; DMSO-d₆): δ 11.67 (s, 1H), 8.02 (s, 1H), 7.68-7.62 (m, 3H), 7.32-7.29 (m, 2H), 7.09-7.04 (m, 2H), 3.98 (s, 3H), 3.82 (s, 3H), 2.26 (s, 2H).

### 6-chloro-3-(4′-(difluoromethoxy)-[1,1′-biphenyl]-4-yl)-7-methoxy-2-methylquinolin-4(1H)-one (ELQ-600)

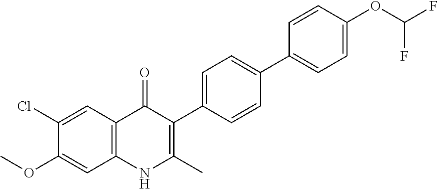 [SMILES: COc1cc2[nH]c(C)c(-c3ccc(-c4ccc(OC(F)F)cc4)cc3)c(=O)c2cc1Cl]

Following the general procedure for the hydrolysis of the 4-chloro quinolines, a mixture of 15h (230 mg, 0.5 mmol, 1 eq), KOAc (490 mg, 5.0 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give pure ELQ-600 (165 mg, 75% yield) as a white solid. ¹H-NMR (400 MHz; DMSO-d₆): δ 11.74 (s, 1H), 8.02 (s, 1H), 7.77 (broad d, J=8.7 Hz, 2H), 7.67 (broad d, J=8.3 Hz, 2H), 7.31 (t, J=73.8 Hz, 1H)), 7.34 (broad d, J=8.3 Hz, 2H), 7.29 (broad d, J=8.6 Hz, 2H), 7.07 (s, 1H), 3.96 (s, 3H), 2.25 (s, 3H).

### 6-chloro-7-methoxy-2-methyl-3-(3′-(trifluoromethyl)-[1,1′-biphenyl]-4-yl)quinolin-4(1H)-one (ELQ-646)

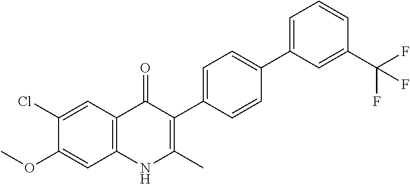 [SMILES: COc1cc2[nH]c(C)c(-c3ccc(-c4cccc(C(F)(F)F)c4)cc3)c(=O)c2cc1Cl]

Following the general procedure for the hydrolysis of the 4-chloro quinolines, a mixture of 15i (87 mg, 0.188 mmol, 1 eq), KOAc (184 mg, 1.82 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give ELQ-646 (30 mg, 36% yield) as a white solid. The product is 95-98% pure by NMR and HPLC. ¹H-NMR (400 MHz; DMSO-d₆): δ 11.70 (s, 1H), 8.07-8.04 (m, 1H), 8.02 (s, 2H), 7.80-7.73 (m, 4H), 7.41-7.37 (m, 2H), 7.10 (s, 1H), 3.98 (s, 3H), 2.28 (s, 3H).

### 6-chloro-7-methoxy-2-methyl-3-(3′-(trifluoromethoxy)-[1,1′-biphenyl]-4-yl)quinolin-4(1H)-one (ELQ-604)

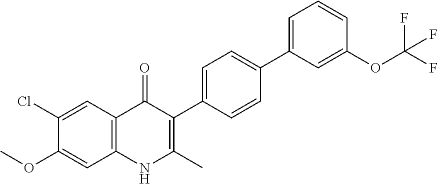 [SMILES: COc1cc2[nH]c(C)c(-c3ccc(-c4cccc(OC(F)(F)F)c4)cc3)c(=O)c2cc1Cl]

Following the general procedure for the hydrolysis of the 4-chloro quinolines, a mixture of 15j (476 mg, 1.0 mmol, 1 eq), KOAc (980 mg, 10.0 mmol, 10 eq) and glacial acetic acid (5 ml) was heated for 16 h. After cooling to room temperature, the reaction mixture was poured into ice water (20 ml). The resulting precipitate was filtered washed with water (3×10 ml), acetone (2×10 ml), DCM (2×10 ml), hexane (10 ml) and airdried to give crude ELQ-604 (350 mg). The product was crystallized from DMF to give ELQ-604 (200 mg, 43% yield). NMR and HPLC indicated that the product was about 95-98% pure. ¹H-NMR (400 MHz; DMSO-d₆): δ 11.70 (s, 1H), 8.02 (s, 1H), 7.79 (ddd, J=7.9, 1.7, 0.9 Hz, 1H), 7.76-7.73 (m, 2H), 7.69 (s, 1H), 7.63 (t, J=8.0 Hz, 1H), 7.40-7.36 (m, 3H), 7.09 (s, 1H), 3.98 (s, 3H), 2.27 (s, 3H).

### ((6-chloro-7-methoxy-2-methyl-3-(4′-(trifluoromethoxy)-[1,1′-biphenyl]-4-yl)quinolin-4-yl)oxy)methyl ethyl carbonate (ELQ-598)

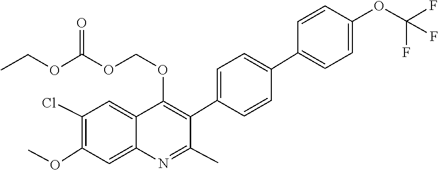 [SMILES: CCOC(=O)OCOc1c(-c2ccc(-c3ccc(OC(F)(F)F)cc3)cc2)c(C)nc2cc(OC)c(Cl)cc12]

A stirred mixture of ELQ-596 (460 mg, 1.0 mmol, 1 eq), tetra butyl ammonium iodide (742 mg, 2.0 mmol, 2 eq), chloromethyl ethylcarbonate (278 mg, 2.0 mmol, 2 eq) and dried K₂CO₃ (278 mg, 2.0 mmol, 2 eq) in DMF (25 ml) was heated at 60° C. for 24 hours when TLC showed no more starting material remained. The mixture was cooled to room temperature, filtered and the filtrate concentrated to dryness to give 700 mg of brown oil. The resulting residue was stirred with ethyl acetate (50 ml) for 30 minutes and the insoluble tetra butyl ammonium iodide filtered and wash with ethyl acetate (3×10 ml). The filtrate was concentrated to dryness and purified by flash chromatography using a gradient of ethyl acetate/hexane (1/1) as eluent to give pure ELQ-598 (412 mg, 73% yield) as a white solid. HPLC shows 1 peak with a purity greater than 98%. GC-MS shows 1 peak M⁺=561 (53%), 459 (100%). ¹H-NMR (400 MHz; CDCl₃): δ 8.08 (s, 1H), 7.74-7.70 (m, 4H), 7.51-7.48 (m, 2H), 7.46 (s, 1H), 7.37-7.34 (m, 2H), 5.31 (s, 2H), 4.13 (q, J=7.1 Hz, 2H), 4.07 (s, 3H), 2.56 (s, 3H), 1.22 (t, J=7.1 Hz, 3H).

### 6-Dichloro-7-methoxy-2-methyl-3-(4-(4,4,5,5-tetramethyl-1,3,2-dioxaborolan-2-yl)phenyl)quinoline

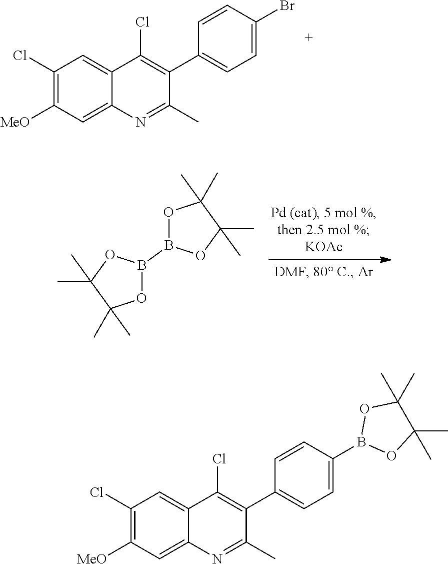

A mixture of 3-(4-bromophenyl)-4,6-dichloro-7-methoxy-2-methylquinoline (7.08 g, 0.18 mol), bis(pinacolato)diboron (1.4 eq, 6.34 g, 0.025 mol), and potassium acetate (3.0 eq, 5.24 g, 0.0534 mol) in 250 mL N,N-dimethylformamide was degassed by bubbling argon through a glass tube inserted under the liquid surface for 20 minutes at room temperature. [1,1′-Bis(diphenylphosphino)ferrocene]-dichloropalladium (II) (5 mol %, 0.65 g, 0.00089 mol) was added, followed by heating at 80° C. under an atmosphere of argon. After 72 hours, TLC and GC/MS indicated that unreacted quinoline starting material was still present. The reaction was cooled to room temperature and again degassed, followed by the addition of further [1,1′-bis(diphenylphosphino)ferrocene]-dichloropalladium (II) (2.4 mol %, 0.32 g, 0.00044 mol). The reaction was again heated at 80° C. under an atmosphere of argon for 72 hours. Although TLC and GC/MS showed that a small amount of unreacted 3-(4-bromophenyl)-4,6-dichloro-7-methoxy-2-methylquinoline still remained, the reaction was removed from the heat, filtered through Celite, and concentrated under reduced pressure with heating. The resulting black residue was taken up in dichloromethane (250 mL) and filtered through Celite. The dark filtrate was concentrated under reduced pressure with heating, affording a black sludge. This material was again taken up in dichloromethane (300 mL) and washed with 5% brine (2×100 mL), then 10% brine (100 mL). The pooled organic layers were dried (MgSO₄) and evaporated under reduced pressure with warming, affording a black solid (11.44 g). This material was taken up in 15 mL dichloromethane and filtered through a plug of silica gel (100 g, pre-wetted with dichloromethane), washing with 98/2 v/v dichloromethane/ethyl acetate until no more product eluted by TLC. Evaporation of the filtrate afforded a pale greenish gray solid (7.22 g). Automated flash chromatography on silica, eluting with a gradient of 100% dichloromethane to 98/2 v/v dichloromethane/ethyl acetate, afforded the desired product (R_{f}=0.21, 98/2 v/v dichloromethane/ethyl acetate) as an off-white solid (3.66 g, 46%, ¹H-NMR (400 MHz; CDCl₃): δ 8.23 (s, 1H), 7.97-7.94 (m, 2H), 7.46 (s, 1H), 7.30-7.27 (m, 2H), 4.06 (s, 3H), 2.43 (s, 3H), 1.38 (s, 12H).)

### 4,6-Dichloro-7-methoxy-2-methyl-3-(3′-(pentafluorosulfanyl)-[1,1′-biphenyl]-4-yl)quinoline

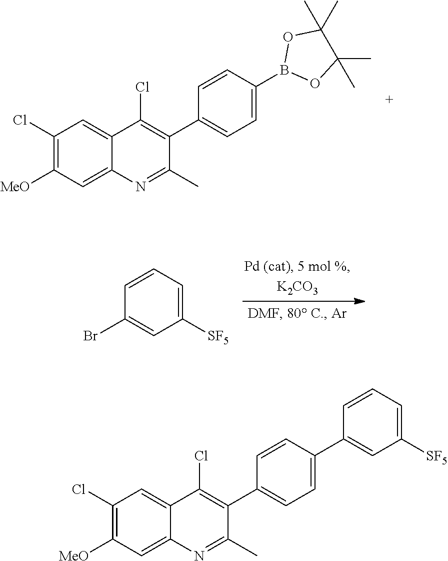

A mixture of 4,6-dichloro-7-methoxy-2-methyl-3-(4-(4,4,5,5-tetramethyl-1,3,2-dioxaborolan-2-yl)phenyl)quinoline (0.46 g, 0.0010 mol), anhydrous potassium carbonate (2.0 eq, 0.0021 mol, 0.29 g), and meta-bromophenylsulfur pentafluoride (1.3 eq, 0.0013 mol, 0.38 g) in N,N-dimethylformamide (55 mL) was degassed by bubbling argon through a glass tube under the liquid surface for 20 minutes at room temperature. [1,1′-Bis(diphenylphosphino)ferrocene]-dichloropalladium (II) (5 mol %, 0.038 g, 0.000052 mol) was added, followed by heating at 80° C. under an atmosphere of argon for 22 hours. The cooled reaction mixture was filtered through Celite. The filtrate was concentrated under reduced pressure with heating, and the resulting dark solid was taken up in dichloromethane (125 mL) and again filtered through Celite. The filtrate was adsorbed onto silica and purified by flash chromatography, eluting with a gradient of 95/5 to 77/23 v/v hexanes/ethyl acetate. The desired product (R_{f}=0.41 (3/2 v/v hexanes/ethyl acetate, silica) was obtained as a white solid (0.45 g, 70%, ¹H-NMR (400 MHz; CDCl₃): δ 8.26 (s, 1H), 8.06-8.04 (m, 1H), 7.83-7.81 (m, 1H), 7.78 (ddd, J=8.3, 2.2, 1.0 Hz, 1H), 7.74-7.71 (m, 2H), 7.61-7.57 (m, 1H), 7.48 (s, 1H), 7.42-7.38 (m, 2H), 4.08 (s, 3H), 2.50 (s, 3H)).

### 6-Chloro-7-methoxy-2-methyl-3-(3′-(pentafluorosulfanyl)-[1,1′-biphenyl]-4-yl)quinolin-4(1H)-one (ELQ-662)

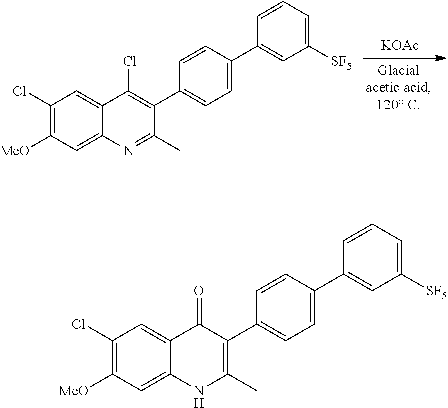

4,6-Dichloro-7-methoxy-2-methyl-3-(3′-(pentafluorosulfanyl)-[1,1′-biphenyl]-4-yl)quinoline (0.45 g, 0.00086 mol) and potassium acetate (10 eq, 0.0086 mol, 0.85 g) were heated in glacial acetic acid (9 mL) at 120° C. for 6 hours. After cooling, the reaction mixture was chilled at 5° C. for 30 minutes. Vacuum filtration, rinsing with excess water followed by acetone (5×1.5 mL), afforded the desired product as fine white crystals (0.21 g, 48%, ¹H-NMR (400 MHz; DMSO-d₆): δ 11.75 (s, 1H), 8.14-8.11 (m, 1H), 8.05-8.02 (m, 2H), 7.92 (ddd, J=8.3, 2.3, 0.8 Hz, 1H), 7.77-7.72 (m, 3H), 7.41-7.38 (m, 2H), 7.09 (s, 1H), 3.97 (s, 3H), 2.26 (s, 3H)).

### ((6-Chloro-7-methoxy-2-methyl-3-(3′-(pentafluoro-λ⁶-sulfaneyl)-[1,1′-biphenyl]-4-yl)quinolin-4-yl)oxy)methyl ethyl carbonate (ELQ-674)

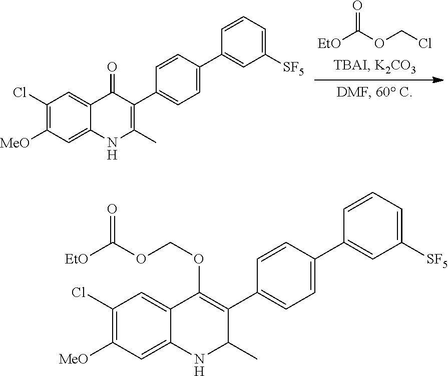

6-Chloro-7-methoxy-2-methyl-3-(3′-(pentafluoro-λ⁶-sulfaneyl)-[1,1′-biphenyl]-4-yl)quinolin-4(1H)-one (0.173 g, 0.00034 mol) was combined with tetrabutyl ammonium iodide (2.0 eq, 0.00069 mol, 0.25 g) and anhydrous potassium carbonate (2.0 eq, 0.00069 mol, 0.095 g) in 15 mL DMF. After stirring briefly at room temperature, chloromethyl ethyl carbonate (2.0 eq, 0.00069 mol, 0.095 g) was added as a solution in 1 mL DMF. The reaction was allowed to stir at 60° C., sealed with a needle vented septum, for 24 hours, whereupon TLC indicated that reaction was complete. The cooled reaction mixture was vacuum filtered to remove solids, and the solvent was removed from the filtrate under reduced pressure with heating. The residue was taken up in 50 mL ethyl acetate and stirred, resulting in precipitation of tetrabutyl ammonium iodide; this was removed by vacuum filtration, and the solvent was removed from the filtrate under reduced pressure with warming. Automated flash chromatography of the residue on silica, eluting with a gradient of 90:10 to 65:35 v:v hexanes:ethyl acetate afforded the desired product (R_{F}=0.40, 1:1 v:v hexanes:ethyl acetate) as a white solid (90 mg, 44%, ¹H-NMR (400 MHz; CDCl₃): δ 8.06 (s, 1H), 8.06-8.04 (m, 1H), 7.82-7.77 (m, 2H), 7.74-7.71 (m, 2H), 7.62-7.56 (m, 1H), 7.53-7.50 (m, 2H), 7.45 (s, 1H), 5.30 (s, 2H), 4.11 (q, J=7.1 Hz, 2H), 4.06 (s, 3H), 2.54 (s, 3H), 1.21 (t, J=7.1 Hz, 3H)).

### 4,6-Dichloro-7-methoxy-2-methyl-3-(4′-(pentafluorosulfanyl)-[1,1′-biphenyl]-4-yl)quinoline

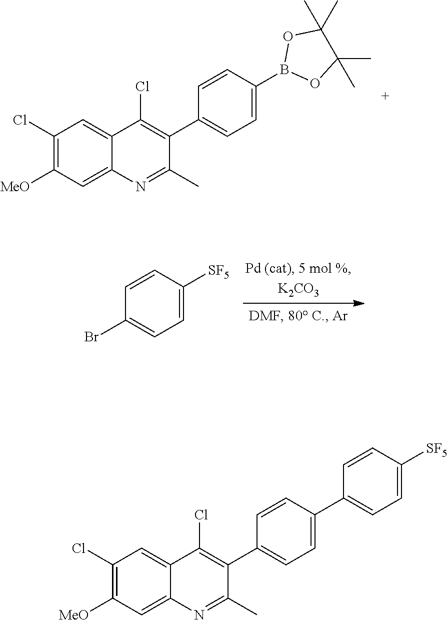

A mixture of 4,6-dichloro-7-methoxy-2-methyl-3-(4-(4,4,5,5-tetramethyl-1,3,2-dioxaborolan-2-yl)phenyl)quinoline (0.46 g, 0.0010 mol), anhydrous potassium carbonate (2.0 eq, 0.0021 mol, 0.29 g), and para-bromophenylsulfur pentafluoride (1.3 eq, 0.0013 mol, 0.38 g) in N,N-dimethylformamide (75 mL) was degassed by bubbling argon through a glass tube under the liquid surface for 20 minutes at room temperature. [1,1′-Bis(diphenylphosphino)ferrocene]-dichloropalladium (II) (5 mol %, 0.038 g, 0.000052 mol) was added, followed by heating at 80° C. under an atmosphere of argon for 22 hours. The cooled reaction mixture was filtered through Celite. The filtrate was concentrated under reduced pressure with heating, and the resulting dark solid was taken up in dichloromethane (125 mL) and again filtered through Celite. The filtrate was adsorbed onto silica and purified by flash chromatography, eluting with a gradient of 95/5 to 75/25 v/v hexanes/ethyl acetate. This afforded the desired product (R_{f}=0.43 (3/2 v/v hexanes/ethyl acetate, silica) as an off-white solid (0.38 g, 83%, ¹H-NMR (400 MHz; CDCl₃): δ 8.25 (s, 1H), 7.89-7.86 (m, 2H), 7.77-7.72 (m, 4H), 7.48 (s, 1H), 7.41-7.38 (m, 2H), 4.08 (s, 3H), 2.50 (s, 3H)).

### 6-Chloro-7-methoxy-2-methyl-3-(4′-(pentafluorosulfanyl)-[1,1′-biphenyl]-4-yl)quinolin-4(1H)-one (ELQ-663)

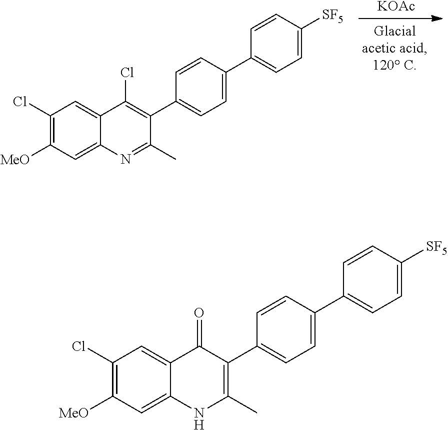

4,6-Dichloro-7-methoxy-2-methyl-3-(4′-(pentafluorosulfanyl)-[1,1′-biphenyl]-4-yl)quinoline (0.38 g, 0.00073 mol) and potassium acetate (10 eq, 0.0073 mol, 0.72 g) were heated in glacial acetic acid (12 mL) at 120° C. for 6 hours. After cooling, the reaction mixture was chilled at 5° C. for 30 minutes. Vacuum filtration, rinsing with excess water followed by acetone (5×1.5 mL), afforded the desired product as silver crystals (0.26 g, 72%, ¹H-NMR (400 MHz; DMSO-d₆): δ 11.71 (s, 1H), 8.02 (s, 1H), 8.01-7.98 (m, 2H), 7.97-7.93 (m, 2H), 7.78-7.75 (m, 2H), 7.42-7.39 (m, 2H), 7.09 (s, 1H), 3.97 (s, 3H), 2.27 (s, 3H)).

### ((6-Chloro-7-methoxy-2-methyl-3-(4′-(pentafluoro-λ⁶-sulfaneyl)-[1,1′-biphenyl]-4-yl)quinolin-4-yl)oxy)methyl ethyl carbonate (ELQ-674)

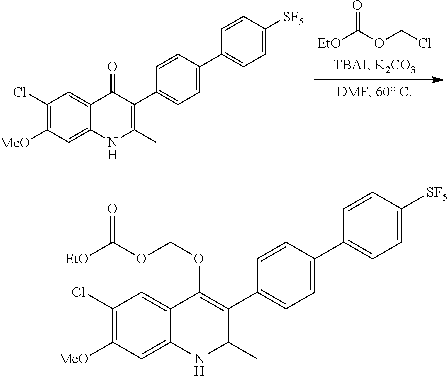

6-Chloro-7-methoxy-2-methyl-3-(4′-(pentafluoro-λ⁶-sulfaneyl)-[1,1′-biphenyl]-4-yl)quinolin-4(1H)-one (0.166 g, 0.00033 mol) was combined with tetrabutyl ammonium iodide (2.0 eq, 0.00066 mol, 0.24 g) and anhydrous potassium carbonate (1.0 eq, 0.00033 mol, 0.046 g) in 15 mL DMF. After stirring briefly at room temperature, chloromethyl ethyl carbonate (1.0 eq, 0.00033 mol, 0.046 g) was added as a solution in 1 mL DMF. The reaction was allowed to stir at 60° C., sealed with a needle vented septum, for 24 hours. Although a small amount of unreacted starting material was still present by TLC, the reaction mixture was cooled, vacuum filtered to remove solids, and the solvent was removed from the filtrate under reduced pressure with heating. The residue was taken up in 40 mL ethyl acetate and stirred, resulting in precipitation of tetrabutyl ammonium iodide; this was removed by vacuum filtration, and the solvent was removed from the filtrate under reduced pressure with warming. Automated flash chromatography of the residue on silica, eluting with a gradient of 9:1 to 7:3 v:v hexanes:ethyl acetate afforded the desired product as a white solid (0.17 g, 86%, ¹H-NMR (400 MHz; CDCl₃): δ 8.06 (s, 1H), 7.90-7.86 (m, 2H), 7.76-7.72 (m, 4H), 7.52-7.49 (m, 2H), 7.45 (s, 1H), 5.30 (s, 2H), 4.10 (q, J=7.1 Hz, 2H), 4.06 (s, 3H), 2.54 (s, 3H), 1.21 (t, J=7.1 Hz, 3H)).

### 4,6-Dichloro-7-methoxy-2-methyl-3-(4′-(trifluoroethoxy)-[1,1′-biphenyl]-4-yl)quinoline

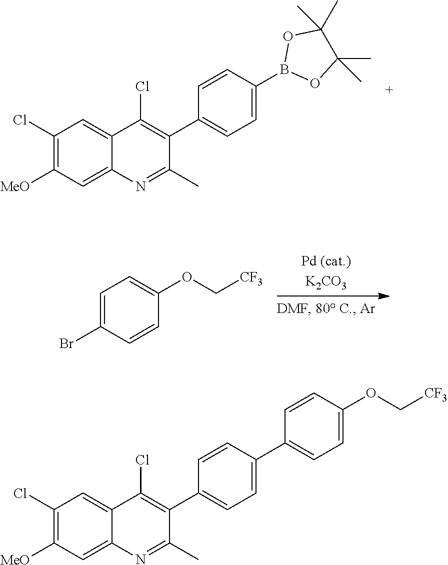 [SMILES: COc1cc2nc(C)c(-c3ccc(B4OC(C)(C)C(C)(C)O4)cc3)c(Cl)c2cc1Cl ; FC(F)(F)COc1ccc(Br)cc1 ; COc1cc2nc(C)c(-c3ccc(-c4ccc(OCC(F)(F)F)cc4)cc3)c(Cl)c2cc1Cl]

A mixture of 4,6-dichloro-7-methoxy-2-methyl-3-(4-(4,4,5,5-tetramethyl-1,3,2-dioxaborolan-2-yl)phenyl)quinoline (0.48 g, 0.0011 mol), anhydrous potassium carbonate (2.0 eq, 0.0022 mol, 0.30 g), and 1-bromo-4-(2,2,2-trifluoroethoxy)benzene (1.3 eq., 0.0014 mol, 0.36 g) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.040 g, 0.000055 mol) was added, followed by heating at 80° C. under an atmosphere of argon for 16 hours. The cooled reaction mixture was filtered through Celite. The filtrate was concentrated under reduced pressure with heating, and the resulting dark solid was taken up in dichloromethane (125 mL) and again filtered through Celite. The filtrate was adsorbed onto silica and purified by flash chromatography, eluting with a gradient of 100/0 to 75/25 v/v hexanes/ethyl acetate. Concentrated fractions were combined and taken up in ethyl acetate (50 mL) and dichloromethane (50 mL). The filtrate was concentrated under reduced pressure with heating, and the resulting dark solid was adsorbed onto silica and purified by flash chromatography again, eluting with a gradient of 100/0 to 80/20 v/v hexanes/ethyl acetate. This afforded the desired product (R_{f}=0.39 (3/2 v/v hexanes/ethyl acetate, silica) as an off-white solid (0.39 g, 72%, ¹H-NMR (400 MHz; CDCl₃): δ 8.25 (s, 1H), 7.71-7.68 (m, 2H), 7.67-7.64 (m, 2H), 7.47 (s, 1H), 7.35-7.32 (m, 2H), 7.08-7.05 (m, 2H), 4.43 (q, J=8.1 Hz, 2H), 4.07 (s, 3H), 2.50 (s, 3H)).

### 6-Chloro-7-methoxy-2-methyl-3-(4′(trifluoroethoxy)-[1,1′-biphenyl]-4-yl)quinolin-4(1H)-one (ELQ-670)

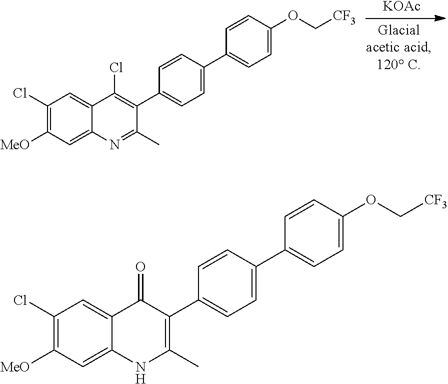 [SMILES: COc1cc2nc(C)c(-c3ccc(-c4ccc(OCC(F)(F)F)cc4)cc3)c(Cl)c2cc1Cl ; COc1cc2[nH]c(C)c(-c3ccc(-c4ccc(OCC(F)(F)F)cc4)cc3)c(=O)c2cc1Cl ; "KOAc"]

4,6-Dichloro-7-methoxy-2-methyl-3-(4′-(trifluoroethoxy)-[1,1′-biphenyl]-4-yl)quinoline (0.39 g, 0.00079 mol) and potassium acetate (10 eq., 0.0079 mol, 0.78 g) were heated in glacial acetic acid (18 mL) at 120° C. for 20 hours. After cooling, the reaction mixture was chilled at 5° C. for one hour. Vacuum filtration, rinsing with excess water followed by acetone (3×1.5 mL), afforded the desired product as an off-white solid (0.06 g, 11.5%, ¹H-NMR (400 MHz; DMSO-d₆): δ 11.67 (s, 1H), 8.01 (s, 1H), 7.71-7.69 (m, 2H), 7.66-7.64 (m, J=8.3 Hz, 2H), 7.33-7.30 (m, J=8.2 Hz, 2H), 7.18-7.16 (m, 2H), 7.08 (s, 1H), 4.83 (q, J=8.9 Hz, 2H), 3.97 (s, 3H), 2.26 (s, 3H)).

### 4,6-Dichloro-7-methoxy-2-methyl-3-(4′-((trifluoromethyl)thio)-[1,1′-biphenyl]-4-yl)quinoline

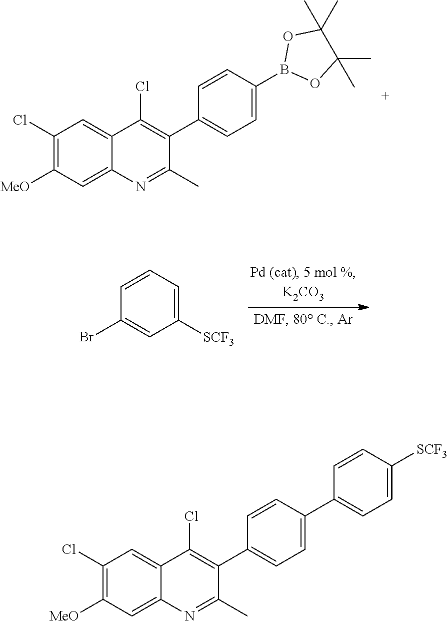

A mixture of 4,6-dichloro-7-methoxy-2-methyl-3-(4-(4,4,5,5-tetramethyl-1,3,2-dioxaborolan-2-yl)phenyl)quinoline (0.54 g, 0.0012 mol), anhydrous potassium carbonate (2.0 eq, 0.0024 mol, 0.33 g), and 4-bromo-(trifluoromethylthio)benzene (1.3 eq, 0.0013 mol, 0.38 g) in N,N-dimethylformamide (80 mL) was degassed by bubbling argon through a glass tube under the liquid surface for 30 minutes at room temperature. [1,1′-Bis(diphenylphosphino)ferrocene]-dichloropalladium (II) (5 mol %, 0.044 g, 0.000060 mol) was added, followed by heating at 80° C. under an atmosphere of argon for 24 hours. The cooled reaction mixture was filtered through Celite. The filtrate was concentrated under reduced pressure with heating, and the resulting dark solid was taken up in dichloromethane (300 mL) and again filtered through Celite. The filtrate was adsorbed onto silica and purified by flash chromatography, eluting with a gradient of 100% dichloromethane to 98/2 v/v dichloromethane/ethyl acetate. This afforded the desired product (R_{f}=0.47, 98/2 v/v dichloromethane/ethyl acetate, silica) as a white solid (0.47 g, 79%, ¹H-NMR (400 MHz; CDCl₃): δ 8.25 (s, 1H), 7.78-7.72 (m, 6H), 7.47 (s, 1H), 7.40-7.37 (m, 2H), 4.07 (s, 3H), 2.50 (s, 3H), ¹⁹F NMR (376 MHz; CDCl₃): δ −42.7).

### 6-Chloro-7-methoxy-2-methyl-3-(4′-((trifluoromethyl)thio)-[1,1′-biphenyl]-4-yl)quinolin-4(1H)-one

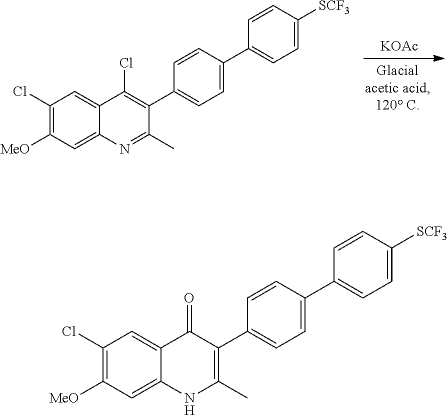

4,6-Dichloro-7-methoxy-2-methyl-4-(4′-(trifluoromethylthio)-[1,1′-biphenyl]-4-yl)quinoline (0.47 g, 0.00095 mol) and potassium acetate (10 eq, 0.0095 mol, 0.93 g) were heated in glacial acetic acid (10 mL) at 120° C. for 2 hours. After cooling, the reaction mixture was chilled at 5° C. for 20 minutes. Vacuum filtration, rinsing with excess water followed by acetone (3×2 mL), afforded the desired product as cream crystals (0.31 g, 69%, ¹H-NMR (400 MHz; DMSO-d₆): δ 11.71 (s, 1H), 8.02 (s, 1H), 7.91-7.89 (m, 2H), 7.85-7.80 (m, 2H), 7.77-7.75 (m, 2H), 7.40-7.38 (m, 2H), 7.09 (s, 1H), 3.98 (s, 3H), 2.27 (s, 3H). ¹⁹F NMR (376 MHz; DMSO): δ −42.0).

### 4,6-Dichloro-7-methoxy-2-methyl-3-(3′((trifluoromethyl)thio)-[1,1′-biphenyl]-4-yl)quinoline

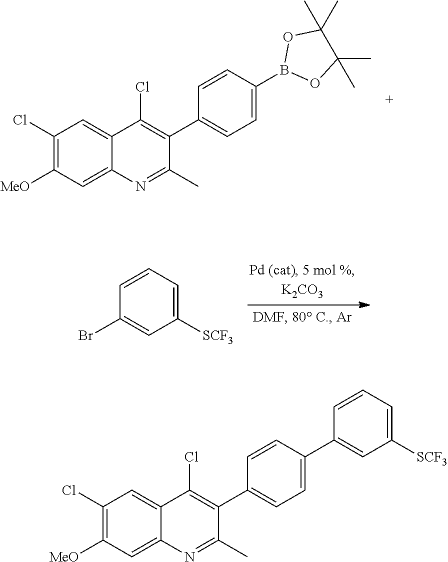

A mixture of 4,6-dichloro-7-methoxy-2-methyl-3-(4-(4,4,5,5-tetramethyl-1,3,2-dioxaborolan-2-yl)phenyl)quinoline (0.57 g, 0.0013 mol), anhydrous potassium carbonate (2.0 eq, 0.0026 mol, 0.36 g), and 3-bromophenyltrifluoromethyl sulfide (1.3 eq., 0.0017 mol, 0.43 g) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.047 g, 0.00006 mol) was added, followed by heating at 80° C. under an atmosphere of argon for 23 hours. The cooled reaction mixture was filtered through Celite. The filtrate was concentrated under reduced pressure with heating, and the resulting dark solid was taken up in dichloromethane (125 mL) and again filtered through Celite. The filtrate was adsorbed onto silica and purified by flash chromatography, eluting with a gradient of 100/0 to 92/8 v/v dichloromethane/ethyl acetate. This afforded the desired product (R_{f}=0.61, (98/2 v/v dichloromethane/ethyl acetate, silica) as an off-white solid (1.0 g, containing residual solvent) that was used without drying in the following step.

### 6-Dichloro-7-methoxy-2-methyl-3-(3′((trifluoromethyl)thio)-[1,1′-biphenyl]-4-yl)quinolin-4(1H)-one (ELQ-678)

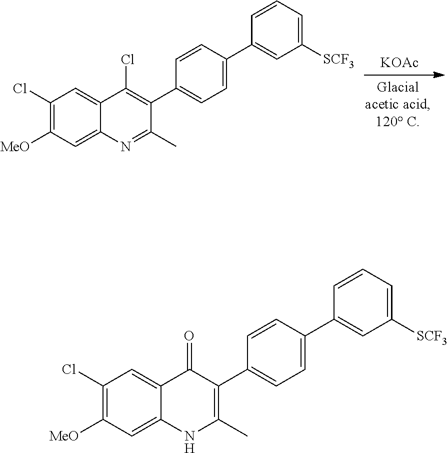

4,6-dichloro-7-methoxy-2-methyl-3-(3′((trifluoromethyl)thio)-[1,1′-biphenyl]-4-yl)quinoline (1.0 g, containing residual solvent) and potassium acetate (0.02 mol, 1.99 g) were heated in glacial acetic acid (15 mL) at 120° C. for 28 hours. After cooling, the reaction mixture was chilled at 5° C. for 1.5 hours. Vacuum filtration, rinsing with excess water followed by acetone (4×1.5 mL), afforded the desired product as grey crystals (0.40 g, 65% over two steps; ¹H-NMR (400 MHz; DMSO-d₆): δ 11.70 (s, 1H), 8.04-8.02 (m, 1H), 8.02 (s, 1H), 7.99-7.96 (m, 1H), 7.76-7.72 (m, 3H), 7.69-7.64 (m, 1H), 7.40-7.37 (m, 2H), 7.09 (s, 1H), 3.98 (s, 3H), 2.27 (s, 3H); 19^{F} NMR (376 MHz; DMSO): δ −41.9).

### 4,6-Dichloro-7-methoxy-2-methyl-3-(4′-(methylsulfonyl)-[1,1′-biphenyl]-4-yl)quinoline

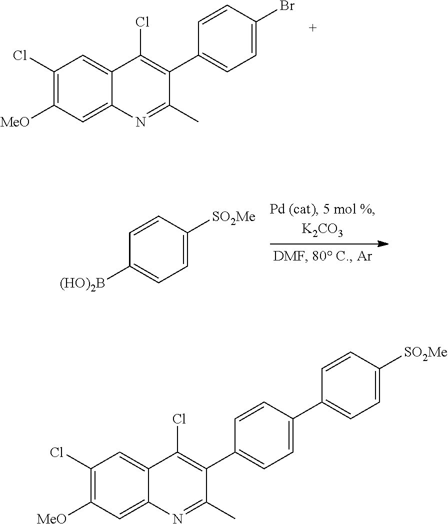 [SMILES: COc1cc2nc(C)c(-c3ccc(Br)cc3)c(Cl)c2cc1Cl ; CS(=O)(=O)c1ccc(B(O)O)cc1 ; COc1cc2nc(C)c(-c3ccc(-c4ccc(S(C)(=O)=O)cc4)cc3)c(Cl)c2cc1Cl]

A mixture of 3-(4-bromophenyl)-4,6-dichloro-7-methoxy-2-methylquinoline (1.00 g, 0.0025 mol), 4-(methylsulfonyl)boronic acid (0.60 g, 0.0030 mol, 1.2 eq), and anhydrous potassium carbonate (2.0 eq, 0.0050 mol, 0.69 g), in N,N-dimethylformamide (130 mL) was degassed by bubbling argon through a glass tube under the liquid surface for 25 minutes at room temperature. [1,1′-Bis(diphenylphosphino)ferrocene]-dichloropalladium (II) (5 mol %, 0.091 g, 0.000125 mol) was added, followed by heating at 80° C. under an atmosphere of argon for 4 days. The cooled reaction mixture was filtered through Celite. The filtrate was concentrated under reduced pressure with heating, and the resulting dark solid was taken up in dichloromethane (150 mL) and again filtered through Celite. The filtrate was adsorbed onto silica and purified by flash chromatography, eluting with a gradient of 85/15 to 30/70 v/v hexanes/ethyl acetate. This afforded the desired product (R_{f}=0.25 (3/2 v/v hexanes/ethyl acetate, silica) as a white solid (0.42 g, 36%, ¹H-NMR (400 MHz; DMSO-d₆): δ 8.20 (s, 1H), 8.08-8.03 (m, 4H), 7.96-7.93 (m, 2H), 7.66 (s, 1H), 7.56-7.53 (m, 2H), 4.07 (s, 3H), 3.29 (s, 3H), 2.43 (s, 3H)).

### 6-Chloro-7-methoxy-2-methyl-3-(4′-(methylsulfonyl)-[1,1′-biphenyl]-4-yl)quinolin-4(1H)-one (ELQ-658)

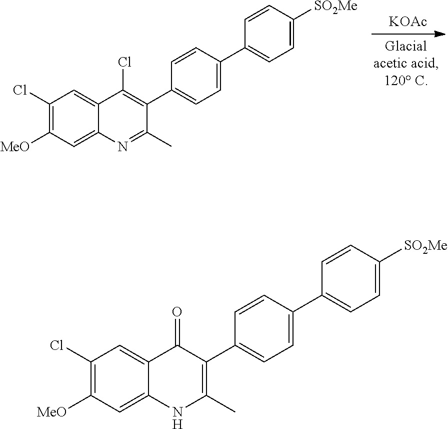 [SMILES: COc1cc2nc(C)c(-c3ccc(-c4ccc(S(C)(=O)=O)cc4)cc3)c(Cl)c2cc1Cl ; COc1cc2[nH]c(C)c(-c3ccc(-c4ccc(S(C)(=O)=O)cc4)cc3)c(=O)c2cc1Cl ; "KOAc"]

4,6-dichloro-7-methoxy-2-methyl-3-(4′-(methylsulfonyl)-[1,1′-biphenyl]-4-yl)quinoline (0.42 g, 0.00090 mol) and potassium acetate (10 eq, 0.0090 mol, 0.88 g) were heated in glacial acetic acid (10 mL) at 120° C. for 3 hours. After cooling, the reaction mixture was chilled at 5° C. for 1h. Vacuum filtration, rinsing with excess water followed by acetone (3×2 mL), afforded the desired product as fine, white crystals (0.24 g, 59%, ¹H-NMR (400 MHz; DMSO-d₆): δ 11.71 (s, 1H), 8.04-7.99 (m, 5H), 7.80-7.78 (m, 2H), 7.42-7.40 (m, 2H), 7.09 (s, 1H), 3.97 (s, 3H), 3.27 (s, 3H), 2.27 (s, 3H)).

### 4,6-Dichloro-7-methoxy-2-methyl-3-(3′-(2,2,2-trifluoroethoxy)-[1,1′-biphenyl]-4-yl)quinoline

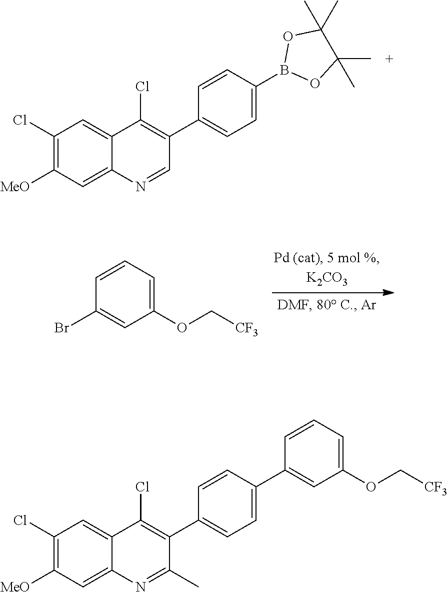 [SMILES: COc1cc2ncc(-c3ccc(B4OC(C)(C)C(C)(C)O4)cc3)c(Cl)c2cc1Cl ; FC(F)(F)COc1cccc(Br)c1 ; COc1cc2nc(C)c(-c3ccc(-c4cccc(OCC(F)(F)F)c4)cc3)c(Cl)c2cc1Cl]

A mixture of 4,6-dichloro-7-methoxy-2-methyl-3-(4-(4,4,5,5-tetramethyl-1,3,2-dioxaborolan-2-yl)phenyl)quinoline (0.48 g, 0.0011 mol), anhydrous potassium carbonate (2.0 eq, 0.0022 mol, 0.30 g), and 1-bromo-4-(2,2,2-trifluoroethoxy)benzene (1.3 eq., 0.0014 mol, 0.36 g) in N,N-dimethylformamide (50 mL) was degassed by bubbling argon through a glass tube under the liquid surface for 20 minutes at room temperature. [1,1′-Bis(diphenylphosphino)ferrocene]-dichloropalladium (II) (5 mol %, 0.040 g, 0.000055 mol) was added, followed by heating at 80° C. under an atmosphere of argon for 23 hours. The cooled reaction mixture was filtered through Celite. The filtrate was concentrated under reduced pressure with heating, and the resulting dark solid was taken up in dichloromethane (125 mL) and again filtered through Celite. The filtrate was adsorbed onto silica and purified by flash chromatography, eluting with a gradient of 95/5 to 72/28 v/v hexanes/ethyl acetate. The desired product was obtained as a white solid (R_{f}=0.38 (7/3 v/v hexanes/ethyl acetate, silica), 0.30 g, 55%, ¹H-NMR (400 MHz; CDCl₃): δ 8.25 (s, 1H), 7.71-7.68 (m, 2H), 7.67-7.64 (m, 2H), 7.47 (s, 1H), 7.35-7.32 (m, 2H), 7.08-7.05 (m, 2H), 4.43 (q, J=8.1 Hz, 2H), 4.07 (s, 3H), 2.50 (s, 3H)).

### 6-Chloro-7-methoxy-2-methyl-3-(3′-(2,2,2-trifluoroethoxy)-[1,1′-biphenyl]-4-yl)quinolin-4(1H)-one (ELQ-673)

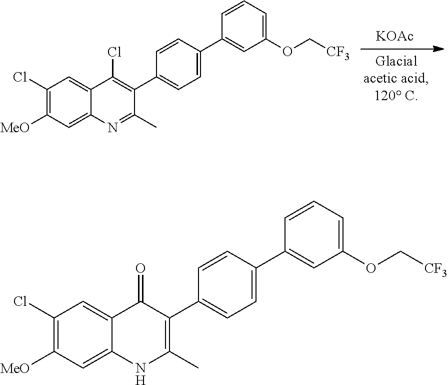 [SMILES: COc1cc2nc(C)c(-c3ccc(-c4cccc(OCC(F)(F)F)c4)cc3)c(Cl)c2cc1Cl ; COc1cc2[nH]c(C)c(-c3ccc(-c4cccc(OCC(F)(F)F)c4)cc3)c(=O)c2cc1Cl ; "KOAc"]

4,6-Dichloro-7-methoxy-2-methyl-3-(3′-(2,2,2-trifluoroethoxy)-[1,1′-biphenyl]-4-yl)quinoline (0.30 g, 0.00061 mol) and potassium acetate (10 eq., 0.0061 mol, 0.59 g) were heated in glacial acetic acid (10 mL) at 120° C. for 22 hours. After cooling, the reaction mixture was poured into 60 mL water. After stirring 3 minutes, the resulting solid was recovered by vacuum filtration, rinsing with excess water followed by acetone (2×2 mL). This afforded the desired product as a white solid (0.14 g, 47%, ¹H-NMR (400 MHz; DMSO-d₆): δ 11.67 (s, 1H), 8.01 (s, 1H), 7.71-7.69 (m, 2H), 7.66-7.64 (m, J=8.3 Hz, 2H), 7.33-7.30 (m, J=8.2 Hz, 2H), 7.18-7.16 (m, 2H), 7.08 (s, 1H), 4.83 (q, J=8.9 Hz, 2H), 3.97 (s, 3H), 2.26 (s, 3H)).

### 4′-(4,6-Dichloro-7-methoxy-2-methylquinolin-3-yl)-[1,1′-biphenyl]-4-sulfonamide

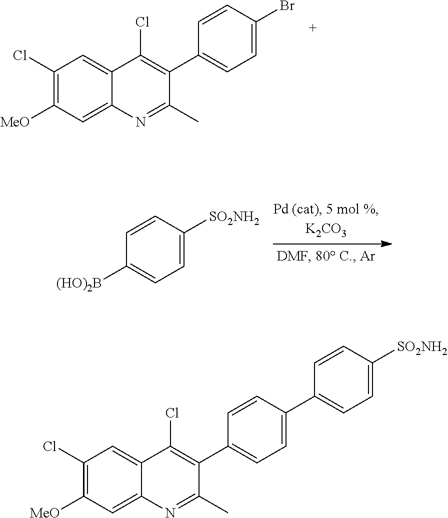 [SMILES: COc1cc2nc(C)c(-c3ccc(Br)cc3)c(Cl)c2cc1Cl ; NS(=O)(=O)c1ccc(B(O)O)cc1 ; COc1cc2nc(C)c(-c3ccc(-c4ccc(S(N)(=O)=O)cc4)cc3)c(Cl)c2cc1Cl]

A mixture of 3-(4-bromophenyl)-4,6-dichloro-7-methoxy-2-methylquinoline (1.00 g, 0.0025 mol), 4-aminosulfonylphenylboronic acid (0.66 g, 0.0033 mol, 1.3 eq), and anhydrous potassium carbonate (2.0 eq, 0.0050 mol, 0.69 g), in N,N-dimethylformamide (130 mL) was degassed by bubbling argon through a glass tube under the liquid surface for 20 minutes at room temperature. [1,1′-Bis(diphenylphosphino)ferrocene]-dichloropalladium (II) (5 mol %, 0.091 g, 0.000125 mol) was added, followed by heating at 80° C. under an atmosphere of argon for 24 hours. The cooled reaction mixture was filtered through Celite. The filtrate was concentrated under reduced pressure with heating, and the resulting dark solid was swirled in dichloromethane (125 mL) and vacuum filtered. Because precipitation was observed in the filtrate, this was concentrated to 90 mL and again vacuum filtered. The resulting pale tan solid was the desired product in sufficient purity for the next reaction (0.30 g, 26%, ¹H-NMR (400 MHz; DMSO-d₆): δ 8.20 (s, 1H), 8.00-7.90 (m, 6H), 7.65 (s, 1H), 7.54-7.51 (m, 2H), 7.44 (br s, 2H), 4.07 (s, 3H), 2.43 (s, 3H)).

### 4′-(6-Chloro-7-methoxy-2-methyl-4-oxo-1,4-dihydroquinolin-3-yl)-[1,1′-biphenyl]-4-sulfonamide (ELQ-680)

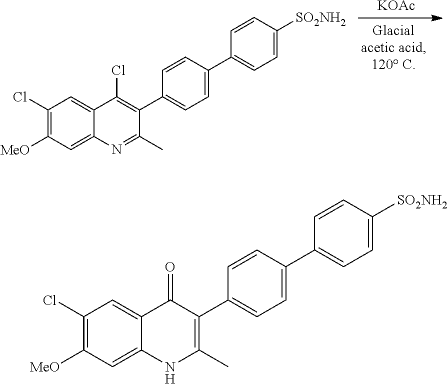 [SMILES: COc1cc2nc(C)c(-c3ccc(-c4ccc(S(N)(=O)=O)cc4)cc3)c(Cl)c2cc1Cl ; COc1cc2[nH]c(C)c(-c3ccc(-c4ccc(S(N)(=O)=O)cc4)cc3)c(=O)c2cc1Cl ; "KOAc"]

4′-(4,6-Dichloro-7-methoxy-2-methylquinolin-3-yl)-[1,1′-biphenyl]-4-sulfonamide (0.30 g, 0.00064 mol) and potassium acetate (10 eq, 0.0064 mol, 0.63 g) were heated in glacial acetic acid (10 mL) at 120° C. for 20 hours. After cooling, the reaction mixture was chilled at 5° C. for 20 minutes. Vacuum filtration, rinsing with excess water followed by acetone (3×1.5 mL), afforded the desired product as a grayish beige powder (0.21 g, 71%, ¹H-NMR (400 MHz; DMSO-d₆): δ 11.70 (s, 1H), 8.02 (s, 1H), 7.94-7.92 (m, 4H), 7.77-7.75 (m, 2H), 7.40-7.38 (m, 4H), 7.09 (s, 1H), 3.97 (s, 3H), 2.27 (s, 3H)).

### 4,6-Dichloro-7-methoxy-2-methyl-3-(4-(6-(trifluoromethyl)pyridin-3-yl)phenyl)quinoline

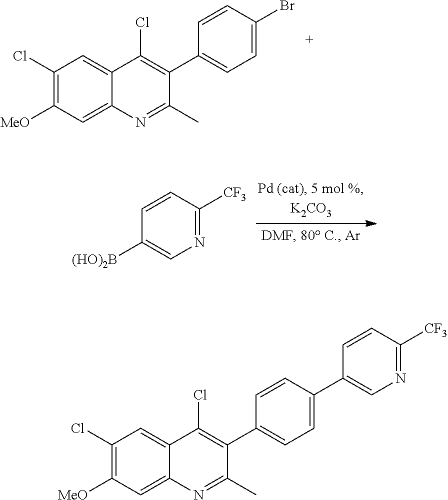 [SMILES: COc1cc2nc(C)c(-c3ccc(Br)cc3)c(Cl)c2cc1Cl ; OB(O)c1ccc(C(F)(F)F)nc1 ; COc1cc2nc(C)c(-c3ccc(-c4ccc(C(F)(F)F)nc4)cc3)c(Cl)c2cc1Cl]

A mixture of 3-(4-bromophenyl)-4,6-dichloro-7-methoxy-2-methylquinoline (0.85 g, 0.0021 mol), 2-(trifluoromethyl)pyridyl-5-boronic acid (1.3 eq., 0.0028 mol, 0.53 g), and anhydrous potassium carbonate (2.0 eq, 0.0042 mol, 0.58g) in N,N-dimethylformamide (100 mL) was stirred at room temperature for 20 minutes while degassing by bubbling argon through a glass tube under the liquid surface. [1,1′-bis(diphenylphosphino)ferrocene]-dichloropalladium (II) (5 mol %, 0.077 g, 0.00011 mol) was added and the reaction was allowed to heat at 80° C. under argon for 19 hours. The cooled reaction mixture was filtered through Celite, followed by concentration of the filtrate under reduced pressure with heating. The resulting solid was taken up in 110 mL DCM, followed by vacuum filtration. Automated flash chromatography of the evaporated filtrate on silica, eluting with a gradient of 9:1 to 72:28 v:v hexanes:ethyl acetate, afforded the desired product (R_{f}=0.35, 6:4 v:v hexanes:ethyl acetate on silica) as a white powder (0.37 g, 38%, ¹H-NMR (400 MHz; CDCl₃): δ 9.07-9.03 (m, 1H), 8.26 (s, 1H), 8.16-8.13 (m, 1H), 7.83-7.80 (m, 1H), 7.78-7.75 (m, 2H), 7.48 (s, 1H), 7.46-7.43 (m, 2H), 4.08 (s, 3H), 2.50 (s, 3H). ¹⁹F NMR (376 MHz; CDCl3): δ −67.7).

### 6-Chloro-7-methoxy-2-methyl-3-(4-(6-(trifluoromethyl)pyridin-3-yl)phenyl)quinolin-4(1H)-one (ELQ-683)

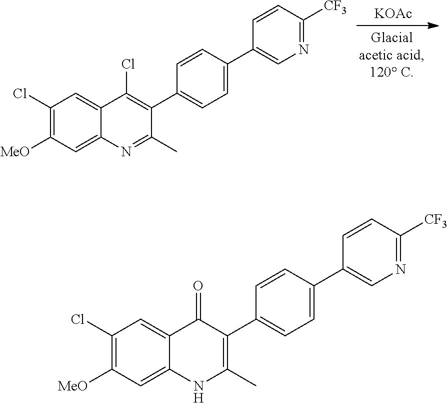 [SMILES: COc1cc2nc(C)c(-c3ccc(-c4ccc(C(F)(F)F)nc4)cc3)c(Cl)c2cc1Cl ; COc1cc2[nH]c(C)c(-c3ccc(-c4ccc(C(F)(F)F)nc4)cc3)c(=O)c2cc1Cl ; "KOAc"]

4,6-Dichloro-7-methoxy-2-methyl-3-(4-(6-(trifluoromethyl)pyridin-3-yl)phenyl)quinoline (0.37 g, 0.00080 mol) and anhydrous potassium acetate (10 eq, 0.0080 mol, 0.79g) were stirred in glacial acetic acid (15 mL) at 120° C. for 20 hours. After cooling, the reaction mixture was additionally chilled at 5° C. for 20 minutes, followed by vacuum filtration, rinsing with excess water followed by acetone (3×1.5 mL). The desired product was obtained as a cream powder (0.23 g, 65%, ¹H-NMR (400 MHz; DMSO-d₆): δ 11.72 (s, 1H), 9.18-9.14 (m, 1H), 8.44-8.41 (m, 1H), 8.02 (s, 1H), 8.02-7.99 (m, 1H), 7.87-7.85 (m, 2H), 7.45-7.43 (m, 2H), 7.09 (s, 1H), 3.98 (s, 3H), 2.28 (s, 3H). 19-F NMR (376 MHz; DMSO): δ −66.2).

### 4,6-Dichloro-7-methoxy-2-methyl-3-(4-(5-(trifluoromethoxy)pyridin-2-yl)phenyl)quinoline

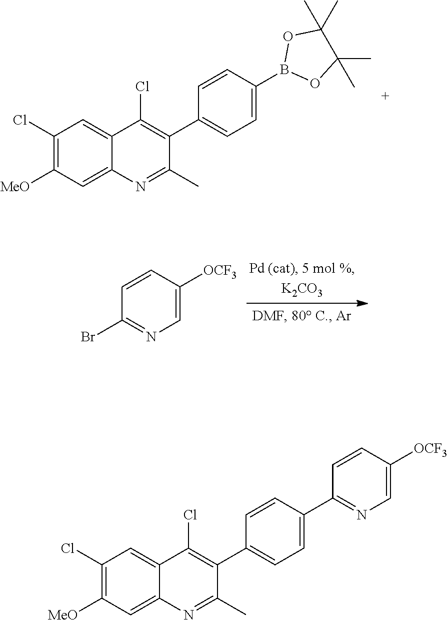 [SMILES: COc1cc2nc(C)c(-c3ccc(B4OC(C)(C)C(C)(C)O4)cc3)c(Cl)c2cc1Cl ; FC(F)(F)Oc1ccc(Br)nc1 ; COc1cc2nc(C)c(-c3ccc(-c4ccc(OC(F)(F)F)cn4)cc3)c(Cl)c2cc1Cl]

A mixture of 4,6-dichloro-7-methoxy-2-methyl-3-(4-(4,4,5,5-tetramethyl-1,3,2-dioxaborolan-2-yl)phenyl)quinoline (0.50 g, 0.0011 mol), anhydrous potassium carbonate (2.0 eq, 0.0022 mol, 0.30 g), and 2-bromo-5-(trifluoromethoxy)benzene (1.3 eq., 0.0015 mol, 0.35 g) in N,N-dimethylformamide (80 mL) was degassed by bubbling argon through a glass tube inserted under the liquid surface for 20 minutes at room temperature. [1,1′-Bis(diphenylphosphino)ferrocene]-dichloropalladium (II) (5 mol %, 0.040 g, 0.000055 mol) was added, followed by heating at 80° C. under an atmosphere of argon for 4 days. The cooled reaction mixture was filtered through Celite. The filtrate was concentrated under reduced pressure with heating, and the resulting dark solid was taken up in dichloromethane (100 mL) and again filtered through Celite. After evaporation of the filtrate, the residue was purified by automated flash chromatography on silica, eluting with a gradient of 1:0 to 6:4 v:v hexanes:ethyl acetate, followed by a second flash chromatography on the same silica gel column, eluting with a gradient of 1:0 to 75:25 v:v hexanes:ethyl acetate. The desired product (R_{f}=15, 8:2 v:v hexanes:ethyl acetate on silica) was obtained as a white powder (0.29 g, 55%, ¹H-NMR (400 MHz; CDCl₃): δ 8.67-8.66 (m, 1H), 8.25 (s, 1H), 8.14-8.11 (m, 2H), 7.88-7.86 (m, 1H), 7.70-7.67 (m, 1H), 7.48 (s, 1H), 7.42-7.39 (m, 2H), 4.07 (s, 3H), 2.49 (s, 3H)); 19-F NMR (376 MHz; CDCl3): δ −58.1).

### 6-Chloro-7-methoxy-2-methyl-3-(4-(5-(trifluoromethoxy)pyridin-2-yl)phenyl)quinolin-4(1H)-one (ELQ-681)

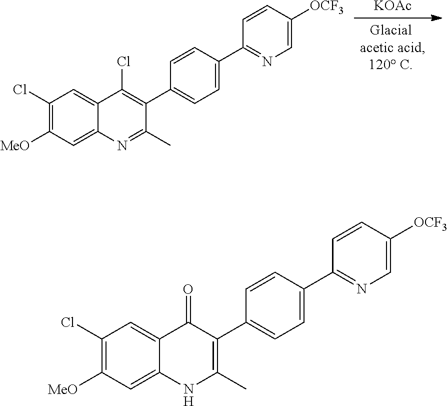 [SMILES: COc1cc2nc(C)c(-c3ccc(-c4ccc(OC(F)(F)F)cn4)cc3)c(Cl)c2cc1Cl ; COc1cc2[nH]c(C)c(-c3ccc(-c4ccc(OC(F)(F)F)cn4)cc3)c(=O)c2cc1Cl ; "KOAc"]

4,6-dichloro-7-methoxy-2-methyl-3-(4-(5-(trifluoromethoxy)pyridin-2-yl)phenyl)quinoline (0.29 g, 0.00061 mol) and anhydrous potassium acetate (10 eq, 0.0061 mol, 0.60 g) were heated at 120° C. in 10 mL glacial acetic acid for 20 hours. The cooled reaction mixture was chilled at 5° C. for 14 hours. Vacuum filtration, rinsing with excess water followed by acetone (3×1.5 mL), afforded the desired product as a white powder (0.17 g, 60% ¹H-NMR (400 MHz; DMSO-d₆): δ 11.72 (s, 1H), 8.77-8.77 (m, 1H), 8.18-8.16 (m, 1H), 8.13-8.10 (m, 2H), 8.02 (s, 1H), 8.02-7.99 (m, 1H), 7.42-7.39 (m, 2H), 7.10 (s, 1H), 3.98 (s, 3H), 2.27 (s, 3H). 19-F NMR (376 MHz; DMSO): δ −57.1).

### 4,6-Dichloro-3-(3′-ethoxy-[1,1′-biphenyl]-4-yl)-7-methoxy-2-methylquinoline

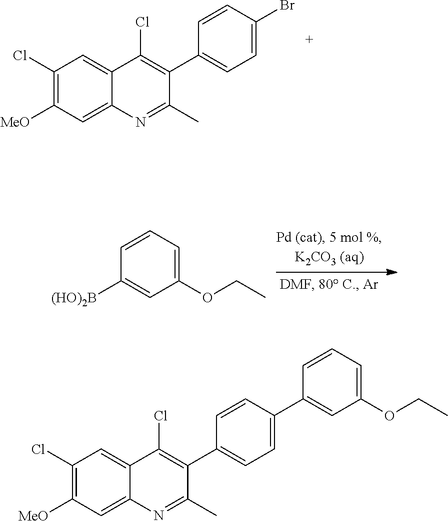 [SMILES: COc1cc2nc(C)c(-c3ccc(Br)cc3)c(Cl)c2cc1Cl ; CCOc1cccc(B(O)O)c1 ; CCOc1cccc(-c2ccc(-c3c(C)nc4cc(OC)c(Cl)cc4c3Cl)cc2)c1]

A mixture of 3-(4-bromophenyl)-4,6-dichloro-7-methoxy-2-methylquinoline (0.70 g, 0.0018 mol), 3-ethoxyphenylboronic acid (1.2 eq, 0.0022 mol, 0.37 g), and aqueous potassium carbonate (2.0 eq, 0.0036 mol, 0.50 g of anhydrous potassium carbonate dissolved in 1.8 mL water) in N,N-dimethylformamide (100 mL) was degassed by bubbling argon through a glass tube under the liquid surface for 20 minutes at room temperature. [1,1′-Bis(diphenylphosphino)ferrocene]-dichloropalladium (II) (5 mol %, 0.066 g, 0.000090 mol) was added, followed by heating at 80° C. under an atmosphere of argon for 20 hours. The cooled reaction mixture was filtered through Celite. The filtrate was concentrated under reduced pressure with heating, and the resulting dark solid was taken up in dichloromethane (100 mL), vacuum filtered, and purified by flash chromatography, eluting with a gradient of 1:0 to 75:25 v:v hexanes:ethyl acetate. This afforded the desired product (R_{f}=0.35, 7:3 v:v hexanes:ethyl acetate, silica) as a white solid (0.34 g, 43%, ¹H-NMR (400 MHz; CDCl₃): δ 8.28 (s, 1H), 7.77-7.74 (m, 2H), 7.50 (s, 1H), 7.41 (t, J=7.9 Hz, 1H), 7.37-7.34 (m, 2H), 7.28 (ddd, J=7.6, 1.7, 1.0 Hz, 3H), 7.24 (t, J=2.0 Hz, 1H), 6.95 (ddd, J=8.2, 2.5, 0.9 Hz, 1H), 4.17 (t, J=7.0 Hz, 3H), 4.10 (s, 3H), 2.53 (s, 3H), 1.49 (t, J=7.0 Hz, 3H).)

### 6-Chloro-3-(3′-ethoxy-[1,1′-biphenyl]-4-yl)-7-methoxy-2-methylquinolin-4(1H)-one (ELQ-691)

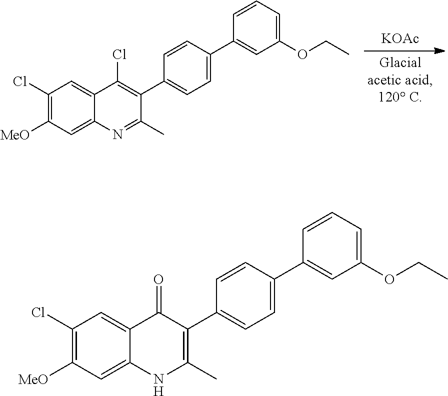 [SMILES: CCOc1cccc(-c2ccc(-c3c(C)nc4cc(OC)c(Cl)cc4c3Cl)cc2)c1 ; CCOc1cccc(-c2ccc(-c3c(C)[nH]c4cc(OC)c(Cl)cc4c3=O)cc2)c1 ; "KOAc"]

4,6-dichloro-3-(3′-ethoxy-[1,1′-biphenyl]-4-yl)-7-methoxy-2-methylquinoline (0.34 g, 0.00078 mol) and anhydrous potassium acetate (10 eq, 0.77 g, 0.0078 mol) were heated at 120° C. in glacial acetic acid (10 mL) for 23 hours. The cooled reaction mixture was further chilled at 5° C. overnight, then vacuum filtered, rinsing with excess water followed by acetone (3×1 mL), affording a white solid (the desired product; 0.20 g, 61%, ¹H-NMR (400 MHz; DMSO-d₆): δ 11.68 (s, 1H), 8.01 (s, 1H), 7.70-7.67 (m, 2H), 7.41-7.35 (m, 1H), 7.34-7.31 (m, 2H), 7.26 (ddd, J=7.7, 1.6, 1.0 Hz, 1H), 7.23-7.20 (m, 1H), 7.08 (s, 1H), 6.93 (ddd, J=8.2, 2.5, 0.9 Hz, 1H), 4.12 (q, J=7.0 Hz, 2H), 3.97 (s, 3H), 2.26 (s, 3H), 1.37 (t, J=7.0 Hz, 3H).)

### 4,6-Dichloro-7-methoxy-2-methyl-3-(4-(pyridin-4-yl)phenyl)quinoline

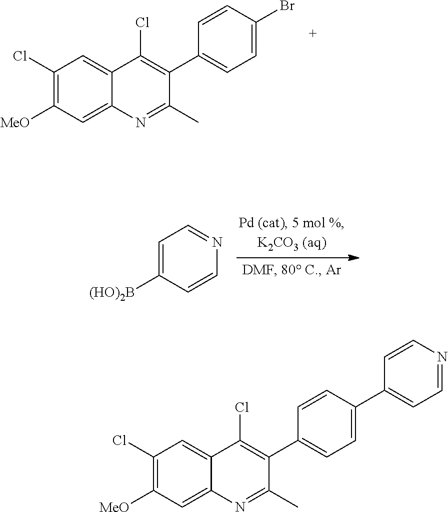 [SMILES: COc1cc2nc(C)c(-c3ccc(Br)cc3)c(Cl)c2cc1Cl ; OB(O)c1ccncc1 ; COc1cc2nc(C)c(-c3ccc(-c4ccncc4)cc3)c(Cl)c2cc1Cl]

A mixture of 3-(4-bromophenyl)-4,6-dichloro-7-methoxy-2-methylquinoline (0.71 g, 0.0018 mol), pyridine-4-boronic acid (1.3 eq, 0.0024 mol, 0.29 g), and aqueous potassium carbonate (2.0 eq, 0.0036 mol, 0.50 g of anhydrous potassium carbonate dissolved in 1.3 mL water) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.066 g, 0.000090 mol) was added, followed by heating at 80° C. under an atmosphere of argon for 20 hours. The cooled reaction mixture was filtered through Celite. The filtrate was concentrated under reduced pressure with heating, and the resulting dark solid was taken up in dichloromethane (100 mL) and vacuum filtered. The evaporated filtrate was purified by flash chromatography, eluting with a gradient of 85:15 to 0:100 v:v hexanes:ethyl acetate. This afforded the desired product (R_{f}=0.3, 100% ethyl acetate, silica) as a white solid (0.17 g, 24%, ¹H-NMR (400 MHz; CDCl₃): δ 8.73-8.71 (m, 2H), 8.25 (s, 1H), 7.81-7.78 (m, 2H), 7.61-7.59 (m, 2H), 7.47 (s, 1H), 7.43-7.40 (m, 2H), 4.08 (s, 3H), 2.50 (s, 3H)).

### 6-Chloro-7-methoxy-2-methyl-3-(4-(pyridin-4-yl)phenyl)quinolin-4(1H)-one (ELQ-714)

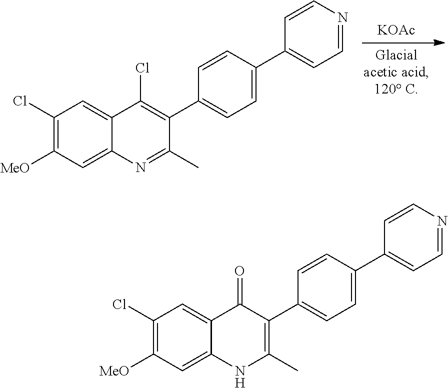 [SMILES: COc1cc2nc(C)c(-c3ccc(-c4ccncc4)cc3)c(Cl)c2cc1Cl ; COc1cc2[nH]c(C)c(-c3ccc(-c4ccncc4)cc3)c(=O)c2cc1Cl ; "KOAc"]

4,6-dichloro-7-methoxy-2-methyl-3-(4-(pyridin-4-yl)phenyl)quinoline (0.17 g, 0.00043 mol) and anhydrous potassium acetate (10 eq, 0.0043 mol, 0.42 g) were heated at 120° C. in glacial acetic acid (10 mL) for 24 hours. After cooling to room temperature, the reaction mixture was chilled overnight at 5° C., followed by vacuum filtration, rinsing with excess water followed by acetone (3×0.75 mL). The desired product was obtained as a pale yellow solid (50 mg). Additional clean product was recovered from the filtrate (27 mg) for a total of 77 mg desired product (48%, ¹H-NMR (400 MHz; DMSO-d₆): δ 11.79 (s, 1H), 8.76-8.74 (m, 2H), 8.02 (s, 1H), 8.00-7.98 (m, 2H), 7.92-7.90 (m, 2H), 7.46-7.44 (m, 2H), 7.11 (s, 1H), 3.98 (s, 3H), 2.27 (s, 3H)).

### 4,6-Dichloro-3-(2′-chloro-[1,1′-biphenyl]-4-yl)-7-methoxy-2-methylquinoline

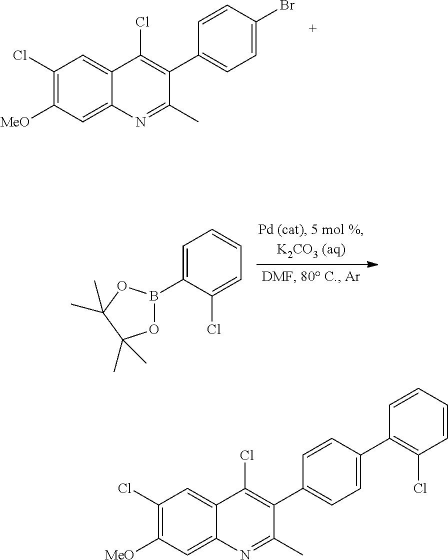 [SMILES: COc1cc2nc(C)c(-c3ccc(Br)cc3)c(Cl)c2cc1Cl ; CC1(C)OB(c2ccccc2Cl)OC1(C)C ; COc1cc2nc(C)c(-c3ccc(-c4ccccc4Cl)cc3)c(Cl)c2cc1Cl]

A mixture of 3-(4-bromophenyl)-4,6-dichloro-7-methoxy-2-methylquinoline (0.70 g, 0.0018 mol), 2-chlorophenylboronic acid pinacol ester (1.3 eq, 0.0023 mol, 0.55 g), and aqueous potassium carbonate (2.0 eq, 0.0032 mol, 0.44 g of anhydrous potassium carbonate dissolved in 1.6 mL water) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.066 g, 0.000090 mol) was added, followed by heating at 80° C. under an atmosphere of argon for 24 hours. The cooled reaction mixture was filtered through Celite. The filtrate was concentrated under reduced pressure with heating, and the resulting dark solid was taken up in dichloromethane (150 mL) and vacuum filtered. Automated flash chromatography of the residue from evaporation of the filtrate, eluting with a gradient of 95:5 to 82:18 v:v hexanes:ethyl acetate, did not effectively separate the desired product (R_{F}=0.24, 8:2 v:v hexanes:ethyl acetate) from a significant side product (R_{F}=0.21, 8:2 v:v hexanes:ethyl acetate) resulting from double addition of the boronic ester to the quinoline (replacing chlorine); this side product was observed by GC/MS with m/z=503, t_{R}=21 minutes (temperature program: 250° C. for 2 minutes followed by temperature increase of 30° C./minute to 300° C., DB5 column). The impure mixture (0.45g) was used without further purification in the following reaction.

### 6-Chloro-3-(2′-chloro-[1,1′-biphenyl]-4-yl)-7-methoxy-2-methylquinolin-4(1H)-one (ELQ-713)

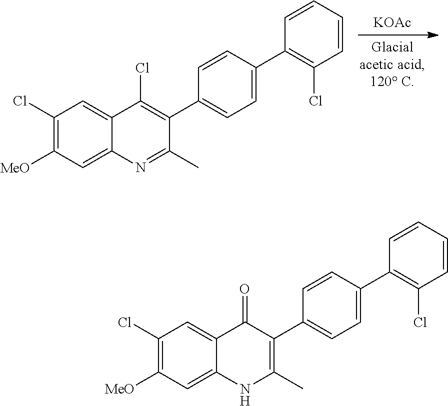 [SMILES: COc1cc2nc(C)c(-c3ccc(-c4ccccc4Cl)cc3)c(Cl)c2cc1Cl ; COc1cc2[nH]c(C)c(-c3ccc(-c4ccccc4Cl)cc3)c(=O)c2cc1Cl ; "KOAc"]

4,6-Dichloro-3-(2′-chloro-[1,1′-biphenyl]-4-yl)-7-methoxy-2-methylquinoline (0.15 g of impure material, see above) was heated at 110° C. in 10 mL glacial acetic acid with anhydrous potassium acetate (0.34 g, 0.0035 mol) for 24 hours. After cooling, the reaction mixture was chilled at 5° C., then vacuum filtered, rinsing with excess water followed by acetone (2×1.5 mL). The desired product was afforded as a white solid (0.0668g). Separately, another portion of 4,6-dichloro-3-(2′-chloro-[1,1′-biphenyl]-4-yl)-7-methoxy-2-methylquinoline (0.30 g of impure material from the same reaction, above) was treated in the same manner with anhydrous potassium acetate (0.69 g) and glacial acetic acid (10 mL), obtaining 0.116 g of a cream solid that was also the desired product (total yield 0.18 g, 23% over two steps from 3-(4-bromophenyl)-4,6-dichloro-7-methoxy-2-methylquinoline, ¹H-NMR (400 MHz; DMSO-d₆): δ 11.70 (s, 1H), 8.02 (s, 1H), 7.60-7.58 (m, 1H), 7.50-7.39 (m, 5H), 7.36-7.33 (m, 2H), 7.09 (s, 1H), 3.97 (s, 3H), 2.27 (s, 3H)).

### 4,6-Dichloro-7-methoxy-2-methyl-3-(4′-nitro-[1,1′-biphenyl]-4-yl)quinoline

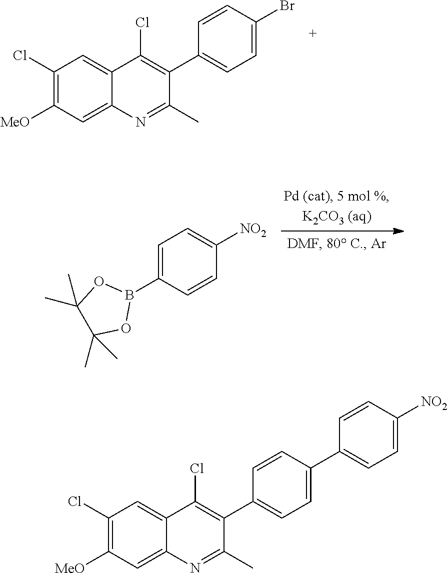 [SMILES: COc1cc2nc(C)c(-c3ccc(Br)cc3)c(Cl)c2cc1Cl ; CC1(C)OB(c2ccc([N+](=O)[O-])cc2)OC1(C)C ; COc1cc2nc(C)c(-c3ccc(-c4ccc([N+](=O)[O-])cc4)cc3)c(Cl)c2cc1Cl]

A mixture of 3-(4-bromophenyl)-4,6-dichloro-7-methoxy-2-methylquinoline (0.70 g, 0.0018 mol), 4-nitrophenylboronic acid pinacol ester (1.3 eq, 0.0023 mol, 0.57 g), and aqueous potassium carbonate (2.0 eq, 0.0036 mol, 0.50 g of anhydrous potassium carbonate dissolved in 1.8 mL water) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.066 g, 0.000090 mol) was added, followed by heating at 80° C. under an atmosphere of argon for 21 hours. The cooled reaction mixture was vacuum filtered, and the filtrate was concentrated under reduced pressure with heating. The residue was taken up in dichloromethane (125 mL) and vacuum filtered. Automated flash chromatography of the evaporated filtrate on silica, eluting with a gradient of 9:1 to 1:1 v:v hexanes:ethyl acetate, afforded the desired product as an off-white solid (0.07 g, 9%, ¹H-NMR (400 MHz; CDCl₃): δ 8.37-8.34 (m, 2H), 8.26 (s, 1H), 7.86-7.83 (m, 2H), 7.80-7.77 (m, 2H), 7.48 (s, 1H), 7.44-7.41 (m, 2H), 4.08 (s, 3H), 2.50 (s, 3H)).

### 6-Chloro-7-methoxy-2-methyl-3-(4′-nitro-[1,1′-biphenyl]-4-yl)quinolin-4(1H)-one (ELQ-715)

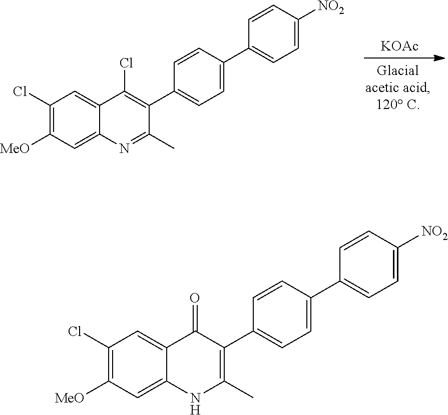 [SMILES: COc1cc2nc(C)c(-c3ccc(-c4ccc([N+](=O)[O-])cc4)cc3)c(Cl)c2cc1Cl ; COc1cc2[nH]c(C)c(-c3ccc(-c4ccc([N+](=O)[O-])cc4)cc3)c(=O)c2cc1Cl ; "KOAc"]

4,6-Dichloro-7-methoxy-2-methyl-3-(4′-nitro-[1,1′-biphenyl]-4-yl)quinoline (0.07 g, 0.00016 mol) and anhydrous potassium acetate (10 eq, 0.00016 mol, 0.016 g) were heated in glacial acetic acid (10 mL) at 120° C. for 21 hours. The cooled reaction was chilled at 5° C. overnight, then vacuum filtered, rinsing with excess water followed by acetone (3×3 mL) to obtain the desired product as a beige powder (14 mg, 21%, ¹H-NMR (400 MHz; DMSO-d₆): δ 11.72 (s, 1H), 8.35-8.32 (m, 2H), 8.05-8.02 (m, 3H), 7.85-7.81 (m, 2H), 7.44-7.41 (m, 2H), 7.09 (s, 1H), 3.98 (s, 3H), 2.28 (s, 3H)).

### 4,6-Dichloro-3-(2′,6′-dimethyl-[1,1′-biphenyl]-4-yl)-7-methoxy-2-methylquinoline

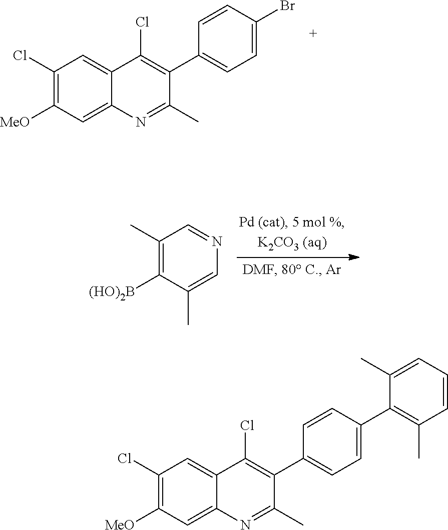 [SMILES: COc1cc2nc(C)c(-c3ccc(Br)cc3)c(Cl)c2cc1Cl ; Cc1cncc(C)c1B(O)O ; COc1cc2nc(C)c(-c3ccc(-c4c(C)cccc4C)cc3)c(Cl)c2cc1Cl]

A mixture of 3-(4-bromophenyl)-4,6-dichloro-7-methoxy-2-methylquinoline (0.70 g, 0.0018 mol), 2,6-dimethylphenylboronic acid (2.0 eq, 0.0036 mol, 0.54 g), and aqueous potassium carbonate (2.0 eq, 0.0036 mol, 0.50 g of anhydrous potassium carbonate dissolved in 1.8 mL water) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.066 g, 0.000090 mol) was added, followed by heating at 80° C. under an atmosphere of argon for 12 days. Although the reaction was not complete, it was removed from the heat. The cooled reaction mixture was vacuum filtered, and the solid obtained was rinsed with DMF followed by excess water and finally, acetone (5 mL). The resulting ash-gray solid (0.33 g) was boiled in 35 mL DMF and allowed to cool slowly, resulting in the formation of white crystals as well as a small amount of dark precipitate that sinks. Vacuum filtration afforded white crystals mixed with a small amount of dark precipitate. GC/MS shows only the m/z of the desired product (421.1) at t_{R}=12.659 min, 200° C. for 2 minutes, then 30° C./minute increase to 300° C., DB5 column. This material (0.19 g) was used without further analysis or purification in the next reaction.

### 6-Chloro-3-(2′,6′-dimethyl-[1,1′-biphenyl]-4-yl)-7-methoxy-2-methylquinolin-4(1H)-one (ELQ-729)

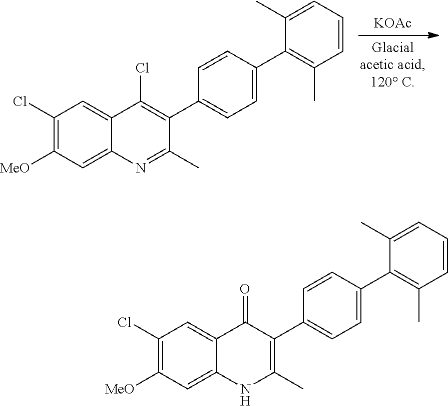 [SMILES: COc1cc2nc(C)c(-c3ccc(-c4c(C)cccc4C)cc3)c(Cl)c2cc1Cl ; COc1cc2[nH]c(C)c(-c3ccc(-c4c(C)cccc4C)cc3)c(=O)c2cc1Cl ; "KOAc"]

4,6-Dichloro-3-(2′,6′-dimethyl-[1,1′-biphenyl]-4-yl)-7-methoxy-2-methylquinoline (0.19 g of crude crystallized material from the preceding reaction, 0.19 g) and anhydrous potassium acetate (0.44 g, 0.0045 mol) were heated at 110° C. in glacial acetic acid (15 mL) for 5 days. The hot reaction mixture was vacuum filtered to remove a small amount of gray solid, and the cooled filtrate was chilled at 5° C., then vacuum filtered, rinsing with excess water followed by acetone (3×1 mL). The resulting solid (79 mg) was recrystallized from 1.5 mL DMF to afford the desired product as sparkling off-white crystals (63 mg, 9% over two steps from 3-(4-bromophenyl)-4,6-dichloro-7-methoxy-2-methylquinoline, ¹H-NMR (400 MHz; DMSO-d₆): δ 11.68 (s, 1H), 8.02 (s, 1H), 7.34-7.31 (m, 2H), 7.19-7.12 (m, 5H), 7.09 (s, 1H), 3.97 (s, 3H), 2.27 (s, 3H), 2.04 (s, 6H)).

### 4,6-Dichloro-7-methoxy-2-methyl-3-(2′-methyl-[1,1′-biphenyl]-4-yl)quinoline

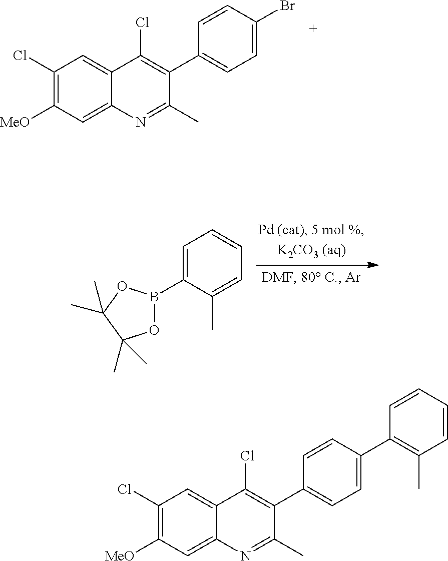 [SMILES: COc1cc2nc(C)c(-c3ccc(Br)cc3)c(Cl)c2cc1Cl ; Cc1ccccc1B1OC(C)(C)C(C)(C)O1 ; COc1cc2nc(C)c(-c3ccc(-c4ccccc4C)cc3)c(Cl)c2cc1Cl]

A mixture of 3-(4-bromophenyl)-4,6-dichloro-7-methoxy-2-methylquinoline (1.00 g, 0.0025 mol), 2-methylphenylboronic acid pinacol ester (1.4 eq, 0.0035 mol, 0.77 g), and aqueous potassium carbonate (2.0 eq, 0.0050 mol, 0.69 g of anhydrous potassium carbonate dissolved in 2.5 mL water) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.091 g, 0.000125 mol) was added, followed by heating at 80° C. under an atmosphere of argon for 1 day. The cooled reaction mixture was vacuum filtered through Celite, and the filtrate was concentrated under reduced pressure with heating. The residue was taken up in 125 mL dichloromethane and vacuum filtered, and the evaporated filtrate was purified by automated flash chromatography on silica gel, eluting with a gradient of 95:5 to 81:19 v:v hexanes:ethyl acetate to afford the desired product as a solid (R_{f}=0.31, 7:3 v:v hexanes:ethyl acetate, silica, 0.08 g, 80%, ¹H-NMR (400 MHz; CDCl₃): δ 8.26 (s, 1H), 7.48-7.45 (m, 3H), 7.35-7.28 (m, 6H), 4.07 (s, 3H), 2.52 (s, 3H), 2.36 (s, 3H)).

### 6-Chloro-7-methoxy-2-methyl-3-(2′-methyl-[1,1′-biphenyl]-4-yl)quinolin-4(1H)-one (ELQ-718)

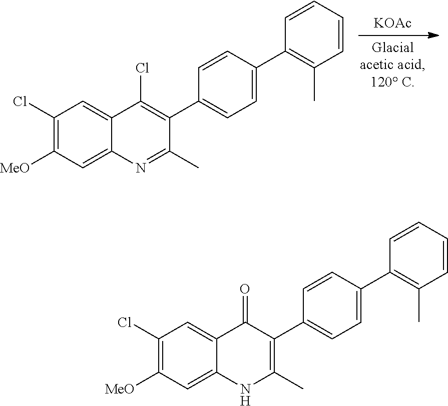 [SMILES: COc1cc2nc(C)c(-c3ccc(-c4ccccc4C)cc3)c(Cl)c2cc1Cl ; COc1cc2[nH]c(C)c(-c3ccc(-c4ccccc4C)cc3)c(=O)c2cc1Cl ; "KOAc"]

4,6-Dichloro-7-methoxy-2-methyl-3-(2′-methyl-[1,1′-biphenyl]-4-yl)quinoline (0.08 g, 0.00020 mol) and anhydrous potassium acetate (10 eq, 0.0020 mol, 0.19g) were heated at 120° C. in glacial acetic acid (10 mL) for 21 hours. After cooling to room temperature, the reaction mixture was chilled for 30 minutes at 5° C. The desired product was recovered by vacuum filtration, rinsing with excess water followed by 2×3 mL acetone. The resulting solid (34 mg) was recrystallized from N,N-dimethylformamide (2 mL). The desired product was obtained as a gray powder (17 mg, 22%, ¹H-NMR (400 MHz; DMSO-d₆): δ 11.68 (s, 1H), 8.02 (s, 1H), 7.38-7.26 (m, 8H), 7.09 (s, 1H), 3.97 (s, 3H), 2.31 (s, 3H), 2.27 (s, 3H)).

### 4,6-Dichloro-7-methoxy-2-methyl-3-(2′,3′,4′-trifluoro-[1,1′-biphenyl]-4-yl)quinoline

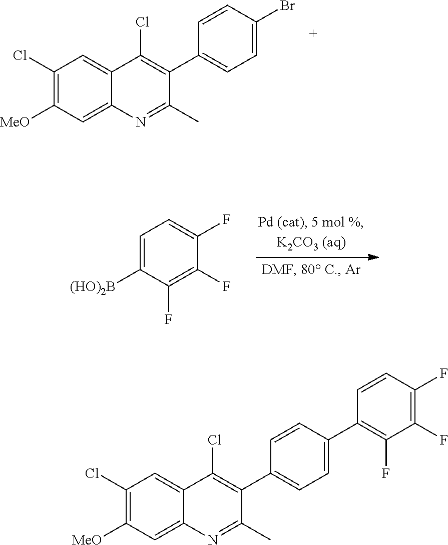 [SMILES: COc1cc2nc(C)c(-c3ccc(Br)cc3)c(Cl)c2cc1Cl ; OB(O)c1ccc(F)c(F)c1F ; COc1cc2nc(C)c(-c3ccc(-c4ccc(F)c(F)c4F)cc3)c(Cl)c2cc1Cl]

A mixture of 3-(4-bromophenyl)-4,6-dichloro-7-methoxy-2-methylquinoline (0.70 g, 0.0018 mol), 2,3,4-trifluorophenylboronic acid (1.2 eq, 0.0022 mol, 0.39 g), and aqueous potassium carbonate (2.0 eq, 0.0036 mol, 0.50 g of anhydrous potassium carbonate dissolved in 1.8 mL water) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.065 g, 0.000090 mol) was added, followed by heating at 80° C. under an atmosphere of argon for 20 hours. The cooled reaction mixture was vacuum filtered, and the resulting solid was rinsed with additional DMF followed by water. After air drying, the white solid thus obtained was taken up in boiling DMF (50 mL) and vacuum filtered while hot, and the filtrate was then concentrated by boiling to 30 mL followed by slow cooling. Vacuum filtration afforded the desired product as tan crystals (0.39 g, 48%, ¹H-NMR (400 MHz; CDCl₃): δ 8.25 (s, 1H), 7.66-7.64 (m, 2H), 7.47 (s, 1H), 7.38-7.36 (m, 2H), 7.27-7.23 (m, ˜1H, overlaps CDCl₃ solvent residual peak), 7.12-7.06 (m, 1H), 4.07 (s, 3H), 2.50 (s, 3H).

19-F NMR (376 MHz; CDCl3): δ −135.1, −138.4, −159.7).

### 6-Chloro-7-methoxy-2-methyl-3-(2′,3′,4′-trifluoro-[1,1′-biphenyl]-4-yl)quinolin-4(1H)-one (ELQ-747)

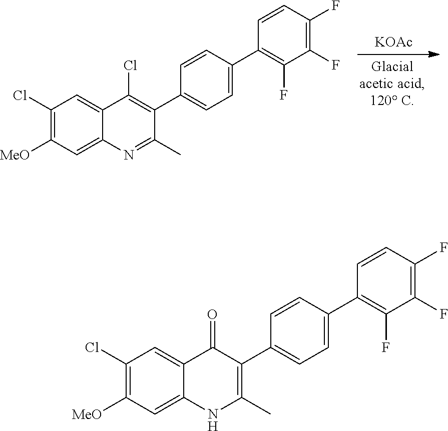 [SMILES: COc1cc2nc(C)c(-c3ccc(-c4ccc(F)c(F)c4F)cc3)c(Cl)c2cc1Cl ; COc1cc2[nH]c(C)c(-c3ccc(-c4ccc(F)c(F)c4F)cc3)c(=O)c2cc1Cl ; "KOAc"]

4,6-Dichloro-7-methoxy-2-methyl-3-(2′,3′,4′-trifluoro-[1,1′-biphenyl]-4-yl)quinoline (0.39 g, 0.00087 mol) and anhydrous potassium acetate (10 eq, 0.0087 mol, 0.86 g) were heated at 120° C. in glacial acetic acid (10 mL) for 2 days. After cooling to room temperature, the reaction mixture was chilled at 5° C. for 2 hours. Vacuum filtration, rinsing with excess water followed by acetone (3×1.5 mL), afforded the desired product as a beige powder (0.28 g, 76%, ¹H-NMR (400 MHz; DMSO-d₆): δ 11.71 (s, 1H), 8.01 (s, 1H), 7.59-7.56 (m, 2H), 7.50-7.42 (m, 2H), 7.40-7.37 (m, 2H), 7.09 (s, 1H), 3.97 (s, 3H), 2.27 (s, 3H).

¹⁹F NMR (376 MHz; DMSO): δ −136.4, −139.6, −161.0).

### 4-Chloro-6-fluoro-7-methoxy-2-methyl-3-(2′,3′,4′-trifluoro-[1,1′-biphenyl]-4-yl)quinoline

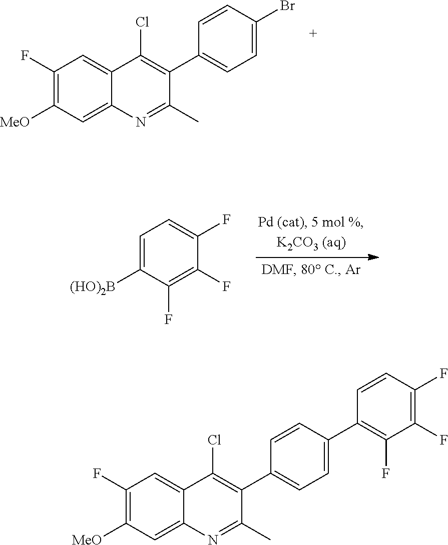 [SMILES: COc1cc2nc(C)c(-c3ccc(Br)cc3)c(Cl)c2cc1F ; OB(O)c1ccc(F)c(F)c1F ; COc1cc2nc(C)c(-c3ccc(-c4ccc(F)c(F)c4F)cc3)c(Cl)c2cc1F]

A mixture of 3-(4-bromophenyl)-4-chloro-6-fluoro-7-methoxy-2-methylquinoline (0.70 g, 0.0018 mol), 2,3,4-trifluorophenylboronic acid (1.2 eq, 0.0022 mol, 0.39 g), and aqueous potassium carbonate (2.0 eq, 0.0036 mol, 0.50 g of anhydrous potassium carbonate dissolved in 1.8 mL water) in N,N-dimethylformamide (80 mL) was degassed by bubbling argon through a glass tube under the liquid surface for 20 minutes at room temperature. [1,1′-Bis(diphenylphosphino)ferrocene]-dichloropalladium (II) (5 mol %, 0.065 g, 0.000090 mol) was added, followed by heating at 80° C. under an atmosphere of argon for 20 hours. The cooled reaction mixture was vacuum filtered, and the resulting solid was rinsed with additional DMF followed by water. After air drying, the cream crystals thus obtained (0.89 g) was taken up in boiling DMF (60 mL) and vacuum filtered while hot, and the filtrate was then concentrated by boiling to 40 mL followed by slow cooling. Vacuum filtration, rinsing with DMF (10 mL) followed by acetone (15 mL), afforded the desired product as white crystals (0.41 g). Meanwhile, the filtrate from initial filtration of the reaction mixture was concentrated under reduced pressure with heating. The residue was taken up in 125 mL dichloromethane and vacuum filtered, followed by automated flash chromatography of the evaporated filtrate on silica gel, eluting with a gradient of 93:7 to 70:30 v:v hexanes:ethyl acetate; this also afforded the desired product (R_{f}=0.24, 7:3 v:v hexanes:ethyl acetate, silica) as a white solid (0.23 g, total yield from crystallization and chromatography 0.64 g, 82%, ¹H-NMR (400 MHz; CDCl₃): δ 7.86 (d, J_{F}=11.8 Hz, 1H), 7.66-7.63 (m, 2H), 7.50 (d, J_{F}=8.1 Hz, 1H), 7.39-7.36 (m, 2H), 7.29-7.23 (m, ˜1H, overlaps CDCl₃ solvent residual peak), 7.12-7.06 (m, 1H), 4.06 (s, 3H), 2.50 (s, 3H), ¹⁹F NMR (376 MHz; CDCl3): δ −131.3, −135.1, −138.4, −159.8).

### 6-Fluoro-7-methoxy-2-methyl-3-(2′,3′,4′-trifluoro-[1,1′-biphenyl]-4-yl)quinolin-4(1H)-one (ELQ-748)

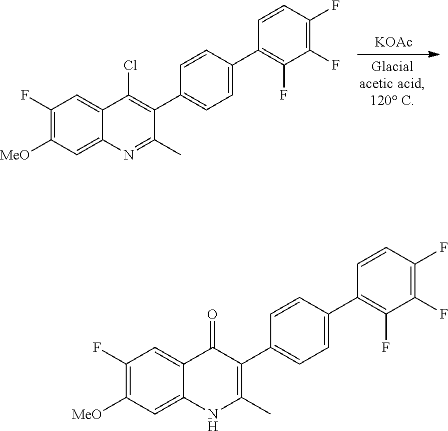 [SMILES: COc1cc2nc(C)c(-c3ccc(-c4ccc(F)c(F)c4F)cc3)c(Cl)c2cc1F ; COc1cc2[nH]c(C)c(-c3ccc(-c4ccc(F)c(F)c4F)cc3)c(=O)c2cc1F ; "KOAc"]

4-Chloro-6-fluoro-7-methoxy-2-methyl-3-(2′,3′,4′-trifluoro-[1,1′-biphenyl]-4-yl)quinoline (0.41 g, 0.00095 mol) and anhydrous potassium acetate (10 eq, 0.0095 mol, 0.93 g) were heated at 120° C. in glacial acetic acid (10 mL) for 2 days. After cooling to room temperature, the reaction mixture was chilled at 5° C. for 2 hours. Vacuum filtration, rinsing with excess water followed by acetone (3×1.5 mL), afforded the desired product as a beige powder (0.34 g, 82%, ¹H-NMR (400 MHz; DMSO-d₆): δ 11.69 (s, 1H), 7.72 (d, J_{F}=11.7 Hz, 1H), 7.59-7.56 (m, 2H), 7.51-7.42 (m, 2H), 7.40-7.37 (m, 2H), 7.12 (d, J_{F}=7.3 Hz, 1H), 3.96 (s, 3H), 2.26 (s, 3H), ¹⁹F NMR (376 MHz; DMSO): δ −136.4, −139.4, −139.6, −161.0).

### 4-Chloro-6-fluoro-7-methoxy-2-methyl-3-(4-(6-(trifluoromethyl)pyridin-3-yl)phenyl)quinoline

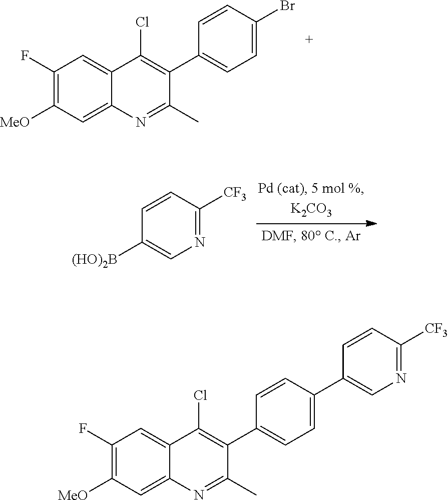 [SMILES: COc1cc2nc(C)c(-c3ccc(Br)cc3)c(Cl)c2cc1F ; OB(O)c1ccc(C(F)(F)F)nc1 ; COc1cc2nc(C)c(-c3ccc(-c4ccc(C(F)(F)F)nc4)cc3)c(Cl)c2cc1F]

A mixture of 3-(4-bromophenyl)-4-chloro-6-fluoro-7-methoxy-2-methylquinoline (0.57 g, 0.0015 mol), 2-(trifluoromethyl)pyridine-5-boronic acid (1.2 eq, 0.0018 mol, 0.34 g), and aqueous potassium carbonate (2.0 eq, 0.0030 mol, 0.41 g of anhydrous potassium carbonate dissolved in 1.5 mL water) in N,N-dimethylformamide (60 mL) was degassed by bubbling argon through a glass tube under the liquid surface for 20 minutes at room temperature. [1,1′-Bis(diphenylphosphino)ferrocene]-dichloropalladium (II) (5 mol %, 0.055 g, 0.000075 mol) was added, followed by heating at 80° C. under an atmosphere of argon for 4 days. The cooled reaction mixture was vacuum filtered, and the filtrate was evaporated under reduced pressure with heating. The residue was taken up in dichloromethane (125 mL) and vacuum filtered. Automated flash chromatography of the evaporated filtrate on silica gel, eluting with a gradient of 85:15 to 63:37 v:v hexanes:ethyl acetate. The desired product was obtained as a white, crystalline solid (0.43 g, 64%, ¹H-NMR (400 MHz; CDCl₃): δ 9.07-9.04 (m, 1H), 8.16-8.13 (m, 1H), 7.86 (d, J_{F}=11.8 Hz, 1H), 7.82-7.80 (m, 1H), 7.78-7.75 (m, 2H), 7.51 (d, J_{F}=8.1 Hz, 1H), 7.46-7.43 (m, 2H), 4.07 (s, 3H), 2.50 (s, 3H), ¹⁹F NMR (376 MHz; CDCl3): δ −67.7, −131.1).

### 6-Fluoro-7-methoxy-2-methyl-3-(4-(6-(trifluoromethyl)pyridin-3-yl)phenyl)quinolin-4(1H)-one (ELQ-758)

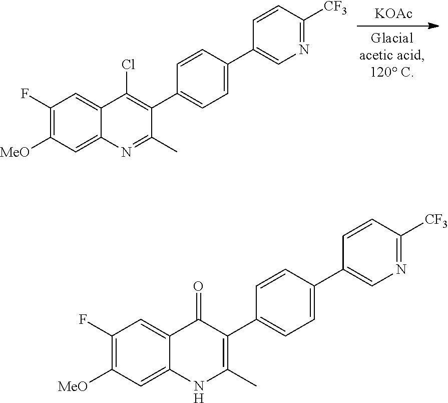 [SMILES: COc1cc2nc(C)c(-c3ccc(-c4ccc(C(F)(F)F)nc4)cc3)c(Cl)c2cc1F ; COc1cc2[nH]c(C)c(-c3ccc(-c4ccc(C(F)(F)F)nc4)cc3)c(=O)c2cc1F ; "KOAc"]

4-chloro-6-fluoro-7-methoxy-2-methyl-3-(4-(6-(trifluoromethyl)pyridin-3-yl)phenyl) quinoline (0.43 g, 0.00096 mol) and anhydrous potassium acetate (10 eq, 0.0096 mol, 0.94g) were heated at 120° C. in glacial acetic acid (10 mL) for 1 day. After cooling, the reaction mixture was chilled at 5° C. overnight. Vacuum filtration, rinsing with excess water followed by acetone (3×1.5 mL), afforded the desired product as beige crystals (0.32 g, 78%, 1H-NMR (400 MHZ; DMSO-d₆): δ 11.69 (s, 1H), 9.17-9.15 (m, 1H), 8.44-8.41 (m, 1H), 8.03-7.98 (m, 1H), 7.87-7.84 (m, 2H), 7.72 (d, J_{F}=11.7 Hz, 1H), 7.45-7.42 (m, 2H), 7.12 (d, J_{F}=7.3 Hz, 1H), 3.97 (s, 3H), 2.27 (s, 3H), 19F NMR (376 MHz; DMSO): δ −66.2, −139.4).

### Synthesis of ELQ Biphenyls

Compounds of Formula (I) may be prepared as illustrated in Scheme 8, below.

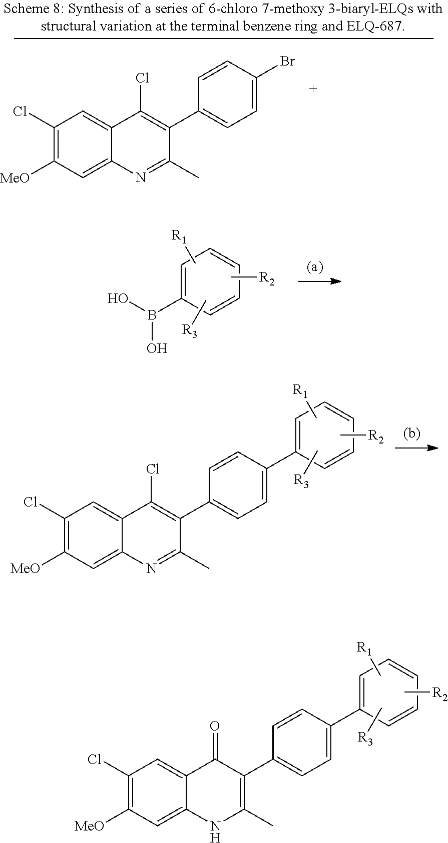

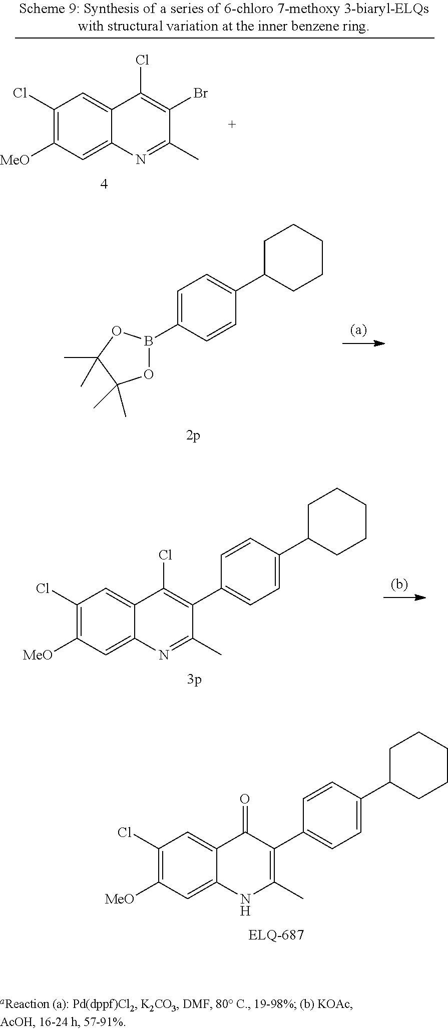 [SMILES: COc1cc2nc(C)c(Br)c(Cl)c2cc1Cl ; CC1(C)OB(c2ccc(C3CCCCC3)cc2)OC1(C)C ; COc1cc2nc(C)c(-c3ccc(C4CCCCC4)cc3)c(Cl)c2cc1Cl ; COc1cc2[nH]c(C)c(-c3ccc(C4CCCCC4)cc3)c(=O)c2cc1Cl]

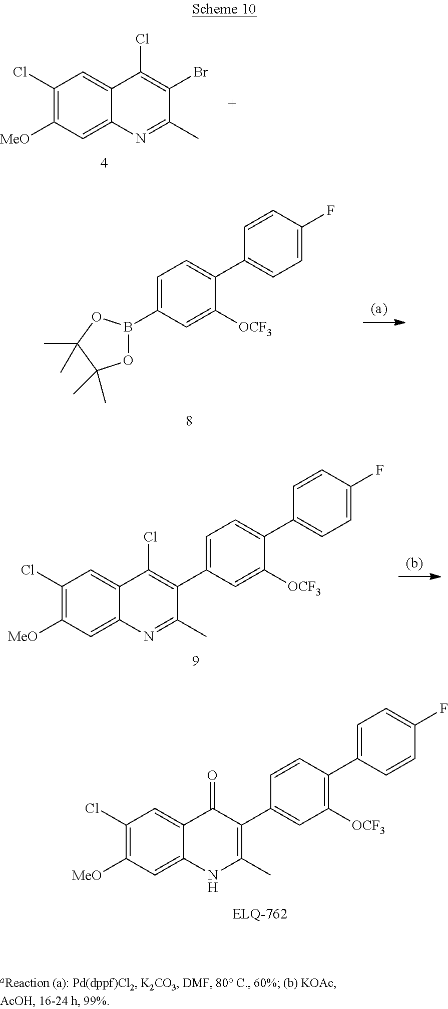 [SMILES: COc1cc2nc(C)c(Br)c(Cl)c2cc1Cl ; CC1(C)OB(c2ccc(-c3ccc(F)cc3)c(OC(F)(F)F)c2)OC1(C)C ; COc1cc2nc(C)c(-c3ccc(-c4ccc(F)cc4)c(OC(F)(F)F)c3)c(Cl)c2cc1Cl ; COc1cc2[nH]c(C)c(-c3ccc(-c4ccc(F)cc4)c(OC(F)(F)F)c3)c(=O)c2cc1Cl]

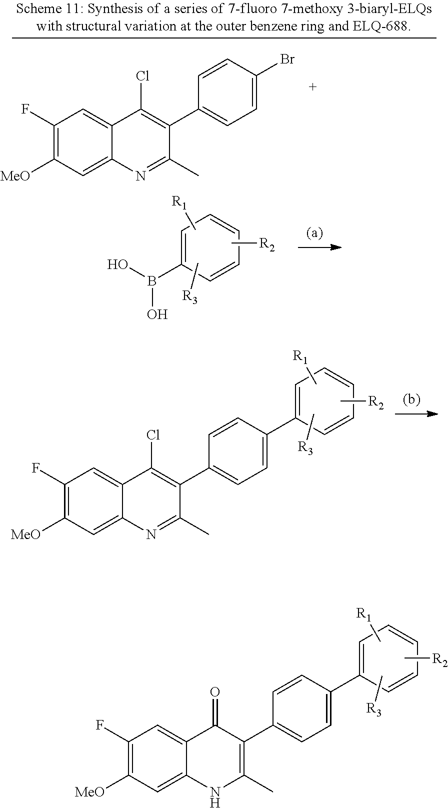

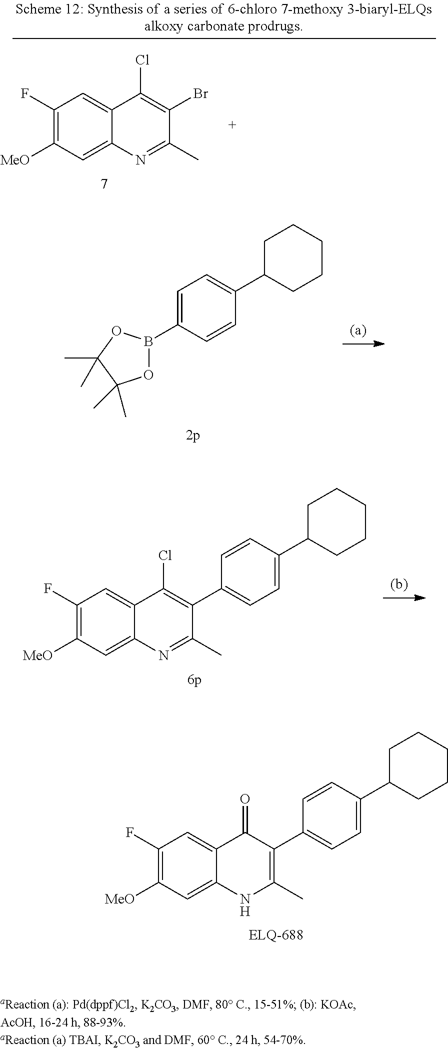 [SMILES: COc1cc2nc(C)c(Br)c(Cl)c2cc1F ; CC1(C)OB(c2ccc(C3CCCCC3)cc2)OC1(C)C ; COc1cc2nc(C)c(-c3ccc(C4CCCCC4)cc3)c(Cl)c2cc1F ; COc1cc2[nH]c(C)c(-c3ccc(C4CCCCC4)cc3)c(=O)c2cc1F]

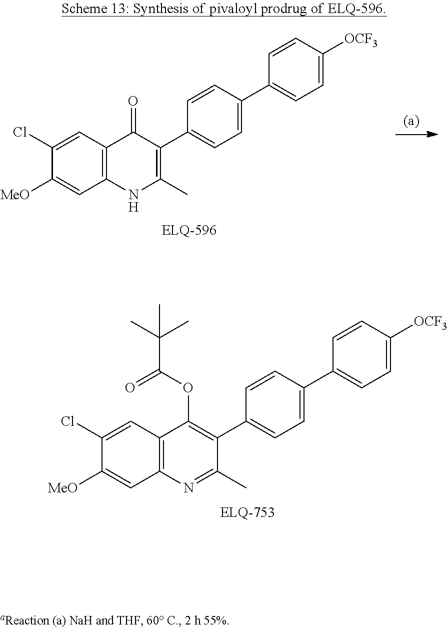 [SMILES: COc1cc2[nH]c(C)c(-c3ccc(-c4ccc(OC(F)(F)F)cc4)cc3)c(=O)c2cc1Cl ; COc1cc2nc(C)c(-c3ccc(-c4ccc(OC(F)(F)F)cc4)cc3)c(OC(=O)C(C)(C)C)c2cc1Cl]

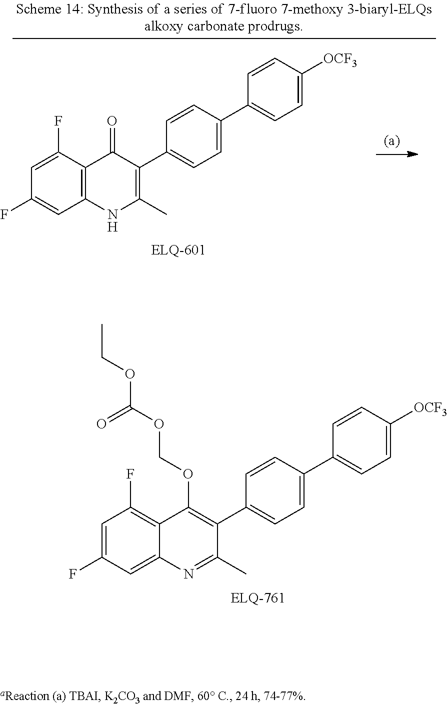 [SMILES: Cc1[nH]c2cc(F)cc(F)c2c(=O)c1-c1ccc(-c2ccc(OC(F)(F)F)cc2)cc1 ; CCOC(=O)OCOc1c(-c2ccc(-c3ccc(OC(F)(F)F)cc3)cc2)c(C)nc2cc(F)cc(F)c12]

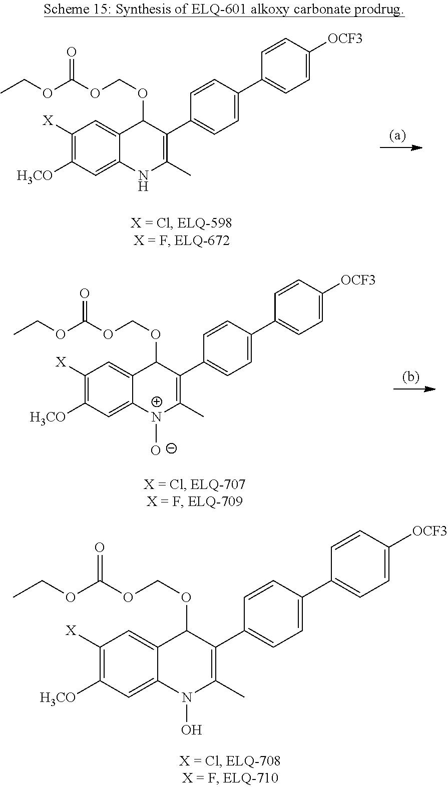

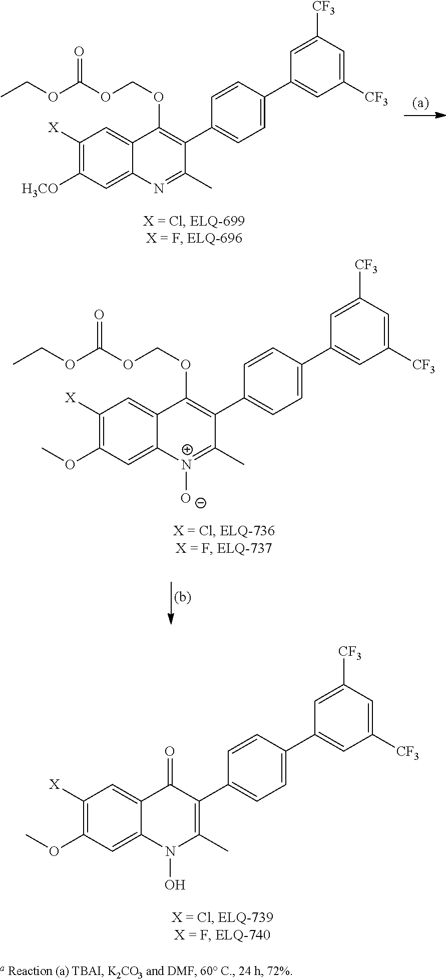

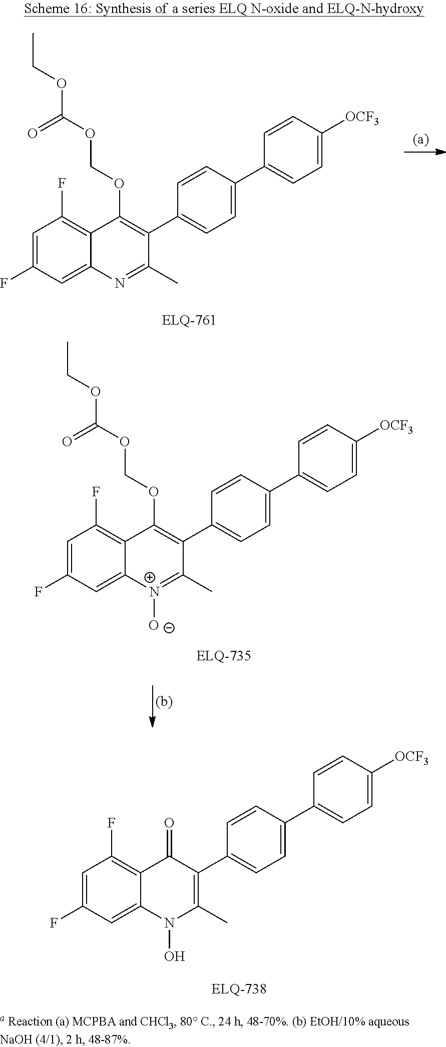 [SMILES: CCOC(=O)OCOc1c(-c2ccc(-c3ccc(OC(F)(F)F)cc3)cc2)c(C)nc2cc(F)cc(F)c12 ; CCOC(=O)OCOc1c(-c2ccc(-c3ccc(OC(F)(F)F)cc3)cc2)c(C)[n+]([O-])c2cc(F)cc(F)c12 ; Cc1c(-c2ccc(-c3ccc(OC(F)(F)F)cc3)cc2)c(=O)c2c(F)cc(F)cc2n1O]

### Chemical Synthesis Procedures.

Unless otherwise stated all chemicals and reagents were from Sigma-Aldrich Chemical Company in St. Louis, MO (USA), Combi-Blocks, San Diego (CA), or TCI America, Portland (OR) and were used as received. 3-(4-bromophenyl)-4,6-dichloro-7-methoxy-2-methylquinoline (1), 3-bromo-4,6-dichloro-7-methoxy-2-methylquinoline (4), 3-(4-bromophenyl)-4-chloro-6-fluoro-7-methoxy-2-methylquinoline (5), 3-(4-bromophenyl)-4-chloro-6-fluoro-7-methoxy-2-methylquinoline (7), ELQ-596, ELQ-598, ELQ-650 and ELQ-601 were obtained as previously reported. Melting points were obtained in the Optimelt Automated Melting point system from Stanford Research Systems, Sunnyvale, CA (USA). Analytical TLC utilized Merck 60F-254 250 micron precoated silica gel plates and spots were visualized under 254 nm UV light. GC-MS was obtained using an Agilent Technologies 7890B gas chromatograph (30 m, DBS column set at either 100° C. or 200° C. for 2 min, then at 30° C./min to 300° C. with inlet temperature set at 250° C.) with an Agilent Technologies 5977A mass-selective detector operating at 70 eV. Flash chromatography over silica gel column was performed using an Isolera One flash chromatography system from Biotage, Uppsala, Sweden. ¹H-NMR spectra were obtained using a Bruker 400 MHz Avance NEO NanoBay NMR spectrometer operating at 400.14 MHz. The NMR raw data were analyzed using the iNMR Spectrum Analyst software. ¹H chemical shifts are reported in parts per million (ppm) relative to internal tetramethylsilane (TMS) standard or residual solvent peak. Coupling constant values (J) are reported in hertz (Hz). Decoupled ¹⁹F operating at 376 MHz was also obtained for compounds containing fluorine (data not shown). HPLC analyses were performed using an Agilent 1260 Infinity instrument with detection at 254 nm and a Phenomenex, Luna® 5 μm C8(2) 100 Å reverse phase LC column 150×4.6 mm at 40° C., and eluted with a gradient of A/B at 25%:75% to A/B at 10%:90% (A: 0.05% formic acid in milliQ water, B: 0.05% formic acid in methanol). All compounds were at least >95% pure for in vitro testing and >98% pure for in vivo testing as determined by GC-MS, ¹H-NMR and HPLC.

General Procedure A for the synthesis of the biphenyl quinolines (3a-p). A stirred mixture of quinoline 1(1 eq), substituted phenyl boronic acids (1.1-1.2 eq) 2a, 2c, 2e-2g, 2j-2m, 15g, or pinacol esters 2b, 2d, 2h, 2i, 2n and 2o, aqueous 2M K₂CO₃ (2 eq) and Pd(dppf)Cl₂ (0.05 eq), in DMF was deoxygenated by bubbling argon through the solution for 15 minutes. The stirred reaction mixture was then heated at 80° C. under argon until no more starting material 1 remained as determined by GC-MS. The reaction was cooled to room temperature and filtered through celite, and DMF was removed in vacuo. The resulting black oily solid was resuspended in DCM and stirred vigorously at room temperature for 30 minutes, filtered through celite, and concentrated to dryness. The residue was taken up with 3-5 ml of DCM, if all the solid was dissolved then the product was purified by flash chromatography. In instances where the products were not soluble in methylene chloride, they were filtered, washed with DCM and the filtrates were further purified by flash chromatography to give additional material.

General Procedure B for the hydrolysis of the 4-chloro quinolines. A stirred mixture of the 4-chloro quinolines (3a-o, 1 eq), potassium acetate (KOAc, 10 eq) and glacial acetic acid was heated at 120° C. in a loosely capped reaction vial for 16-24 h. After cooling to room temperature, the reaction mixture was poured into ice water (20-30 ml). The resulting precipitate was filtered washed with water (3×15 ml), acetone (3×10 ml), DCM (3×10 ml), hexane (3×10 ml) and air-dried to give the desired product. If the products were less than 98% pure by NMR and HPLC they can be obtained in pure form by crystallization from DMF.

General Procedure C for the synthesis of the alkoxy carbonate pro-drug: A stirred mixture of 4-(1H)-quinolone ELQ (1 eq), tetrabutylammonium iodide (TBAI) (2 eq), dry K₂CO₃ (2 eq) and chloromethyl ethylcarbonate (2 eq) in DMF was heated at 60° C. for 24 h. The mixture was cooled to room temperature, filtered and the filtrate concentrated to dryness to give an oil. The resulting residue was stirred with ethyl acetate for 30 minutes and the insoluble TBAI filtered and washed with ethyl acetate. The filtrate was concentrated to dryness and purified by flash chromatography using a gradient of ethyl acetate/hexane as eluent to give the desired prodrug. If the resulting prodrugs were less than 98% by GM-MS and NMR they can be obtained in pure form by crystallization from hexane/ethyl acetate.

### General Procedure D for the Synthesis the N-Oxide Prodrugs:

To a stirred solution of ELQ alkoxy carbonate (1 eq) in chloroform was added MCPBA (1.5 eq) and the solution was heated to 90° C. for 24 hours. After cooling to room temperature, the yellow solution was concentrated to dryness under vacuo and was purified by flash chromatography over silica gel.

### General Procedure E for the Hydrolysis of the N-Oxide Prodrugs:

A solution of the alkoxy carbonate nitrone in ethanol/aqueous 10% NaOH (4/1) was heated for 2 h at 60° C. some white precipitate was formed. The solution was concentrated to dryness. The resulting solid was then washed with water (3×10 ml), DCM (3×10 ml) and air dried to give the desired N-hydroxy ELQ.

3-(3′,5′-bis(trifluoromethyl)-[1,1′-biphenyl]-4-yl)-4,6-dichloro-7-methoxy-2-methylquinoline (3a): Following the general procedure A, a mixture of 1 (794 mg, 2.0 mmol, 1 eq), 2a (568 mg, 2.2 mmol, 1.1 eq), aqueous K₂CO₃ (2 ml, 2 eq), Pd(dppf)Cl₂ (73 mg, 0.1 mmol, 0.05 eq) and DMF (75 ml) was heated for 16 h to give crude 3a (1.09 gm) as a black solid. DCM (15 ml) was added and the precipitate was filtered washed with methylene chloride (2×5 ml) to give pure 3a (305 mg) as a white solid, second crop from DCM give another pure 3a (126 mg). The mother liquor was further purified by flash chromatography using a gradient of ethyl acetate/hexane (3/7) as the eluting solvent to yield additional 3a (48 mg) for a combined yield of 3a (479 mg, 45% yield). GC-MS shows one peak M⁺=529 (100%). ¹H-NMR (400 MHz; CDCl₃): δ 8.25 (s, 1H), 8.12-8.11 (m, 2H), 7.91-7.90 (m, 1H), 7.78-7.75 (m, 2H), 7.48 (s, 1H), 7.45-7.42 (m, 2H), 4.08 (s, 3H), 2.50 (s, 3H).

4,6-dichloro-3-(4′-cyclohexyl-[1,1′-biphenyl]-4-yl)-7-methoxy-2-methylquinoline (3b): Following the general procedure A, a mixture of 1 (794 mg, 2.0 mmol, 1 eq), 2b (630 mg, 2.2 mmol, 1.1 eq), aqueous K₂CO₃ (2 ml, 2 eq), Pd(dppf)Cl₂ (73 mg, 0.1 mmol, 0.05 eq) and DMF (75 ml) was heated for 48 h to give crude 3a (1.03 gm) as a black solid. The product was purified twice by flash chromatography using a gradient of ethyl acetate/hexane (1/9) as the eluting solvent to give 3b (529 mg). The product was crystallized in ethyl acetate/hexane to give pure 3b (400 mg, 42% yield) as a light yellow solid. GC-MS shows one peak M⁺=475 (42%). ¹H-NMR (400 MHz; CDCl₃): δ 8.24 (s, 1H), 7.72 (d, J=8.1 Hz, 2H), 7.61 (d, J=8.1 Hz, 2H), 7.46 (s, 1H), 7.33-7.31 (m, 4H), 4.07 (s, 3H), 2.60-2.53 (m, 1H), 2.50 (s, 3H), 1.95-1.86 (m, 4H), 1.79-1.76 (m, 1H), 1.53-1.36 (m, 4H), 1.35-1.24 (m, 1H).

4,6-dichloro-3-(2′-chloro-4′-(trifluoromethoxy)-[1,1′-biphenyl]-4-yl)-7-methoxy-2-methylquinoline (3c): Following the general procedure A, a mixture of 1 (794 mg, 2.0 mmol, 1 eq), 2c (528 mg, 2.2 mmol, 1.1 eq), aqueous K₂CO₃ (2 ml, 2 eq), Pd(dppf)Cl₂ (73 mg, 0.1 mmol, 0.05 eq) and DMF (75 ml) was heated for 48 h to give crude 3c (1.0 gm) as a black solid. The product was purified by flash chromatography using a gradient of ethyl acetate/hexane (2/8) as the eluting solvent to give ˜98% pure as determine by GC-MS and NMR 3c (190 mg, 19% yield) as a white solid. GC-MS shows one peak M⁺=511 (100%). ¹H-NMR (400 MHz; CDCl₃): δ 8.26 (s, 1H), 7.57-7.55 (m, 2H), 7.50-7.46 (m, 2H), 7.41 (s, 1H), 7.36-7.34 (m, 2H), 7.26-7.23 (m, 2H), 4.08 (s, 3H), 2.51 (s, 3H).

4,6-dichloro-7-methoxy-2-methyl-3-(2′-methyl-4′-(trifluoromethoxy)-[1,1′-biphenyl]-4-yl)quinoline (3d): Following the general procedure A, a mixture of 1 (794 mg, 2.0 mmol, 1 eq), 2d (664 mg, 2.2 mmol, 1.1 eq), aqueous K₂CO₃ (2 ml, 2 eq), Pd(dppf)Cl₂ (73 mg, 0.1 mmol, 0.05 eq) and DMF (75 ml) was heated for 16 h to give crude 3d (1.26 gm) as a black solid. The product was purified by flash chromatography using a gradient of ethyl acetate/hexane (2/8) as the eluting solvent to give 3d (654 mg) as a white solid. The product was crystallized in ethyl acetate to give pure 3d (431 mg, 44% yield). GC-MS shows one peak M⁺=491 (100%). ¹H-NMR (400 MHz; CDCl₃): δ 8.26 (s, 1H), 7.48 (s, 1H), 7.45-7.44 (m, 1H), 7.43-7.42 (t, 1H), 7.34-7.31 (m, 3H), 7.17-7.12 (m, 2H), 4.08 (s, 3H), 2.51 (s, 3H), 2.36 (s, 3H).

4,6-dichloro-7-methoxy-2-methyl-3-(2′-(trifluoromethoxy)-[1,1′-biphenyl]-4-yl)quinoline (3e): Following the general procedure A, a mixture of 1 (794 mg, 2.0 mmol, 1 eq), 2e (494 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 24 h to give crude 3e (2.65 gm) as a black solid. The product was purified by flash chromatography using a gradient of ethyl acetate/hexane (2/8) as the eluting solvent to give 3e (638 mg, 67% yield) as a white solid. GC-MS shows one peak M⁺=477 (100%). ¹H-NMR (400 MHz; CDCl₃): δ 8.25 (s, 1H), 7.62-7.59 (m, 2H), 7.57-7.54 (m, 1H), 7.48 (s, 1H), 7.46-7.39 (m, 3H), 7.36-7.33 (m, 2H), 4.08 (s, 3H), 2.50 (s, 3H).

4,6-dichloro-3-(2′-fluoro-[1,1′-biphenyl]-4-yl)-7-methoxy-2-methylquinoline (3f): Following the general procedure A, a mixture of 1 (794 mg, 2.0 mmol, 1 eq), 2f (494 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 24 h to give crude 3f (738 mg) as a black solid. The product was purified by flash chromatography using a gradient of ethyl acetate/hexane (2/8) as the eluting solvent to give 3f (369 mg, 45% yield) as a white solid. GC-MS shows one peak M⁺=411 (100%). ¹H-NMR (400 MHz; CDCl₃): δ 8.25 (s, 1H), 7.72-7.69 (m, 2H), 7.55 (td, J=7.8, 1.8 Hz, 1H), 7.47 (s, 1H), 7.37-7.34 (m, 3H including CDCl3), 7.28-7.18 (m, 3H), 4.07 (s, 3H), 2.51 (s, 3H).

4,6-dichloro-7-methoxy-2-methyl-3-(2′-(trifluoromethyl)-[1,1′-biphenyl]-4-yl)quinoline (3g): Following the general procedure A, a mixture of 1 (794 mg, 2.0 mmol, 1 eq), 2g (456 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 10 days to give crude 3g (1.65 g) as a black solid. The product was purified by flash chromatography using a gradient of ethyl acetate/hexane (2/8) as the eluting solvent to give 3g (41 mg) as a white solid. The overlap fraction was concentrated in vacuo and the solid was crystallized in ethyl acetate and hexane to give an additional 3g (200 mg) for a combined 3g (241 mg, 22% yield). GC-MS shows one peak M⁺=461 (100%). ¹H-NMR (400 MHz; CDCl₃): δ 8.26 (s, 1H), 7.79 (dd, J=7.8, 0.5 Hz, 1H), 7.64-7.60 (m, 1H), 7.53-7.50 (m, 1H), 7.48-7.46 (m, 4H), 7.32-7.29 (m, 2H), 4.07 (s, 3H), 2.50 (s, 3H).

4,6-dichloro-3-(3′,5′-difluoro-4′-(trifluoromethoxy)-[1,1′-biphenyl]-4-yl)-7-methoxy-2-methylquinoline (3h): Following the general procedure A, a mixture of 1 (794 mg, 2.0 mmol, 1 eq), 2h (778 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 24 h to give crude 3h (1.55 gm) as a black solid. The product was purified by flash chromatography using a gradient of ethyl acetate/hexane (2/8) as the eluting solvent to give ˜95% pure as determine by GC-MS and NMR 3h (677 mg, 66% yield) as a white solid. GC-MS shows one peak M⁺=513 (100%). ¹H-NMR (400 MHz; CDCl₃): δ 8.25 (s, 1H), 7.69-7.66 (m, 2H), 7.47 (s, 1H), 7.41-7.38 (m, 2H), 7.34-7.31 (m, 2H), 4.07 (s, 3H), 2.48 (s, 3H).

4,6-dichloro-3-(3′,5′-difluoro-4′-methoxy-[1,1′-biphenyl]-4-yl)-7-methoxy-2-methylquinoline (3i): Following the general procedure A, a mixture of 1 (794 mg, 2.0 mmol, 1 eq), 2i (648 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 24 h to give crude 3i (1.48 gm) as a black solid. The product was purified by flash chromatography using a gradient of ethyl acetate/hexane (2/8) as the eluting solvent to give ˜95% pure as determine by GC-MS and NMR 3i (903 mg, 98% yield) as a white solid. GC-MS shows one peak M⁺=459 (100%). ¹H-NMR (400 MHz; CDCl₃): δ 8.25 (s, 1H), 7.67-7.63 (m, 2H), 7.47 (s, 1H), 7.37-7.33 (m, 2H), 7.24-7.22 (m, 2H), 4.07-4.06 (m, 6H), 2.49 (s, 3H).

4,6-dichloro-7-methoxy-3-(2′-methoxy-5′-(trifluoromethoxy)-[1,1′-biphenyl]-4-yl)-2-methylquinoline (3j): Following the general procedure A, a mixture of 1 (1.19 gm, 3.0 mmol, 1 eq), 2j (850 mg, 3.6 mmol, 1.2 eq), aqueous K₂CO₃ (3 ml, 6.0 mmol, 2 eq), Pd(dppf)Cl₂ (73 mg, 0.1 mmol, 0.05 eq) and DMF (150 ml) was heated for 18 h to give crude 3j (2.1 gm) as a black solid. The product was purified by flash chromatography using a gradient of ethyl acetate/hexane (2/8) as the eluting solvent to give 3j (976 mg, 64% yield) as a white solid. GC-MS shows one peak M⁺=507 (100%). ¹H-NMR (400 MHz; CDCl₃): δ 8.26 (s, 1H), 7.68-7.65 (m, 2H), 7.47 (s, 1H), 7.34-7.31 (m, 3H), 7.23-7.20 (m, 1H), 7.00 (d, J=9.0 Hz, 1H), 4.07 (s, 3H), 3.88 (s, 3H), 2.52 (s, 3H).

4,6-dichloro-7-methoxy-3-(2′-methoxy-[1,1′-biphenyl]-4-yl)-2-methylquinoline (3k): Following the general procedure A, a mixture of 1 (794 mg, 2.0 mmol, 1 eq), 2k (365 mg, 2.4 mmol, 1.2 eq), aqueous K₂CO₃ (2 ml, 2 eq), Pd(dppf)Cl₂ (73 mg, 0.1 mmol, 0.05 eq) and DMF (100 ml) was heated for 48 h to give crude 3k (1.2 gm) as a black solid. The product was purified by flash chromatography using a gradient of ethyl acetate/hexane (2/8) as the eluting solvent to give 3k (540 mg, 64% yield) as a white solid. GC-MS shows one peak M⁺=423 (100%). (400 MHz; CDCl₃): δ 8.26 (s, 1H), 7.70-7.67 (m, 2H), 7.47 (s, 1H), 7.43 (dd, J=7.5, 1.7 Hz, 1H), 7.36 (ddd, J=8.2, 7.4, 1.8 Hz, 1H), 7.31-7.28 (m, 2H), 7.09-7.02 (m, 2H), 4.07 (s, 3H), 3.87 (s, 3H), 2.53 (s, 3H).

4,6-dichloro-7-methoxy-3-(2′-methoxy-4′-(trifluoromethyl)-[1,1′-biphenyl]-4-yl)-2-methylquinoline (31): Following the general procedure A, a mixture of 1 (794 mg, 2.0 mmol, 1 eq), 21(528 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 18 h to give crude 31 (1.0 gm) as a yellow solid. The product was purified by flash chromatography using a gradient of ethyl acetate/hexane (2/8) as the eluting solvent to give 31(618 mg, 63% yield) as a white solid. GC-MS shows one peak M⁺=491 (100%). ¹H-NMR (400 MHz; CDCl₃): δ 8.26 (s, 1H), 7.69-7.66 (m, 2H), 7.54-7.52 (m, 1H), 7.47 (s, 1H), 7.35-7.32 (m, 3H), 7.23 (s, 1H), 4.07 (s, 3H), 2.51 (s, 3H).

4,6-dichloro-3-(4′-fluoro-2′-methoxy-[1,1′-biphenyl]-4-yl)-7-methoxy-2-methylquinoline (3m): Following the general procedure A, a mixture of 1 (794 mg, 2.0 mmol, 1 eq), 2m (408 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 18 h. When the mixture was filtered over celite, white insoluble solid found on top of the celite was separated and airdried to give pure 3m (503 mg, 57% yield) as a white solid. GC-MS shows one peak M⁺=441 (100%). ¹H-NMR (400 MHz; CDCl₃): δ 8.25 (s, 1H), 7.65-7.62 (m, 2H), 7.47 (s, 1H), 7.39-7.35 (m, 1H), 7.31-7.28 (m, 2H), 6.80-6.74 (m, 2H), 4.07 (s, 3H), 3.86 (s, 3H), 2.52 (s, 3H).

4,6-dichloro-7-methoxy-3-(2′-methoxy-4′-(trifluoromethoxy)-[1,1′-biphenyl]-4-yl)-2-methylquinoline (3n): Following the general procedure A, a mixture of 1 (794 mg, 2.0 mmol, 1 eq), 2n (763 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 36 h to give crude 3n (1.40 gm) as a black solid. The product was purified by flash chromatography using a gradient of ethyl acetate/hexane (2/8) as the eluting solvent to give 3n (597 mg) as a white solid. The product was crystallized in DCM/hexane to give pure 3n (360 mg), second crop from the mother liquor give an additional 3n (120 mg) for a total of pure 3n (480 mg, 47% yield) as a white solid. GC-MS shows one peak M⁺=445 (100%). ¹H-NMR (400 MHz; CDCl₃): δ 8.26 (s, 1H), 7.66-7.63 (m, 2H), 7.47 (s, 1H), 7.42 (d, J=8.4 Hz, 1H), 7.33-7.29 (m, 2H), 6.96-6.93 (m, 1H), 6.86 (d, J=1.7 Hz, 1H), 4.07 (s, 3H), 3.88 (s, 3H), 2.52 (s, 3H).

4′-(4,6-dichloro-7-methoxy-2-methylquinolin-3-yl)-2-methoxy-[1,1′-biphenyl]-4-carbonitrile (30): Following the general procedure A, a mixture of 1 (794 mg, 2.0 mmol, 1 eq), 30 (622 mg, 2.4 mmol, 1.2 eq), aqueous K₂CO₃ (2 ml, 2 eq), Pd(dppf)Cl₂ (73 mg, 0.1 mmol, 0.05 eq) and DMF (75 ml) was heated for 110 h to give crude 30 (1.0 g) as a black solid. The product was purified by flash chromatography using a gradient of ethyl acetate/hexane (3/7) as the eluting solvent to give 3o (581 mg) as a white solid. The product was crystallized in ethyl acetate/DCM to give pure 30 (485 mg, 54% yield) as a white solid. GC-MS shows one peak M⁺=448 (100%). ¹H-NMR (400 MHz; CDCl₃): δ 8.25 (s, 1H), 7.68-7.65 (m, 2H), 7.51 (d, J=7.8 Hz, 1H), 7.47 (s, 1H), 7.39 (dd, J=7.8, 1.5 Hz, 1H), 7.36-7.32 (m, 2H), 7.25 (d, J=1.5 Hz, 1H), 4.07 (s, 3H), 3.91 (s, 3H), 2.51 (s, 3H).

4,6-dichloro-3-(4-cyclohexylphenyl)-7-methoxy-2-methylquinoline (3p): Following the general procedure A, a mixture of 4 (321 mg, 1.0 mmol, 1 eq), 2p (300 mg, 1.05 mmol, 1.05 eq), aqueous K₂CO₃ (1 ml, 2 eq), Pd(dppf)Cl₂ (37 mg, 0.1 mmol, 0.05 eq) and DMF (25 ml) was heated for 16 h and kept at room temperature for 72 h. After the mixture was filtered over celite white solid on top of the celite was separated to give pure 3p (90 mg) as a white solid. The filtrate was concentrated in vacuo to dryness and was purified by flash chromatography using a gradient of ethyl acetate/hexane (1/9) as the eluting solvent to give 3p (89 mg). The product was further crystallized in DMF to give pure 3p (26 mg) for a combined total 3p (116 mg, 29% yield). GC-MS shows one peak M⁺=399 (100%). ¹H-NMR (400 MHz; CDCl₃): δ 8.23 (s, 1H), 7.45 (s, 1H), 7.34-7.31 (m, 2H), 7.18-7.16 (m, 2H), 4.06 (s, 3H), 2.62-2.55 (m, 1H), 2.45 (s, 3H), 1.99-1.96 (m, 2H), 1.90-1.87 (m, 2H), 1.81-1.76 (m, 1H), 1.51-1.25 (m, 6H).

3-(3′,5′-bis(trifluoromethyl)-[1,1′-biphenyl]-4-yl)-4-chloro-6-fluoro-7-methoxy-2-methylquinoline (6a): Following the general procedure A, a mixture of 5 (3.81 g, 10.0 mmol, 1 eq), 2a (2.84 g, 11.0 mmol, 1.1 eq), aqueous K₂CO₃ (20 ml, 2 eq), Pd(dppf)Cl₂ (366 mg, 0.5 mmol, 0.05 eq) and DMF (200 ml) was heated for 16 h to give crude 6a (5.82 g) as a black solid. The product was purified by flash chromatography using a gradient of ethyl acetate/hexane (3/7) as the eluting solvent. During the evaporation of the solvent under vacuo of the combined fractions white precipitate was formed. The product was filtered and washed with cold hexane to give pure 6a (2.46 g, 48% yield) as a white solid. GC-MS shows one peak M⁺=513 (100%). ¹H-NMR (400 MHz; CDCl₃): δ 8.14 (s, 2H), 7.93 (s, 1H), 7.87 (d, J=11.8 Hz, 1H), 7.80-7.78 (m, 2H), 7.52 (d, J=8.1 Hz, 1H), 7.47-7.45 (m, 2H), 4.08 (s, 3H), 2.52 (s, 3H).

4-chloro-3-(4′-cyclohexyl-[1,1′-biphenyl]-4-yl)-6-fluoro-7-methoxy-2-methylquinoline (6b): Following the general procedure A, a mixture of 5 (3.81 g, 10 mmol, 1 eq), 2b (3.43 g, 12.0 mmol, 1.2 eq), aqueous K₂CO₃ (20 ml, 2 eq), Pd(dppf)Cl₂ (366 mg, 0.5 mmol, 0.05 eq) and DMF (200 ml) was heated for 16 h to give crude 6b as a black solid. The product was purified by flash chromatography using a gradient of ethyl acetate/hexane (3/7) as the eluting solvent to give 6b (1.28 g). The product was further crystallized in ethyl acetate/hexane to give pure 6b (698 mg, 15% yield) as white crystal. GC-MS shows one peak M⁺=459 (100%). ¹H-NMR (400 MHz; CDCl₃): δ 7.88 (d, J=11.9 Hz, 1H), 7.74 (d, J=8.2 Hz, 2H), 7.64 (d, J=8.2 Hz, 2H), 7.52 (d, J=8.1 Hz, 1H), 7.35 (dd, J=8.3, 2.1 Hz, 4H), 4.08 (s, 3H), 2.62-2.55 (m, 1H), 2.52 (s, 3H), 1.98-1.79 (m, 5H), 1.56-1.26 (m, 5H).

4,6-dichloro-3-(4′-fluoro-2-(trifluoromethoxy)-[1,1′-biphenyl]-4-yl)-7-methoxy-2-methylquinoline (9): Following the general procedure A, a mixture of 4 (386 mg, 1.05 mmol, 1 eq), 8 (440 mg, 1.15 mmol, 1.1 eq), aqueous K₂CO₃ (1.1 ml, 2 eq), Pd(dppf)Cl₂ (38 mg, 0.5 mmol, 0.05 eq) and DMF (25 ml) was heated for 48 h to give crude 9 (671 mg) as a black solid. The product was purified by flash chromatography using a gradient of ethyl acetate/hexane (2/8) as the eluting solvent to give 9 (401 mg) and was further crystallized in ethyl acetate/hexane to give pure 9 (310 mg, 60% yield) as white solid. GC-MS shows one peak M⁺=495 (100%). ¹H-NMR (400 MHz; CDCl₃): δ 8.27 (s, 1H), 7.59-7.54 (m, 3H), 7.50 (s, 1H), 7.32-7.30 (m, 2H), 7.23-7.18 (m, 2H), 4.10 (s, 3H), 2.54 (s, 3H).

### 4-chloro-3-(4-cyclohexylphenyl)-6-fluoro-7-methoxy-2-methylquinoline (6p)

Following the general procedure A, a mixture of 5 (305 mg, 1.0 mmol, 1 eq), 2p (300 mg, 1.05 mmol, 1.05 eq), aqueous K₂CO₃ (1 ml, 2 eq), Pd(dppf)Cl₂ (37 mg, 0.05 mmol, 0.05 eq) and DMF (25 ml) was heated for 16 h and kept at room temperature for 72 h. After the mixture was filtered over celite white solid on top of the celite was separated to give pure 6p (97 mg) as a white solid. The filtrate was concentrated in vacuo to dryness and was purified by flash chromatography using a gradient of ethyl acetate/hexane (1/9) as the eluting solvent to give 6p (228 mg). The product was further crystallized in DMF to give pure 6p (97 mg) for a combined total 3p (194 mg, 51% yield). GC-MS shows one peak M⁺=383 (100%). ¹H-NMR (400 MHz; CDCl₃): δ 7.84 (d, J=12.0 Hz, 1H), 7.48 (d, J=8.1 Hz, 1H), 7.34-7.31 (m, 2H), 7.18-7.15 (m, 2H), 4.05 (s, 3H), 2.62-2.55 (m, 1H), 1.99-1.76 (m, 5H), 1.54-1.26 (m, 5H).

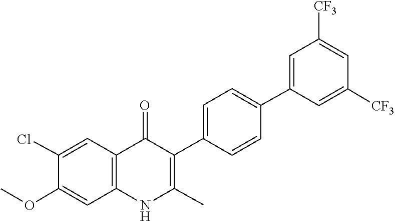 [SMILES: COc1cc2[nH]c(C)c(-c3ccc(-c4cc(C(F)(F)F)cc(C(F)(F)F)c4)cc3)c(=O)c2cc1Cl]

3-(3′,5′-bis(trifluoromethyl)-[1,1′-biphenyl]-4-yl)-6-chloro-7-methoxy-2-methylquinolin-4(1H)-one (ELQ-689): Following the general procedure B, a mixture of 3a (690 mg, 1.30 mmol, 1 eq), KOAc, (1.28 g, 13 mmol, 10 eq), glacial acetic acid (5 ml) was heated for 16 h to give pure ELQ-689 (532 mg, 80% yield) as a white solid. ¹H-NMR (400 MHz; DMSO-d₆): δ 11.73 (s, 1H), 8.39 (s, 2H), 8.10 (s, 1H), 8.03 (s, 1H), 7.92-7.90 (m, 2H), 7.44-7.42 (m, 2H), 7.10 (s, 1H), 3.98 (s, 3H), 2.28 (s, 3H).

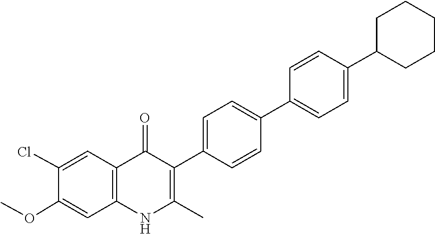 [SMILES: COc1cc2[nH]c(C)c(-c3ccc(-c4ccc(C5CCCCC5)cc4)cc3)c(=O)c2cc1Cl]

6-chloro-3-(4′-cyclohexyl-[1,1′-biphenyl]-4-yl)-7-methoxy-2-methylquinolin-4(1H)-one (ELQ-690): Following the general procedure B, a mixture of 3b (238 mg, 0.5 mmol, 1 eq), KOAc, (490 mg, 5 mmol, 10 eq), glacial acetic acid (2 ml) was heated for 18 h to give pure ELQ-690 (207 mg, 90% yield) as a white solid. ¹H-NMR (400 MHz; DMSO-d₆): δ 11.72-11.67 (m, 1H), 8.02 (s, 1H), 7.67-7.62 (m, 4H), 7.35-7.31 (m, 4H), 7.09 (s, 1H), 3.98 (s, 3H), 2.69-2.67 (m, 0.5), 2.35-2.33 (m, 0.5), 2.25 (s, 3H), 1.86-1.81 (m, 4H), 1.75-1.70 (m, 1H), 1.48-1.34 (m, 5H), 1.32-1.23 (m, 1H).

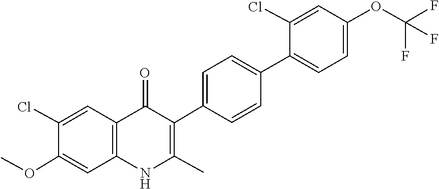 [SMILES: COc1cc2[nH]c(C)c(-c3ccc(-c4ccc(OC(F)(F)F)cc4Cl)cc3)c(=O)c2cc1Cl]

6-chloro-3-(2′-chloro-4′-(trifluoromethoxy)-[1,1′-biphenyl]-4-yl)-7-methoxy-2-methylquinolin-4(1H)-one (ELQ-692): Following the general procedure B, a mixture of 3c (190 mg, 0.37 mmol, 1 eq), KOAc, (363 mg, 3.7 mmol, 10 eq), glacial acetic acid (5 ml) was heated for 24 h to give pure ELQ-692 (161 mg, 88% yield) as a white solid. ¹H-NMR (400 MHz; DMSO-d₆): δ 11.72 (s, 1H), 8.02 (s, 1H), 7.72-7.72 (m, 1H), 7.63 (d, J=8.5 Hz, 1H), 7.50-7.48 (m, 3H), 7.37 (d, J=8.2 Hz, 2H), 7.09 (s, 1H), 3.98 (s, 3H), 2.28 (s, 3H). M.P. 337.8-338.9° C. with decomposition.

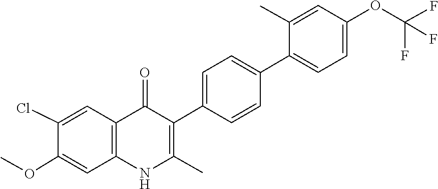 [SMILES: COc1cc2[nH]c(C)c(-c3ccc(-c4ccc(OC(F)(F)F)cc4C)cc3)c(=O)c2cc1Cl]

6-chloro-7-methoxy-2-methyl-3-(2′-methyl-4′-(trifluoromethoxy)-[1,1′-biphenyl]-4-yl)quinolin-4(1H)-one (ELQ-693): Following the general procedure B, a mixture of 3d (430 mg, 0.87 mmol, 1 eq), KOAc, (853 mg, 8.7 mmol, 10 eq), glacial acetic acid (4 ml) was heated for 24 h to give pure ELQ-693 (334 mg, 81% yield) as a white solid. ¹H-NMR (400 MHz; DMSO-d₆): δ 11.69 (s, 1H), 8.02 (s, 1H), 7.40-7.33 (m, 6H), 7.28-7.26 (m, 1H), 7.08 (s, 1H), 3.97 (s, 3H), 2.34 (s, 3H), 2.27 (s, 3H). M.P. 325.9-327.1° C. with decomposition.

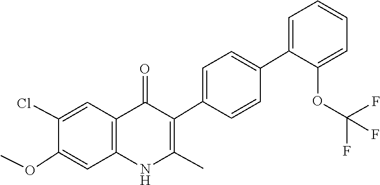 [SMILES: COc1cc2[nH]c(C)c(-c3ccc(-c4ccccc4OC(F)(F)F)cc3)c(=O)c2cc1Cl]

6-chloro-7-methoxy-2-methyl-3-(2′-(trifluoromethoxy)-[1,1′-biphenyl]-4-yl)quinolin-4(1H)-one (ELQ-702): Following the general procedure B, a mixture of 3e (638 mg, 1.33 mmol, 1 eq), KOAc, (1.30 g, 13.3 mmol, 10 eq), glacial acetic acid (5 ml) was heated for 24 h to give pure ELQ-702 (334 mg, 76% yield) as a white solid. ¹H-NMR (400 MHz; DMSO-d₆): δ 11.71 (s, 1H), 8.02 (s, 1H), 7.62-7.60 (m, 1H), 7.55-7.48 (m, J=2.3 Hz, 5H), 7.38-7.35 (m, 2H), 7.09 (s, 1H), 3.98 (s, 3H), 2.26 (s, 3H). M.P. 293.1-294.3° C. with decomposition.

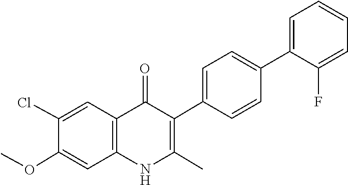 [SMILES: COc1cc2[nH]c(C)c(-c3ccc(-c4ccccc4F)cc3)c(=O)c2cc1Cl]

6-chloro-3-(2′-fluoro-[1,1′-biphenyl]-4-yl)-7-methoxy-2-methylquinolin-4(1H)-one (ELQ-703): Following the general procedure B, a mixture of 3f (369 mg, 0.90 mmol, 1 eq), KOAc, (882 mg, 9.0 mmol, 10 eq), glacial acetic acid (5 ml) was heated for 24 h to give pure ELQ-703 (262 mg, 74% yield) as a white solid. ¹H-NMR (400 MHz; DMSO-d₆): δ 11.72 (s, 1H), 8.02 (s, 1H), 7.62-7.57 (m, 3H), 7.45-7.32 (m, 5H), 7.08 (s, 1H), 3.97 (s, 3H), 2.27 (s, 3H). M.P. 370.4-371° C. with decomposition.

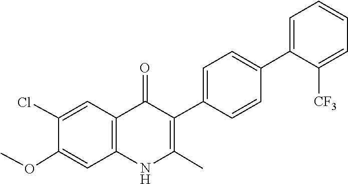 [SMILES: COc1cc2[nH]c(C)c(-c3ccc(-c4ccccc4C(F)(F)F)cc3)c(=O)c2cc1Cl]

6-chloro-7-methoxy-2-methyl-3-(2′-(trifluoromethyl)-[1,1′-biphenyl]-4-yl)quinolin-4(1H)-one (ELQ-704): Following the general procedure B, a mixture of 3g (200 mg, 0.43 mmol, 1 eq), KOAc, (421 mg, 4.3 mmol, 10 eq), glacial acetic acid (5 ml) was heated for 24 h to give pure ELQ-704 (152 mg, 80% yield) as a white solid. ¹H-NMR (400 MHz; DMSO-d₆): δ 11.74 (s, 1H), 8.02 (s, 1H), 7.87-7.85 (m, 1H), 7.77-7.74 (m, 1H), 7.65-7.62 (m, 1H), 7.49-7.47 (m, 1H), 7.33 (s, 4H), 7.09 (s, 1H), 3.98 (s, 3H), 2.26 (s, 3H). M.P. 350.7-351.3° C. with decomposition.

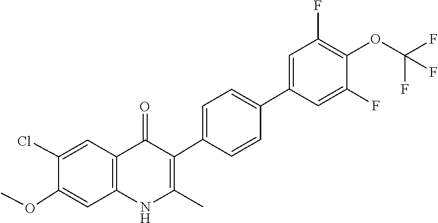 [SMILES: COc1cc2[nH]c(C)c(-c3ccc(-c4cc(F)c(OC(F)(F)F)c(F)c4)cc3)c(=O)c2cc1Cl]

6-chloro-3-(3′,5′-difluoro-4′-(trifluoromethoxy)-[1,1′-biphenyl]-4-yl)-7-methoxy-2-methylquinolin-4(1H)-one (ELQ-717): Following the general procedure B, a mixture of 3h (677 mg, 1.3 mmol, 1 eq), KOAc, (1.27 g, 13 mmol, 10 eq), glacial acetic acid (10 ml) was heated for 24 h to give pure ELQ-717 (507 mg, 79% yield) as a white solid. ¹H-NMR (400 MHz; DMSO-d₆): δ 11.72 (s, 1H), 8.02 (s, 1H), 7.85-7.80 (m, J=11.6, 9.0 Hz, 4H), 7.39 (d, J=8.4 Hz, 2H), 7.09 (s, 1H), 3.98 (s, 3H), 2.26 (s, 3H).

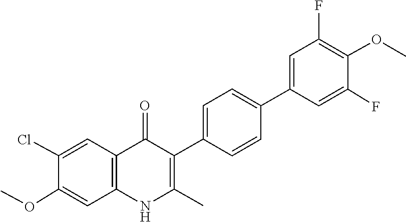

6-chloro-3-(3′,5′-difluoro-4′-methoxy-[1,1′-biphenyl]-4-yl)-7-methoxy-2-methylquinolin-4(1H)-one (ELQ-716): Following the general procedure B, a mixture of 3i (917 mg, 1.98 mmol, 1 eq), KOAc, (1.95 g, 19.8 mmol, 10 eq), glacial acetic acid (15 ml) was heated for 18 h to give pure ELQ-716 (500 mg, 57% yield) as a white solid. ¹H-NMR (400 MHz; DMSO-d₆): δ 8.01 (s, 1H), 7.72 (d, J=8.2 Hz, 2H), 7.56 (d, J=9.8 Hz, 2H), 7.34 (d, J=8.2 Hz, 2H), 7.08 (s, 1H), 3.98-3.97 (2s, 6H), 2.25 (s, 3H).

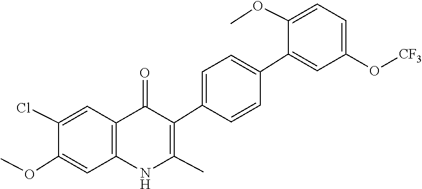 [SMILES: COc1cc2[nH]c(C)c(-c3ccc(-c4cc(OC(F)(F)F)ccc4OC)cc3)c(=O)c2cc1Cl]

6-chloro-7-methoxy-3-(2′-methoxy-5′-(trifluoromethoxy)-[1,1′-biphenyl]-4-yl)-2-methylquinolin-4(1H)-one (ELQ-727): Following the general B, a mixture of 3j (976 mg, 1.92 mmol, 1 eq), KOAc, (1.88 g, 19.2 mmol, 10 eq), glacial acetic acid (10 ml) was heated for 18 h to give pure ELQ-727 (697 mg, 74% yield) as a white solid. ¹H-NMR (400 MHz; DMSO-d₆): δ 11.69 (s, 1H), 8.02 (s, 1H), 7.54-7.52 (m, 2H), 7.38-7.30 (m, 4H), 7.24-7.22 (m, 1H), 7.09 (s, 1H), 3.96-3.95 (m, 3H), 3.88-3.82 (m, 3H), 2.31-2.24 (m, 3H). M.P. 297.3-298.1° C. with decomposition.

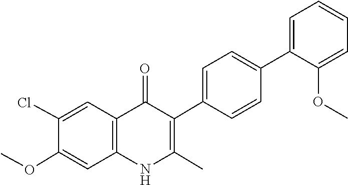

6-chloro-7-methoxy-3-(2′-methoxy-[1,1′-biphenyl]-4-yl)-2-methylquinolin-4(1H)-one (ELQ-728): Following the general procedure B, a mixture of 3k (540 mg, 1.27 mmol, 1 eq), KOAc, (1.24 g, 12.7 mmol, 10 eq), glacial acetic acid (5 ml) was heated for 18 h to give pure ELQ-728 (467 mg, 91% yield) as a white solid. ¹H-NMR (400 MHz; DMSO-d₆): δ 11.67 (s, 1H), 8.02 (s, 1H), 7.51-7.49 (m, 2H), 7.38-7.34 (m, 2H), 7.29-7.26 (m, 2H), 7.15-7.13 (m, 1H), 7.09 (s, 1H), 7.06 (td, J=7.4, 1.0 Hz, 1H), 3.98 (s, 3H), 3.81 (s, 3H), 2.27 (s, 3H). M.P. 327.3-327.9° C. with decomposition.

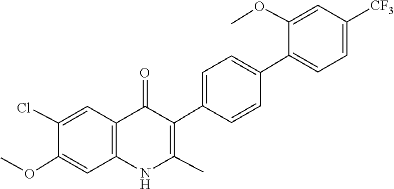 [SMILES: COc1cc2[nH]c(C)c(-c3ccc(-c4ccc(C(F)(F)F)cc4OC)cc3)c(=O)c2cc1Cl]

6-chloro-7-methoxy-3-(2′-methoxy-4′-(trifluoromethyl)-[1,1′-biphenyl]-4-yl)-2-methylquinolin-4(1H)-one (ELQ-742): Following the general procedure B, a mixture of 31 (600 mg, 1.22 mmol, 1 eq), KOAc, (1.20 g, 12.2 mmol, 10 eq), glacial acetic acid (10 ml) was heated for 18 h to give pure ELQ-742 (455 mg, 79% yield) as a white solid. ¹H-NMR (400 MHz; DMSO-d₆): δ 11.70 (s, 1H), 8.02 (s, 1H), 7.59-7.53 (m, 4H), 7.42-7.40 (m, 2H), 7.34-7.31 (m, 2H), 7.09 (s, 1H), 3.98 (s, 3H), 3.90 (s, 3H), 2.27 (s, 3H). M.P. 350.7-351.2° C. with decomposition.

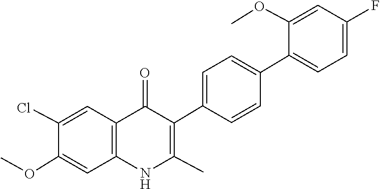 [SMILES: COc1cc2[nH]c(C)c(-c3ccc(-c4ccc(F)cc4OC)cc3)c(=O)c2cc1Cl]

6-chloro-3-(4′-fluoro-2′-methoxy-[1,1′-biphenyl]-4-yl)-7-methoxy-2-methylquinolin-4(1H)-one (ELQ-743): Following the general procedure B, a mixture of 3m (500 mg, 1.13 mmol, 1 eq), KOAc, (1.11 g, 11.3 mmol, 10 eq), glacial acetic acid (10 ml) was heated for 18 h to give ELQ-743 (455 mg). The product was further crystallized from DMF to pure ELQ-743 (290 mg, 50% yield) as a white solid. ¹H-NMR (400 MHz; DMSO-d₆): δ 11.67 (s, 1H), 8.02 (s, 1H), 7.47 (d, J=8.2 Hz, 2H), 7.37 (dd, J=8.4, 7.0 Hz, 1H), 7.27 (d, J=8.2 Hz, 2H), 7.08 (s, 1H), 7.04 (dd, J=11.5, 2.4 Hz, 1H), 6.88 (td, J=8.4, 2.5 Hz, 1H), 3.97 (s, 3H), 3.83 (s, 3H), 2.26 (s, 3H). M.P. 360.7-361.1° C. with decomposition.

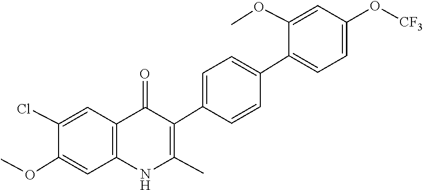 [SMILES: COc1cc2[nH]c(C)c(-c3ccc(-c4ccc(OC(F)(F)F)cc4OC)cc3)c(=O)c2cc1Cl]

6-chloro-7-methoxy-3-(2′-methoxy-4′-(trifluoromethoxy)-[1,1′-biphenyl]-4-yl)-2-methylquinolin-4(1H)-one (ELQ-744): Following the general procedure B, a mixture of 3n (380 mg, 0.75 mmol, 1 eq), KOAc, (735 mg, 7.5 mmol, 10 eq), glacial acetic acid (10 ml) was for 18 h to give ELQ-744 (314 mg, 85% yield) as a white solid. ¹H-NMR (400 MHz; DMSO-d₆): δ 8.02 (s, 1H), 7.52-7.49 (m, 2H), 7.47 (d, J=8.4 Hz, 1H), 7.31-7.28 (m, 2H), 7.13 (d, J=2.0 Hz, 1H), 7.09 (s, 1H), 7.06-7.03 (m, J=1.2 Hz, 1H), 3.98 (s, 3H), 3.85 (s, 3H). M.P. 344.5-345.1° C. with decomposition.

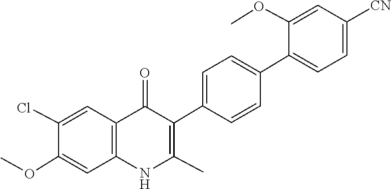 [SMILES: COc1cc2[nH]c(C)c(-c3ccc(-c4ccc(C#N)cc4OC)cc3)c(=O)c2cc1Cl]

4′-(6-chloro-7-methoxy-2-methyl-4-oxo-1,4-dihydroquinolin-3-yl)-2-methoxy-[1,1′-biphenyl]-4-carbonitrile (ELQ-745): Following the general procedure B, a mixture of 3o (449 mg, 1.0 mmol, 1 eq), KOAc, (980 mg, 10.0 mmol, 10 eq), glacial acetic acid (10 ml) was heated for 18 h to give ELQ-745 (384 mg, 89% yield) as a white solid. ¹H-NMR (400 MHz; DMSO-d₆): δ 11.69 (s, 1H), 8.02 (s, 1H), 7.61 (d, J=1.3 Hz, 1H), 7.57-7.51 (m, 4H), 7.34-7.31 (m, 2H), 7.09 (s, 1H), 3.98 (s, 3H), 3.88 (s, 3H), 2.27 (s, 3H). M.P. 352.7-353.2° C. with decomposition.

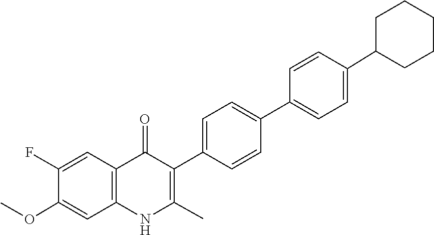 [SMILES: COc1cc2[nH]c(C)c(-c3ccc(-c4ccc(C5CCCCC5)cc4)cc3)c(=O)c2cc1F]

3-(4′-cyclohexyl-[1,1′-biphenyl]-4-yl)-6-fluoro-7-methoxy-2-methylquinolin-4(1H)-one (ELQ-694): Following the general procedure B, a mixture of 6a (2.05 g, 4.0 mmol, 1 eq), KOAc, (3.90 g, 40.0 mmol, 10 eq), glacial acetic acid (10 ml) was heated for 24 h to give ELQ-694 (1.76 g, 89% yield) as a white solid. ¹H-NMR (400 MHz; DMSO-d₆): δ 11.71 (s, 1H), 8.38 (s, 2H), 8.10 (s, 1H), 7.91-7.88 (m, 2H), 7.73 (d, J=11.7 Hz, 1H), 7.44-7.40 (m, 2H), 7.11 (d, J=7.3 Hz, 1H), 3.96 (s, 3H), 2.27 (s, 3H).

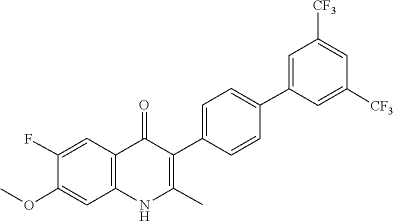 [SMILES: COc1cc2[nH]c(C)c(-c3ccc(-c4cc(C(F)(F)F)cc(C(F)(F)F)c4)cc3)c(=O)c2cc1F]

3-(3′,5′-bis(trifluoromethyl)-[1,1′-biphenyl]-4-yl)-6-fluoro-7-methoxy-2-methylquinolin-4(1H)-one (ELQ-697): Following the general procedure B, a mixture of 6b (698 mg, 1.52 mmol, 1 eq), KOAc, (1.49 g, 15.2 mmol, 10 eq), glacial acetic acid (10 ml) was heated for 24 h to give ELQ-697 (623 mg, 93% yield) as a white solid. ¹H-NMR (400 MHz; DMSO-d₆): δ 11.68 (s, 1H), 7.72 (d, J=11.7 Hz, 1H), 7.67-7.61 (m, 4H), 7.34-7.30 (m, 4H), 7.12 (d, J=7.4 Hz, 1H), 3.97 (s, 3H), 2.59-2.54 (m, 1H), 2.26 (s, 3H), 1.85-1.71 (m, 5H), 1.51-1.24 (m, 5H).

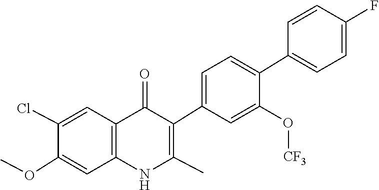 [SMILES: COc1cc2[nH]c(C)c(-c3ccc(-c4ccc(F)cc4)c(OC(F)(F)F)c3)c(=O)c2cc1Cl]

6-chloro-3-(4′-fluoro-2-(trifluoromethoxy)-[1,1′-biphenyl]-4-yl)-7-methoxy-2-methylquinolin-4(1H)-one (ELQ-762): Following the general procedure B, a mixture of 9 (310 mg, 0.63 mmol, 1 eq), KOAc, (517 mg, 6.3 mmol, 10 eq), glacial acetic acid (5 ml) was heated for 24 h to give ELQ-762 (300 mg, 99% yield) as a white solid. ¹H-NMR (400 MHz; DMSO-d₆): δ 12.21-11.94 (m, 1H), 8.03 (s, 1H), 7.60-7.54 (m, 3H), 7.42-7.33 (m, 4H), 7.18-7.15 (m, 1H), 4.00-3.95 (m, 3H), 2.32-2.32 (m, 3H). M.P. 300-301° C.

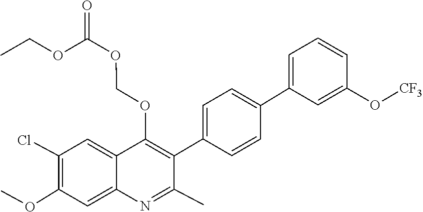 [SMILES: CCOC(=O)OCOc1c(-c2ccc(-c3cccc(OC(F)(F)F)c3)cc2)c(C)nc2cc(OC)c(Cl)cc12]

((6-chloro-7-methoxy-2-methyl-3-(3′-(trifluoromethoxy)-[1,1′-biphenyl]-4-yl)quinolin-4-yl)oxy)methyl ethyl carbonate (ELQ-652): Following the general procedure C, using a mixture of ELQ-604 (150 mg, 0.33 mmol, 1 eq), TBAI (244 mg, 0.66 mmol, 2 eq), dry K₂CO₃ (92 mg, 0.66 mmol, 2 eq) and chloromethyl ethylcarbonate (91.7 mg, 0.66 mmol, 2 eq) in DMF (15 ml) to give crude ELQ-652 (193 mg). The product was purified by flash chromatography using ethyl acetate/hexane (3/7) followed by crystallization in ethyl acetate/hexane to give pure ELQ-652 (100 mg, yield 54%) as a white solid. GC-MS shows one peak M⁺=561 (45%), M⁺=459 (100%). ¹H-NMR (400 MHz; CDCl₃): δ 8.08 (s, 1H), 7.76-7.73 (m, 2H), 7.65-7.63 (m, 1H), 7.56-7.49 (m, 5H), 7.47 (s, 1H), 5.32 (s, 2H), 5.31 (s), 4.13 (q, J=7.1 Hz, 2H), 4.08 (s, 3H), 2.56 (s, 3H), 1.23 (t, J=7.1 Hz, 3H).

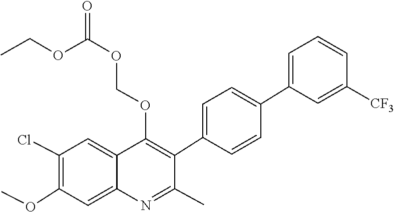 [SMILES: CCOC(=O)OCOc1c(-c2ccc(-c3cccc(C(F)(F)F)c3)cc2)c(C)nc2cc(OC)c(Cl)cc12]

((6-chloro-7-methoxy-2-methyl-3-(3′-(trifluoromethyl)-[1,1′-biphenyl]-4-yl)quinolin-4-yl)oxy)methyl ethyl carbonate (ELQ-671): Following the general procedure C, using a mixture of ELQ-646 (150 mg, 0.34 mmol, 1 eq), TBAI (251 mg, 0.68 mmol, 2 eq), dry K₂CO₃ (95 mg, 0.68 mmol, 2 eq) and chloromethyl ethylcarbonate (95 mg, 0.68 mmol, 2 eq) in DMF (30 ml) to give crude ELQ-671 (188 mg). The product was purified by flash chromatography using ethyl acetate/hexane (3/7) followed by crystallization in ethyl acetate/hexane to give pure ELQ-671 (91 mg) and a second crop (23 mg) for a total ELQ-671 (114 mg, yield 61%) as a white solid. GC-MS shows one peak M⁺=545 (37%), M⁺=443 (100%). ¹H-NMR (400 MHz; CDCl₃): δ 8.08 (s, 1H), 7.96-7.95 (m, 1H), 7.90-7.87 (m, 1H), 7.79-7.76 (m, 2H), 7.70-7.62 (m, 2H), 7.54-7.51 (m, 2H), 7.47 (s, 1H), 5.32 (s, 2H), 4.13 (q, J=7.1 Hz, 2H), 4.08 (s, 3H), 2.57 (s, 3H), 1.23 (t, J=7.1 Hz, 3H).

((3-(3′,5′-bis(trifluoromethyl)-[1,1′-biphenyl]-4-yl)-6-chloro-7-methoxy-2-methylquinolin-4-yl)oxy)methyl ethyl carbonate (ELQ-699): Following the general procedure C, using a mixture of ELQ-689 (511 mg, 1.0 mmol, 1 eq), TBAI (738 mg, 2.0 mmol, 2 eq), dry K₂CO₃ (278 mg, 2.0 mmol, 2 eq) and chloromethyl ethylcarbonate (278 mg, 2.0 mmol, 2 eq) in DMF (50 ml) to give crude ELQ-699 (707 mg). The product was purified by flash chromatography using ethyl acetate/hexane (2/8) followed by crystallization in ethyl acetate/hexane to give pure ELQ-699 (400 mg, yield 65%) as a white solid. GC-MS shows one peak M⁺=613 (30%), M⁺=511 (100%). ¹H-NMR (400 MHz; CDCl₃): δ 8.11 (s, 2H), 8.06 (s, 1H), 7.91 (s, 1H), 7.77 (d, J=8.0 Hz, 2H), 7.55 (d, J=7.9 Hz, 2H), 7.45 (s, 1H), 5.30 (s, 2H), 4.11 (q, J=7.1 Hz, 2H), 4.06 (s, 3H), 2.54 (s, 3H), 1.21 (t, J=7.1 Hz, 3H).

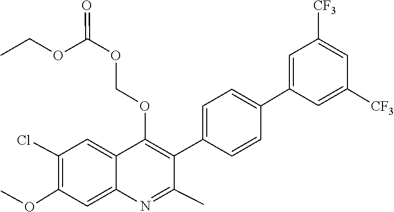 [SMILES: CCOC(=O)OCOc1c(-c2ccc(-c3cc(C(F)(F)F)cc(C(F)(F)F)c3)cc2)c(C)nc2cc(OC)c(Cl)cc12]

((6-chloro-7-methoxy-2-methyl-3-(2′-(trifluoromethoxy)-[1,1′-biphenyl]-4-yl)quinolin-4-yl)oxy)methyl ethyl carbonate (ELQ-711): Following the general procedure C, using a mixture of ELQ-702 (230 mg, 0.5 mmol, 1 eq), TBAI (369 mg, 1.0 mmol, 2 eq), dry K₂CO₃ (139 mg, 1.0 mmol, 2 eq) and chloromethyl ethylcarbonate (139 mg, 2.0 mmol, 2 eq) in DMF (25 ml) to give crude ELQ-711 (737 mg). The product was purified by flash chromatography using ethyl acetate/hexane (2/8) followed by crystallization in ethyl acetate/hexane to give pure ELQ-711 (160 mg, yield 57%) as a white solid. GC-MS shows one peak M⁺=561 (33%), M⁺=459 (100%). ¹H-NMR (400 MHz; CDCl₃): δ 8.09 (s, 1H), 7.64-7.61 (m, 2H), 7.57-7.55 (m, 1H), 7.50-7.40 (m, 5H), 5.27 (s, 2H), 4.16 (q, J=7.1 Hz, 2H), 4.08 (s, 3H), 2.58 (s, 3H), 1.25 (t, J=7.1 Hz, 3H).

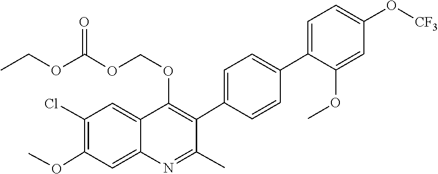 [SMILES: CCOC(=O)OCOc1c(-c2ccc(-c3ccc(OC(F)(F)F)cc3OC)cc2)c(C)nc2cc(OC)c(Cl)cc12]

((6-chloro-7-methoxy-3-(2′-methoxy-4′-(trifluoromethoxy)-[1,1′-biphenyl]-4-yl)-2-methylquinolin-4-yl)oxy)methyl ethyl carbonate (ELQ-749): Following the general procedure C, using a mixture of ELQ-744 (980 mg, 2.0 mmol, 1 eq), TBAI (1.48 g, 4.0 mmol, 2 eq), dry K₂CO₃ (556 mg, 4.0 mmol, 2 eq) and chloromethyl ethylcarbonate (556 mg, 4.0 mmol, 2 eq) in DMF (100 ml) to give crude ELQ-749 (737 mg). The product was purified by flash chromatography using ethyl acetate/hexane (4/6) followed by crystallization in ethyl acetate/hexane to give pure ELQ-749 (830 mg, yield 70%) as a light-yellow crystal. GC-MS shows one peak M⁺=591 (48%), M⁺=489 (100%). ¹H-NMR (400 MHz; CDCl₃): δ 8.06 (s, 1H), 7.66-7.63 (m, 2H), 7.44-7.40 (m, 4H), 6.96-6.93 (m, J=1.1 Hz, 1H), 6.87-6.86 (m, 1H), 5.29 (s, 2H), 4.13 (q, J=7.1 Hz, 2H), 4.05 (s, 3H), 3.88 (s, 3H), 2.56 (s, 3H), 1.22 (t, J=7.1 Hz, 3H).

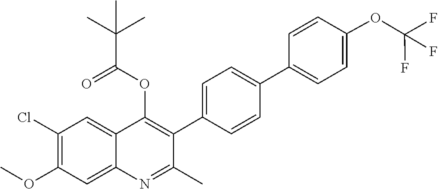 [SMILES: COc1cc2nc(C)c(-c3ccc(-c4ccc(OC(F)(F)F)cc4)cc3)c(OC(=O)C(C)(C)C)c2cc1Cl]

6-chloro-7-methoxy-2-methyl-3-(4′-(trifluoromethoxy)-[1,1′-biphenyl]-4-yl)quinolin-4-yl pivalate (ELQ-753): A mixture of ELQ-596 (1.84 g, 4.0 mmol, 1 eq) and sodium hydride 60% oil suspension (320 mg, 8.0 mmol, 2 eq) in dry THF (75 ml) was heated at 60° C. for 30 minutes. Then pivaloyl chloride (968 mg, 8.0 mmol, 2 eq) was added and the cloudy solution was heated for another 2 hours. After cooling to room temperature water (2 ml) was added resulting in a formation of yellow sticky solid. This was filtered and the sticky solid washed with ethyl acetate (3×10 ml). The combined filtrate was concentrated in vaccuo to give crude ELQ-753 (2.41 mg) as a yellow solid. The product was purified by flash chromatography using ethyl acetate/hexane (2/8) followed by crystallization in ethyl acetate to give pure ELQ-753 (1.20 g, yield 55%) as a white crystal. GC-MS shows one peak M⁺=543 (5%), M⁺=57 (100%). ¹H-NMR (400 MHz; CDCl₃): δ 7.67 (s, 1H), 7.66-7.65 (m, 2H), 7.64-7.63 (m, 2H), 7.50 (s, 1H), 7.35-7.33 (m, 2H), 7.32-7.31 (m, 2H), 4.06 (s, 3H), 2.52 (s, 3H), 1.08 (s, 9H).

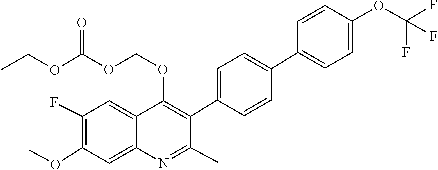 [SMILES: CCOC(=O)OCOc1c(-c2ccc(-c3ccc(OC(F)(F)F)cc3)cc2)c(C)nc2cc(OC)c(F)cc12]

Ethyl (((6-fluoro-7-methoxy-2-methyl-3-(4′-(trifluoromethoxy)-[1,1′-biphenyl]-4-yl)quinolin-4-yl)oxy)methyl) carbonate (ELQ-672): Following the general procedure C, using a mixture of ELQ-650 (1.77 g, 4.0 mmol, 1 eq), TBAI (2.95 g, 8.0 mmol, 2 eq), dry K₂CO₃ (1.11 g, 8.0 mmol, 2 eq) and chloromethyl ethylcarbonate (1.11 g, 8.0 mmol, 2 eq) in DMF (150 ml) to give crude ELQ-672 (2.75 g). The product was purified by flash chromatography using ethyl acetate/hexane (2/8) followed by crystallization in ethyl acetate/hexane to give pure ELQ-650 (1.62 g, yield 74%) as a white crystal. GC-MS shows one peak M⁺=545 (40%), M⁺=443 (100%). ¹H-NMR (400 MHz; CDCl₃): δ 7.72-7.66 (m, 5H), 7.49-7.46 (m, 3H), 7.35-7.33 (m, 2H), 5.29 (s, 2H), 4.09 (q, J=7.1 Hz, 2H), 4.04 (s, 3H), 2.54 (s, 3H), 1.20 (t, J=7.1 Hz, 3H).

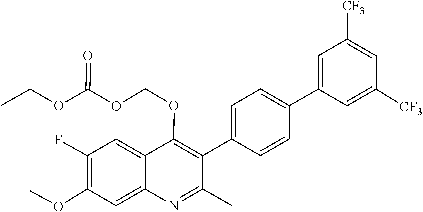 [SMILES: CCOC(=O)OCOc1c(-c2ccc(-c3cc(C(F)(F)F)cc(C(F)(F)F)c3)cc2)c(C)nc2cc(OC)c(F)cc12]

((3-(3′,5′-bis(trifluoromethyl)-[1,1′-biphenyl]-4-yl)-6-fluoro-7-methoxy-2-methylquinolin-4-yl)oxy)methyl ethyl carbonate (ELQ-696): Following the general procedure C, using a mixture of ELQ-694 (495 mg, 1.0 mmol, 1 eq), TBAI (738 g, 2.0 mmol, 2 eq), dry K₂CO₃ (278 mg, 2.0 mmol, 2 eq) and chloromethyl ethylcarbonate (278 mg, 2.0 mmol, 2 eq) in DMF (50 ml) to give crude ELQ-696 (816 mg). The product was purified by flash chromatography using ethyl acetate/hexane (2/8) followed by trituration with hexane and cool to 4° C. for 12 h to give pure ELQ-696 (459 mg, yield 77%) as a white solid. GC-MS shows one peak M⁺=597 (25%), M⁺=495 (100%). ¹H-NMR (400 MHz; CDCl₃): δ 8.12-8.11 (m, 2H), 7.92-7.91 (m, 1H), 7.78-7.75 (m, 2H), 7.68 (d, J=11.7 Hz, 1H), 7.57-7.54 (m, 2H), 7.48 (d, J=8.0 Hz, 1H), 5.30 (s, 2H), 4.09 (q, J=7.1 Hz, 2H), 4.05 (s, 3H), 2.54 (s, 3H), 1.20 (t, J=7.1 Hz, 3H).

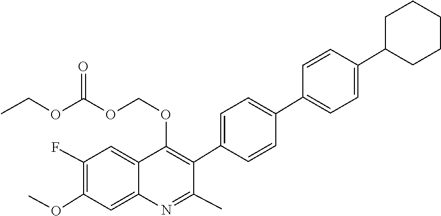 [SMILES: CCOC(=O)OCOc1c(-c2ccc(-c3ccc(C4CCCCC4)cc3)cc2)c(C)nc2cc(OC)c(F)cc12]

((3-(4′-cyclohexyl-[1,1′-biphenyl]-4-yl)-6-fluoro-7-methoxy-2-methylquinolin-4-yl)oxy)methyl ethyl carbonate (ELQ-698): Following the general procedure C, using a mixture of ELQ-697 (442 mg, 1.0 mmol, 1 eq), TBAI (738 g, 2.0 mmol, 2 eq), dry K₂CO₃ (278 mg, 2.0 mmol, 2 eq) and chloromethyl ethylcarbonate (278 mg, 2.0 mmol, 2 eq) in DMF (50 ml) to give crude ELQ-698 (816 mg). The product was purified by flash chromatography using ethyl acetate/hexane (2/8) give pure ELQ-698 (420 mg, yield 77%) as a white solid. GC-MS shows one peak M⁺=543 (50 15%), M⁺=207 (100%). ¹H-NMR (400 MHz; CDCl₃): δ 7.74-7.70 (m, 2H), 7.67 (d, J=11.7 Hz, 1H), 7.63-7.60 (m, 2H), 7.47 (d, J=8.0 Hz, 1H), 7.45-7.42 (m, 2H), 7.35-7.31 (m, 2H), 5.27 (s, 2H), 4.09 (q, J=7.1 Hz, 2H), 4.04 (s, 3H), 2.60-2.57 (m, 1H), 2.54 (s, 3H), 1.95-1.75 (m, 5H), 1.53-1.26 (m, 5H), 1.20 (t, J=7.1 Hz, 3H).

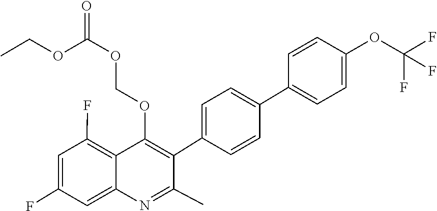 [SMILES: CCOC(=O)OCOc1c(-c2ccc(-c3ccc(OC(F)(F)F)cc3)cc2)c(C)nc2cc(F)cc(F)c12]

((5,7-difluoro-2-methyl-3-(4′-(trifluoromethoxy)-[1,1′-biphenyl]-4-yl)quinolin-4-yl)oxy)methyl ethyl carbonate (ELQ-761): Following the general procedure C, using a mixture of ELQ-601 (431 mg, 1.0 mmol, 1 eq), TBAI (738 g, 2.0 mmol, 2 eq), dry K₂CO₃ (278 mg, 2.0 mmol, 2 eq) and chloromethyl ethylcarbonate (278 mg, 2.0 mmol, 2 eq) in DMF (50 ml) to give crude ELQ-761 (521 mg). The product was purified by flash chromatography using ethyl acetate/hexane (2/8) give pure ELQ-761 (382 mg, yield 72%) as a white solid. GC-MS shows one peak M⁺=533 (25%), M⁺=431 (100%). ¹H-NMR (400 MHz; CDCl₃): δ 7.73-7.70 (m, 4H), 7.56 (ddd, J=9.7, 2.5, 1.4 Hz, 1H), 7.49-7.46 (m, 2H), 7.37-7.35 (m, 2H), 7.04 (ddd, J=11.8, 9.0, 2.6 Hz, 1H), 5.41 (d, J=1.1 Hz, 2H), 4.04 (q, J=7.1 Hz, 2H), 2.57 (s, 3H), 1.21 (t, J=7.1 Hz, 3H).

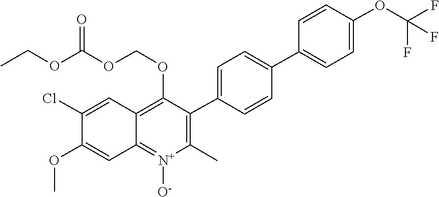 [SMILES: CCOC(=O)OCOc1c(-c2ccc(-c3ccc(OC(F)(F)F)cc3)cc2)c(C)[n+]([O-])c2cc(OC)c(Cl)cc12]

6-chloro-4-(((ethoxycarbonyl)oxy)methoxy)-7-methoxy-2-methyl-3-(4′-(trifluoromethoxy)-[1,1′-biphenyl]-4-yl)quinoline 1-oxide (ELQ-707): Following the general procedure D, using a solution of ELQ-598 (562 mg, 1.0 mmol, 1 eq) and MCPBA (260 mg, 1.5 mmol, 1.5 eq) in chloroform (25 ml) to give crude ELQ-707 (1.0 g). The crude ELQ-707 was dissolved in DCM (5 ml) cooled at 4° C. for 12 h, filtered and the filtrate was purified by flash chromatography using ethyl acetate/DCM (6/4) followed by crystallization in ethyl acetate/hexane to give pure ELQ-707 (397 mg, yield 69%) as a yellow crystal. ¹H-NMR (400 MHz; CDCl₃): δ 8.25 (s, 1H), 8.11 (s, 1H), 7.74-7.67 (m, 4H), 7.48-7.45 (m, 2H), 7.36-7.33 (m, 2H), 5.25 (s, 2H), 4.13 (s, 3H), 4.08 (q, J=7.1 Hz, 2H), 2.57 (s, 3H), 1.18 (t, J=7.1 Hz, 3H).

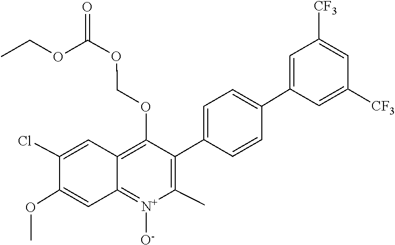 [SMILES: CCOC(=O)OCOc1c(-c2ccc(-c3cc(C(F)(F)F)cc(C(F)(F)F)c3)cc2)c(C)[n+]([O-])c2cc(OC)c(Cl)cc12]

3-(3′,5′-bis(trifluoromethyl)-[1,1′-biphenyl]-4-yl)-6-chloro-4-(((ethoxycarbonyl)oxy)methoxy)-7-methoxy-2-methylquinoline 1-oxide (ELQ-736): Following the general procedure D, using a solution of ELQ-699 (250 mg, 0.41 mmol, 1 eq) and MCPBA (106 mg, 1.5 mmol, 1.5 eq) in chloroform (25 ml) to give crude ELQ-707 (1.0 g). The product was purified by flash chromatography using ethyl acetate/DCM (6/4) followed by crystallization in ethyl acetate/hexane to give pure ELQ-736 (180 mg, yield 70%) as a white solid. ¹H-NMR (400 MHz; CDCl₃): δ 8.27 (s, 1H), 8.14 (s, 1H), 8.13 (s, 2H), 7.95 (s, 1H), 7.83-7.80 (m, 2H), 7.58-7.56 (m, 2H), 5.28 (s, 2H), 4.16 (s, 3H), 4.11 (q, J=7.1 Hz, 2H), 2.59 (s, 3H), 1.21 (t, J=7.1 Hz, 3H).

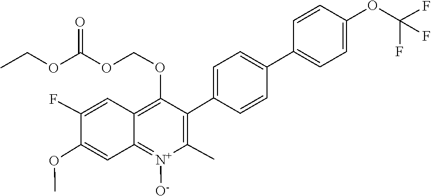 [SMILES: CCOC(=O)OCOc1c(-c2ccc(-c3ccc(OC(F)(F)F)cc3)cc2)c(C)[n+]([O-])c2cc(OC)c(F)cc12]

4-(((ethoxycarbonyl)oxy)methoxy)-6-fluoro-7-methoxy-2-methyl-3-(4′-(trifluoromethoxy)-[1,1′-biphenyl]-4-yl)quinoline 1-oxide (ELQ-709): Following the general procedure D, using a solution of ELQ-672 (545 mg, 1.0 mmol, 1 eq) and MCPBA (260 mg, 1.5 mmol, 1.5 eq) in chloroform (25 ml) to give crude ELQ-709 (898 mg). The product was purified by flash chromatography using ethyl acetate/DCM (6/4) followed by crystallization in ethyl acetate/hexane to give pure ELQ-709 (352 mg, yield 63%) as a white solid. ¹H-NMR (400 MHz; CDCl₃): δ 8.31 (d, J=8.0 Hz, 1H), 7.77-7.69 (m, 5H), 7.50-7.48 (m, 2H), 7.38-7.35 (m, 2H), 5.26 (s, 2H), 4.13 (s, 3H), 4.09 (q, J=7.1 Hz, 2H), 2.59 (s, 3H), 1.20 (t, J=7.1 Hz, 3H).

3-(3′,5′-bis(trifluoromethyl)-[1,1′-biphenyl]-4-yl)-4-(((ethoxycarbonyl)oxy)methoxy)-6-fluoro-7-methoxy-2-methylquinoline 1-oxide (ELQ-737): Following the general procedure D, using a solution of ELQ-696 (245 mg, 0.41 mmol, 1 eq) and MCPBA (105 mg, 1.5 mmol, 1.5 eq) in chloroform (25 ml) to give crude ELQ-709 (376 mg). The product was purified by flash chromatography using ethyl acetate/DCM (6/4) followed by crystallization in ethyl acetate/hexane to give pure ELQ-737 (121 mg, yield 48%) as a white crystal. ¹H-NMR (400 MHz; CDCl₃): δ 8.31 (d, J=8.0 Hz, 1H), 8.13 (s, 2H), 7.95 (s, 1H), 7.82-7.75 (m, 3H), 7.58-7.56 (m, 2H), 5.28 (s, 2H), 4.14 (s, 3H), 4.09 (q, J=7.1 Hz, 2H), 2.58 (s, 3H), 1.20 (t, J=7.1 Hz, 3H).

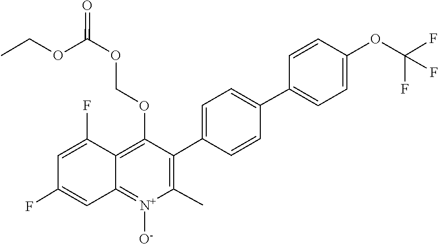 [SMILES: CCOC(=O)OCOc1c(-c2ccc(-c3ccc(OC(F)(F)F)cc3)cc2)c(C)[n+]([O-])c2cc(F)cc(F)c12]

4-(((ethoxycarbonyl)oxy)methoxy)-5,7-difluoro-2-methyl-3-(4′-(trifluoromethoxy)-[1,1′-biphenyl]-4-yl)quinoline 1-oxide (ELQ-735): Following the general procedure D, using a solution of ELQ-761 (382 mg, 0.72 mmol, 1 eq) and MCPBA (187 mg, 1.5 mmol, 1.5 eq) in chloroform (25 ml) to give crude ELQ-735 (581 mg). The product was purified by flash chromatography using ethyl acetate/DCM (6/4) followed by crystallization in ethyl acetate/hexane to give pure ELQ-735 (235 mg, yield 59%) as a white crystal.

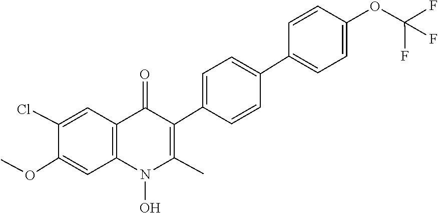 [SMILES: COc1cc2c(cc1Cl)c(=O)c(-c1ccc(-c3ccc(OC(F)(F)F)cc3)cc1)c(C)n2O]

6-chloro-1-hydroxy-7-methoxy-2-methyl-3-(4′-(trifluoromethoxy)-[1,1′-biphenyl]-4-yl)quinolin-4(1H)-one (ELQ-708): Following the general procedure E, using a solution of ELQ-707 (145 mg, 0.25 mmol, 1 eq) in ethanol/aqueous 10% NaOH (10 ml) to give pure ELQ-708 (92 mg, yield 77%) as a brown solid. ¹H-NMR (400 MHz; DMSO-d₆): δ 8.05 (s, 1H), 7.85-7.83 (m, 2H), 7.74 (broad s, 1H), 7.66-7.64 (m, 2H), 7.46 (d, J=8.1 Hz, 2H), 7.32 (d, J=7.9 Hz, 2H), 3.88 (s, 3H), 2.28 (s, 3H).

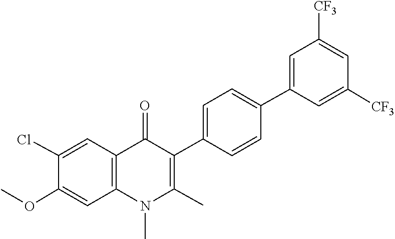 [SMILES: COc1cc2c(cc1Cl)c(=O)c(-c1ccc(-c3cc(C(F)(F)F)cc(C(F)(F)F)c3)cc1)c(C)n2C]

3-(3′,5′-bis(trifluoromethyl)-[1,1′-biphenyl]-4-yl)-6-chloro-1-hydroxy-7-methoxy-2-methylquinolin-4(1H)-one (ELQ-739): Following the general procedure E, using a solution of ELQ-736 (100 mg, 0.16 mmol, 1 eq) in ethanol/aqueous 10% NaOH (10 ml) to give pure ELQ-739 (72 mg, yield 86%) as a red solid. ¹H-NMR (400 MHz; DMSO-d₆): δ 8.37 (s, 2H), 8.07-8.06 (m, J=4.9 Hz, 2H), 7.84 (d, J=8.3 Hz, 2H), 7.77 (broad s, 1H), 7.40 (d, J=8.0 Hz, 2H), 3.90 (s, 3H), 2.29 (s, 3H). 6-fluoro-1-hydroxy-7-methoxy-2-methyl-3-(4′-(trifluoromethoxy)-[1,1′-biphenyl]-4-yl)quinolin-4(1H)-one (ELQ-710): Following the general procedure E, using a solution of ELQ-709 (140 mg, 0.25 mmol, 1 eq) in ethanol/aqueous 10% NaOH (10 ml) to give pure ELQ-710 (100 mg, yield 87%) as a white solid. ¹H-NMR (400 MHz; DMSO-d₆): δ 7.84 (d, J=8.7 Hz, 2H), 7.71 (d, J=12.0 Hz, 1H), 7.65 (d, J=8.2 Hz, 2H), 7.53 (broad s, 1H), 7.46 (d, J=8.1 Hz, 2H), 7.27 (d, J=7.9 Hz, 2H), 3.77 (s, 3H), 2.24 (s, 3H).

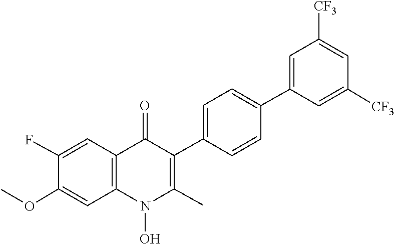 [SMILES: COc1cc2c(cc1F)c(=O)c(-c1ccc(-c3cc(C(F)(F)F)cc(C(F)(F)F)c3)cc1)c(C)n2O]

3-(3′,5′-bis(trifluoromethyl)-[1,1′-biphenyl]-4-yl)-6-fluoro-1-hydroxy-7-methoxy-2-methylquinolin-4(1H)-one (ELQ-740): Following the general procedure E, using a solution of ELQ-737 (100 mg, 0.16 mmol, 1 eq) in ethanol/aqueous 10% NaOH (10 ml) to give pure ELQ-740 (68 mg, yield 83%) as a white solid. ¹H-NMR (400 MHz; DMSO-d₆): δ 12.06 (s, 1H), 8.39 (s, 2H), 8.11 (s, 1H), 7.92 (d, J=8.1 Hz, 2H), 7.81 (d, J=11.4 Hz, 1H), 7.46-7.40 (m, 3H), 4.02 (s, 3H), 2.35 (s, 3H).

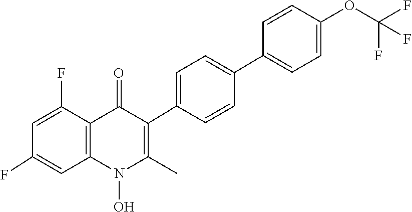 [SMILES: Cc1c(-c2ccc(-c3ccc(OC(F)(F)F)cc3)cc2)c(=O)c2c(F)cc(F)cc2n1O]

5,7-difluoro-1-hydroxy-2-methyl-3-(4′-(trifluoromethoxy)-[1,1′-biphenyl]-4-yl)quinolin-4(1H)-one (ELQ-738): Following the general procedure E, using a solution of ELQ-735 (100 mg, 0.16 mmol, 1 eq) in ethanol/aqueous 10% NaOH (10 ml) to give pure ELQ-738 (54 mg, yield 48%) as a yellow solid. ¹H-NMR (400 MHz; DMSO-d₆): δ 7.85-7.79 (m, 3H), 7.67-7.65 (m, 2H), 7.47-7.44 (m, 2H), 7.33-7.31 (m, 2H), 6.82-6.74 (m, 1H), 2.26 (s, 3H).

### Ethyl (Z)-3-((3-methoxyphenyl)imino)-2-(4′-(trifluoromethoxy)-[1,1′-biphenyl]-4-yl)butanoate

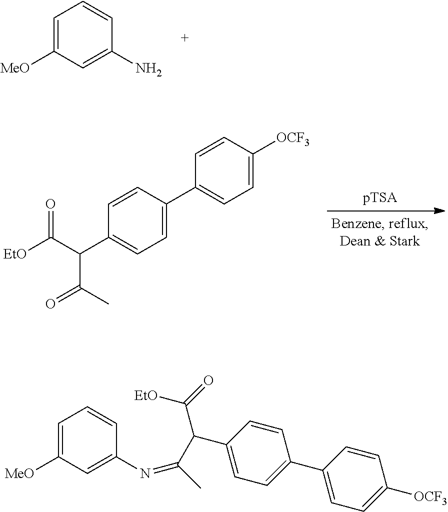 [SMILES: COc1cccc(N)c1 ; CCOC(=O)C(C(C)=O)c1ccc(-c2ccc(OC(F)(F)F)cc2)cc1 ; CCOC(=O)C(/C(C)=N\c1cccc(OC)c1)c1ccc(-c2ccc(OC(F)(F)F)cc2)cc1]

meta-Anisidine (1.10 g, 0.0089 mol) was combined with ethyl 3-oxo-2-(4′-(trifluoromethoxy)-[1,1′-biphenyl]-4-yl)butanoate (3.44 g of a 10:1 mol:mol mixture with para-toluenesulfonic acid monohydrate, thus 3.27 g, 0.0089 mol of ethyl 3-oxo-2-(4′-(trifluoromethoxy)-[1,1′-biphenyl]-4-yl)butanoate and 0.17 g, 0.00089 mol of para-toluenesulfonic acid monohydrate). This mixture was allowed to reflux in benzene (75 mL) under Dean Stark conditions for three days. The solvent was removed under reduced pressure with warming, and the residue (a stiff, brown oil) was used without purification or analysis in the ensuing reaction.

### 7-Methoxy-2-methyl-3-(4′-(trifluoromethoxy)-[1,1′-biphenyl]-4-yl)quinolin-4(1H)-one (ELQ-685)

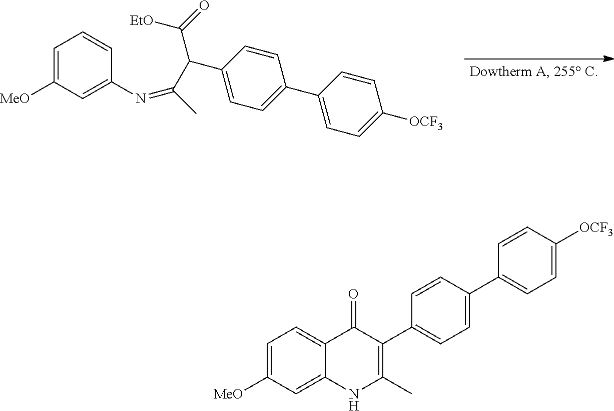 [SMILES: CCOC(=O)C(/C(C)=N\c1cccc(OC)c1)c1ccc(-c2ccc(OC(F)(F)F)cc2)cc1 ; COc1ccc2c(=O)c(-c3ccc(-c4ccc(OC(F)(F)F)cc4)cc3)c(C)[nH]c2c1]

Ethyl (Z)-3-((3-methoxyphenyl)imino)-2-(4′-(trifluoromethoxy)-[1,1′-biphenyl]-4-yl)butanoate (the crude product of the preceding reaction) was taken up in hot Dowtherm A (8 mL followed by an additional 7 mL used to rinse the flask), and was added gradually to boiling Dowtherm A (100 mL, 255° C.) over the course of 8 minutes. After a total of 11 minutes' heating, the mixture was allowed to cool, stirring, to room temperature. Hexanes (300 mL) were added with stirring, and the resulting solid was recovered by vacuum filtration, rinsing with excess hexanes followed by acetone (50 mL). The crude product (a cream solid, 1.76 g) was recrystallized from N,N-dimethylformamide (15 mL), affording 1.25 g of the desired product; a second crop was also recovered from the mother liquor (0.23 g, a total of 1.48 g fine, nearly white crystals; yield 39% over two steps from meta-anisidine, mp: 389.8-392.1° C. (dec.), ¹H-NMR (400 MHz; DMSO-d6): δ 11.51 (s, 1H), 7.99 (d, J=8.8 Hz, 1H), 7.87-7.83 (m, 2H), 7.71-7.68 (m, 2H), 7.48-7.46 (m, 2H), 7.37-7.34 (m, 2H), 6.93-6.89 (m, 2H), 3.87 (s, 3H), 2.25 (s, 3H); ¹⁹F NMR (376 MHz; DMSO): δ −56.7).

### Ethyl (((7-methoxy-2-methyl-3-(4′-(trifluoromethoxy)-[1,1′-biphenyl]-4-yl)quinolin-4-yl)oxy)methyl) carbonate (ELQ-695)

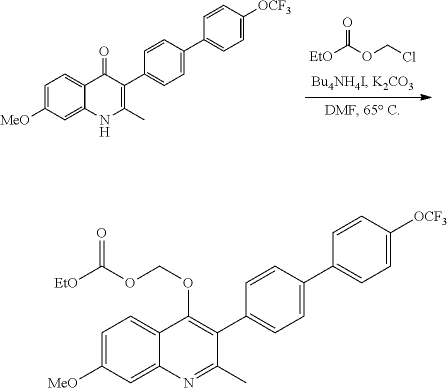 [SMILES: COc1ccc2c(=O)c(-c3ccc(-c4ccc(OC(F)(F)F)cc4)cc3)c(C)[nH]c2c1 ; CCOC(=O)OCCl ; CCOC(=O)OCOc1c(-c2ccc(-c3ccc(OC(F)(F)F)cc3)cc2)c(C)nc2cc(OC)ccc12]

To a stirred mixture of 7-methoxy-2-methyl-3-(4′-(trifluoromethoxy)-[1,1′-biphenyl]-4-yl)quinolin-4(1H)-one (ELQ-685; 0.45 g, 0.0011 mol), tetrabutyl ammonium iodide (2.0 eq., 0.0022 mol, 0.81 g), and anhydrous potassium carbonate (2.0 eq., 0.0022 mol, 0.30 g) in N,N-dimethylformamide (17 mL) was added chloromethyl ethyl carbonate (2.0 eq., 0.0022 mol, 0.30 g). This mixture was allowed to heat at 60° C. for 21 hours. The cooled reaction mixture was vacuum filtered to remove solids, and the filtrate was concentrated under reduced pressure with heating. The residue was taken up in ethyl acetate (25 mL), followed by vacuum filtration to remove precipitate. The filtrate was adsorbed onto silica and purified by flash column chromatography on silica gel, eluting with a gradient of 100% hexanes to 68/32 v/v hexanes/ethyl acetate. The desired product was obtained as a pale yellow oil that crystallized to a white solid upon standing (0.20g, yield 34%, R_{f}=0.32, 6:4 v:v hexanes:ethyl acetate, silica; mp: 111.0-112.0° C., ¹H-NMR (400 MHz; CDCl₃): δ 7.96 (d, J=9.2 Hz, 1H), 7.71-7.68 (m, 4H), 7.50-7.47 (m, 2H), 7.39 (d, J=2.5 Hz, 1H), 7.35-7.33 (m, 2H), 7.17 (dd, J=9.1, 2.5 Hz, 1H), 5.33 (s, 2H), 4.04 (q, J=7.1 Hz, 2H), 3.97 (s, 3H), 2.55 (s, 3H), 1.16 (t, J=7.1 Hz, 3H)).

### Ethyl (Z)-2-(4-bromophenyl)-3-((3-methoxyphenyl)amino)but-2-enoate

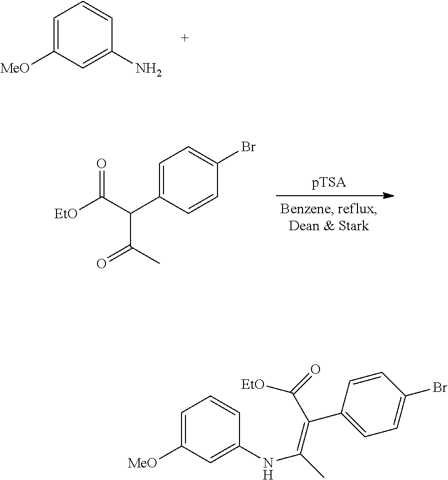 [SMILES: COc1cccc(N)c1 ; CCOC(=O)C(C(C)=O)c1ccc(Br)cc1 ; CCOC(=O)/C(=C(/C)Nc1cccc(OC)c1)c1ccc(Br)cc1]

meta-Anisidine (14.18 g, 0.115 mol) and ethyl 2-(4-bromophenyl)-3-oxobutanoate (35.03 g of a 10:1 mol:mol mixture with para-toluenesulfonic acid monohydrate, thus 32.82 g, 0.115 mol, 1 eq of ethyl 2-(4-bromophenyl)-3-oxobutanoate and 2.19 g, 0.0115 mol, 0.1 eq of pTSA) were combined in benzene (150 mL) and heated at reflux under Dean & Stark conditions for 2 days. The solvent was then removed under reduced pressure with warming, and the resulting crude material (a thick, reddish brown oil) was used without purification or analysis in the following reaction.

### 3-(4-Bromophenyl)-7-methoxy-2-methylquinolin-4(1H)-one

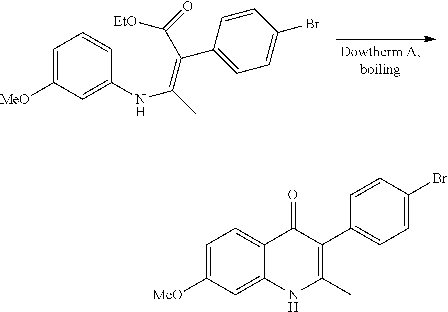 [SMILES: CCOC(=O)/C(=C(/C)Nc1cccc(OC)c1)c1ccc(Br)cc1 ; COc1ccc2c(=O)c(-c3ccc(Br)cc3)c(C)[nH]c2c1]

Ethyl (Z)-2-(4-bromophenyl)-3-((3-methoxyphenyl)amino)but-2-enoate was taken up in hot Dowtherm A (20 mL followed by an additional 10 mL used to rinse the flask), and was added gradually to boiling Dowtherm A (220 mL, 255° C.) over the course of 8 minutes. After a total of 11 minutes' heating, the mixture was allowed to cool, stirring, to room temperature. Hexanes (300 mL) were added and the resulting sticky, amber precipitate was recovered by vacuum filtration, rinsing with hexanes followed by ethyl acetate (20 mL) and finally, acetone (150 mL). This afforded the desired product as a beige powder (19.70 g, 50% over two steps from meta-anisidine; ¹H-NMR (400 MHz; DMSO-d₆): δ 11.51 (s, 1H), 7.99-7.96 (m, 1H), 7.58-7.55 (m, 2H), 7.22-7.19 (m, 2H), 6.92-6.89 (m, 2H), 3.86 (s, 3H), 2.20 (s, 3H)).

### 3-(4-Bromophenyl)-4-chloro-7-methoxy-2-methylquinoline

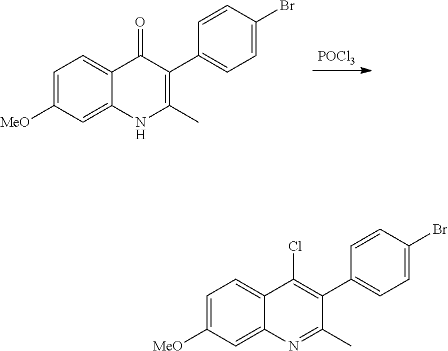

To a mixture of 3-(4-Bromophenyl)-7-methoxy-2-methylquinolin-4(1H)-one (19.70 g, 0.054 mol) and chloroform (125 mL) was added phosphorus oxychloride (3.0 eq, 0.162 mol, 15.1 mL). The reaction was stirred at reflux for 2 days. After cooling, the reaction was poured onto ice (total volume 500 mL) and stirred vigorously for 20 minutes. Additional chloroform (30 mL) and water (100 mL) were added to aid mixing and dissolve solid, and stirring was continued for a further 10 minutes. The mixture was then made basic by the addition of 50% w/w aqueous sodium hydroxide followed by stirring for 30 minutes. The biphasic mixture was vacuum filtered and the filtrate (further diluted with water, 150 mL) was separated. The aqueous layer was further extracted with chloroform (100 mL, then 2×75 mL). The pooled organic layers were rinsed with brine (75 mL), dried (MgSO₄) and evaporated under reduced pressure with warming, affording a greenish gray solid (21.21 g). This material was recrystallized from ethyl acetate (125 mL), affording the desired product as grayish tan crystals (13.84 g); a second crop (2.92 g) and third crop (0.83 g) were also the desired product (total yield 17.59 g, 90%; ¹H-NMR (400 MHz; CDCl₃): δ 8.10 (d, J=9.2 Hz, 1H), 7.66-7.62 (m, 2H), 7.39 (d, J=2.5 Hz, 1H), 7.26-7.23 (m, overlaps solvent signal, estimated 1H), 7.18-7.15 (m, 2H), 3.97 (s, 3H), 2.45 (s, 3H)).

### 3-(3′,5′-bis(Trifluoromethyl)-[1,1′-biphenyl]-4-yl)-4-chloro-7-methoxy-2-methylquinoline

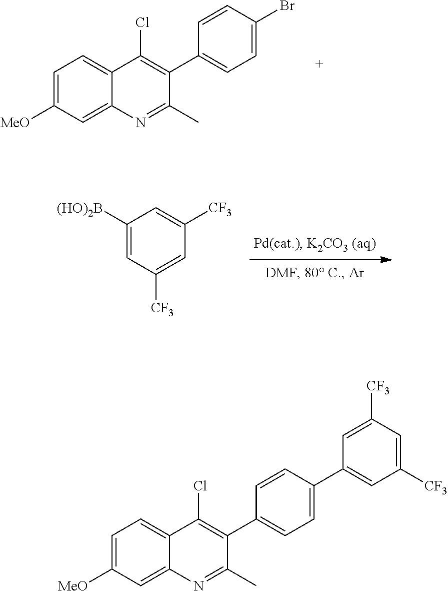 [SMILES: COc1ccc2c(Cl)c(-c3ccc(Br)cc3)c(C)nc2c1 ; OB(O)c1cc(C(F)(F)F)cc(C(F)(F)F)c1 ; COc1ccc2c(Cl)c(-c3ccc(-c4cc(C(F)(F)F)cc(C(F)(F)F)c4)cc3)c(C)nc2c1]

A mixture of 3-(4-bromophenyl)-4-chloro-7-methoxy-2-methylquinoline (1.00 g, 0.0028 mol), 3,5-bis(trifluoromethyl)phenylboronic acid (1.3 eq., 0.0036 mol, 0.92 g), and anhydrous potassium carbonate (2.0 eq, 0.0056 mol, 0.58g, dissolved in water, 3.6 mL) in N,N-dimethylformamide (80 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.10 g, 0.00014 mol) was added and the reaction was allowed to heat at 80° C. under argon for 3 days. The cooled reaction mixture was vacuum filtered, followed by concentration of the filtrate under reduced pressure with heating. The resulting solid was taken up in 125 mL DCM, followed by vacuum filtration. Automated flash chromatography of the evaporated filtrate on silica, eluting with a gradient of 1:0 to 82:18 v:v hexanes:ethyl acetate, isolated the desired product (R_{f}=0.24, 5:2 v:v hexanes:ethyl acetate on silica) mixed with the major side product (3-(3′,5′-bis(trifluoromethyl)-[1,1′-biphenyl]-4-yl)-4-(3,5-bis(trifluoromethyl)phenyl)-6-chloro-7-methoxy-2-methylquinoline, resulting from double addition of 3,5-bis(trifluoromethyl)phenylboronic acid); total 1.11 g. This mixture was used without further purification in the next reaction.

### 3-(3′,5′-bis(trifluoromethyl)-[1,1′-biphenyl]-4-yl)-7-methoxy-2-methylquinolin-4(1H)-one (ELQ-731)

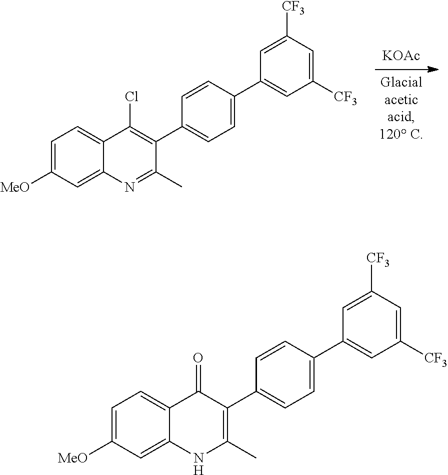 [SMILES: COc1ccc2c(Cl)c(-c3ccc(-c4cc(C(F)(F)F)cc(C(F)(F)F)c4)cc3)c(C)nc2c1 ; COc1ccc2c(=O)c(-c3ccc(-c4cc(C(F)(F)F)cc(C(F)(F)F)c4)cc3)c(C)[nH]c2c1 ; "KOAc"]

3-(3′,5′-bis(Trifluoromethyl)-[1,1′-biphenyl]-4-yl)-4-chloro-7-methoxy-2-methylquinoline (1.11 g, containing both the desired starting material and its side product) and anhydrous potassium acetate (0.021 mol, 2.05 g) were combined in glacial acetic acid (15 mL) and allowed to heat, stirring, for 1 day at 110° C. After cooling to room temperature, the solid that formed in the reaction mixture was recovered by vacuum filtration, rinsing with excess water followed by 3 mL acetone. This afforded the desired product as a white powder (0.84 g, 63% over two steps from 3-(4-bromophenyl)-4-chloro-7-methoxy-2-methylquinoline, ¹H-NMR (400 MHz; DMSO-d₆): δ 11.53 (s, 1H), 8.41-8.37 (m, 2H), 8.11-8.08 (m, 1H), 8.00 (d, J=9.0 Hz, 1H), 7.91-7.88 (m, 2H), 7.43-7.40 (m, 2H), 6.94-6.90 (m, 2H), 3.88 (s, 3H), 2.26 (s, 3H); 19-F NMR (376 MHz; DMSO): δ −61.2).

### 4-Chloro-7-methoxy-2-methyl-3-(3′-(trifluoromethyl)-[1,1′-biphenyl]-4-yl)quinoline

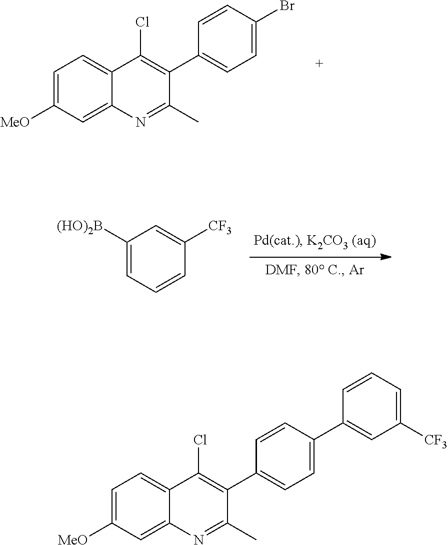 [SMILES: COc1ccc2c(Cl)c(-c3ccc(Br)cc3)c(C)nc2c1 ; OB(O)c1cccc(C(F)(F)F)c1 ; COc1ccc2c(Cl)c(-c3ccc(-c4cccc(C(F)(F)F)c4)cc3)c(C)nc2c1]

A mixture of 3-(4-bromophenyl)-4-chloro-7-methoxy-2-methylquinoline (1.00 g, 0.0028 mol), 3-(trifluoromethyl)phenylboronic acid (1.3 eq., 0.0036 mol, 0.68 g), and anhydrous potassium carbonate (2.0 eq, 0.0056 mol, 0.58g, dissolved in water, 3.6 mL) in N,N-dimethylformamide (80 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.10 g, 0.00014 mol) was added and the reaction was allowed to heat at 80° C. under argon for 3 days. The cooled reaction mixture was vacuum filtered, followed by concentration of the filtrate under reduced pressure with heating. The resulting solid was taken up in 125 mL DCM, followed by vacuum filtration. Automated flash chromatography of the evaporated filtrate on silica, eluting with a gradient of 1:0 to 4:1 v-v hexanes:ethyl acetate, isolated the desired product (R_{f}=0.42, 5:2 v:v hexanes:ethyl acetate on silica) as a white solid (0.79 g, 61%, ¹H-NMR (400 MHz; CDCl₃): δ 8.12 (d, J=9.2 Hz, 1H), 7.95-7.92 (m, 1H), 7.89-7.84 (m, 1H), 7.76-7.73 (m, 2H), 7.66-7.58 (m, 2H), 7.42-7.38 (m, 3H), 7.28-7.25 (m, 1H), 3.98 (s, 3H), 2.51 (s, 3H); 19-F NMR (376 MHz; CDCl3): δ −62.6).

### 7-Methoxy-2-methyl-3-(3′-(trifluoromethyl)-[1,1′-biphenyl]-4-yl)quinolin-4(1H)-one (ELQ-730)

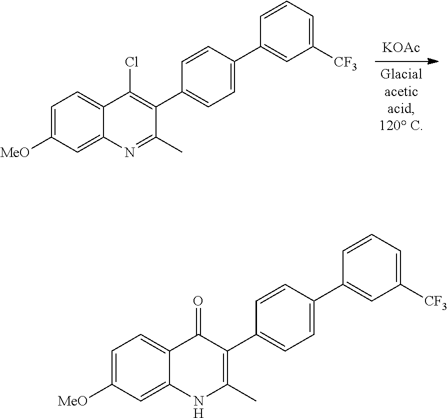 [SMILES: COc1ccc2c(Cl)c(-c3ccc(-c4cccc(C(F)(F)F)c4)cc3)c(C)nc2c1 ; COc1ccc2c(=O)c(-c3ccc(-c4cccc(C(F)(F)F)c4)cc3)c(C)[nH]c2c1 ; "KOAc"]

4-Chloro-7-methoxy-2-methyl-3-(3′-(trifluoromethyl)-[1,1′-biphenyl]-4-yl)quinoline (0.79 g, 0.0017 mol) and anhydrous potassium acetate (10 eq, 0.017 mol, 1.67 g) were combined in glacial acetic acid (15 mL) and allowed to heat, stirring, for 1 day at 110° C. After cooling to room temperature, the solid that formed in the reaction mixture was recovered by vacuum filtration, rinsing with excess water followed by 3 mL acetone. This afforded the desired product as a white powder (0.54 g, 78%, ¹H-NMR (400 MHz; DMSO-d₆): δ 11.52 (s, 1H), 8.06-7.99 (m, 3H), 7.78-7.73 (m, 4H), 7.39-7.37 (m, 2H), 6.94-6.90 (m, 2H), 3.87 (s, 3H), 2.26 (s, 3H); 19-F NMR (376 MHz; DMSO): δ −61.0).

### 4-chloro-7-methoxy-2-methyl-3-(3′-(trifluoromethoxy)-[1,1′-biphenyl]-4-yl)quinoline

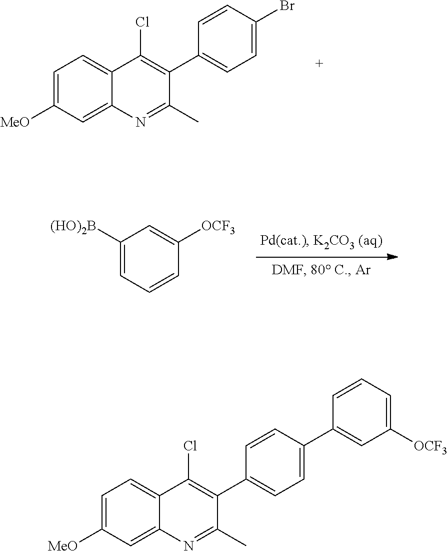 [SMILES: COc1ccc2c(Cl)c(-c3ccc(Br)cc3)c(C)nc2c1 ; OB(O)c1cccc(OC(F)(F)F)c1 ; COc1ccc2c(Cl)c(-c3ccc(-c4cccc(OC(F)(F)F)c4)cc3)c(C)nc2c1]

A mixture of 3-(4-bromophenyl)-4-chloro-7-methoxy-2-methylquinoline (0.70 g, 0.0019 mol), 3-(trifluoromethoxy)phenyboronic acid (1.3 eq., 0.0025 mol, 0.52 g), and anhydrous potassium carbonate (2.0 eq, 0.0038 mol, 0.52g, dissolved in water, 1.9 mL) in N,N-dimethylformamide (80 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.070 g, 0.000095 mol) was added and the reaction was allowed to heat at 80° C. under argon for 23 hours. The cooled reaction mixture was vacuum filtered, followed by concentration of the filtrate under reduced pressure with heating. The resulting solid was taken up in 100 mL DCM, followed by vacuum filtration. Automated flash chromatography of the evaporated filtrate on silica, eluting with a gradient of 95:5 to 82:18 v:v hexanes:ethyl acetate, isolated the desired product (R_{f}=0.32, 7:3 v:v hexanes:ethyl acetate on silica) as a white solid (0.24 g, 28%, ¹H-NMR (400 MHz; CDCl₃): δ 8.13 (d, J=9.2 Hz, 1H), 7.73-7.70 (m, 2H), 7.62 (ddd, J=7.8, 1.7, 1.0 Hz, 1H), 7.54-7.48 (m, 2H), 7.41 (d, J=2.5 Hz, 1H), 7.40-7.37 (m, 2H), 7.28-7.23 (m, overlaps residual solvent signal, estimated 2H), 3.98 (s, 3H), 2.50 (s, 3H); 19-F NMR (376 MHz; CDCl3): δ −57.7).

### 7-methoxy-2-methyl-3-(3′-(trifluoromethoxy)-[1,1′-biphenyl]-4-yl)quinolin-4(1H)-one (ELQ-732)

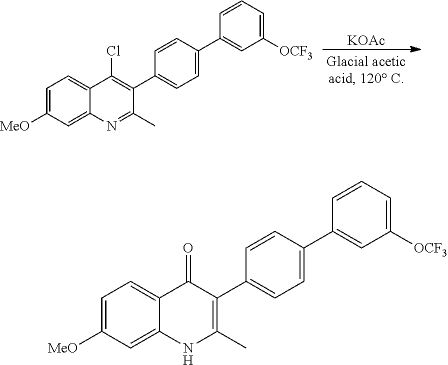 [SMILES: COc1ccc2c(Cl)c(-c3ccc(-c4cccc(OC(F)(F)F)c4)cc3)c(C)nc2c1 ; COc1ccc2c(=O)c(-c3ccc(-c4cccc(OC(F)(F)F)c4)cc3)c(C)[nH]c2c1 ; "KOAc"]

4-Chloro-7-methoxy-2-methyl-3-(3′-(trifluoromethoxy)-[1,1′-biphenyl]-4-yl)quinoline (0.24 g, 0.00054 mol) and anhydrous potassium acetate (10 eq, 0.0054 mol, 0.53 g) were combined in glacial acetic acid (11 mL) and allowed to heat, stirring, for 22 hours at 120° C. After cooling to room temperature, the solid that formed in the reaction mixture was recovered by vacuum filtration, rinsing with excess water followed by 3 mL acetone. This afforded the desired product as white crystals (0.17 g, 74%, ¹H-NMR (400 MHz; DMSO-d₆): δ 11.51 (s, 1H), 7.99 (d, J=8.8 Hz, 1H), 7.78 (ddd, J=7.9, 1.7, 0.9 Hz, 1H), 7.75-7.72 (m, 2H), 7.70-7.67 (m, 1H), 7.65-7.59 (m, 1H), 7.39-7.35 (m, 3H), 6.93-6.89 (m, 2H), 3.87 (s, 3H), 2.25 (s, 3H); 19-F NMR (376 MHz; DMSO): δ −56.6).

### 4-chloro-3-(3′,5′-difluoro-4′-(trifluoromethoxy)-[1,1′-biphenyl]-4-yl)-7-methoxy-2-methylquinoline

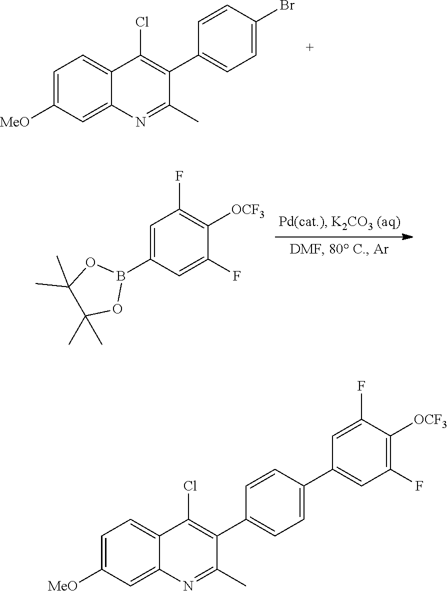 [SMILES: COc1ccc2c(Cl)c(-c3ccc(Br)cc3)c(C)nc2c1 ; "+" ; CC1(C)OB(c2cc(F)c(OC(F)(F)F)c(F)c2)OC1(C)C ; COc1ccc2c(Cl)c(-c3ccc(-c4cc(F)c(OC(F)(F)F)c(F)c4)cc3)c(C)nc2c1]

A mixture of 3-(4-bromophenyl)-4-chloro-7-methoxy-2-methylquinoline (0.70 g, 0.0019 mol), 2-(3,5-difluoro-4-(trifluoromethoxy)phenyl)-4,4,5,5-tetramethyl-1,3,2-dioxaborolane (1.2 eq., 0.0023 mol, 0.75 g), and anhydrous potassium carbonate (2.0 eq, 0.0038 mol, 0.53g, dissolved in water, 1.9 mL) in N,N-dimethylformamide (80 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.070 g, 0.000095 mol) was added and the reaction was allowed to heat at 80° C. under argon for 4 days. The cooled reaction mixture was vacuum filtered, followed by concentration of the filtrate under reduced pressure with heating. The resulting solid was taken up in 100 mL DCM, followed by vacuum filtration. Automated flash chromatography of the evaporated filtrate on silica, eluting with a gradient of 95:5 to 83:17 v:v hexanes:ethyl acetate, isolated the desired product (R_{f}=0.29, 7:3 v:v hexanes:ethyl acetate on silica) as a white, crystalline solid (0.67 g, 74%, ¹H-NMR (400 MHz; CDCl₃): δ 8.12 (d, J=9.2 Hz, 1H), 7.68-7.65 (m, 2H), 7.41-7.38 (m, 3H), 7.35-7.30 (m, 2H), 7.28-7.25 (m, overlaps residual solvent signal, estimated 1H), 3.98 (s, 3H), 2.49 (s, 3H); 19-F NMR (376 MHz; CDCl3): δ −124.2).

### 3-(3′,5′-difluoro-4′-(trifluoromethoxy)-[1,1′-biphenyl]-4-yl)-7-methoxy-2-methylquinolin-4(1H)-one (ELQ-733)

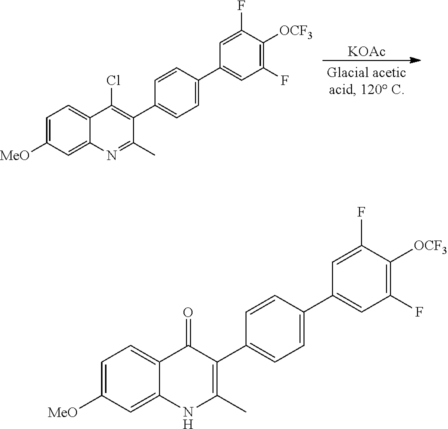 [SMILES: COc1ccc2c(Cl)c(-c3ccc(-c4cc(F)c(OC(F)(F)F)c(F)c4)cc3)c(C)nc2c1 ; COc1ccc2c(=O)c(-c3ccc(-c4cc(F)c(OC(F)(F)F)c(F)c4)cc3)c(C)[nH]c2c1 ; "KOAc"]

4-Chloro-3-(3′,5′-difluoro-4′-(trifluoromethoxy)-[1,1′-biphenyl]-4-yl)-7-methoxy-2-methylquinoline (0.67 g, 0.0014 mol) and anhydrous potassium acetate (10 eq, 0.014 mol, 1.37 g) were combined in glacial acetic acid (11 mL) and allowed to heat, stirring, for 22 hours at 120° C. After cooling to room temperature, the solid that formed in the reaction mixture was recovered by vacuum filtration, rinsing with excess water followed by 3 mL acetone. This afforded the desired product as white crystals (0.52 g, 81%, ¹H-NMR (400 MHz; DMSO-d₆): δ 11.52 (s, 1H), 7.99 (d, J=8.7 Hz, 1H), 7.85-7.79 (m, 4H), 7.40-7.37 (m, 2H), 6.93-6.89 (m, 2H), 3.87 (s, 3H), 2.25 (s, 3H); 19-F NMR (376 MHz; DMSO): δ −59.0).

### 4-chloro-3-(3′,5′-difluoro-4′-methoxy-[1,1′-biphenyl]-4-yl)-7-methoxy-2-methylquinoline

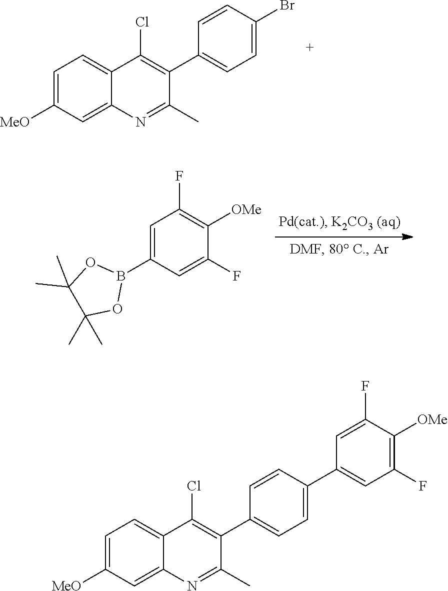 [SMILES: COc1ccc2c(Cl)c(-c3ccc(Br)cc3)c(C)nc2c1 ; "+" ; COc1c(F)cc(B2OC(C)(C)C(C)(C)O2)cc1F ; COc1ccc2c(Cl)c(-c3ccc(-c4cc(F)c(OC)c(F)c4)cc3)c(C)nc2c1]

A mixture of 3-(4-bromophenyl)-4-chloro-7-methoxy-2-methylquinoline (0.70 g, 0.0019 mol), 2-(3,5-difluoro-4-(methoxy)phenyl-4,4,5,5-tetramethyl-1,3,2-dioxaborolane (1.2 eq., 0.0023 mol, 0.62 g), and anhydrous potassium carbonate (2.0 eq, 0.0038 mol, 0.53g, dissolved in water, 1.9 mL) in N,N-dimethylformamide (80 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.070 g, 0.000095 mol) was added and the reaction was allowed to heat at 80° C. under argon for 4 days. The cooled reaction mixture was vacuum filtered, followed by concentration of the filtrate under reduced pressure with heating. The resulting solid was taken up in 100 mL DCM, followed by vacuum filtration. Automated flash chromatography of the evaporated filtrate on silica, eluting with a gradient of 95:5 to 82:18 v:v hexanes:ethyl acetate, isolated the desired product (R_{f}=0.32, 7:3 v:v hexanes:ethyl acetate on silica) as a white, crystalline solid (0.72 g, 89%, ¹H-NMR (400 MHz; CDCl₃): δ 8.12 (d, J=9.2 Hz, 1H), 7.66-7.63 (m, 2H), 7.41 (d, J=2.5 Hz, 1H), 7.37-7.34 (m, 2H), 7.28-7.20 (m, overlaps residual solvent signal, estimated 3H), 4.07 (s, 3H), 3.98 (s, 3H), 2.49 (s, 3H); 19-F NMR (376 MHz; CDCl3): δ −128.3).

### 3-(3′,5′-difluoro-4′-methoxy-[1,1′-biphenyl]-4-yl)-7-methoxy-2-methylquinolin-4(1H)-one (ELQ-734)

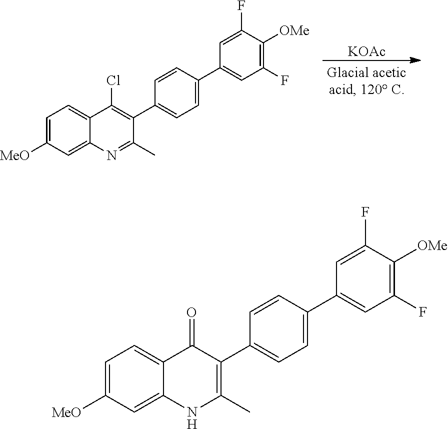 [SMILES: COc1ccc2c(Cl)c(-c3ccc(-c4cc(F)c(OC)c(F)c4)cc3)c(C)nc2c1 ; COc1ccc2c(=O)c(-c3ccc(-c4cc(F)c(OC)c(F)c4)cc3)c(C)[nH]c2c1 ; "KOAc"]

4-Chloro-3-(3′,5′-difluoro-4′-methoxy-[1,1′-biphenyl]-4-yl)-7-methoxy-2-methylquinoline (0.72 g, 0.0017 mol) and anhydrous potassium acetate (10 eq, 0.017 mol, 1.67 g) were combined in glacial acetic acid (11 mL) and allowed to heat, stirring, for 22 hours at 120° C. After cooling to room temperature, the solid that formed in the reaction mixture was recovered by vacuum filtration, rinsing with excess water followed by 3 mL acetone. This afforded the desired product as white crystals (0.50 g, 71%, ¹H-NMR (400 MHz; DMSO-d₆): δ 11.50 (s, 1H), 8.00-7.98 (m, 1H), 7.73-7.70 (m, 2H), 7.59-7.53 (m, 2H), 7.35-7.32 (m, 2H), 6.93-6.89 (m, 2H), 3.97 (s, 3H), 3.87 (s, 3H), 2.24 (s, 3H); 19-F NMR (376 MHz; DMSO): δ −128.4).

### 4-chloro-3-(4′-fluoro-2′-methoxy-[1,1′-biphenyl]-4-yl)-7-methoxy-2-methylquinoline

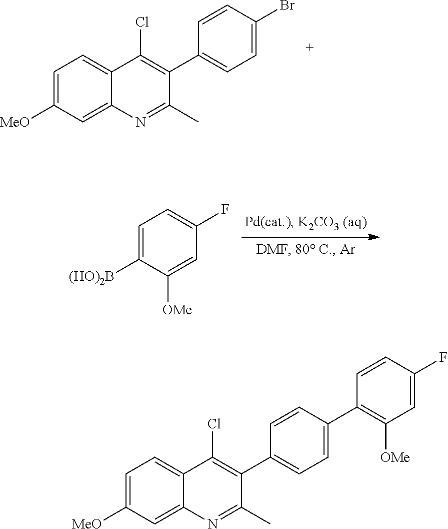 [SMILES: COc1ccc2c(Cl)c(-c3ccc(Br)cc3)c(C)nc2c1 ; "+" ; COc1cc(F)ccc1B(O)O ; COc1ccc2c(Cl)c(-c3ccc(-c4ccc(F)cc4OC)cc3)c(C)nc2c1]

A mixture of 3-(4-bromophenyl)-4-chloro-7-methoxy-2-methylquinoline (0.70 g, 0.0019 mol), 2-methoxy-4-(trifluoromethyl)phenylboronic acid (1.2 eq., 0.0023 mol, 0.51 g), and anhydrous potassium carbonate (2.0 eq, 0.0038 mol, 0.53g, dissolved in water, 1.9 mL) in N,N-dimethylformamide (80 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.070 g, 0.000095 mol) was added and the reaction was allowed to heat at 80° C. under argon for 20 hours. The cooled reaction mixture was vacuum filtered, followed by concentration of the filtrate under reduced pressure with heating. The resulting solid was taken up in 120 mL DCM, followed by vacuum filtration. Automated flash chromatography of the evaporated filtrate on silica, eluting with a gradient of 93:7 to 79:21 v:v hexanes:ethyl acetate, isolated the desired product (R_{f}=0.26, 7:3 v:v hexanes:ethyl acetate on silica) as a white, crystalline solid (0.70 g, 90%, ¹H-NMR (400 MHz; CDCl₃): δ 8.13 (d, J=9.2 Hz, 1H), 7.64-7.61 (m, 2H), 7.41 (d, J=2.5 Hz, 1H), 7.40-7.36 (m, 1H), 7.32-7.29 (m, 2H), 7.27-7.24 (m, overlaps residual solvent signal, estimated 1H), 6.80-6.73 (m, 2H), 3.98 (s, 3H), 3.86 (s, 3H), 2.52 (s, 3H); 19-F NMR (376 MHz; CDCl3): δ −112.0).

### 3-(4′-Fluoro-2′-methoxy-[1,1′-biphenyl]-4-yl)-7-methoxy-2-methylquinolin-4(1H)-one (ELQ-743)

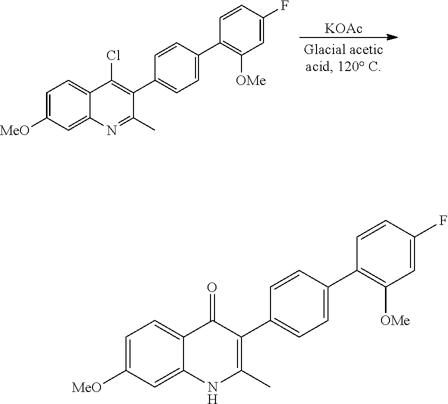 [SMILES: COc1ccc2c(Cl)c(-c3ccc(-c4ccc(F)cc4OC)cc3)c(C)nc2c1 ; COc1ccc2c(=O)c(-c3ccc(-c4ccc(F)cc4OC)cc3)c(C)[nH]c2c1 ; "KOAc"]

4-Chloro-3-(4′-fluoro-2′-methoxy-[1,1′-biphenyl]-4-yl)-7-methoxy-2-methylquinoline (0.70 g, 0.0017 mol) and anhydrous potassium acetate (10 eq, 0.017 mol, 1.67 g) were combined in glacial acetic acid (10 mL) and allowed to heat, stirring, for 23 hours at 120° C. After cooling to room temperature, the solid that formed in the reaction mixture was recovered by vacuum filtration, rinsing with excess water followed by 3×1.5 mL acetone. This afforded the desired product as a white powder (0.57 g, 86%, ¹H-NMR (400 MHz; DMSO-d₆): δ 11.48 (s, 1H), 7.99 (d, J=8.8 Hz, 1H), 7.49-7.43 (m, 2H), 7.40-7.34 (m, 1H), 7.29-7.24 (m, 2H), 7.05-7.02 (m, 1H), 6.95-6.85 (m, 3H), 3.87 (s, 3H), 3.82 (s, 3H), 2.25 (s, 3H); 19-F NMR (376 MHz; DMSO): δ −112.3).

### 4-Chloro-7-methoxy-3-(2′-methoxy-4′-(trifluoromethyl)-[1,1′-biphenyl]-4-yl)-2-methylquinoline

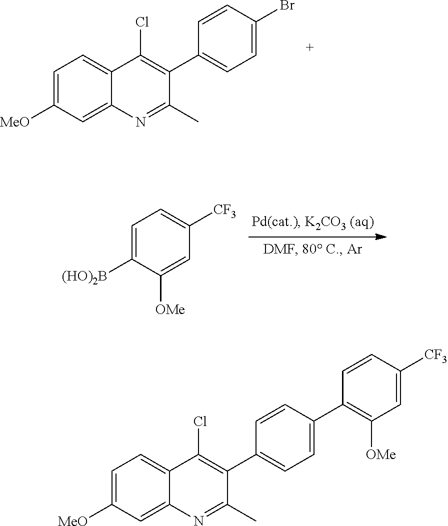 [SMILES: COc1ccc2c(Cl)c(-c3ccc(Br)cc3)c(C)nc2c1 ; "+" ; COc1cc(C(F)(F)F)ccc1B(O)O ; COc1ccc2c(Cl)c(-c3ccc(-c4ccc(C(F)(F)F)cc4OC)cc3)c(C)nc2c1]

A mixture of 3-(4-bromophenyl)-4-chloro-7-methoxy-2-methylquinoline (0.70 g, 0.0019 mol), 2-methoxy-4-fluorophenylboronic acid (1.2 eq., 0.0023 mol, 0.39 g), and anhydrous potassium carbonate (2.0 eq, 0.0038 mol, 0.53g, dissolved in water, 1.9 mL) in N,N-dimethylformamide (80 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.070 g, 0.000095 mol) was added and the reaction was allowed to heat at 80° C. under argon for 20 hours. The cooled reaction mixture was vacuum filtered, followed by concentration of the filtrate under reduced pressure with heating. The resulting solid was taken up in 120 mL DCM, followed by vacuum filtration. Automated flash chromatography of the evaporated filtrate on silica, eluting with a gradient of 93:7 to 77:23 v:v hexanes:ethyl acetate, isolated the desired product (R_{f}=0.26, 7:3 v:v hexanes:ethyl acetate on silica) as a white, crystalline solid (0.64 g, 74%, ¹H-NMR (400 MHz; CDCl₃): δ 8.13 (d, J=9.2 Hz, 1H), 7.68-7.65 (m, 2H), 7.54-7.52 (m, 1H), 7.41 (d, J=2.5 Hz, 1H), 7.36-7.33 (m, 3H), 7.27-7.25 (m, overlaps solvent residual peak, 1H), 7.24-7.21 (s, 1H), 3.98 (s, 3H), 3.92 (s, 3H), 2.53 (s, 3H); 19-F NMR (376 MHz; CDCl3): δ −62.5).

### 4-Chloro-7-methoxy-3-(2′-methoxy-4′-(trifluoromethyl)-[1,1′-biphenyl]-4-yl)-2-methylquinoline (ELQ-744)

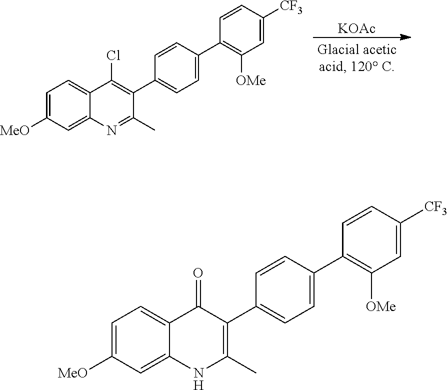 [SMILES: COc1ccc2c(Cl)c(-c3ccc(-c4ccc(C(F)(F)F)cc4OC)cc3)c(C)nc2c1 ; COc1ccc2c(=O)c(-c3ccc(-c4ccc(C(F)(F)F)cc4OC)cc3)c(C)[nH]c2c1 ; "KOAc"]

4-Chloro-7-methoxy-3-(2′-methoxy-4′-(trifluoromethyl)-[1,1′-biphenyl]-4-yl)-2-methylquinoline (0.64 g, 0.0014 mol) and anhydrous potassium acetate (10 eq, 0.014 mol, 1.37 g) were combined in glacial acetic acid (10 mL) and allowed to heat, stirring, for 23 hours at 120° C. After cooling to room temperature, the solid that formed in the reaction mixture was recovered by vacuum filtration, rinsing with excess water followed by 3×1.5 mL acetone. This afforded the desired product as a white powder (0.55 g, 89%, ¹H-NMR (400 MHz; DMSO-d₆): δ 11.51 (s, 1H), 8.00 (d, J=8.8 Hz, 1H), 7.60-7.52 (m, 3H), 7.43-7.40 (m, 2H), 7.32-7.30 (m, 2H), 6.93-6.89 (m, 2H), 3.90 (s, 3H), 3.87 (s, 3H), 2.26 (s, 3H); 19-F NMR (376 MHz; DMSO): δ −60.8).

### 4-(((Ethoxycarbonyl)oxy)methoxy)-7-methoxy-2-methyl-3-(4′-(trifluoromethoxy)-[1,1′-biphenyl]-4-yl)quinoline 1-oxide (ELQ-754)

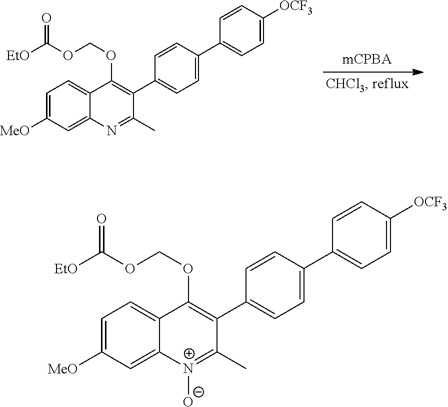 [SMILES: CCOC(=O)OCOc1c(-c2ccc(-c3ccc(OC(F)(F)F)cc3)cc2)c(C)nc2cc(OC)ccc12 ; CCOC(=O)OCOc1c(-c2ccc(-c3ccc(OC(F)(F)F)cc3)cc2)c(C)[n+]([O-])c2cc(OC)ccc12]

To ethyl (((7-methoxy-2-methyl-3-(4′-(trifluoromethoxy)-[1,1′-biphenyl]-4-yl)quinolin-4-yl)oxy)methyl) carbonate (0.53 g, 0.0010 mol) in chloroform (50 mL) was added 3-chloroperoxybenzoic acid (1.5 eq, 0.26 g, 0.0015 mol). The reaction was allowed to heat at reflux for 20 hours. After cooling, the reaction was evaporated under reduced pressure with warming and the residue was purified by chromatography on silica, eluting with a gradient of 88:12 to 0:100 v:v dichloromethane:ethyl acetate (product R_{f}=0.09, 100% ethyl acetate). The resulting white solid (0.43 g) was additionally recrystallized from a mixture of ethyl acetate (1.5 mL) and hexanes (3.5 mL). This afforded the desired product as off-white needles (0.31 g, 57%, ¹H-NMR (400 MHz; CDCl₃): δ 8.18 (d, J=2.5 Hz, 1H), 8.02 (d, J=9.2 Hz, 1H), 7.73-7.67 (m, 4H), 7.49-7.46 (m, 2H), 7.36-7.33 (m, 2H), 7.29-7.27 (m, overlaps solvent residual signal, estimated 1H), 5.28 (s, 2H), 4.05-3.99 (m, 5H), 2.59 (s, 3H), 1.14 (t, J=7.1 Hz, 3H)).

### 1-hydroxy-7-methoxy-2-methyl-3-(4′-(trifluoromethoxy)-[1,1′-biphenyl]-4-yl)quinolin-4(1H)-one (ELQ-755)

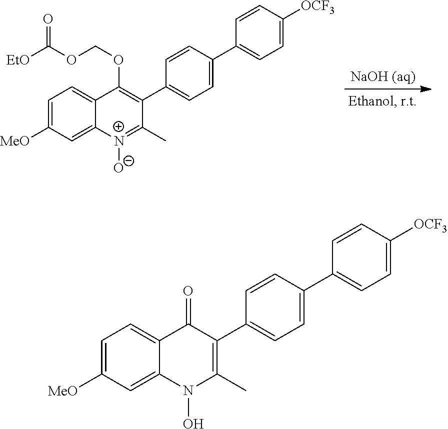 [SMILES: CCOC(=O)OCOc1c(-c2ccc(-c3ccc(OC(F)(F)F)cc3)cc2)c(C)[n+]([O-])c2cc(OC)ccc12 ; COc1ccc2c(=O)c(-c3ccc(-c4ccc(OC(F)(F)F)cc4)cc3)c(C)n(O)c2c1]

4-(((Ethoxycarbonyl)oxy)methoxy)-7-methoxy-2-methyl-3-(4′-(trifluoromethoxy)-[1,1′-biphenyl]-4-yl)quinoline 1-oxide (0.28 g, 0.00066 mol) was dissolved by stirring in 10 mL of absolute ethanol. Aqueous sodium hydroxide (0.54 mL of a 10% solution, thus 3.3 eq, 0.0015 mol, 0.06 g NaOH) was added while stirring at room temperature. After 105 minutes, the reaction was concentrated to 2 mL, then poured into 80 mL of water. This mixture was allowed to stir overnight, followed by vacuum filtration; after rinsing with water and allowing to remain on suction for 1 hour, the resulting white powder was additionally rinsed with dichloromethane (3×0.5 mL). This afforded the desired product as an off-white powder (0.17 g, 58%, ¹H-NMR (400 MHz; DMSO-d₆): δ 11.77 (s, 1H), 8.07 (d, J=8.9 Hz, 1H), 7.86-7.83 (m, 2H), 7.75-7.68 (m, 2H), 7.51-7.45 (m, 2H), 7.37-7.33 (m, 2H), 7.25-7.20 (m, 1H), 6.98 (dd, J=8.9, 2.1 Hz, 1H), 3.92 (s, 3H), 2.33 (s, 3H); 19-F NMR (376 MHz; DMSO): δ −56.7).

### Ethyl (Z)-3-((4-chloro-3,5-difluorophenyl)amino)-2-(4′-(trifluoromethoxy)-[1,1′-biphenyl]-4-yl)but-2-enoate

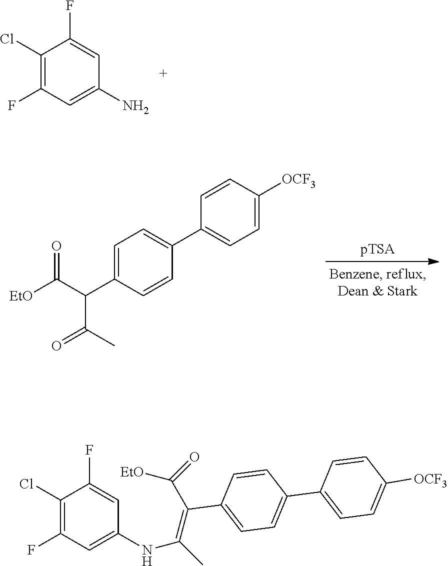 [SMILES: Nc1cc(F)c(Cl)c(F)c1 ; "+" ; CCOC(=O)C(C(C)=O)c1ccc(-c2ccc(OC(F)(F)F)cc2)cc1 ; CCOC(=O)/C(=C(/C)Nc1cc(F)c(Cl)c(F)c1)c1ccc(-c2ccc(OC(F)(F)F)cc2)cc1]

4-Chloro-3,5-difluoroaniline (0.82 g, 0.0050 mol) was combined with a mixture of ethyl 3-oxo-2-(4′-(trifluoromethoxy)-[1,1′-biphenyl]-4-yl)butanoate (1.0 eq, 1.84 g, 0.0050 mol) and para-toluenesulfonic acid monohydrate (0.1 eq, 0.11 g, 0.00058 mol) in benzene (70 mL). This mixture was allowed to reflux under Dean Stark conditions for two days. The solvent was removed under reduced pressure with warming, and the residue (a reddish brown oil) was used without purification or analysis in the ensuing reaction.

### 6-Chloro-5,7-difluoro-2-methyl-3-(4′-(trifluoromethoxy)-[1,1′-biphenyl]-4-yl)quinolin-4(1H)-one (ELQ-648)

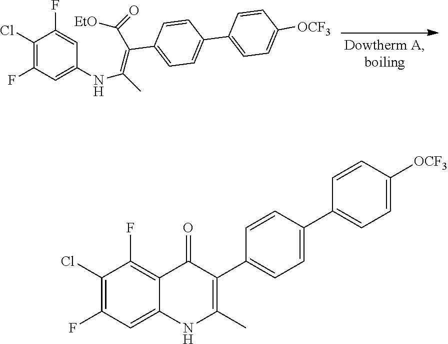 [SMILES: CCOC(=O)/C(=C(/C)Nc1cc(F)c(Cl)c(F)c1)c1ccc(-c2ccc(OC(F)(F)F)cc2)cc1 ; Cc1[nH]c2cc(F)c(Cl)c(F)c2c(=O)c1-c1ccc(-c2ccc(OC(F)(F)F)cc2)cc1]

Ethyl (Z)-3-((4-chloro-3,5-difluorophenyl)amino)-2-(4′-(trifluoromethoxy)-[1,1′-biphenyl]-4-yl)but-2-enoate (the crude product of the preceding reaction) was taken up in hot Dowtherm A (8 mL followed by an additional 7 mL used to rinse the flask), and was added gradually to boiling Dowtherm A (80 mL, 255° C.) over the course of 7 minutes. After a total of 10 minutes' heating, the mixture was allowed to cool, stirring, to room temperature. Hexanes (300 mL) were added with stirring, and the resulting solid was recovered by vacuum filtration, rinsing with hexanes (50 mL) followed by ethyl acetate (3×10 mL), additional hexanes (2×10 mL). The crude product (a pale pink, sparkling solid) was recrystallized from N,N-dimethylformamide (5 mL), affording 0.31 g of the desired product; a second crop afforded an additional 0.13 g (total yield over two steps from 4-chloro-3,5-difluoroaniline 0.44 g, 19%, ¹H-NMR (400 MHz; DMSO-d₆): δ 11.91 (s, 1H), 7.87-7.83 (m, 2H), 7.73-7.70 (m, 2H), 7.48-7.45 (m, 2H), 7.36-7.29 (m, 3H), 2.23 (s, 3H), 19-F NMR (376 MHz; DMSO): δ −56.7, −109.5, −112.4).

### 1-(4′-(4,6-Dichloro-7-methoxy-2-methylquinolin-3-yl)-4-(trifluoromethoxy)-[1,1′-biphenyl]-2-yl)pyrrolidin-2-one

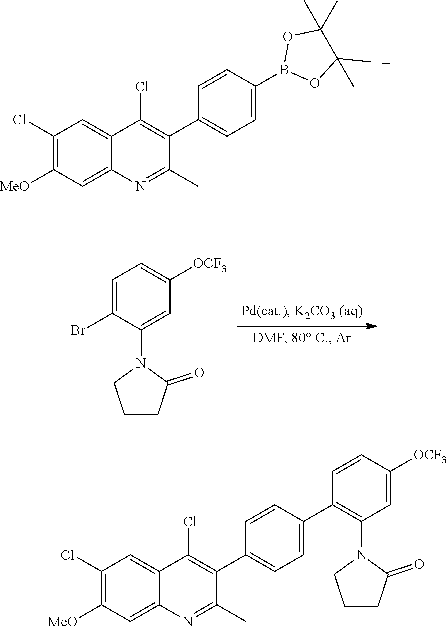

A mixture of 4,6-dichloro-7-methoxy-2-methyl-3-(4-(4,4,5,5-tetramethyl-1,3,2-dioxaborolan-2-yl)phenyl)quinoline (0.41 g, 0.00093 mol), 1-[2-bromo-5-(trifluoromethoxy)phenyl]-2-pyrrolidinone (1.08 eq., 0.0010 mol, 0.33 g), and anhydrous potassium carbonate (2.0 eq, 0.0019 mol, 0.26 g, dissolved in water, 0.93 mL) in N,N-dimethylformamide (50 mL) was stirred at room temperature for 20 minutes while degassing by bubbling argon through a glass tube under the liquid surface. [1,1′-bis(Diphenylphosphino)ferrocene]-dichloropalladium (II) (5 mol %, 0.034 g, 0.000047 mol) was added and the reaction was allowed to heat at 80° C. under argon for 19 hours. The cooled reaction mixture was vacuum filtered, followed by concentration of the filtrate under reduced pressure with heating. The resulting solid was taken up in 100 mL DCM, followed by vacuum filtration. Automated flash chromatography of the evaporated filtrate on silica, eluting with a gradient of 85:15 to 15:85 v:v hexanes:ethyl acetate, isolated the desired product (R_{f}=0.0.28, 3:7 v:v hexanes:ethyl acetate on silica) as a white, crystalline solid (0.28 g, 54%, ¹H-NMR (400 MHz; CDCl₃): δ 8.27 (s, 1H), 7.56-7.49 (m, 4H), 7.37-7.29 (m, 4H), 4.10 (s, 3H), 3.34 (t, J=6.9 Hz, 2H), 2.51 (s, 3H), 2.47 (t, J=8.1 Hz, 2H), 1.99-1.92 (m, 2H); 19-F NMR (376 MHz; CDCl3): δ −57.7).

### 6-chloro-7-methoxy-2-methyl-3-(2′-(2-oxopyrrolidin-1-yl)-4′-(trifluoromethoxy)-[1,1′-biphenyl]-4-yl)quinolin-4(1H)-one (ELQ-787)

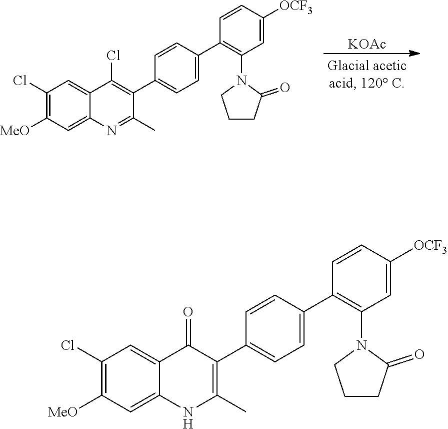 [SMILES: COc1cc2nc(C)c(-c3ccc(-c4ccc(OC(F)(F)F)cc4N4CCCC4=O)cc3)c(Cl)c2cc1Cl ; COc1cc2[nH]c(C)c(-c3ccc(-c4ccc(OC(F)(F)F)cc4N4CCCC4=O)cc3)c(=O)c2cc1Cl ; "KOAc"]

1-(4′-(4,6-Dichloro-7-methoxy-2-methylquinolin-3-yl)-4-(trifluoromethoxy)-[1,1′-biphenyl]-2-yl)pyrrolidin-2-one (0.28 g, 0.00050 mol) and anhydrous potassium acetate (10 eq, 0.0050 mol, 0.49 g) were heated in glacial acetic acid (10 mL) for 22 hours at 120° C. After cooling to room temperature, the reaction mixture was poured into water (60 mL) and vacuum filtered, rinsing with excess water followed by 3×1.5 mL acetone. The resulting white powder was the desired product (0.16 g, 56%, ¹H-NMR (400 MHz; DMSO-d₆): δ 11.69 (s, 1H), 8.01 (s, 1H), 7.60 (dd, J=8.2, 0.7 Hz, 1H), 7.47-7.43 (m, 2H), 7.36-7.32 (m, 4H), 7.08 (s, 1H), 3.97 (s, 3H), 3.35-3.33 (m, overlaps water signal, estimated 2H), 2.28 (t, J=8.0 Hz, 2H), 2.24 (s, 3H), 1.91-1.83 (m, 2H); 19-F NMR (376 MHz; DMSO): δ −56.7).

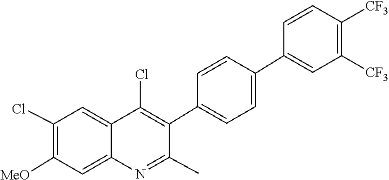 [SMILES: COc1cc2nc(C)c(-c3ccc(-c4ccc(C(F)(F)F)c(C(F)(F)F)c4)cc3)c(Cl)c2cc1Cl]

3-(3′,4′-bis(trifluoromethyl)-[1,1′-biphenyl]-4-yl)-4,6-dichloro-7-methoxy-2-methylquinoline (3t): Following the general procedure A, a mixture of 1 (794 mg, 2.0 mmol, 1 eq), 2-(3,4-bis(trifluoromethyl)phenyl)-4,4,5,5-tetramethyl-1,3,2-dioxaborolane (748 mg, 2.2 mmol, 1.1 eq), aqueous K₂CO₃ (2 ml, 2 eq), Pd(dppf)Cl₂ (73 mg, 0.1 mmol, 0.05 eq) and DMF (75 ml) was heated for 18 h to give crude 3t (1.20 g) as a black solid. DCM (20 ml) was added, the precipitate was filtered washed with methylene chloride (2×5 ml) to give pure 3t (210 mg) as a white solid, second crop from DCM gives another pure 3t (420 mg) as a white solid for a combined yield of 31 (630 mg, 59% yield). GC-MS shows one peak M⁺=529 (100%). ¹H-NMR (400 MHz; CDCl₃): δ 8.28 (s, 1H), 8.17 (s, 1H), 8.00-7.99 (m, 2H), 7.81-7.78 (m, 2H), 7.50 (s, 1H), 7.47-7.44 (m, 2H), 4.10 (s, 3H), 2.52 (s, 3H).

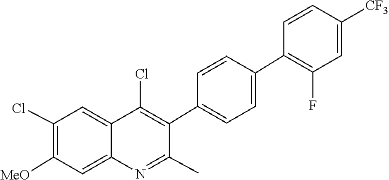 [SMILES: COc1cc2nc(C)c(-c3ccc(-c4ccc(C(F)(F)F)cc4F)cc3)c(Cl)c2cc1Cl]

4,6-dichloro-3-(2′-fluoro-4′-(trifluoromethyl)-[1,1′-biphenyl]-4-yl)-7-methoxy-2-methylquinoline (3u): Following the general procedure A, a mixture of 1 (1.59 gm, 4.0 mmol, 1 eq), (2-fluoro-4-(trifluoromethyl)phenyl)boronic acid (915 mg, 4.4 mmol, 1.1 eq), aqueous K₂CO₃ (4 ml, 2 eq), Pd(dppf)Cl₂ (146 mg, 0.2 mmol, 0.05 eq) and DMF (150 ml) was heated for 24 h to give crude 3u (2.02 gm) as a yellow solid. DCM (20 ml) was added and the precipitate was filtered washed with methylene chloride (2×5 ml) to give pure 3u (580 mg) as a white solid. The mother liquor was further purified by flash chromatography using a gradient of ethyl acetate/hexane (3/7) as the eluting solvent to yield additional 3u (802 mg). The combined product was further crystalized from DCM/ethyl acetate to 3u (1.10 gm, 57% yield) as a white solid. The product is pure enough for the next step (˜95% pure by NMR). GC-MS shows one peak M⁺=479 (100%). ¹H-NMR (400 MHz; CDCl₃): δ 8.28 (s, 1H), 7.75-7.68 (m, 3H), 7.56 (dd, J=8.1, 0.9 Hz, 1H), 7.52-7.50 (m, 2H), 7.43-7.41 (m, 2H), 4.10 (s, 3H), 2.53 (s, 3H).

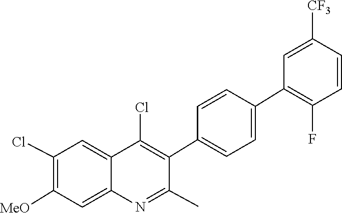 [SMILES: COc1cc2nc(C)c(-c3ccc(-c4cc(C(F)(F)F)ccc4F)cc3)c(Cl)c2cc1Cl]

4,6-dichloro-3-(2′-fluoro-5′-(trifluoromethyl)-[1,1′-biphenyl]-4-yl)-7-methoxy-2-methylquinoline (3v): Following the general procedure A, a mixture of 1 (1.59 gm, 4.0 mmol, 1 eq), (2-fluoro-5-(trifluoromethyl)phenyl)boronic acid (915 mg, 4.4 mmol, 1.1 eq), aqueous K₂CO₃ (4 ml, 2 eq), Pd(dppf)Cl₂ (146 mg, 0.2 mmol, 0.05 eq) and DMF (150 ml) was heated for 24 h to give crude 3u (1.47 gm) as a yellow solid. The product was purified by flash chromatography using a gradient of ethyl acetate/hexane (3/7) as the eluting solvent to give 3v (850 mg, 44% yield) as a white solid. The product is pure enough for the next step (˜90% pure by NMR). GC-MS shows one peak M⁺=479 (100%). ¹H-NMR (400 MHz; CDCl₃): δ 8.28 (s, 1H), 7.87-7.85 (m, 1H), 7.75-7.72 (m, 2H), 7.69-7.65 (m, 1H), 7.50 (s, 1H), 7.43-7.40 (m, 2H), 7.37-7.32 (m, 1H), 4.10 (s, 3H), 2.53 (s, 3H).

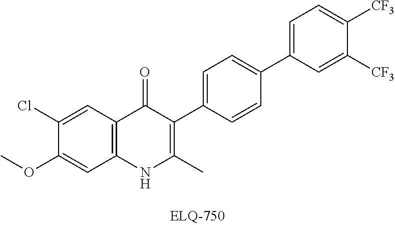 [SMILES: COc1cc2[nH]c(C)c(-c3ccc(-c4ccc(C(F)(F)F)c(C(F)(F)F)c4)cc3)c(=O)c2cc1Cl]

3-(3′,4′-bis(trifluoromethyl)-[1,1′-biphenyl]-4-yl)-6-chloro-7-methoxy-2-methylquinolin-4(1H)-one (ELQ-750): Following the general procedure B, a mixture of 31 (210 mg, 0.40 mmol, 1 eq), KOAc, (392 mg, 4.0 mmol, 10 eq), glacial acetic acid (5 ml) was heated for 24 h to give pure ELQ-750 (420 mg, 69% yield) as a white solid. ¹H-NMR (400 MHz; DMSO-d₆): δ 11.74 (s, 1H), 8.29-8.27 (m, 2H), 8.16-8.14 (m, 1H), 8.03 (s, 1H), 7.89-7.86 (m, 2H), 7.46-7.43 (m, 2H), 7.10 (s, 1H), 3.98 (s, 3H), 2.28 (s, 3H).

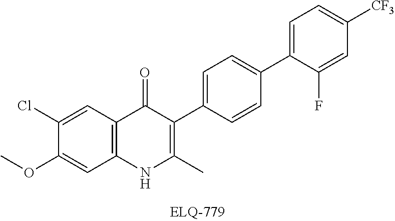 [SMILES: COc1cc2[nH]c(C)c(-c3ccc(-c4ccc(C(F)(F)F)cc4F)cc3)c(=O)c2cc1Cl]

6-chloro-3-(2′-fluoro-4′-(trifluoromethyl)-[1,1′-biphenyl]-4-yl)-7-methoxy-2-methylquinolin-4(1H)-one (ELQ-779): Following the general procedure B, a mixture of 3u (1.10 gm, 2.3 mmol, 1 eq), KOAc, (2.25 gm, 23.0 mmol, 10 eq), glacial acetic acid (20 ml) was heated for 24 h to give ELQ-750 (945 mg) as a white solid. The product was crystallized in DMF to give pure ELQ-750 (820 mg, 77% yield) as a white solid. ¹H-NMR (400 MHz; DMSO-d₆): δ 11.72 (s, 1H), 8.02 (s, 1H), 7.87-7.82 (m, 2H), 7.73-7.70 (m, 1H), 7.66-7.63 (m, 2H), 7.43-7.40 (m, 2H), 7.10 (s, 1H), 3.98 (s, 3H), 2.28 (s, 3H).

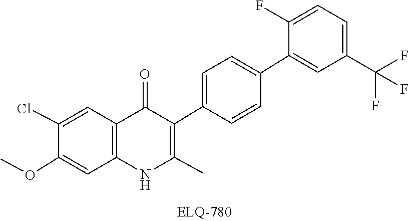 [SMILES: COc1cc2[nH]c(C)c(-c3ccc(-c4cc(C(F)(F)F)ccc4F)cc3)c(=O)c2cc1Cl]

6-chloro-3-(2′-fluoro-5′-(trifluoromethyl)-[1,1′-biphenyl]-4-yl)-7-methoxy-2-methylquinolin-4(1H)-one (ELQ-780): Following the general procedure B, a mixture of 3v (850 mg, 1.77 mmol, 1 eq), KOAc, (1.73 gm, 17.7 mmol, 10 eq), glacial acetic acid (20 ml) was heated for 24 h to give ELQ-780 (650 mg) as a white solid. The product was crystallized from DMF to give ELQ-780 (431 mg, 47% yield, ˜95% pure by NMR). ¹H-NMR (400 MHz; DMSO-d₆): δ 11.72 (s, 1H), 8.02 (s, 1H), 7.96-7.93 (m, 1H), 7.86-7.82 (m, 1H), 7.66-7.59 (m, 3H), 7.42-7.39 (m, 2H), 7.09 (s, 1H), 3.98 (s, 3H), 2.28 (s, 3H).

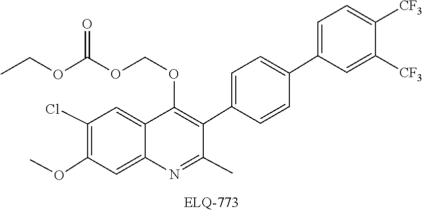 [SMILES: CCOC(=O)OCOc1c(-c2ccc(-c3ccc(C(F)(F)F)c(C(F)(F)F)c3)cc2)c(C)nc2cc(OC)c(Cl)cc12]

((3-(3′,4′-bis(trifluoromethyl)-[1,1′-biphenyl]-4-yl)-6-chloro-7-methoxy-2-methylquinolin-4-yl)oxy)methyl ethyl carbonate (ELQ-773): Following the general procedure C, using a mixture of ELQ-750 (256 mg, 0.5 mmol, 1 eq), TBAI (370 mg, 1.0 mmol, 2 eq), dry K₂CO₃ (139 mg, 1.0 mmol, 2 eq) and chloromethyl ethylcarbonate (139 mg, 1.0 mmol, 2 eq) in DMF (25 ml) to give crude ELQ-773 (370 mg). The product was purified by flash chromatography using ethyl acetate/hexane (1/1) to pure ELQ-773 (247 mg, 80% yield). GC-MS shows one peak M⁺=613 (25%), M⁺=511 (100%). ¹H-NMR (400 MHz; CDCl₃): δ 8.14 (s, 1H), 8.06 (s, 1H), 7.97 (s, 2H), 7.78-7.75 (m, 2H), 7.56-7.53 (m, 2H), 7.45 (s, 1H), 5.30 (s, 2H), 4.11 (q, J=7.1 Hz, 2H), 4.06 (s, 3H), 2.54 (s, 3H), 1.21 (t, J=7.1 Hz, 3H).

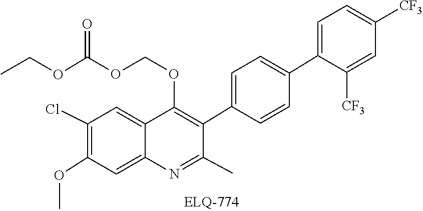 [SMILES: CCOC(=O)OCOc1c(-c2ccc(-c3ccc(C(F)(F)F)cc3C(F)(F)F)cc2)c(C)nc2cc(OC)c(Cl)cc12]

((3-(2′,4′-bis(trifluoromethyl)-[1,1′-biphenyl]-4-yl)-6-chloro-7-methoxy-2-methylquinolin-4-yl)oxy)methyl ethyl carbonate (ELQ-774): Following the general procedure C, using a mixture of ELQ-763 (256 mg, 0.5 mmol, 1 eq), TBAI (370 mg, 1.0 mmol, 2 eq), dry K₂CO₃ (139 mg, 1.0 mmol, 2 eq) and chloromethyl ethylcarbonate (139 mg, 1.0 mmol, 2 eq) in DMF (25 ml) to give crude ELQ-774 (305 mg). The product was purified by flash chromatography using ethyl acetate/hexane (1/1) to pure ELQ-774 (237 mg, 77% yield). GC-MS shows one peak M⁺=613 (25%), M⁺=511 (100%). ¹H-NMR (400 MHz; CDCl₃): δ 8.07 (s, 1H), 8.05 (s, 1H), 7.90-7.88 (m, 1H), 7.61 (d, J=8.0 Hz, 1H), 7.48-7.45 (m, 5H), 5.26 (s, 2H), 4.15 (q, J=7.1 Hz, 2H), 4.06 (s, 3H), 2.56 (s, 3H), 1.23 (t, J=7.1 Hz, 3H).

### In Vitro Metabolic Stability Assay

Murine Microsomal Stability. Metabolic stability studies of ELQ-596 was performed at ChemPartner, Shanghai, China. The drug was incubated at 37° C. and 1 μM concentration in murine liver microsomes (Corning) for 45 minutes at a protein concentration of 0.5 mg/mL in potassium phosphate buffer at pH 7.4 containing 1.0 mM EDTA. The metabolic reaction was initiated by addition of NADPH and quenched with ice-cold acetonitrile at 0, 5, 15, 25, and 45 minutes. The progress of compound metabolism was followed by LC-MS/MS (ESI positive ion, LC-MS/MS-034(API-6500+)) using a C₁₈ stationary phase (ACQUITY UPLC BEH C₁₈ (2.1 Å˜50 mm, 1.7 μm)) and a MeOH/water mobile phase containing 0.25% FA and 1 mM NH4OAc. Imipramine or Osalmid were used as internal standards, and ketanserin was used as a control drug with intermediate stability. Concentration versus time data for each compound were fitted to an exponential decay function to determine the first-order rate constant for substrate depletion, which was then used to calculate the degradation half-life (t_{1/2}) and predicted intrinsic clearance value (CIᵢₙₜ) from an assumed murine hepatic blood flow of 90 mL/min/kg.

Plasmodium falciparum Culture. Laboratory strains of P. falciparum were cultured in human erythrocytes by standard methods. The parasites were grown in culture medium with fresh human erythrocytes maintained at 2% hematocrit at 37° C. in low-oxygen conditions (5% O₂, 5% CO₂, 90% and balance N₂). The culture medium used was RPMI-1640 with 25 mg/L gentamicin sulfate, 45 mg/L Albumax II, 10 mM glucose, and 25 mM HEPES buffer. Cultures were maintained at less than 10% parasitemia by transfer of infected cells to fresh erythrocytes and culture medium every 3 or 4 days. The P. falciparum strains used in these experiments include the following: D6 (MRA-285/BEI Resources, deposited by Dr. Dennis Kyle) with modest resistance to mefloquine but generally drug sensitive; Dd2 (MRA-150/BEI Resources, deposited by Dr. David Walliker) with resistance to chloroquine, mefloquine and pyrimethamine; D1 is a subclone of Dd2 that was selected for resistance to ELQ-300; and Tm90-C2B was isolated from a patient enrolled in an atovaquone clinical trial in Thailand upon recrudescence after cessation of drug treatment (obtained from Drs. Dennis Kyle and Victor Melendez, WRAIR).

In vitro drug susceptibility assays. SYBR green I assay. In vitro antiplasmodial activity was assessed using a published SYBR Green I fluorescence-based method. The drugs were added to 96-well plates using 2-fold serial dilutions in complete medium. The initial range was from 2.5 μM to 2 nM. Asynchronous P. falciparum parasites were diluted with uninfected erythrocytes and added to the wells to give a final culture volume of 100 μl at 2% hematocrit and 0.2% parasitemia. The plates were incubated for 72 h at 37° C. The parasites were then lysed by adding 100 μl of SYBR green I lysis buffer containing 0.2 μl/ml SYBR green I dye (10,000×) in 20 mM Tris (pH 7.5), 5 mM EDTA, 0.008% (wt/vol) saponin, and 0.08% (vol/vol) Triton X-100. The plates were incubated at room temperature for an hour in the dark. The fluorescence signal, correlating to parasite DNA, was measured using a SpectraMax iD3 iD5 Multi-Mode Microplate Reader, with excitation and emission wavelength bands centered at 497 and 520 nm, respectively. The 50% inhibitory concentrations (IC₅₀) were determined by non-linear regression analysis using GraphPad Prism software. Drugs were assayed in quadruplicate and the results were averaged during analysis to give a final IC₅₀ value together with standard deviations and 95% confidence intervals. Atovaquone and ELQ-300 were used as internal controls to verify cross-resistance and parasite strain integrity. If the IC₅₀ value fell outside of the initial tested range then the range was adjusted up or down and the assay was repeated.

In Vivo Efficacy against Murine Malaria. The P. yoelii 4-day test monitors suppression of patent infection in female CF1 mice. The test began with the inoculation (iv) of parasitized erythrocytes (3.5×10⁴/P. yoelli) (from a donor animal) on the first day of the experiment (D0). After 24 hr, test drugs (including ELQ-596 and prodrug ELQ-598) were administered daily by gavage for 4 successive days. Initially the 3-biaryl-ELQs were tested at doses of 0.0025, 0.005, 0.01, 0.03, 0.1, 0.3, 1.0 and 10 mg/kg/day, including a vehicle-only (PEG400) control. After completion of drug treatment, a blood sample was collected (by pricking the tail vein) for determination of parasite burden beginning on the day after the final dose (D5). Percent parasitemia is assessed by direct microscopic analysis of Giemsa-stained blood smears. Drug activity was recorded as % suppression of parasite burden relative to drug-free controls. Animals with observable parasitemia following the experiment were euthanized; animals cleared of parasites from the bloodstream were observed daily with assessment of parasitemia performed weekly until day 30, at which point the animal(s) were scored as cured of infection. Typically, the percentage parasitemia in untreated control animals on Day 5 of the “4-day test” is between 20 and 25%. Non-linear regression analysis is used for objective determination of ED₅₀'s and ED₉₀'s from the accumulated data as well as the Non-Recrudescence Dose (NRD). The 4-day test protocol was reviewed and approved by the local IACUC board at the Portland VA Medical Center. Experiments were performed with 4 mice per group to ensure statistical accuracy. Control drugs for these experiments included ELQ-300 and prodrug ELQ-331.

In Vivo Single-Dose Efficacy against Murine Malaria. The effectiveness of selected 3-biarylELQs and prodrugs was assessed vs. the blood stage infection for single dose cures. Mice are infected iv with 3.5×10⁴ P. yoelii infected RBCs as described for the 4-day test above. Drug administration occurred on the day after inoculation (Day 1). Test agents were dissolved in PEG-400 and administered ig once. On the 5th day blood films were prepared and % parasitemia was assessed. Animals remaining parasite-free for 30 days after drug administration were considered cured. The initial dosing range was: 0.5, 1, 2.5, 5, 10 mg/kg, including a control. Experiments were performed with 4 mice per group to ensure statistical accuracy. The reported parameter for these studies is the lowest single dose that provides a cure to all 4 animals in the group. ELQ-331 served as a positive control in these studies to directly compare with prodrug ELQ-598.

In Vivo Prophylaxis against Murine Malaria—whole animal bioluminescence. ELQ-598 was evaluated for liver stage activity in vivo at the Portland VA with a Perkin-Elmer IVIS instrument. This well-characterized assay uses in vivo imaging to demonstrate liver stage activity in a murine model. In brief, luciferase/GFP expressing P. yoelii sporozoites were reared Anopheles stephensii at the OHSU insectary (Dr. Brandon Wilder). Mice were inoculated with 10,000 sporozoites via tail vein injection of CF1 mice treated with or without drug (dissolved in PEG400) one hour after inoculation. In vivo imaging assessments were taken at 24-, 48-, and 72-hours post-injection and the luciferase signal from drug treated mice was compared to the luciferase signal derived from vehicle treated mice. Imaging of any luciferase expressing liver stage parasites followed the administration of 150 mg/kg luciferin i.p. (150 μl volume) via a 25-gauge needle and syringe prior to each time point. At 3 to 5 minutes' post luciferin administration the mice were anesthetized with isoflurane gas when imaging began. Additional monitoring of blood stage infection was conducted after IVIS assessment for a 30-day period to confirm true causal prophylaxis against P. yoelii challenge. Outcomes from this assay included full causal prophylaxis where all animals showed a negative liver stage signal, partial causal prophylaxis where less than 100% of the animals exhibited a negative liver signal, suppressive prophylaxis where a positive liver stage signal was observed followed by a negative blood stage signal, and a delay in patency where blood stage parasitemia was delayed in drug-treated animals compared to vehicle animals. Testing involved the use of 4 animals per group for statistical accuracy. ELQ-331 was used as a positive control.

Isolation of Plasmodium falciparum Mitochondria and Ubiquinol-Cytochrome c Oxidoreductase Assay.

### Human Cytochrome bc₁ Assays.

### Mitochondrial Toxicity of ELQ-300 and ELQ-331.

A sub-series of biphenyl ELQ compounds, having a 7-methoxy-6-hydro substitution pattern on the quinolone ring system, have a positive attribute that distinguishes them from prior ELQs even within the 3-biaryl-ELQ series. This sub-series is exemplified by ELQ-685. While ELQ-596 exhibits cross resistance in the ELQ-300 resistant P. falciparum clone D1 (which we infer to indicate targeting of the Q site of cytochrome bc₁ complex, see Table A), and atovaquone exhibits cross resistance in the clinical isolate Tm90-C2B of P. falciparum which contains a mutation in the distant Qo site of cytochrome bc₁, ELQ-685 and its analogs exhibit low nanomolar IC₅₀ values vs. drug sensitive and ELQ-300® and Atovaquone® P. falciparum strains. That ELQ-685 is equipotent vs. multidrug resistant strains of P. falciparum (e.g., Dd2) as well as strains harboring resistance to ELQ-300 (D1) and Atovaquone (Tm90-C2B) suggests that it may be targeting both Qₒ and Qᵢ sites in a docking orientation that is unique and not affected by mutated residues in either site. An advantage to such dual site targeting agents is that resistance is likely to be very difficult to achieve, given that a parasite would have to evolve with simultaneous mutations in both the Qₒ and Qᵢ sites.

Structure activity profile of selected 3-biaryl-ELQs vs. drug sensitive (1D6) and drug resistant (Dd2, C2B3, and D1) strains of Plasmodium falciparum.

| | | | Melting | P. falciparum IC₅₀ values, nM | | | | |
|---|---|---|---|---|---|---|---|---|
| | | | point, | | | Tm90- | | Cytotoxicity, |
| Code | MW | cLogP | ° C. | D6 | Dd2 | C2B | D1 | HepG2, nM |
| Atovaquone | 367 | 6.1 | 216-219 | 0.08 | 0.4 | >1,000 | 0.4 | NT |
| | | | | (0.07 to | (0.3 to | | (0.4 to | |
| | | | | 0.09) | 0.4) | | 0.5) | |
| ELQ-300 | 476 | 5.2 | 317 | 3.3 | 4.2 | 2.5 | 168 | >10,000 |
| | | | | (3.0 to | (3.6 to | (2.2 to | | |
| | | | | 3.5) | 4.9) | 2.8) | | |
| ELQ-596 | 459 | 5.0 | 365 | 0.3 | 0.4 | 0.2 | 449 | >10,000 |
| | | | | (0.3 to | (0.3 to | (0.2 to | (346 to | |
| | | | | 0.4) | 0.4) | 0.2) | 659) | |
| ELQ-598 | 562 | 6.9 | 136 | 1.5 | 4.4 | 0.8 | 734 | >10,000 |
| (prodrug) | | | | 1.3 to | 4.1 to | 0.7 to | 667 to | |
| | | | | 1.7 | 4.8 | 0.9 | 817 | |
| ELQ-685 | 425 | 4.5 | 391 | 5.8 | 8.5 | 5.2 | 3.5 | NT |
| | | | | (5.2 to | (7.2 to | (4.5 to | (3.1 to | |
| | | | | 6.4) | 10.0) | 6.1) | 4.0) | |
| ELQ-695 | 528 | 6.4 | 112 | 20.9 | 22.3 | 10.1 | 8.4 | NT |
| (prodrug) | | | | (19.3 to | (19.7 to | (8.7 to | (7.8 to | |
| | | | | 22.7) | 25.6) | 11.9) | 9.2) | |
| ELQ-730 | 406 | 4.6 | NT | 5.02 | 5.33 | 4.05 | 3.82 | NT |
| | | | | (4.73 to | (4.73 to | (3.55 to | (3.47 to | |
| | | | | 5.32) | 6.04) | 4.63) | 4.20) | |
| ELQ-731 | 477 | 5.3 | NT | 3.93 | 4.73 | 2.05 | 5.56 | NT |
| | | | | (3.09 to | (3.65 to | (1.58 to | (3.85 to | |
| | | | | 5.06) | 6.23) | 2.65) | 8.31) | |
| ELQ-732 | 732 | 4.5 | NT | 15.92 | 16.28 | 14.43 | 10.63 | NT |
| | | | | (14.9 to | (14.6 to | (12.6 to | (9.74 to | |
| | | | | 17.0) | 18.3) | 16.7) | 11.6) | |
| ELQ-733 | 461 | 4.4 | NT | 7.45 | 7.90 | 2.94 | 6.18 | NT |
| | | | | (5.59 to | (6.15 to | (2.44 to | (5.07 to | |
| | | | | 9.93) | 10.3) | 3.56) | 7.60) | |
| ELQ-734 | 407 | 3.3 | NT | 10.91 | 16.26 | 6.40 | 11.56 | NT |
| | | | | (8.42 to | (12.6 to | (5.26 to | (8.05 to | |
| | | | | 14.1) | 21.9) | 7.87) | 17.1) | |
| ELQ-756 | 389 | 3.0 | NT | 3.27 | 4.46 | 3.22 | 2.06 | NT |
| | | | | (3.03 to | (3.76 to | (2.82 to | (1.60 to | |
| | | | | 3.53) | 5.29) | 3.69) | 2.65) | |
| ELQ-757 | 439 | 3.7 | NT | 3.08 | 4.85 | 3.00 | 1.91 | NT |
| | | | | (2.66 to | (3.56 to | (2.33 to | (1.39 to | |
| | | | | 3.57) | 6.59) | 3.88) | 2.59) | |

In viva efficacy. Another positive attribute of ELQ-685 is that in vitro metabolism studies in the presence of pooled murine hepatic microsomes shows complete metabolic stability over the course of a 45-minute incubation (t_{1/2}=>4,000 minutes/performed by Chempartner). Given that ELQ-685 exhibits impressive low nM IC₅₀ values vs. P. falciparum strains in vitro and excellent in vitro metabolic stability we tested it in vivo in a murine malaria model for efficacy using a standard 4-day protocol with P. yoelii inoculation via tail vein injection (Day 0). Animals (4/group) were then dosed with ELQ-695 (in PEG-400) by gavage on Days 1, 2, 3 and 4. On Day 5, smears were prepared from tail blood, stained and examined microscopically. A non-recrudescence dose (NRD) of 3.7 mg/kg/day was determined for ELQ-695.

Efficacy of ELQ-685 prodrug, ELQ-695, in vivo in a mouse model of malaria infection.

| | | Microsomal | | | | |
|---|---|---|---|---|---|---|
| | | stability | Blood stage In vivo efficacy, mg/kg/day | | | |
| | Melting | (murine | | | | Single |
| | Point, | microsomes) | 4-day Peters Test Results | | | dose |
| Code | ° C. | T_{1/2}, minutes | ED₅₀ | ED₉₀ | NRD | cure |
| ELQ- | 110-112 | >4,000 | 0.2 | 0.3 | 3.7 | UND |
| 695 | | | | | | |

MP=melting point; ED₅₀—dose required to suppress parasitemia by 50% relative to untreated controls (4-day Peters test), ED₉₀—dose required to suppress parasitemia by 90% relative to untreated controls (4-day Peters test, P. yoelii Kenya Strain), NRD—non-recrudescence dose (4-day Peters test), and SDC—single dose cure (lowest single dose that provides complete cures of all 4 mice in the group). UND=Experiments are currently underway. Note: Prodrugs were dosed based on molar equivalency to the parent drug.

### REFERENCES

1. World malaria report 2021; World Health Organization: Geneva, 2021.
2. Dondorp, A. M.; Nosten, F.; Yi, P.; Das, D.; Phyo, A. P.; Taming, J.; Lwin, K. M.; Ariey, F.; Hanpithakpong, W.; Lee, S. J.; Ringwald, P.; Silamut, K.; Imwong, M.; Chotivanich, K.; Lim, P.; Herdman, T.; An, S. S.; Yeung, S.; Singhasivanon, P.; Day, N. P.; Lindegardh, N.; Socheat, D.; White, N. J., Artemisinin resistance in Plasmodium falciparum malaria. N Engl J Med 2009, 361 (5), 455-67.
3. Balikagala, B.; Fukuda, N.; Ikeda, M.; Katuro, O. T.; Tachibana, S. I.; Yamauchi, M.; Opio, W.; Emoto, S.; Anywar, D. A.; Kimura, E.; Palacpac, N. M. Q.; Odongo-Aginya, E. I.; Ogwang, M.; Horii, T.; Mita, T., Evidence of Artemisinin-Resistant Malaria in Africa. N Engl J Med 2021, 385 (13), 1163-1171.
4. Burrows, J. N.; Duparc, S.; Gutteridge, W. E.; Hooft van Huijsduijnen, R.; Kaszubska, W.; Macintyre, F.; Mazzuri, S.; Mohrle, J. J.; Wells, T. N. C., New developments in anti-malarial target candidate and product profiles. Malar J 2017, 16 (1), 26.
5. Macintyre, F.; Ramachandruni, H.; Burrows, J. N.; Holm, R.; Thomas, A.; Mohrle, J. J.; Duparc, S.; Hooft van Huijsduijnen, R.; Greenwood, B.; Gutteridge, W. E.; Wells, T. N. C.; Kaszubska, W., Injectable anti-malarials revisited: discovery and development of new agents to protect against malaria. Malar J 2018, 17(1), 402.
6. Nilsen, A.; LaCrue, A. N.; White, K. L.; Forquer, I. P.; Cross, R. M.; Marfurt, J.; Mather, M. W.; Delves, M. J.; Shackleford, D. M.; Saenz, F. E.; Morrisey, J. M.; Steuten, J.; Mutka, T.; Li, Y.; Wirjanata, G.; Ryan, E.; Duffy, S.; Kelly, J. X.; Sebayang, B. F.; Zeeman, A. M.; Noviyanti, R.; Sinden, R. E.; Kocken, C. H. M.; Price, R. N.; Avery, V. M.; Angulo-Barturen, I.; Jimenez-Diaz, M. B.; Ferrer, S.; Herreros, E.; Sanz, L. M.; Gamo, F. J.; Bathurst, I.; Burrows, J. N.; Siegl, P.; Guy, R. K.; Winter, R. W.; Vaidya, A. B.; Charman, S. A.; Kyle, D. E.; Manetsch, R.; Riscoe, M. K., Quinolone-3-diarylethers: a new class of antimalarial drug. Sci Transl Med 2013, 5 (177), 177ra37.
7. van Schalkwyk, D. A.; Riscoe, M. K.; Pou, S.; Winter, R. W.; Nilsen, A.; Duffey, M.; Moon, R. W.; Sutherland, C. J., Novel Endochin-Like Quinolones Exhibit Potent In Vitro Activity against Plasmodium knowlesi but Do Not Synergize with Proguanil. Antimicrob Agents Chemother 2020, 64(5).
8. Stickles, A. M.; de Almeida, M. J.; Morrisey, J. M.; Sheridan, K. A.; Forquer, I. P.; Nilsen, A.; Winter, R. W.; Burrows, J. N.; Fidock, D. A.; Vaidya, A. B.; Riscoe, M. K., Subtle changes in endochin-like quinolone structure alter the site of inhibition within the cytochrome bc1 complex of Plasmodium falciparum. Antimicrob Agents Chemother 2015, 59 (4), 1977-82.
9. Frueh, L.; Li, Y.; Mather, M. W.; Li, Q.; Pou, S.; Nilsen, A.; Winter, R. W.; Forquer, I. P.; Pershing, A. M.; Xie, L. H.; Smilkstein, M. J.; Caridha, D.; Koop, D. R.; Campbell, R. F.; Sciotti, R. J.; Kreishman-Deitrick, M.; Kelly, J. X.; Vesely, B.; Vaidya, A. B.; Riscoe, M. K., Alkoxycarbonate Ester Prodrugs of Preclinical Drug Candidate ELQ-300 for Prophylaxis and Treatment of Malaria. ACS Infect Dis 2017, 3 (10), 728-735.
10. Pou, S.; Dodean, R. A.; Frueh, L.; Liebman, K. M.; Gallagher, R. T.; Jin, H.; Jacobs, R. T.; Nilsen, A.; Stuart, D. R.; Doggett, J. S.; Riscoe, M. K.; Winter, R. W., A New Scalable Synthesis of ELQ-300, ELQ-316, and other Antiparasitic Quinolones. Org Process Res Dev 2021, 25 (8), 1841-1852.
11. DiMasi, J. A.; Grabowski, H. G.; Hansen, R. W., Innovation in the pharmaceutical industry: New estimates of R&D costs. J Health Econ 2016, 47, 20-33.
12. Nilsen, A.; Miley, G. P.; Forquer, I. P.; Mather, M. W.; Katneni, K.; Li, Y.; Pou, S.; Pershing, A. M.; Stickles, A. M.; Ryan, E.; Kelly, J. X.; Doggett, J. S.; White, K. L.; Hinrichs, D. J.; Winter, R. W.; Charman, S. A.; Zakharov, L. N.; Bathurst, I.; Burrows, J. N.; Vaidya, A. B.; Riscoe, M. K., Discovery, synthesis, and optimization of antimalarial 4(1H)-quinolone-3-diarylethers. J Med Chem 2014, 57(9), 3818-34.
13. Heindel, N. D.; Fine, S. A., Alcoholysis of 4-chloroquinolines to 4(1H)-quinolones. The Journal of Organic Chemistry 1970, 35 (3), 796-798.
14. Leung, S. C.; Gibbons, P.; Amewu, R.; Nixon, G. L.; Pidathala, C.; Hong, W. D.; Pacorel, B.; Berry, N. G.; Sharma, R.; Stocks, P. A.; Srivastava, A.; Shone, A. E.; Charoensutthivarakul, S.; Taylor, L.; Berger, O.; Mbekeani, A.; Hill, A.; Fisher, N. E.; Warman, A. J.; Biagini, G. A.; Ward, S. A.; O'Neill, P. M., Identification, design and biological evaluation of heterocyclic quinolones targeting Plasmodium falciparum type II NADH:quinone oxidoreductase (PfNDH2). J Med Chem 2012, 55(5), 1844-57.
15. Conrad, M.; Limpach, L., synthesen von Chinolinderivaten mittelst Acetessigester. Berichte der deutschen chemischen Gesellschaft 1887, 20 (1), 944-948.
16. Conrad, M.; Limpach, L., Synthese von Chinolinderivaten mittelst Acetessigester. Berichte der deutschen chemischen Gesellschaft 1891, 24 (2), 2990-2992.
17. Elderfield, R. C., Heterocyclic Compounds. John Wiley & Sons, Inc.: New York, NY, 1961.
18. Bennett, T. N.; Paguio, M.; Gligorijevic, B.; Seudieu, C.; Kosar, A. D.; Davidson, E.; Roepe, P. D., Novel, rapid, and inexpensive cell-based quantification of antimalarial drug efficacy. Antimicrob Agents Chemother 2004, 48 (5), 1807-10.
19. Smilkstein, M.; Sriwilaijaroen, N.; Kelly, J. X.; Wilairat, P.; Riscoe, M., Simple and inexpensive fluorescence-based technique for high-throughput antimalarial drug screening. Antimicrob Agents Chemother 2004, 48 (5), 1803-6.
20. McConnell, E. V.; Bruzual, I.; Pou, S.; Winter, R.; Dodean, R. A.; Smilkstein, M. J.; Krollenbrock, A.; Nilsen, A.; Zakharov, L. N.; Riscoe, M. K.; Doggett, J. S., Targeted Structure-Activity Analysis of Endochin-like Quinolones Reveals Potent Qi and Qo Site Inhibitors of Toxoplasma gondii and Plasmodium falciparum Cytochrome bc1 and Identifies ELQ-400 as a Remarkably Effective Compound against Acute Experimental Toxoplasmosis. ACS Infect Dis 2018, 4 (11), 1574-1584.
21. Cross, R. M.; Flanigan, D. L.; Monastyrskyi, A.; LaCrue, A. N.; Saenz, F. E.; Maignan, J. R.; Mutka, T. S.; White, K. L.; Shackleford, D. M.; Bathurst, I.; Fronczek, F. R.; Wojtas, L.; Guida, W. C.; Charman, S. A.; Burrows, J. N.; Kyle, D. E.; Manetsch, R., Orally bioavailable 6-chloro-7-methoxy-4(1H)-quinolones efficacious against multiple stages of Plasmodium. J Med Chem 2014, 57(21), 8860-79.

1. A compound of Formula (I): 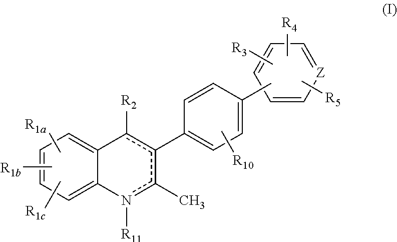
wherein:
each of R₁ₐ, R_{1b}, and R_{1c} is independently selected from the group of H, halogen, CN, C₁-C₆ alkyl, and C₁-C₆ alkoxy;
R₂ is selected from the group of:
a) oxo (═O);
b) —OH;
c) —O—CH₂—O—C(═O)—O—R₆;
d) —O—CH₂—CH₂—O—C(═O)—O—R₆;
e) —O—CH₂(CH₃)—O—C(═O)—O—R₆;
f) —O—C(═O)—CH₂—CH₂—C(═O)—O—R₆;
g) —O—CH₂—O—C(═O)—R₆;
h) —O—(C═O)—R₇;
i) —O—(C═O)—O—R₇;
j) —O—C(O)—NR₈R₉;
k) —O—CH₂—O—C(O)—O—(CH₂)ₙ₁—NR₈R₉;
l) —O—CH₂—O—C(O)—O—(CH₂)ₙ₁—NR₈—C(═O)—O—R₉; and
m) —O—(CH₂)—O—PO₃;
the dashed lines ( [symbol] ) in each instance represent an optional single or double bond;
Z is selected from the group of N and C; and
R₃, R₄, and R₅ are each independently selected from the group of H, halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, 2-pyrrolidinone, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₆ is selected from the group of C₁-C₁₀ alkyl, C₂-C₁₀ alkenyl, C₁-C₁₀ alkynyl, C₃-C₆ cycloalkyl, —(CH₂)ₙ₁—C₃-C₆ cycloalkyl, 3-6-membered heterocyclyl, —(CH₂)ₙ₁-3-6-membered heterocyclyl, phenyl, —(CH₂)ₙ₁-phenyl, and —(CH₂)ₙ₁—NR₈R₉;
R₇ is selected from the group of C₁-C₁₀ alkyl, C₁-C₁₀ alkenyl, C₁-C₁₀ alkynyl, C₃-C₆ cycloalkyl, —(CH₂)ₙ₃—(C₃-C₆ cycloalkyl), —(CH₂)ₙ₃-(3-6 membered heterocyclyl), phenyl, —(CH₂)ₙ₁-phenyl, —(CH₂)ₙ₁—O—(CH₂)ₙ₂—C₁-C₂ alkyl, —(CH₂—CH₂—O)ₙ₁—C₁-C₂ alkyl, and —(CH₂)ₙ₁—NR₈R₉;
R₈ and R₉ are each independently selected from the group of H and C₁-C₆ alkyl;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl;
R₁₁ is selected from the group of H, OH, and O⁻;
n1 and n2 are each integers independently selected from the group of 1, 2, 3, 4, 5, and 6; and
n3 is an integer selected from the group of 0, 1, 2, 3, 4, 5, and 6;
with the proviso that the compound is not a compound selected from the group of 3-[1,1′-Biphenyl]-4-yl-2-methyl-4(1H)-quinolinone (CAS Reg. No. 1354745-30-4), 3-[1,1′-Biphenyl]-4-yl-7-methoxy-2-methyl-4(1H)-quinolinone (CAS Reg. No. 1354745-39-3), 3-[1,1′-Biphenyl]-4-yl-6-chloro-2-methyl-4(1H-quinolinone (CAS Reg. No. 1354745-40-6), 3-[1,1′-Biphenyl]-4-yl-6-fluoro-2-methyl-4(1H)-quinolinone (CAS Reg. No. 1354745-28-0), 3-[1,1′-Biphenyl]-4-yl-5,7-difluoro-2-methyl-4(1H)-quinolinone (CAS Reg. No. 2251119-93-2), 3-[1,1′-Biphenyl]-4-yl-6-fluoro-7-methoxy-2-methyl-4(1H)-quinolinone (CAS Reg. No. 1354745-27-9), 3-[1,1′-Biphenyl]-4-yl-6-chloro-7-methoxy-2-methyl-4(1H)-quinolinone (CAS Reg. No. 1636139-73-5), and 6-fluoro-7-methoxy-2-methyl-3-(3″-(trifluoromethyl)-[1,1′:4′,1″-terphenyl]-4-yl)quinolin-4(1H)-one (CAS Reg. No. 1374758-04-9);
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

2. The compound of claim 1, wherein R₂ is selected from the group of:
a) oxo (═O);
b) —OH;
c) —O—CH₂—O—C(═O)—O—R₆;
d) —O—CH₂—CH₂—O—C(═O)—O—R₆; and
e) —O—CH₂(CH₃)—O—C(═O)—O—R₆;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

3. The compound of claim 1, wherein:
R₂ is selected from the group of oxo (═O) and a moiety of the formula —O—CH₂—O—C(═O)—O—(C₁-C₆ alkyl); and
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₆, R₇, R₈, R₉, R₁₀, and R₁₁ are as defined for Formula (I), above; and
with the proviso that, when R₂ is selected from the group of oxo (═O), then at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

4. The compound of claim 4, wherein R₂ is selected from the group of oxo (═O) and a moiety of the formula —O—CH₂—O—C(═O)—O—(C₁-C₃ alkyl); or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

5. The compound of claim 1 of Formula (II): 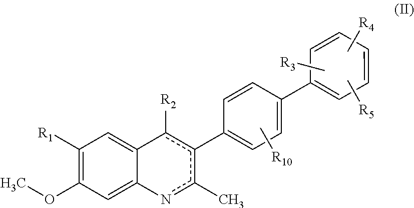
wherein:
R₁ is selected from the group of H, F, and Cl;
R₂ is selected from the group of oxo (═O) and a moiety of the formula —O—CH₂—O—C(═O)—O—(C₁-C₁₀ alkyl);
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆cycloalkyl, and —S—C₃-C₆cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₂ is selected from the group of oxo (═O), then at least one of R₃, R₄, and R₅ is not H;
or a pharmaceutically acceptable salt thereof.
A further embodiment provides a compound of Formula (II), wherein:
R₁ is selected from the group of H, F, and Cl;
R₂ is selected from the group of oxo (═O) and a moiety of the formula —O—CH₂—O—C(═O)—O—(C₁-C₆ alkyl);
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₁-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
the dashed lines ( [symbol] ) in each instance represent an optional single or double bond;
R₁₀ is selected from the group of H, C₁-C₆ alkyl, —O—C₁-C₆ alkyl, C₁-C₄ haloalkyl, —O—C₁-C₄ haloalkyl, and —S—C₁-C₄ haloalkyl;
with the proviso that, when R₂ is selected from the group of oxo (═O), then at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

6. The compound of claim 5, wherein:
R₁ is selected from the group of H, F, and Cl;
R₂ is selected from the group of oxo (═O) and a moiety of the formula —O—CH₂—O—C(═O)—O—(C₁-C₄ alkyl); and
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₂ is selected from the group of oxo (═O), then at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

7. The compound of claim 5, wherein:
R₁ is selected from the group of H, F, and Cl;
R₂ is selected from the group of oxo (═O) and a moiety of the formula —O—CH₂—O—C(═O)—O—(C₁-C₃ alkyl); and
R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₂ is selected from the group of oxo (═O), then at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

8. The compound of claim 1 of Formula (III): 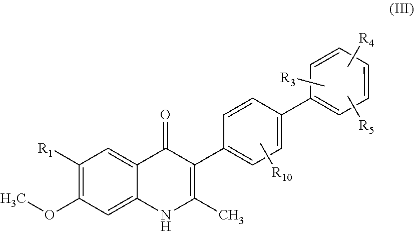
wherein:
R₁ is selected from the group of H, F, and Cl;
R₃, R₄, and R₅ are each independently selected from the group of H, halogen, C₁-C₄ alkyl, —O—C₁-C₄ alkyl, C₁-C₄ haloalkyl, —O—C₁-C₄ haloalkyl, —S—CF₃, —SF₅, CN, 2-pyrrolidinone, —C(O)NH₂, —C(O)NH(C₁-C₄ alkyl), —C(O)N(C₁-C₄ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

9. The compound of claim 8, wherein:
R₁ is selected from the group of H, F, and Cl;
R₃, R₄, and R₅ are each independently selected from the group of H, halogen, C₁-C₃ alkyl, —O—C₁-C₃ alkyl, C₁-C₃ haloalkyl, —O—C₁-C₃ haloalkyl, —S—CF₃, —SF₅, CN, 2-pyrrolidinone, —C(O)NH₂, —C(O)NH(C₁-C₃ alkyl), —C(O)N(C₁-C₃ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂; and
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

10. The compound of claim 8, wherein:
R₁ is selected from the group of H, F, and Cl; and
R₃, R₄, and R₅ are each independently selected from the group of H, halogen, C₁-C₂ alkyl, —O—C₁-C₂ alkyl, C₁-C₂ haloalkyl, —O—C₁-C₂ haloalkyl, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

11. The compound of claim 8, wherein:
R₁ is selected from the group of H, F, and Cl; and
R₃, R₄, and R₅ are each independently selected from the group of H, halogen, methyl, methoxy, CH₂F, CHF₂, CF₃, —O—CH₂F, —O—CHF₂, —O—CF₃, —S—CF₃, —SF₅, CN, 2-pyrrolidinone, —C(O)NH₂, —C(O)NH(C₁-C₂ alkyl), —C(O)N(C₁-C₂ alkyl)₂, —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl, wherein the cycloalkyl rings of the —C(O)NH(C₃-C₆ cycloalkyl), —C(O)NH(—CH₂—C₃-C₆ cycloalkyl), C₃-C₆ cycloalkyl, —O—C₃-C₆ cycloalkyl, and —S—C₃-C₆ cycloalkyl groups are further substituted by 0, 1, 2, or 3 substituents selected from the group of H, OH, oxo (═O), halogen, C₁-C₄ alkyl, —O—C₁-C₄ alkyl, —S—C₁-C₄ alkyl, —SO₂—C₁-C₄ alkyl, C₁-C₄ haloalkyl, —O—C₁-C₄ haloalkyl, —S—C₁-C₄ haloalkyl, SF₃, —SF₅, CN, NO₂, —SO₂—NH₂, —SO₂—NH(C₁-C₄ alkyl), —SO₂—N(C₁-C₄ alkyl)₂, —C(O)NH₂, —C(O)NH(C₁-C₄ alkyl), and —C(O)N(C₁-C₄ alkyl)₂;
R₁₀ is selected from the group of H, halogen, C₁-C₆ alkyl, —O—C₁-C₆ alkyl, C₁-C₄ haloalkyl, —O—C₁-C₄ haloalkyl, and —S—C₁-C₄ haloalkyl; and
the dashed lines ( [symbol] ) in each instance represent an optional single or double bond;
with the proviso that, when R₁₀ is H, at least one of R₃, R₄, and R₅ is not H;
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

12. The compound of claim 1, which is selected from the group of: 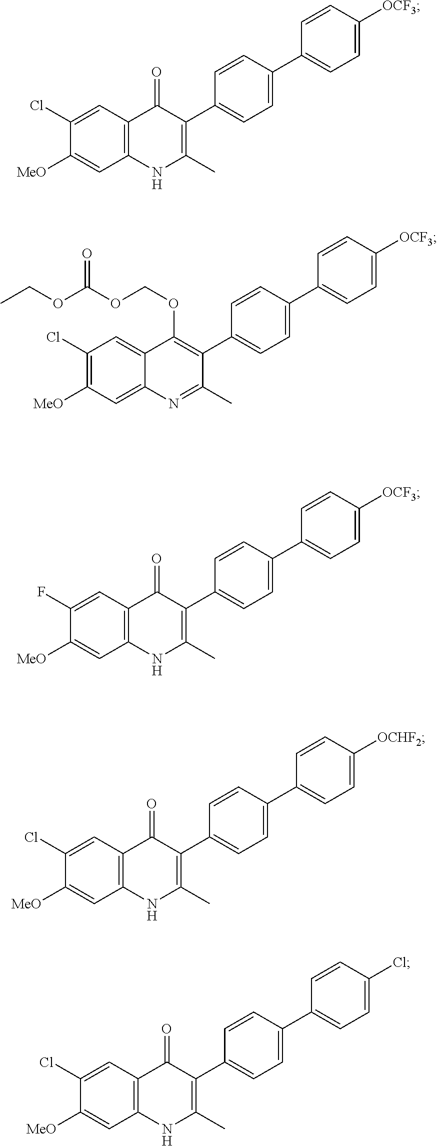 [SMILES: COc1cc2[nH]c(C)c(-c3ccc(-c4ccc(OC(F)(F)F)cc4)cc3)c(=O)c2cc1Cl ; CCOC(=O)OCOc1c(-c2ccc(-c3ccc(OC(F)(F)F)cc3)cc2)c(C)nc2cc(OC)c(Cl)cc12 ; COc1cc2[nH]c(C)c(-c3ccc(-c4ccc(OC(F)(F)F)cc4)cc3)c(=O)c2cc1F ; COc1cc2[nH]c(C)c(-c3ccc(-c4ccc(OC(F)F)cc4)cc3)c(=O)c2cc1Cl ; COc1cc2[nH]c(C)c(-c3ccc(-c4ccc(Cl)cc4)cc3)c(=O)c2cc1Cl] 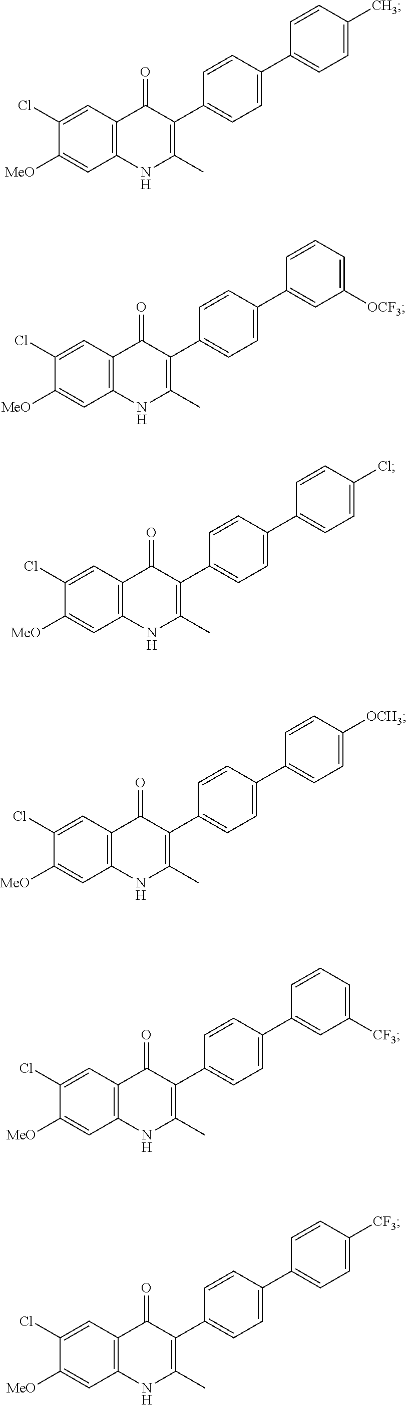 [SMILES: COc1cc2[nH]c(C)c(-c3ccc(-c4ccc(C)cc4)cc3)c(=O)c2cc1Cl ; COc1cc2[nH]c(C)c(-c3ccc(-c4cccc(OC(F)(F)F)c4)cc3)c(=O)c2cc1Cl ; COc1cc2[nH]c(C)c(-c3ccc(-c4ccc(Cl)cc4)cc3)c(=O)c2cc1Cl ; COc1ccc(-c2ccc(-c3c(C)[nH]c4cc(OC)c(Cl)cc4c3=O)cc2)cc1 ; COc1cc2[nH]c(C)c(-c3ccc(-c4cccc(C(F)(F)F)c4)cc3)c(=O)c2cc1Cl ; COc1cc2[nH]c(C)c(-c3ccc(-c4ccc(C(F)(F)F)cc4)cc3)c(=O)c2cc1Cl] 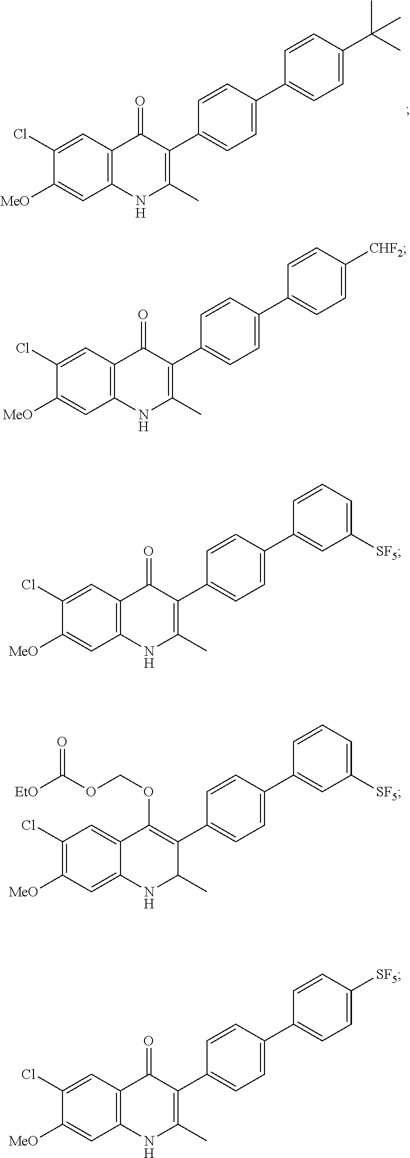 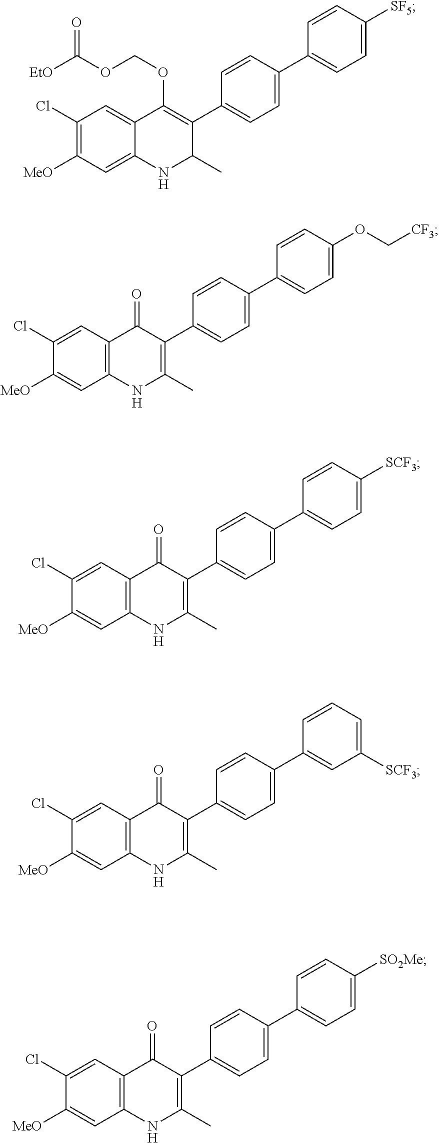 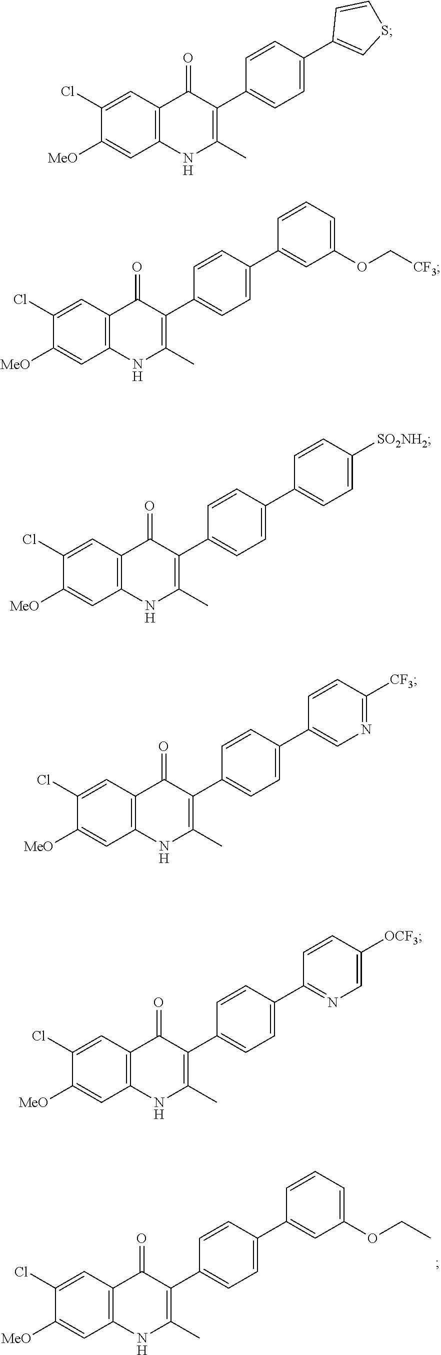 [SMILES: COc1cc2[nH]c(C)c(-c3ccc(-c4ccsc4)cc3)c(=O)c2cc1Cl ; COc1cc2[nH]c(C)c(-c3ccc(-c4cccc(OCC(F)(F)F)c4)cc3)c(=O)c2cc1Cl ; COc1cc2[nH]c(C)c(-c3ccc(-c4ccc(S(N)(=O)=O)cc4)cc3)c(=O)c2cc1Cl ; COc1cc2[nH]c(C)c(-c3ccc(-c4ccc(C(F)(F)F)nc4)cc3)c(=O)c2cc1Cl ; COc1cc2[nH]c(C)c(-c3ccc(-c4ccc(OC(F)(F)F)cn4)cc3)c(=O)c2cc1Cl ; CCOc1cccc(-c2ccc(-c3c(C)[nH]c4cc(OC)c(Cl)cc4c3=O)cc2)c1] 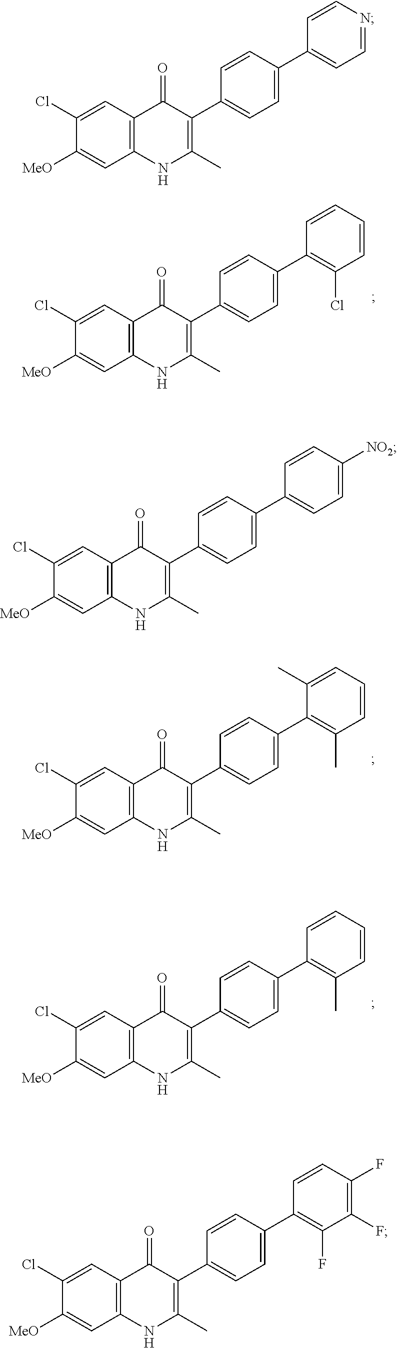 [SMILES: COc1cc2[nH]c(C)c(-c3ccc(-c4ccncc4)cc3)c(=O)c2cc1Cl ; COc1cc2[nH]c(C)c(-c3ccc(-c4ccccc4Cl)cc3)c(=O)c2cc1Cl ; COc1cc2[nH]c(C)c(-c3ccc(-c4ccc([N+](=O)[O-])cc4)cc3)c(=O)c2cc1Cl ; COc1cc2[nH]c(C)c(-c3ccc(-c4c(C)cccc4C)cc3)c(=O)c2cc1Cl ; COc1cc2[nH]c(C)c(-c3ccc(-c4ccccc4C)cc3)c(=O)c2cc1Cl ; COc1cc2[nH]c(C)c(-c3ccc(-c4ccc(F)c(F)c4F)cc3)c(=O)c2cc1Cl] 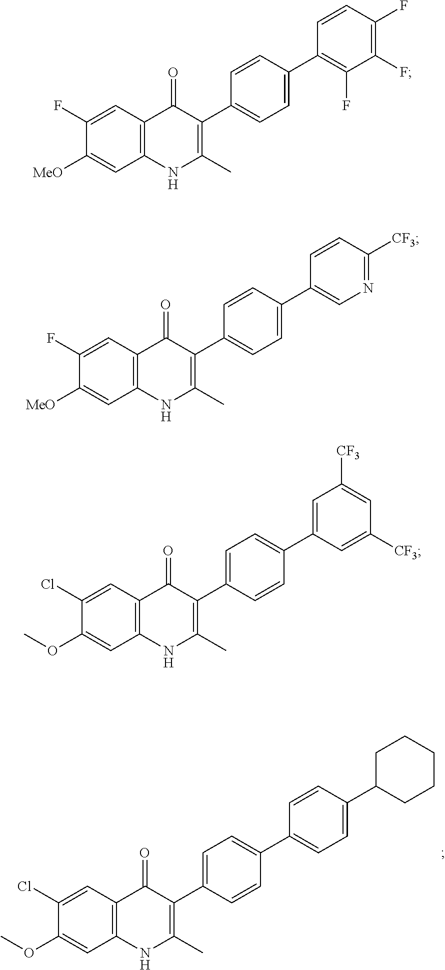 [SMILES: COc1cc2[nH]c(C)c(-c3ccc(-c4ccc(F)c(F)c4F)cc3)c(=O)c2cc1F ; COc1cc2[nH]c(C)c(-c3ccc(-c4ccc(C(F)(F)F)nc4)cc3)c(=O)c2cc1F ; COc1cc2[nH]c(C)c(-c3ccc(-c4cc(C(F)(F)F)cc(C(F)(F)F)c4)cc3)c(=O)c2cc1Cl ; COc1cc2[nH]c(C)c(-c3ccc(-c4ccc(C5CCCCC5)cc4)cc3)c(=O)c2cc1Cl] 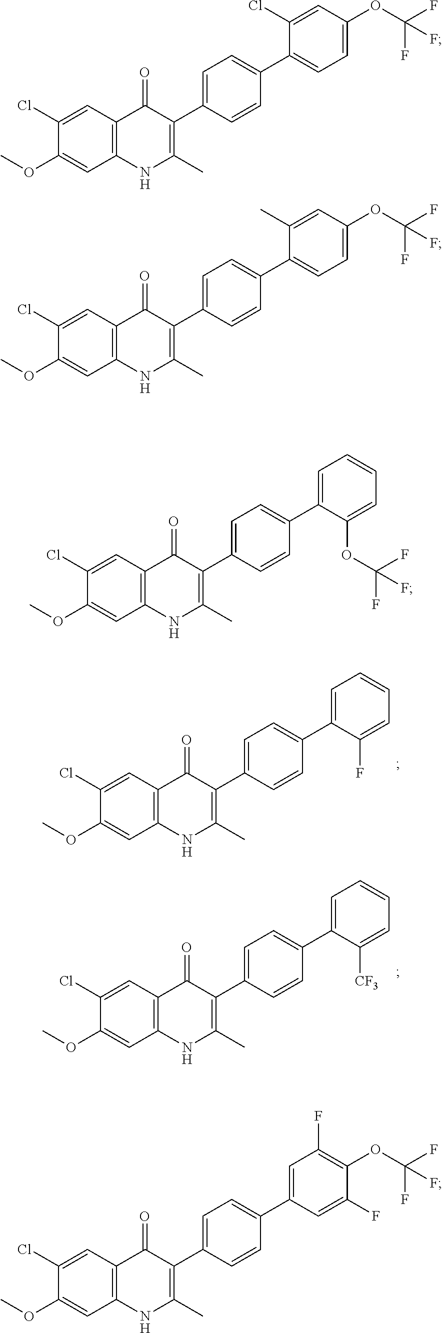 [SMILES: COc1cc2[nH]c(C)c(-c3ccc(-c4ccc(OC(F)(F)F)cc4Cl)cc3)c(=O)c2cc1Cl ; COc1cc2[nH]c(C)c(-c3ccc(-c4ccc(OC(F)(F)F)cc4C)cc3)c(=O)c2cc1Cl ; COc1cc2[nH]c(C)c(-c3ccc(-c4ccccc4OC(F)(F)F)cc3)c(=O)c2cc1Cl ; COc1cc2[nH]c(C)c(-c3ccc(-c4ccccc4F)cc3)c(=O)c2cc1Cl ; COc1cc2[nH]c(C)c(-c3ccc(-c4ccccc4C(F)(F)F)cc3)c(=O)c2cc1Cl ; COc1cc2[nH]c(C)c(-c3ccc(-c4cc(F)c(OC(F)(F)F)c(F)c4)cc3)c(=O)c2cc1Cl] 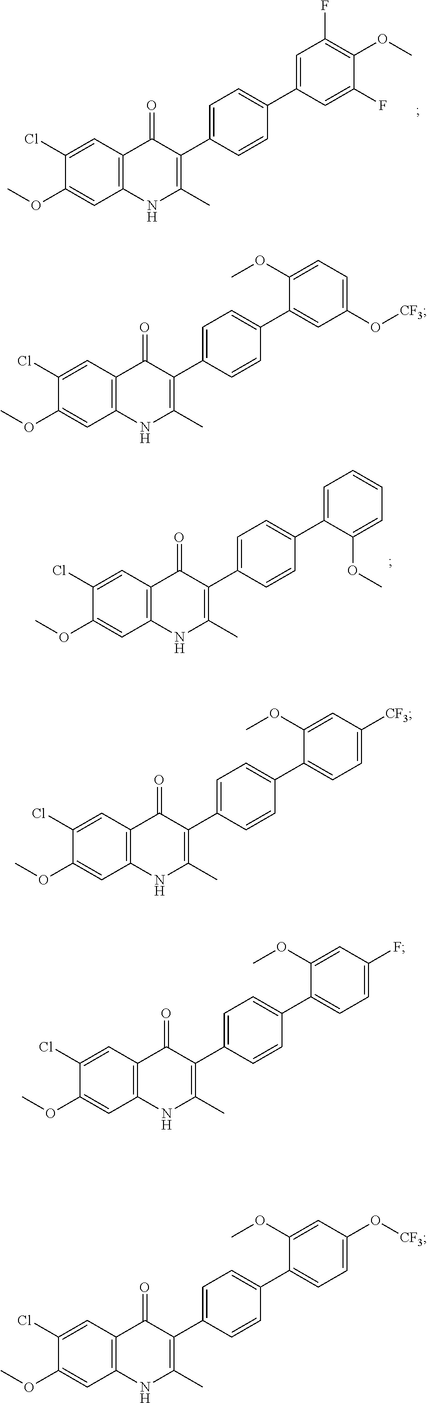 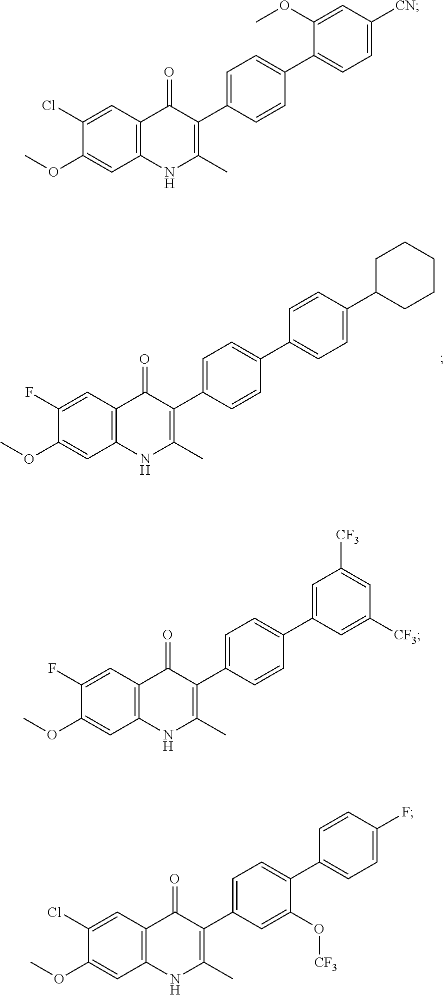 [SMILES: COc1cc2[nH]c(C)c(-c3ccc(-c4ccc(C#N)cc4OC)cc3)c(=O)c2cc1Cl ; COc1cc2[nH]c(C)c(-c3ccc(-c4ccc(C5CCCCC5)cc4)cc3)c(=O)c2cc1F ; COc1cc2[nH]c(C)c(-c3ccc(-c4cc(C(F)(F)F)cc(C(F)(F)F)c4)cc3)c(=O)c2cc1F ; COc1cc2[nH]c(C)c(-c3ccc(-c4ccc(F)cc4)c(OC(F)(F)F)c3)c(=O)c2cc1Cl] 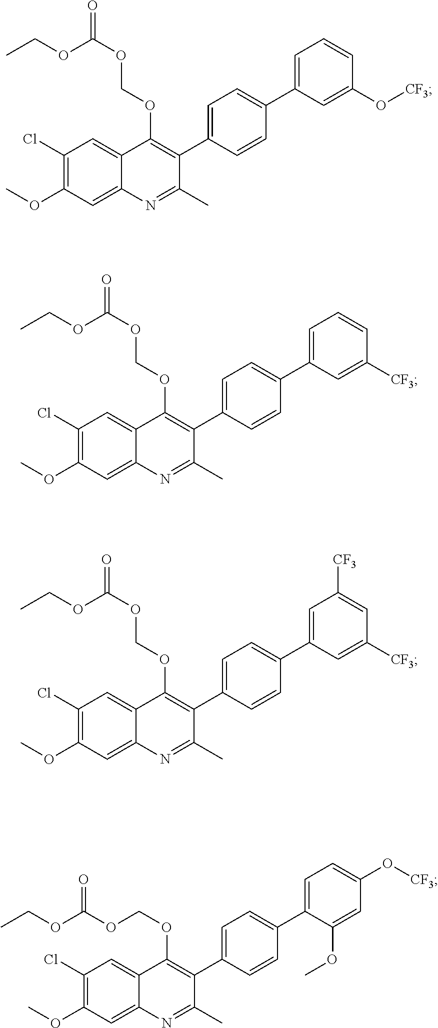 [SMILES: CCOC(=O)OCOc1c(-c2ccc(-c3cccc(OC(F)(F)F)c3)cc2)c(C)nc2cc(OC)c(Cl)cc12 ; CCOC(=O)OCOc1c(-c2ccc(-c3cccc(C(F)(F)F)c3)cc2)c(C)nc2cc(OC)c(Cl)cc12 ; CCOC(=O)OCOc1c(-c2ccc(-c3cc(C(F)(F)F)cc(C(F)(F)F)c3)cc2)c(C)nc2cc(OC)c(Cl)cc12 ; CCOC(=O)OCOc1c(-c2ccc(-c3ccc(OC(F)(F)F)cc3OC)cc2)c(C)nc2cc(OC)c(Cl)cc12] 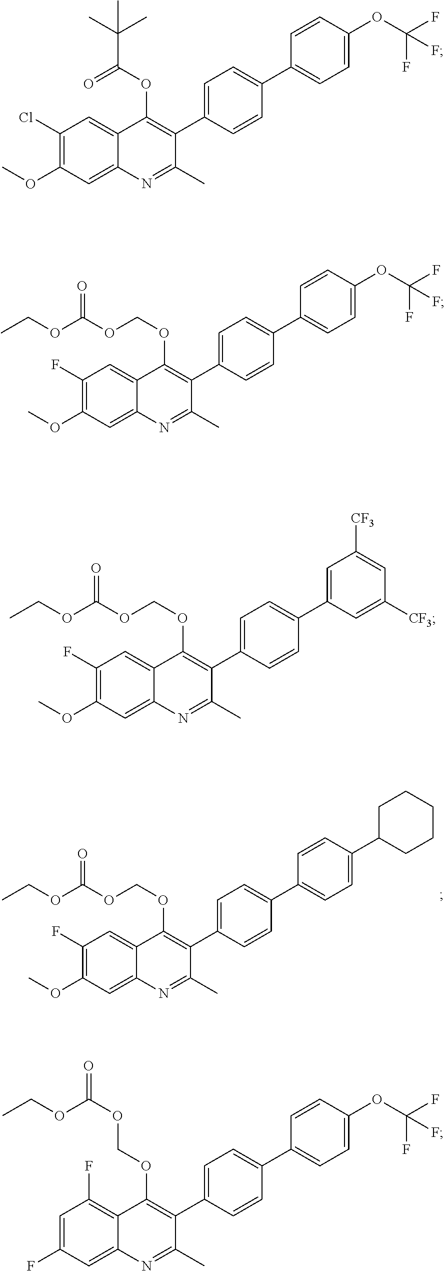 [SMILES: COc1cc2nc(C)c(-c3ccc(-c4ccc(OC(F)(F)F)cc4)cc3)c(OC(=O)C(C)(C)C)c2cc1Cl ; CCOC(=O)OCOc1c(-c2ccc(-c3ccc(OC(F)(F)F)cc3)cc2)c(C)nc2cc(OC)c(F)cc12 ; CCOC(=O)OCOc1c(-c2ccc(-c3cc(C(F)(F)F)cc(C(F)(F)F)c3)cc2)c(C)nc2cc(OC)c(F)cc12 ; CCOC(=O)OCOc1c(-c2ccc(-c3ccc(C4CCCCC4)cc3)cc2)c(C)nc2cc(OC)c(F)cc12 ; CCOC(=O)OCOc1c(-c2ccc(-c3ccc(OC(F)(F)F)cc3)cc2)c(C)nc2cc(F)cc(F)c12] 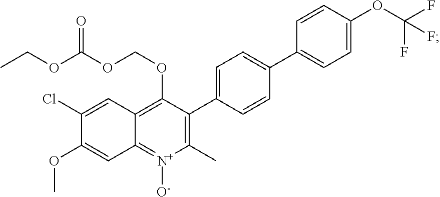 [SMILES: CCOC(=O)OCOc1c(-c2ccc(-c3ccc(OC(F)(F)F)cc3)cc2)c(C)[n+]([O-])c2cc(OC)c(Cl)cc12] 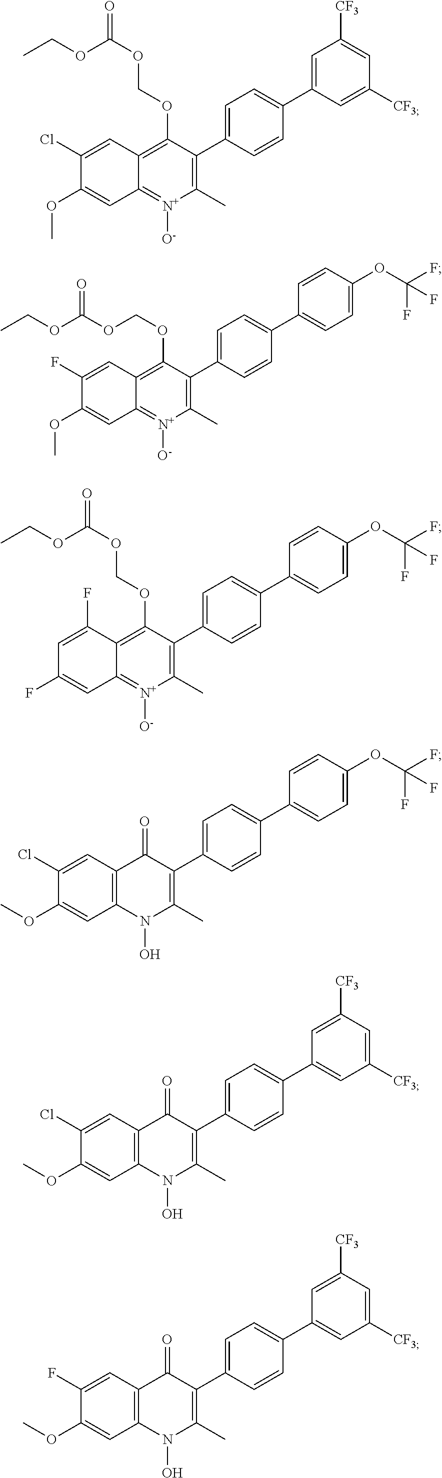 [SMILES: CCOC(=O)OCOc1c(-c2ccc(-c3cc(C(F)(F)F)cc(C(F)(F)F)c3)cc2)c(C)[n+]([O-])c2cc(OC)c(Cl)cc12 ; CCOC(=O)OCOc1c(-c2ccc(-c3ccc(OC(F)(F)F)cc3)cc2)c(C)[n+]([O-])c2cc(OC)c(F)cc12 ; CCOC(=O)OCOc1c(-c2ccc(-c3ccc(OC(F)(F)F)cc3)cc2)c(C)[n+]([O-])c2cc(F)cc(F)c12 ; COc1cc2c(cc1Cl)c(=O)c(-c1ccc(-c3ccc(OC(F)(F)F)cc3)cc1)c(C)n2O ; COc1cc2c(cc1Cl)c(=O)c(-c1ccc(-c3cc(C(F)(F)F)cc(C(F)(F)F)c3)cc1)c(C)n2O ; COc1cc2c(cc1F)c(=O)c(-c1ccc(-c3cc(C(F)(F)F)cc(C(F)(F)F)c3)cc1)c(C)n2O] 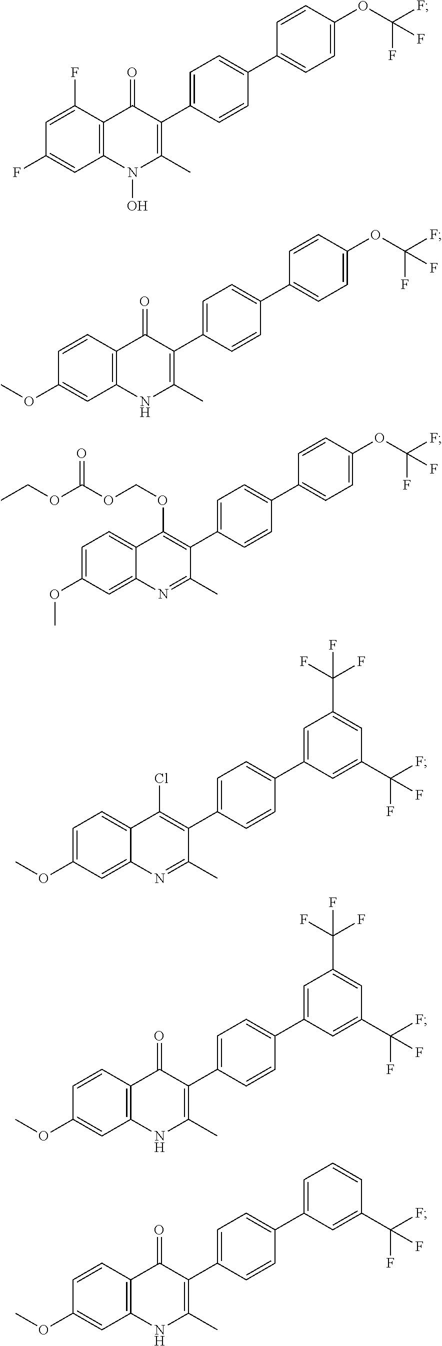 [SMILES: Cc1c(-c2ccc(-c3ccc(OC(F)(F)F)cc3)cc2)c(=O)c2c(F)cc(F)cc2n1O ; COc1ccc2c(=O)c(-c3ccc(-c4ccc(OC(F)(F)F)cc4)cc3)c(C)[nH]c2c1 ; CCOC(=O)OCOc1c(-c2ccc(-c3ccc(OC(F)(F)F)cc3)cc2)c(C)nc2cc(OC)ccc12 ; COc1ccc2c(Cl)c(-c3ccc(-c4cc(C(F)(F)F)cc(C(F)(F)F)c4)cc3)c(C)nc2c1 ; COc1ccc2c(=O)c(-c3ccc(-c4cc(C(F)(F)F)cc(C(F)(F)F)c4)cc3)c(C)[nH]c2c1 ; COc1ccc2c(=O)c(-c3ccc(-c4cccc(C(F)(F)F)c4)cc3)c(C)[nH]c2c1] 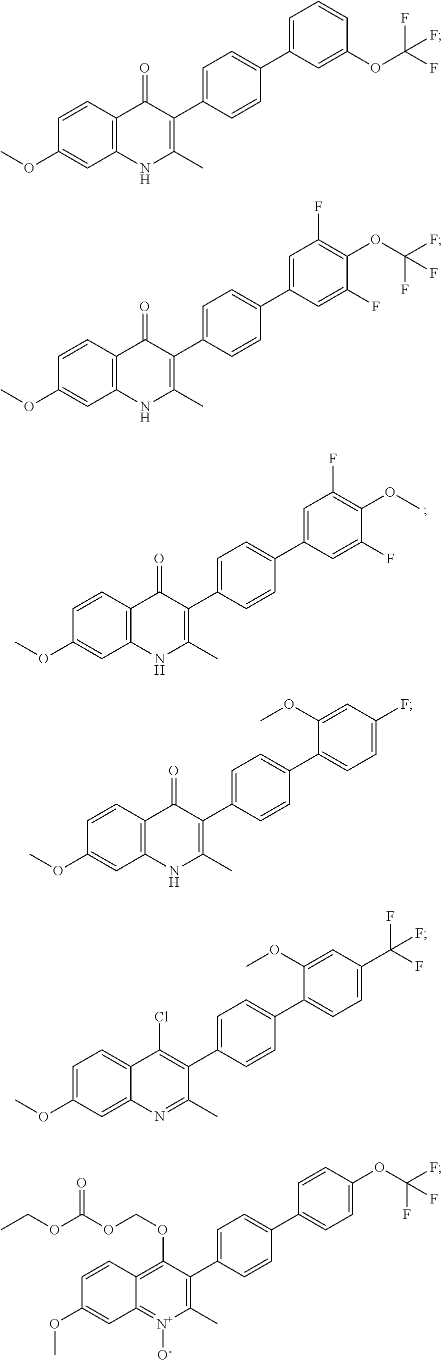 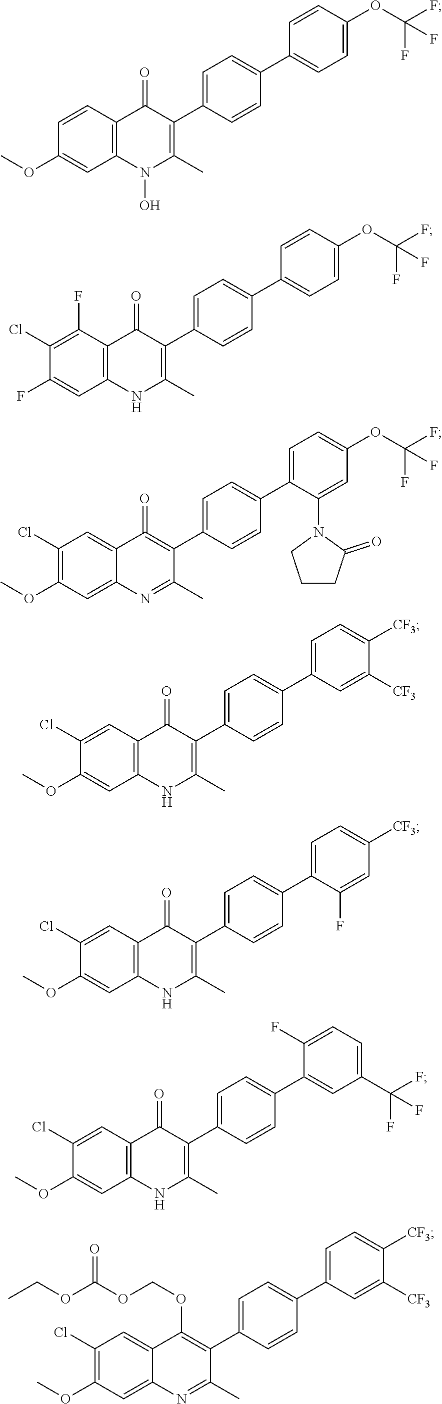 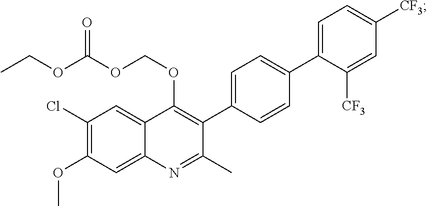 [SMILES: CCOC(=O)OCOc1c(-c2ccc(-c3ccc(C(F)(F)F)cc3C(F)(F)F)cc2)c(C)nc2cc(OC)c(Cl)cc12]
or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof.

13. A pharmaceutical composition comprising a pharmaceutically or therapeutically effective amount of a compound of claim 1, or a pharmaceutically acceptable salt thereof, and a pharmaceutically acceptable carrier or excipient.

14. A method of using a compound of claim 1, or a pharmaceutically acceptable salt, co-crystal, ester, solvate, hydrate, isomer (including optical isomers, racemates, or other mixtures thereof), tautomer, isotope, polymorph, or pharmaceutically acceptable prodrug thereof, in preparation of a medicament.
